(19) United States
(12) Patent Application Publication
(10) Pub. No.: US 2023/0295131 A1
(43) Pub. Date: Sep. 21, 2023

(54) INHIBITORS OF RHO-ASSOCIATED COILED-COIL KINASE

(71) Applicant: Angion Biomedica Corp., Uniondale, NY (US)

(72) Inventors: An-Hu Li, Commack, NY (US); Shashikanth Ponnala, Secaucus, NJ (US); Satish Kumar Sakilam, Harrison, NJ (US); Satishkumar Gadhiya, Jamaica, NY (US); Yao Zong, Port Washington, NY (US); Dong Sung Lim, Fair Lawn, NJ (US); Ying Zhang, Glen Cove, NY (US); Dawoon Jung, Tenafly, NJ (US)

(21) Appl. No.: 18/017,102

(22) PCT Filed: Jul. 20, 2021
(86) PCT No.: PCT/US2021/042419

§ 371 Date: Jan. 20, 2023

Related U.S. Application Data
(60) Provisional application No. 63/054,642, filed on Jul. 21, 2020.

(51) Int. Cl. C07D 403/14; C07D 405/14; C07D 471/04; C07D 409/14; A61P 13/12

(52) U.S. Cl. CPC C07D 403/14; C07D 405/14; C07D 471/04; C07D 409/14; A61P 13/12

(57) ABSTRACT

The present disclosure provides compounds of Formula (I); or a pharmaceutically acceptable salt thereof, wherein each of Ring A, Ring B, R^{a}, R^{b}, L, R¹, R², m, and n is defined herein, pharmaceutical compositions thereof, methods of inhibiting ROCK1 and/or ROCK2, and methods of treating a ROCK1- and/or ROCK2-mediated disease or disorder.

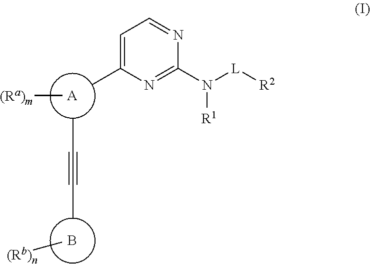

### CROSS REFERENCE TO RELATED APPLICATIONS

This application claims the benefit of U.S. Provisional Application No. 63/054,642, filed Jul. 21, 2020, which is herein incorporated by reference in its entirety.

### BACKGROUND

The Rho-associated coiled-coil kinase (ROCK) family members, consisting of Rho-associated kinase 1 (ROCK1) and Rho-associated kinase 2 (ROCK2), are serine-threonine kinases that are activated by Rho GTPases. Both ROCK1 and ROCK2 are involved in a wide range of cellular processes including actin cytoskeleton organization, smooth muscle cell contraction, adhesion, migration, proliferation, apoptosis and fibrosis (Loirand, G. Rho Kinases in Health and Disease: From Basic Science to Translational Research. Pharmacol. Rev. 2015, 67(4), 1074-95). The ROCK signaling cascade, modulated by fibrogenic growth factors including TGFβ1, angiotensin I, PDGF and endothelin-I, participates in epithelial to mesenchymal transition (Hu, Y. B., Li, X., Liang, G. N., Deng, Z. H., Jiang, H. Y., Zhou, J. H. Roles of Rho/Rock signaling pathway in silica-induced epithelial-mesenchymal transition in human bronchial epithelial cells. Biomed. Environ. Sci. 2013, 26(7), 571-6). Evidence for the potential role of this pathway in renal fibrosis comes from early studies that used pharmacologic inhibition of ROCK with Y-27632 or fasudil, which are selective but ROCK1/2 dual inhibitors, i.e., they inhibit both ROCK 1 and ROCK2 but not other kinases. Use of ROCK1/2 dual inhibitors prevented tubulointerstitial fibrosis in obstructive renal disease, mitigated nephropathy in subtotally nephrectomized, spontaneously hypertensive rats and attenuated glomerulosclerosis in Dahl salt-sensitive rats (Komers, R., Oyama, T. T., Beard, D. R., Tikellis, C., Xu, B., Lotspeich, D. F., Anderson, S. Rho kinase inhibition protects kidneys from diabetic nephropathy without reducing blood pressure. Kidney Int. 2011, 79(4), 432-42. Nagatoya, K., Moriyama, T., Kawada, N., Takeji, M., Oseto, S., Murozono, T., Ando, A., Imai, E., Hori, M. Y-27632 prevents tubulointerstitial fibrosis in mouse kidneys with unilateral ureteral obstruction. Kidney Int. 2002, 61(5), 1684-95. Baba, I., Egi, Y., Utsumi, H., Kakimoto, T., Suzuki, K. Inhibitory effects of fasudil on renal interstitial fibrosis induced by unilateral ureteral obstruction. Mol. Med. Rep. 2015, 12(6), 8010-20. Kolavennu, V., Zeng, L., Peng, H., Wang, Y., Danesh, F. R. Targeting of RhoA/ROCK signaling ameliorates progression of diabetic nephropathy independent of glucose control. Diabetes 2008, 57(3), 714-23).

Regardless of the fact that the two ROCK isoforms are similar, a growing body of evidence from more recent studies with ROCK isoform transgenic animals and ROCK isoform-selective pharmacological inhibitors support the notion that ROCK1 and ROCK2 each have unique functions. Shi et al. (Shi, J., Wu, X., Surma, M., Vemula, S., Zhang, L., Yang, Y., Kapur, R., Wei, L. Distinct roles for ROCK1 and ROCK2 in the regulation of cell detachment. Cell Death Dis. 2013, 4(2), e483. doi: 10.1038/cddis.2013.10), using both genetic and pharmacological approaches, demonstrated that ROCK1, via regulation of MLC2 phosphorylation, is involved in destabilizing the actin cytoskeleton in fibroblasts (i.e., ROCK1 signaling is antifibrotic), whereas ROCK2, via regulation of cofilin phosphorylation, is required for stabilizing fibroblast actin cytoskeleton (i.e., ROCK2 signaling is profibrotic). Consistent with this finding, genome-wide expression profiling of fibroblasts treated with the ROCK2 selective inhibitor, KD025 (SLx-2119), revealed decreased expression of several profibrotic mRNA including that of CTGF (Boerma, M., Fu, Q., Wang, J., Loose, D. S., Bartolozzi, A., Ellis, J. L., McGonigle, S., Paradise, E., Sweetnam, P., Fink, L. M., Vozenin-Brotons, M. C., Hauer-Jensen, M. Comparative gene expression profiling in three primary human cell lines after treatment with a novel inhibitor of Rho kinase or atorvastatin. Blood Coagul. Fibrinolysis 2008, 19(7), 709-718). In a separate study (Zanin-Zhorov, A., Weiss, J. M., Nyuydzefe, M. S., Chen, W., Scher, J. U., Mo, R., Depoil, D., Rao, N., Liu, B., Wei, J., Lucas, S., Koslow, M., Roche, M., Schueller, O., Weiss, S., Poyurovsky, M. V., Tonra, J., Hippen, K. L., Dustin, M. L., Blazar, B. R., Liu, C. J., Waksal, S. D. Selective oral ROCK2 inhibitor down-regulates IL-21 and IL-17 secretion in human T cells via STAT3-dependent mechanism. Proc. Natl. Acad. Sci. USA. 2014, 111(47), 16814-9), KD025 administration decreased expression of pro-inflammatory, fibrosis-linked cytokines and mitigated murine autoimmune disease. Further evidence appearing to support a driving role for ROCK2 in fibrosis, and pertinent to renal disease, is the finding that ROCK1 knockout mice were not protected against ureteral obstruction-related renal fibrosis at either the early (day 5) or late (day 10) disease stage as determined by histology and expression of both mRNA and protein levels of αSMA, collagen types I and III and fibronectin (Fu, P., Liu, F., Su, S., Wang, W., Huang, X. R., Entman, M. L., Schwartz, R. J., Wei, L., Lan, H. Y. Signaling mechanism of renal fibrosis in unilateral ureteral obstructive kidney disease in ROCK1 knockout mice. J. Am. Soc. Nephrol. 2006, 17(11), 3105-14). Although Baba et al. (Baba, I., Egi, Y., Suzuki, K. Partial deletion of the ROCK2 protein fails to reduce renal fibrosis in a unilateral ureteral obstruction model in mice. Mol. Med. Rep. 2016, 13(1), 231-6), demonstrated that half-deletion of ROCK2 also did not prevent UUO-induced renal fibrosis, the discrepancy regarding these data and the one published by Shi et al. (Shi, J., Wu, X., Surma, M., Vemula, S., Zhang, L., Yang, Y., Kapur, R., Wei, L. Distinct roles for ROCK1 and ROCK2 in the regulation of cell detachment. Cell Death Dis. 2013, 4(2), e483. doi: 10.1038/cddis.2013.10), could be attributed to different strain and incomplete genetic ablation (homozygous vs. heterozygous) of the ROCK2 isozyme.

Efficacy aside, need for use of an isoform-selective approach derives from the perspective of drug safety. Since ROCK plays a central role in the organization of the actin cytoskeleton, it might be anticipated that (unnecessary) inhibition of both its isoforms in a chronic setting such as chronic kidney disease (CKD) could cause severe adverse events. Indeed, systemic inhibition of ROCK does bear the risk of significant hypotension and such a strategy needs to be evaluated in terms of risk to benefit ratio (www.hsric.nihr.ac.uk/topics/netarsudil-for-open-angle-glaucoma-or-ocular-hypertension/). For diseases such as glaucoma, which is amenable to local treatment, ROCK isoform selectivity is not mandated and ROCK1/2 dual inhibitors such as netarsudil are dosed into the eye via the intravitreous or intracameral routes (www.hsric.nihr.ac.uk/topics/netarsudil-for-open-angle-glaucoma-or-ocular-hypertension/). Furthermore, drug load in glaucoma is small. With hyperacute indications such as cerebral vasospasm, dosing with fasudil might not pose a significant risk, albeit its use remains to be approved in the United States. Finally, in contrast to use of ROCK1/2 dual inhibitors, the ROCK2-selective inhibitor KD025 has been found to have no hemodynamic or other side effects over 12-16 weeks of dosing in healthy volunteers and patients.

All citations in the present application are incorporated herein by reference in their entireties. The citation of any reference herein should not be construed as an admission that such reference is available as “Prior Art” to the instant application.

### SUMMARY

In some embodiments, the present disclosure encompasses the recognition that there remains a need for the development of novel therapeutics that are capable of inhibiting the activity of ROCK1 and/or ROCK2. In certain embodiments, the present disclosure is directed toward the identification of small organic molecules that exhibit the activity of ROCK1 and/or ROCK2 and are thus useful in the treatment or prevention of conditions or diseases in which inhibition of ROCK1 and/or ROCK2 is desirable.

In some embodiments, the present disclosure provides a compound of Formula I.

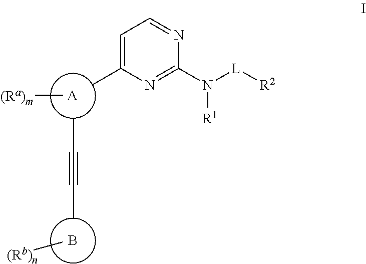

or a pharmaceutically acceptable salt thereof, wherein each of Ring A, Ring B, R^{a}, R^{b}, L, R¹, R², m and n is defined infra.

In some embodiments, the present disclosure provides a pharmaceutical composition comprising a compound of Formula I, or a pharmaceutically acceptable salt thereof, and a pharmaceutically acceptable carrier.

In some embodiments, the present disclosure provides methods of using any of the compounds disclosed herein for inhibiting the activity of ROCK1 and/or ROCK2 in a patient or in a biological sample. In some embodiments, the compounds of the disclosure have antifibrotic activities. In some embodiments, provided compounds and pharmaceutical compositions thereof are inhibitors of ROCK1 and/or ROCK2 activities and are useful in the treatment of any disease, disorder or condition in which prophylactic or therapeutic administration of an inhibitor of ROCK1 and/or ROCK2 would be useful.

In some embodiments, the present disclosure provides a method of inhibiting ROCK1 and/or ROCK2, the method comprising contacting a biological sample with a compound of Formula I, or a pharmaceutically acceptable salt thereof.

In some embodiments, compounds of Formula I are selective inhibitors of ROCK2.

In some embodiments, the present disclosure provides a method of treating or lessening the severity of a disease or condition associated with activity of ROCK1 and/or ROCK2. In certain embodiments, the present disclosure provides a method of treating or lessening the severity of a disease or condition selected from a hepatic disease, a renal disease, a cerebral and/or cerebrovascular disease, a cardiac and/or cardiovascular disease, a pulmonary disease, a dermal disease, a gastrointestinal disease, an ischemic disease, and a fibrotic disease described herein (e.g., fibrotic liver disease, hepatic ischemia-reperfusion injury, cerebral infarction, ischemic heart disease, renal disease or lung (pulmonary) fibrosis). In certain embodiments, provided compounds are useful for treating or lessening the severity of a disease or condition selected from liver fibrosis associated with hepatitis C, hepatitis B, delta hepatitis, chronic alcoholism, non-alcoholic steatohepatitis (NASH), extrahepatic obstructions (stones in the bile duct), cholangiopathies (primary biliary cirrhosis and sclerosing cholangitis), autoimmune liver disease, and inherited metabolic disorders (Wilson's disease, hemochromatosis, and alpha-1 antitrypsin deficiency); cirrhotic or non-cirrhotic hepatocellular carcinoma (HCC); damaged and/or ischemic organs, transplants or grafts; ischemia/reperfusion injury; stroke; cerebrovascular disease; myocardial ischemia; atherosclerosis; renal failure; acute kidney injury (AKI)-related chronic kidney disease (CKD); renal fibrosis or idiopathic pulmonary fibrosis. In certain exemplary embodiments, provided compounds are useful in the treatment of wounds for acceleration of healing; vascularization of a damaged and/or ischemic organ, transplant or graft; amelioration of ischemia/reperfusion injury in the brain, heart, liver, kidney, and other tissues and organs; normalization of myocardial perfusion as a consequence of chronic cardiac ischemia or myocardial infarction; development or augmentation of collateral vessel development after vascular occlusion or to ischemic tissues or organs; fibrotic diseases; hepatic disease including fibrosis and cirrhosis; lung fibrosis; radiocontrast nephropathy; fibrosis secondary to renal obstruction; renal trauma and transplantation; acute or chronic heart failure, renal failure secondary to chronic diabetes and/or hypertension; amyotrophic lateral sclerosis, muscular dystrophy, glaucoma, corneal scarring, macular degeneration, diabetic retinopathy and/or diabetes mellitus.

In some embodiments, the present disclosure provides a method of treating a disease or disorder associated with or mediated by ROCK1 and/or ROCK2, the method comprising administering to a patient in need thereof a compound of Formula I, or a pharmaceutically acceptable salt thereof. Diseases and/or disorders associated with or mediated by ROCK1 and/or ROCK2 are described in greater detail, infra.

These and other aspects of the present disclosure will be apparent from the brief description of the drawing and detailed description of certain aspects of the disclosure, below.

### BRIEF DESCRIPTION OF THE DRAWINGS

FIG. 1A and FIG. 1B depict the in vitro activity of compounds 65 and 93 against TGFβ1-induced hepatic stellate cell (HSC) contraction.

FIG. 2 depicts the in vitro activity of compound 65 against connective tissue growth factor (CTGF) production in embryonic fibroblasts.

FIG. 3 depicts the reduction of liver steatosis in the presence of compound 65 in mice administered a fast-food diet (e.g., FFD+CCl₄+glucose).

FIG. 4 depicts the reduction of NASH in the presence of compound 65 in mice administered a fast-food diet (e.g., FFD+CCl₄+glucose).

FIG. 5A and FIG. 5B depict the increased renal ROCK2 expression in adult male C57BL/6 mice submitted to unilateral ureteral obstruction (UUO).

FIG. 6A and FIG. 6B depict the decreased phosphorylation of renal ROCK2 in the presence of Compound A in adult male C57BL/6 mice submitted to unilateral ureteral obstruction (UUO).

FIG. 7A, FIG. 7B, and FIG. 7C depict the renal antifibrotic effects of Compound A in SV129 mice subjected to subtotal nephrectomy. Treatment with Compound A was associated with decreased kidney hydroxyproline (FIG. 7A) and decreased Masson's trichrome (FIG. 7B) in the absence of changes in MAP (FIG. 7C).

### DEFINITIONS

Compounds of the present disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle”, “carbocyclic”, “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “unsaturated”, as used herein, means that a moiety has one or more units of unsaturation.

As used herein, the term “partially unsaturated”, as used herein, refers to a ring moiety that includes at least one double or triple bond. The term “partially unsaturated”, as used herein, is intended to encompass rings having multiple sites of unsaturation, but is not intended to include aryl or heteroaryl moieties, as herein defined.

The term “lower alkyl”, as used herein, refers to a C₁₋₄ straight or branched alkyl group. Exemplary lower alkyl groups are methyl, ethyl, propyl, isopropyl, butyl, isobutyl, and tert-butyl.

The term “aryl”, as used herein, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments, “aryl” refers to an aromatic ring system which includes, but not limited to, phenyl, biphenyl, naphthyl, anthracyl and the like, which may bear one or more substituents. Also included within the scope of the term “aryl” is a group in which an aromatic ring is fused to one or more non-aromatic rings, such as indanyl, phthalimidyl, naphthimidyl, phenanthridinyl, or tetrahydronaphthyl, and the like.

The term “heteroaryl” as used herein, refers to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” as used herein, refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples of heteroaryl rings on compounds of Formula I and subgenera thereof include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, imidazopyridinyl (e.g., imidazo[1,5-a]pyridinyl), triazolopyridinyl (e.g., [1,2,4]triazolo[4,3-a]pyridinyl), and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted.

As used herein, the terms “heterocycle”, “heterocyclyl”, and “heterocyclic ring” are used interchangeably and refer to a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic.

The terms “halo” and “halogen” as used herein refer to an atom selected from fluorine, chlorine, bromine and iodine.

As described herein, compounds may contain “optionally substituted” moieties. In general, the term “substituted”, whether preceded by the term “optionally” or not, means that one or more hydrogens of the designated moiety of compounds are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)_{0- 4}N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, −CH₂— (5- to 6-membered heteroaryl ring), or a 5- to 6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group of a compound of Formula I, and subgenera thereof, include: —O(CR*₂)₂₋₃O—, wherein each independent occurrence of R*is selected from hydrogen, C₁₋₆ aliphatic which may be substituted as defined below, or an unsubstituted 5- to 6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}2, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R; wherein each R is independently hydrogen, C₁₋₆ aliphatic which may be substituted as defined below, unsubstituted —OPh, or an unsubstituted 5- to 6-membered saturated, partially unsaturated, or aryl ring having 0-4 heteroatoms independently selected from nitrogen, oxygen, or sulfur, or, notwithstanding the definition above, two independent occurrences of R, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the present disclosure. Unless otherwise stated, all tautomeric forms are within the scope of the disclosure. Additionally, unless otherwise stated, the present disclosure also includes compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure. In some embodiments, compounds of this disclosure comprise one or more deuterium atoms.

The term “tautomerization” refers to the phenomenon wherein a proton of one atom of a molecule shifts to another atom. See, Jerry March, Advanced Organic Chemistry: Reactions, Mechanisms and Structures, Fourth Edition, John Wiley & Sons, pages 69-74 (1992). The term “tautomer” as used herein, refers to the compounds produced by the proton shift. For example, compounds of formula A and B can exist as a tautomer as shown below:

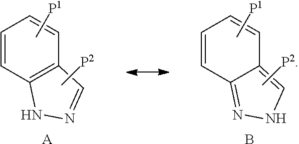

Thus, the present disclosure encompasses the substituted indazolyl compounds, in which the proton on the nitrogen can be attached to either of the two nitrogen atoms.

As used herein the term “biological sample” includes, without limitation, cell cultures or extracts thereof; biopsied material obtained from an animal (e.g., mammal) or extracts thereof, and blood, saliva, urine, feces, semen, tears, or other body fluids or extracts thereof; or purified versions thereof. For example, the term “biological sample” refers to any solid or fluid sample obtained from, excreted by or secreted by any living organism, including single-celled micro-organisms (such as bacteria and yeasts) and multicellular organisms (such as plants and animals, for instance a vertebrate or a mammal, and in particular a healthy or apparently healthy human subject or a human patient affected by a condition or disease to be diagnosed or investigated). The biological sample can be in any form, including a solid material such as a tissue, cells, a cell pellet, a cell extract, cell homogenates, or cell fractions; or a biopsy, or a biological fluid. The biological fluid may be obtained from any site (e.g. blood, saliva (or a mouth wash containing buccal cells), tears, plasma, serum, urine, bile, seminal fluid, cerebrospinal fluid, amniotic fluid, peritoneal fluid, and pleural fluid, or cells therefrom, aqueous or vitreous humor, or any bodily secretion), a transudate, an exudate (e.g. fluid obtained from an abscess or any other site of infection or inflammation), or fluid obtained from a joint (e.g. a normal joint or a joint affected by disease such as rheumatoid arthritis, osteoarthritis, gout or septic arthritis). The biological sample can be obtained from any organ or tissue (including a biopsy or autopsy specimen) or may comprise cells (whether primary cells or cultured cells) or medium conditioned by any cell, tissue or organ. Biological samples may also include sections of tissues such as frozen sections taken for histological purposes. Biological samples also include mixtures of biological molecules including proteins, lipids, carbohydrates and nucleic acids generated by partial or complete fractionation of cell or tissue homogenates. Although the sample is preferably taken from a human subject, biological samples may be from any animal, plant, bacteria, virus, yeast, etc. The term animal, as used herein, refers to humans as well as non-human animals, at any stage of development, including, for example, mammals, birds, reptiles, amphibians, fish, worms and single cells. Cell cultures and live tissue samples are considered to be pluralities of animals. In certain exemplary embodiments, the non-human animal is a mammal (e.g., a rodent, a mouse, a rat, a rabbit, a monkey, a dog, a cat, a sheep, cattle, a primate, or a pig). An animal may be a transgenic animal or a human clone. If desired, the biological sample may be subjected to preliminary processing, including preliminary separation techniques.

### DETAILED DESCRIPTION OF CERTAIN EMBODIMENTS

The present disclosure provides compounds that inhibit the activity of ROCK1 and/or ROCK2. In some embodiments, compounds disclosed herein inhibit both ROCK1 and ROCK2 kinases.

Compounds of this disclosure include those generally set forth above and described specifically herein, and are illustrated in part by the various classes, subgenera and species disclosed herein.

Additionally, the present disclosure provides pharmaceutically acceptable derivatives of the provided compounds, and methods of treating a subject using these compounds, or pharmaceutical compositions thereof.

Compounds

In some embodiments, the present disclosure provides a compound of Formula I:

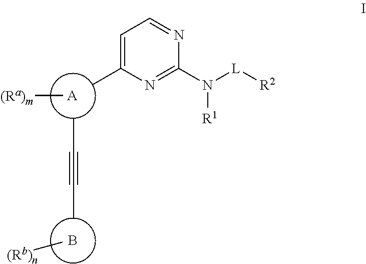

or a pharmaceutically acceptable salt thereof, wherein:
Ring A is selected from phenyl and a 6-membered heteroaryl ring comprising 1-3 nitrogen atoms;
Ring B is selected from phenyl, a 5- to 6-membered heteroaryl ring comprising 1-3 heteroatoms independently selected from nitrogen, oxygen, and sulfur, and a 9- to 10-membered heteroaryl ring comprising 1-4 heteroatoms independently selected from nitrogen, oxygen, and sulfur;
each R^{a} is independently selected from halogen, CN, CO₂R, C(O)NR₂, NR₂, OR, SR, and optionally substituted C₁₋₆ aliphatic;
each R^{b} is independently selected from halogen, CN, CO₂R, C(O)NR₂, NR₂, OR, SR, oxo and optionally substituted C₁₋₆ aliphatic;
R¹ is hydrogen or optionally substituted C₁₋₆ aliphatic;
L is a covalent bond or a bivalent C₁₋₆ straight or branched hydrocarbon chain;
R² is

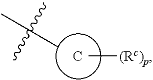

C(O)NR₂, NR₂, OR, or S(═O)ₓR;
Ring C is selected from a 3- to 7-membered cycloaliphatic ring, phenyl, a 3- to 7-membered heterocyclic ring comprising 1-3 heteroatoms independently selected from nitrogen, oxygen, and sulfur, a 5- to 6-membered heteroaryl ring comprising 1-4 heteroatoms independently selected from nitrogen, oxygen, and sulfur, and a 9- to 10-membered heteroaryl ring comprising 1-5 heteroatoms independently selected from nitrogen, oxygen, and sulfur;
each R^{c} is independently selected from halogen, oxo, OR, CO₂R, C(O)NR₂, and optionally substituted C₁₋₆ aliphatic; or:
   two independent occurrences of R^{c}, taken together with their intervening atom(s), form an optionally substituted 5- to 8-membered heterocyclic ring comprising 1-4 heteroatoms independently selected from nitrogen, oxygen, and sulfur;
each R is independently selected from hydrogen and an optionally substituted group selected from C₁₋₆ aliphatic, phenyl, a 7- to 9-membered bridged bicyclic cycloaliphatic ring, and a 3- to 7-membered heterocyclic ring comprising 1-3 heteroatoms independently selected from nitrogen, oxygen, and sulfur; or:
   two independent occurrences of R, taken together with the nitrogen atom to which they are attached, form an optionally substituted 3- to 7-membered heterocyclic ring comprising 0-3 additional heteroatoms independently selected from nitrogen, oxygen, and sulfur;
x is 0-2; and
each of m, n, and p is independently 0-4.

As described above, Ring A is selected from phenyl and a 6-membered heteroaryl ring comprising 1-3 nitrogen atoms. In some embodiments, Ring A is phenyl. In some embodiments, Ring A is a 6-membered heteroaryl ring comprising 1-3 nitrogen atoms. In some embodiments, Ring A is a 6-membered heteroaryl ring comprising 1-2 nitrogen atoms. In some embodiments, Ring A is a 6-membered heteroaryl ring comprising 1 nitrogen atom. In some embodiments, Ring A is a 6-membered heteroaryl ring comprising 2 nitrogen atoms. In some embodiments, Ring A is a 6-membered heteroaryl ring comprising 3 nitrogen atoms. In some embodiments, Ring A is pyrimidinyl. In some embodiments, Ring A is

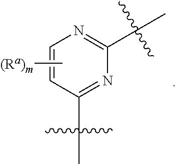

As described above, Ring B is selected from phenyl, a 5- to 6-membered heteroaryl ring comprising 1-3 heteroatoms independently selected from nitrogen, oxygen, and sulfur, and a 9- to 10-membered heteroaryl ring comprising 1-4 heteroatoms independently selected from nitrogen, oxygen, and sulfur. In some embodiments, Ring B is phenyl.

In some embodiments, Ring B is a 5- to 6-membered heteroaryl ring comprising 1-3 heteroatoms independently selected from nitrogen, oxygen, and sulfur, and a 9- to 10-membered heteroaryl ring comprising 1-4 heteroatoms independently selected from nitrogen, oxygen, and sulfur. In some embodiments, Ring B is a 5-membered heteroaryl ring comprising 1-3 heteroatoms independently selected from nitrogen, oxygen, and sulfur. In some embodiments, Ring B is a 5-membered heteroaryl ring comprising 1-2 heteroatoms independently selected from nitrogen, oxygen, and sulfur. In some embodiments, Ring B is a 5-membered heteroaryl ring comprising 1 heteroatom selected from nitrogen, oxygen, and sulfur. In some embodiments, Ring B is a 5-membered heteroaryl ring comprising 2 heteroatoms independently selected from nitrogen, oxygen, and sulfur. In some embodiments, Ring B is a 5-membered heteroaryl ring comprising 3 heteroatom independently selected from nitrogen, oxygen, and sulfur.

In some embodiments, Ring B is a 6-membered heteroaryl ring comprising 1-3 nitrogen atoms. In some embodiments, Ring B is a 6-membered heteroaryl ring comprising 1-2 nitrogen atoms. In some embodiments, Ring B is a 6-membered heteroaryl ring comprising 1 nitrogen atom. In some embodiments, Ring B is a 6-membered heteroaryl ring comprising 2 nitrogen atoms. In some embodiments, Ring B is a 6-membered heteroaryl ring comprising 3 nitrogen atoms.

In some embodiments, Ring B is a 9- to 10-membered heteroaryl ring comprising 1-4 heteroatoms independently selected from nitrogen, oxygen, and sulfur. In some embodiments, Ring B is a 9- to 10-membered heteroaryl ring comprising 1-3 heteroatoms independently selected from nitrogen, oxygen, and sulfur. In some embodiments, Ring B is a 9-membered heteroaryl ring comprising 1-4 heteroatoms independently selected from nitrogen, oxygen, and sulfur. In some embodiments, Ring B is a 9-membered heteroaryl ring comprising 1-3 heteroatoms independently selected from nitrogen, oxygen, and sulfur. In some embodiments, Ring B is a 9-membered heteroaryl ring comprising 1-2 heteroatoms independently selected from nitrogen, oxygen, and sulfur. In some embodiments, Ring B is a 9-membered heteroaryl ring comprising 1 heteroatom selected from nitrogen, oxygen, and sulfur. In some embodiments, Ring B is a 9-membered heteroaryl ring comprising 2 heteroatoms independently selected from nitrogen, oxygen, and sulfur. In some embodiments, Ring B is a 9-membered heteroaryl ring comprising 3 heteroatoms independently selected from nitrogen, oxygen, and sulfur. In some embodiments, Ring B is a 9-membered heteroaryl ring comprising 4 heteroatoms independently selected from nitrogen, oxygen, and sulfur. In some embodiments, Ring B is indazolyl. In some embodiments, Ring B is azaindazolyl. In some embodiments, Ring B is benzimidazolyl. In some embodiments, Ring B is imidazopyridinyl. In some embodiments, Ring B is triazolopyridinyl. In some embodiments, Ring B is selected from

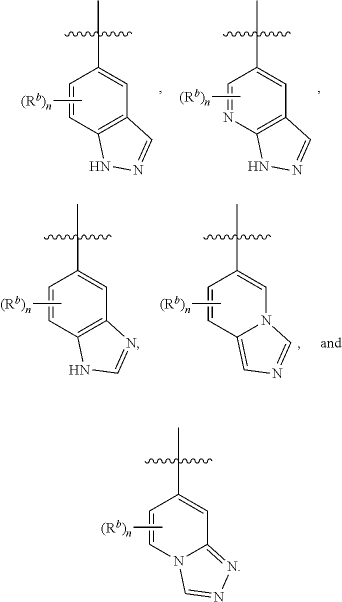

In some embodiments, Ring B is

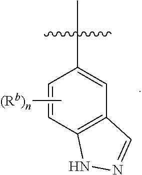

In some embodiments, Ring B is

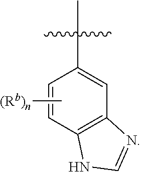

In some embodiments, Ring B is

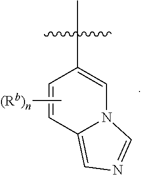

In some embodiments, Ring B is

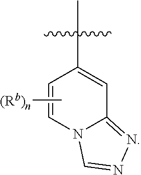

In some embodiments, Ring B is a 10-membered heteroaryl ring comprising 1-4 nitrogen atoms. In some embodiments, Ring B is a 10-membered heteroaryl ring comprising 1-3 nitrogen atoms. In some embodiments, Ring B is a 10-membered heteroaryl ring comprising 1-2 nitrogen atoms. In some embodiments, Ring B is a 10-membered heteroaryl ring comprising 1 nitrogen atom. In some embodiments, Ring B is a 10-membered heteroaryl ring comprising 2 nitrogen atoms. In some embodiments, Ring B is a 10-membered heteroaryl ring comprising 3 nitrogen atoms. In some embodiments, Ring B is a 10-membered heteroaryl ring comprising 4 nitrogen atoms.

As defined above, each R^{a} is independently selected from halogen, CN, CO₂R, C(O)NR₂, NR₂, OR, SR, and optionally substituted C₁₋₆ aliphatic. In some embodiments, R^{a} is halogen. In some embodiments, R^{a} is fluoro. In some embodiments, R^{a} is chloro. In some embodiments, R^{a} is bromo. In some embodiments, R^{a} is CN. In some embodiments, R^{a} is CO₂R. In some embodiments, R^{a} is C(O)NR₂. In some embodiments, R^{a} is NR₂. In some embodiments, R^{a} is OR. In some embodiments, R^{a} is OH. In some embodiments, R^{a} is OCH₃. In some embodiments, R^{a} is OCH₂CH₃. In some embodiments, R^{a} is SR. In some embodiments, R^{a} is CO₂R or C(O)NR₂. In some embodiments, R^{a} is NR₂, OR, or SR. In some embodiments, R^{a} is CN or halogen. In some embodiments, R^{a} is optionally substituted C₁₋₆ aliphatic. In some embodiments, R^{a} is optionally substituted C₁₋₃ aliphatic. In some embodiments, R^{a} is optionally substituted C₁₋₂ aliphatic. In some embodiments, R^{a} is optionally substituted —CH₃. In some embodiments, R^{a} is optionally substituted —CH₂CH₃. In some embodiments, R^{a} is optionally substituted —CH₂CH₂CH₃.

As defined above, each R^{b} is independently selected from halogen, CN, CO₂R, C(O)NR₂, NR₂, OR, SR, oxo and optionally substituted C₁₋₆ aliphatic.

In some embodiments, R^{b} is halogen. In some embodiments, R^{b} is fluoro. In some embodiments, R^{b} is chloro. In some embodiments, R^{b} is bromo.

In some embodiments, R^{b} is CN. In some embodiments, R^{b} is CO₂R. In some embodiments, R^{b} is C(O)NR₂. In some embodiments, R^{b} is NR₂. In some embodiments, R^{b} is OR. In some embodiments, R^{b} is OH. In some embodiments, R^{b} is OCH₃. In some embodiments, R^{b} is OCH₂CH₃. In some embodiments, R^{b} is SR. In some embodiments, R^{b} is CO₂R or C(O)NR₂. In some embodiments, R^{b} is NR₂, OR, or SR.

In some embodiments, R^{b} is OR. In some embodiments, R^{b} is OH. In some embodiments, R^{b} is OCH₃. In some embodiments, R^{b} is OCH₂CH₃.

In some embodiments, R^{b} is optionally substituted C₁₋₆ aliphatic. In some embodiments, R^{b} is optionally substituted C₁₋₃ aliphatic. In some embodiments, R^{b} is optionally substituted C₁₋₂ aliphatic. In some embodiments, R^{b} is optionally substituted —CH₃. In some embodiments, R^{b} is optionally substituted —CH₂CH₃. In some embodiments, R^{b} is optionally substituted —CH₂CH₂CH₃.

As defined above, R¹ is hydrogen or optionally substituted C₁₋₆ aliphatic. In some embodiments, R¹ is hydrogen. In some embodiments, R¹ is optionally substituted C₁₋₆ aliphatic. In some embodiments, R¹ is optionally substituted C₁₋₃ aliphatic. In some embodiments, R¹ is optionally substituted C₁₋₂ aliphatic. In some embodiments, R¹ is optionally substituted —CH₃. In some embodiments, R¹ is optionally substituted —CH₂CH₃. In some embodiments, R¹ is optionally substituted —CH₂CH₂CH₃.

As defined above, L is a covalent bond or a bivalent C₁₋₆ straight or branched hydrocarbon chain. In some embodiments, L is a covalent bond. In some embodiments, L is a bivalent C₁₋₆ straight or branched hydrocarbon chain. In some embodiments, L is a bivalent C₁₋₅ straight or branched hydrocarbon chain. In some embodiments, L is a bivalent C₁₋₄ straight hydrocarbon chain. In some embodiments, L is a bivalent C₁₋₃ straight hydrocarbon chain. In some embodiments, L is a bivalent C₁₋₂ straight hydrocarbon chain. In some embodiments, L is a bivalent C₂₋₆ branched hydrocarbon chain. In some embodiments, L is a bivalent C₂₋₅ branched hydrocarbon chain. In some embodiments, L is a bivalent C₂₋₄ branched hydrocarbon chain. In some embodiments, L is a bivalent C₂₋₃ branched hydrocarbon chain. In some embodiments, L is a bivalent C₃ branched hydrocarbon chain. In some embodiments, L is a bivalent C₄ branched hydrocarbon chain. In some embodiments, L is a bivalent C₅ branched hydrocarbon chain. In some embodiments, L is a bivalent C₆ branched hydrocarbon chain. In some embodiments, L is —CH₂—. In some embodiments, L is —CD₂-. In some embodiments, L is —CH₂CH₂—. In some embodiments, L is —CH(CH₃)—. In some embodiments, L is —CH₂CH(CH₃)—. In some embodiments, L is

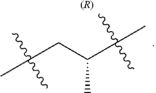 [SMILES: "(R)" ; *C[C@H](*)C]

In some embodiments, L is

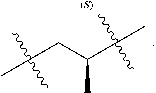 [SMILES: "(S)" ; *C[C@@H](*)C]

In some embodiments, L is —CH₂C(CH₃)₂—.

As defined above, R² is

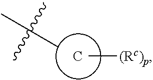

C(O)NR₂, NR₂, OR, or S(═O)ₓR.

In some embodiments, R² is C(O)NR₂, wherein each R is independently selected from hydrogen or an optionally substituted group selected from C₁₋₆ aliphatic and a 7- to 9-membered bridged bicyclic cycloaliphatic ring, or two occurrences of R, taken together with the nitrogen atom to which they are attached, form an optionally substituted 3- to 7-membered heterocyclic ring comprising 0-1 additional heteroatoms independently selected from nitrogen, oxygen, and sulfur.

In some embodiments, R² is C(O)NR₂, wherein each R is independently selected from hydrogen and optionally substituted C₁₋₆ aliphatic. In some embodiments, R² is C(O)NR₂, wherein each R is independently selected from hydrogen and optionally substituted C₁₋₃ aliphatic. In some embodiments, R² is C(O)NR₂, wherein each R is independently selected from hydrogen and optionally substituted C₁₋₂ aliphatic. In some embodiments, R² is C(O)NR₂, wherein each R is independently selected from hydrogen and optionally substituted C₁ aliphatic. Accordingly, in some embodiments, R² is C(O)NR₂, wherein each R is independently selected from hydrogen and CH₃.

In some embodiments, R² is C(O)NR₂, wherein each R is independently optionally substituted C₁₋₃ aliphatic, wherein two occurrences of R, taken together with the nitrogen atom to which they are attached, form an optionally substituted 3- to 7-membered heterocyclic ring comprising 0-1 additional heteroatoms independently selected from nitrogen, oxygen, and sulfur. In some embodiments, R² is C(O)NR₂, wherein each R is independently optionally substituted C₁₋₂ aliphatic, wherein two occurrences of R, taken together with the nitrogen atom to which they are attached, form an optionally substituted 4-membered heterocyclic ring comprising 0 additional heteroatoms. In some embodiments, R² is C(O)NR₂, wherein each R is independently optionally substituted C₁₋₂ aliphatic, wherein two occurrences of R, taken together with the nitrogen atom to which they are attached, form an optionally substituted 5-membered heterocyclic ring comprising 0 additional heteroatoms. In some embodiments, R² is C(O)NR₂, wherein each R is independently optionally substituted C₁₋₂ aliphatic, wherein two occurrences of R, taken together with the nitrogen atom to which they are attached, form an optionally substituted 6-membered heterocyclic ring comprising 1 additional heteroatom selected from nitrogen, oxygen, and sulfur.

In some embodiments, R² is C(O)NR₂, wherein each R is independently selected from hydrogen and a 7- to 9-membered bridged bicyclic cycloaliphatic ring. In some embodiments, R² is C(O)NR₂, wherein each R is independently selected from hydrogen and a 7-membered bridged bicyclic cycloaliphatic ring. In some embodiments, R² is C(O)NR₂, wherein each R is independently selected from hydrogen and a 8-membered bridged bicyclic cycloaliphatic ring. In some embodiments, R² is C(O)NR₂, wherein each R is independently selected from hydrogen and a 9-membered bridged bicyclic cycloaliphatic ring.

In some embodiments, R² is NR₂, wherein each R is independently selected from hydrogen and optionally substituted C₁₋₆ aliphatic. In some embodiments, R² is NR₂, wherein each R is independently selected from hydrogen and optionally substituted C₁₋₃ aliphatic. In some embodiments, R² is NR₂, wherein each R is independently selected from hydrogen and optionally substituted C₁₋₂ aliphatic. In some embodiments, R² is NR₂, wherein each R is independently selected from hydrogen and optionally substituted C₁ aliphatic. In some embodiments, R² is NR₂, wherein each R is hydrogen. In some embodiments, R² is NR, wherein each R is —CH₃.

In some embodiments, R² is OR, wherein R is selected from hydrogen and optionally substituted C₁₋₆ aliphatic. In some embodiments, R² is OR, wherein R is selected from hydrogen and optionally substituted C₁₋₃ aliphatic. In some embodiments, R² is OR, wherein R is selected from hydrogen and optionally substituted C₁₋₂ aliphatic. In some embodiments, R² is OR, wherein R is selected from hydrogen and optionally substituted C₁ aliphatic. In some embodiments, R² is OR, wherein R is hydrogen. In some embodiments, R² is OR, wherein R is —CH₃.

In some embodiments, R² is S(═O)ₓR, wherein R is optionally substituted C₁₋₆ aliphatic. In some embodiments, R² is S(═O)ₓR, wherein R is optionally substituted C₁₋₃ aliphatic. In some embodiments, R² is S(═O)ₓR, wherein R is optionally substituted C₁₋₂ aliphatic. In some embodiments, R² is S(═O)ₓR, wherein R is optionally substituted C₁ aliphatic. In some embodiments, R² is S(═O)ₓR, wherein R is —CH₃.

In some embodiments, R² is

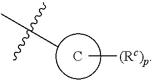

In some embodiments, R² is selected from C(O)NR₂, NR₂, OR, or S(═O)ₓR. In some embodiments, R² is C(O)NR₂. In some embodiments, R² is NR₂. In some embodiments, R² is OR. In some embodiments, R² is S(═O)ₓR.

As defined above, Ring C is selected from a 3- to 7-membered cycloaliphatic ring, phenyl, a 3- to 7-membered heterocyclic ring comprising 1-3 heteroatoms independently selected from nitrogen, oxygen, and sulfur, a 5- to 6-membered heteroaryl ring comprising 1-4 heteroatoms independently selected from nitrogen, oxygen, and sulfur, and a 9- to 10-membered heteroaryl ring comprising 1-5 heteroatoms independently selected from nitrogen, oxygen, and sulfur.

In some embodiments, Ring C is a 3- to 7-membered cycloaliphatic ring. In some embodiments, Ring C is cyclopentyl. In some embodiments, Ring C is

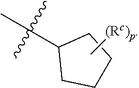

In some embodiments, Ring C is

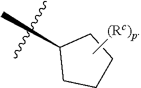

In some embodiments, Ring C is

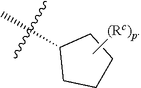

In some embodiments, Ring C is cyclohexyl. In some embodiments, Ring C is

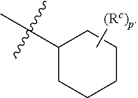

In some embodiments, Ring C is

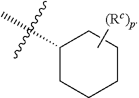

In some embodiments, Ring C is

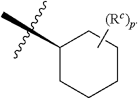

In some embodiments, Ring C is phenyl.

In some embodiments, Ring C is a 3- to 7-membered heterocyclic ring comprising 1-3 heteroatoms independently selected from nitrogen, oxygen, and sulfur. In some embodiments, Ring C is a 3- to 6-membered heterocyclic ring comprising 1-2 heteroatoms independently selected from nitrogen, oxygen, and sulfur. In some embodiments, Ring C is a 3- to 5-membered heterocyclic ring comprising 1-2 heteroatoms independently selected from nitrogen, oxygen, and sulfur. In some embodiments, Ring C is a 3- to 4-membered heterocyclic ring comprising 1 heteroatom selected from nitrogen, oxygen, and sulfur. In some embodiments, Ring C is a 6-membered heterocyclic ring comprising 1-2 heteroatoms independently selected from nitrogen, oxygen, and sulfur. In some embodiments, Ring C is a 6-membered heterocyclic ring comprising 1 heteroatom selected from nitrogen, oxygen, and sulfur. In some embodiments, Ring C is a 5-membered heterocyclic ring comprising 1-2 heteroatoms independently selected from nitrogen, oxygen, and sulfur. In some embodiments, Ring C is a 5-membered heterocyclic ring comprising 1 heteroatom selected from nitrogen, oxygen, and sulfur. In some embodiments, Ring C is

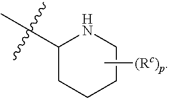

In some embodiments, Ring C is

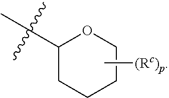

In some embodiments, Ring C is

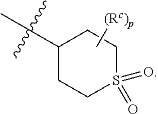

In some embodiments, Ring C is

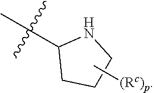

In some embodiments, Ring C is tetrahydrofuranyl. In some embodiments, Ring C is

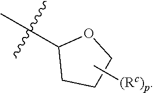

In some embodiments, Ring C is

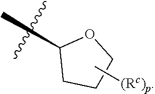

In some embodiments, Ring C is

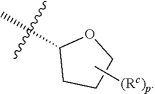

In some embodiments, Ring C is

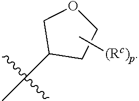

In some embodiments, Ring C is

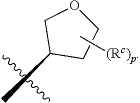

In some embodiments, Ring C is

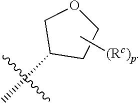

In some embodiments, Ring C is

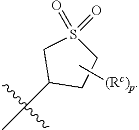

In some embodiments, Ring C is

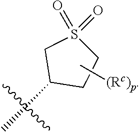

In some embodiments, Ring C is

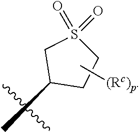

In some embodiments, Ring C is a 5- to 6-membered heteroaryl ring comprising 1-4 heteroatoms independently selected from nitrogen, oxygen, and sulfur. In some embodiments, Ring C is a 5- to 6-membered heteroaryl ring comprising 1-3 heteroatoms independently selected from nitrogen, oxygen, and sulfur. In some embodiments, Ring C is a 5-membered heteroaryl ring comprising 1-3 heteroatoms independently selected from nitrogen, oxygen, and sulfur. In some embodiments, Ring C is a 5-membered heteroaryl ring comprising 1-2 heteroatoms independently selected from nitrogen, oxygen, and sulfur. In some embodiments, Ring C is a 5-membered heteroaryl ring comprising 2 heteroatoms independently selected from nitrogen, oxygen, and sulfur. In some embodiments, Ring C is a 5-membered heteroaryl ring comprising 1 heteroatom selected from nitrogen, oxygen, and sulfur. In some embodiments, Ring C is

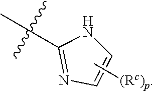

In some embodiments, Ring C is

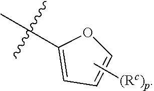

In some embodiments, Ring C is a 6-membered heteroaryl ring comprising 1-3 nitrogen atoms. In some embodiments, Ring C is a 6-membered heteroaryl ring comprising 1-2 nitrogen atoms. In some embodiments, Ring C is pyridinyl. In some embodiments, Ring C is

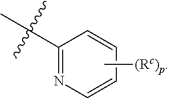

In some embodiments, Ring C is

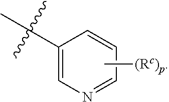

In some embodiments, Ring C is

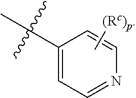

In some embodiments, Ring C is pyrimidinyl. In some embodiments, Ring C is

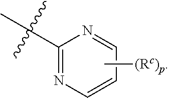

In some embodiments, Ring C is a 9- to 10-membered heteroaryl ring comprising 1-5 heteroatoms independently selected from nitrogen, oxygen, and sulfur. In some embodiments, Ring C is a 9-membered heteroaryl ring comprising 1-5 heteroatoms independently selected from nitrogen, oxygen, and sulfur. In some embodiments, Ring C is a 9-membered heteroaryl ring comprising 1-4 heteroatoms independently selected from nitrogen, oxygen, and sulfur. In some embodiments, Ring C is a 9-membered heteroaryl ring comprising 1-3 heteroatoms independently selected from nitrogen, oxygen, and sulfur. In some embodiments, Ring C is a 9-membered heteroaryl ring comprising 1-2 heteroatoms independently selected from nitrogen, oxygen, and sulfur. In some embodiments, Ring C is a 9-membered heteroaryl ring comprising 2 heteroatoms independently selected from nitrogen, oxygen, and sulfur. In some embodiments, Ring C is a 9-membered heteroaryl ring comprising 1 heteroatom selected from nitrogen, oxygen, and sulfur. In some embodiments, Ring C is benzimidazyl, tetrahydroisoindolinyl, or benzo[d][1,3]dioxolyl. In some embodiments, Ring C is

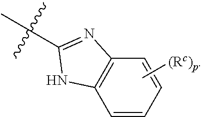

In some embodiments, Ring C is

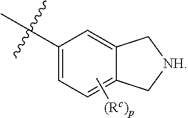

In some embodiments, Ring C is

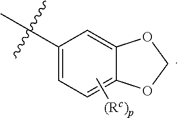

In some embodiments, Ring C is a 10-membered heteroaryl ring comprising 1-5 nitrogen atoms. In some embodiments, Ring C is a 10-membered heteroaryl ring comprising 1-4 nitrogen atoms. In some embodiments, Ring C is a 10-membered heteroacyl ring comprising 1-3 nitrogen atoms. In some embodiments, Ring C is a 10-membered heteroacyl ring comprising 1-2 nitrogen atoms. In some embodiments, Ring C is a 10-membered heteroaryl ring comprising 1 nitrogen atom. In some embodiments, Ring C is

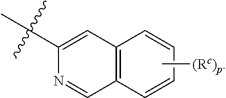

As defined above, each R^{c} is independently selected from halogen, oxo, OR, CO₂R, C(O)N(R)₂, and optionally substituted C₁₋₆ aliphatic, or two independent occurrences of R^{c}, taken together with their intervening atom(s), form an optionally substituted 5- to 8-membered heterocyclic ring comprising 1-4 heteroatoms independently selected from nitrogen, oxygen, and sulfur.

In some embodiments, R^{c} is halogen. In some embodiments, R^{c} is fluoro. In some embodiments, R^{c} is chloro. In some embodiments, R^{c} is bromo.

In some embodiments, R^{c} is oxo.

In some embodiments, R^{c} is OR, wherein R is selected from hydrogen and optionally substituted C₁₋₆ aliphatic. In some embodiments, R^{c} is OR, wherein R is selected from hydrogen and optionally substituted C₁₋₃ aliphatic. In some embodiments, R^{c} is OR, wherein R is selected from hydrogen and optionally substituted C₁₋₂ aliphatic. In some embodiments, R^{c} is OR, wherein R is hydrogen. In some embodiments, R^{c} is OR, wherein R is —CH₃. In some embodiments, R^{c} is OR and two independent occurrences of R^{c}, taken together with their intervening atom(s), form an optionally substituted 5-membered heterocyclic ring comprising 2 heteroatoms independently selected from nitrogen, oxygen, and sulfur.

In some embodiments, R^{c} is CO₂R, wherein R is selected from hydrogen and optionally substituted C₁₋₆ aliphatic. In some embodiments, R^{c} is CO₂R, wherein R is selected from hydrogen and optionally substituted C₁₋₃ aliphatic. In some embodiments, R^{c} is CO₂R, wherein R is selected from hydrogen and optionally substituted C₁₋₂ aliphatic. In some embodiments, R^{c} is CO₂R, wherein R is hydrogen. In some embodiments, R^{c} is CO₂R, wherein R is —CH₃.

In some embodiments, R^{c} is C(O)N(R)₂, wherein each R is selected from hydrogen and optionally substituted C₁₋₆ aliphatic. In some embodiments, R^{c} is C(O)N(R)₂, wherein each R is selected from hydrogen and optionally substituted C₁₋₃ aliphatic. In some embodiments, R^{c} is C(O)N(R)₂, wherein each R is selected from hydrogen and optionally substituted C₁₋₂ aliphatic. In some embodiments, R^{c} is C(O)N(R)₂, wherein each R is hydrogen. In some embodiments, R^{c} is C(O)N(R)₂, wherein each R is —CH₃. In some embodiments, R^{c} is C(O)N(R)₂, wherein each R is selected from hydrogen and —CH₃.

In some embodiments, R^{c} is optionally substituted C₁₋₆ aliphatic. In some embodiments, R^{c} is optionally substituted C₁₋₃ aliphatic. In some embodiments, R^{c} is optionally substituted C₁₋₂ aliphatic. In some embodiments, R^{c} is optionally substituted C₁ aliphatic. Accordingly, in some embodiments, R^{c} is —CH₃.

In some embodiments, R^{c} is C₁₋₆ aliphatic optionally substituted with a group selected from halogen and —(CH₂)₀₋₄OR^{∘}. In some embodiments, R^{c} is C₁₋₃ aliphatic optionally substituted with a group selected from halogen and —(CH₂)₀₋₄OR^{∘}. In some embodiments, R^{c} is C₁₋₃ aliphatic optionally substituted with a group selected from halogen and —OR^{∘}. In some embodiments, R^{c} is C₁₋₃ aliphatic optionally substituted with a group selected from halogen and —OR^{∘}, wherein R^{∘} is selected from hydrogen and C₁₋₆ aliphatic. In some embodiments, R^{c} is —CF₃. In some embodiments, R^{c} is —CH₂OH. In some embodiments, R^{c} is optionally substituted C₁₋₂ aliphatic and two independent occurrences of R^{c}, taken together with their intervening atom(s), form an optionally substituted 5-membered heterocyclic ring comprising 1 heteroatom independently selected from nitrogen, oxygen, and sulfur.

As defined above, each R is independently selected from hydrogen and an optionally substituted group selected from C₁₋₆ aliphatic, phenyl, a 7- to 9-membered bridged bicyclic cycloaliphatic ring, and a 3- to 7-membered heterocyclic ring comprising 1-3 heteroatoms independently selected from nitrogen, oxygen, and sulfur; or two independent occurrences of R, taken together with the nitrogen atom to which they are attached, form an optionally substituted 3- to 7-membered heterocyclic ring comprising 0-3 additional heteroatoms independently selected from nitrogen, oxygen, and sulfur. In some embodiments, R is hydrogen. In some embodiments, R is an optionally substituted group selected from C₁₋₆ aliphatic, phenyl, a 7- to 9-membered bridged bicyclic cycloaliphatic ring, and a 3- to 7-membered heterocyclic ring comprising 1-3 heteroatoms independently selected from nitrogen, oxygen, and sulfur; or two independent occurrences of R, taken together with the nitrogen atom to which they are attached, form an optionally substituted 3- to 7-membered heterocyclic ring comprising 0-3 additional heteroatoms independently selected from nitrogen, oxygen, and sulfur. In some embodiments, R is an optionally substituted group selected from C₁₋₆ aliphatic, phenyl, a 7- to 9-membered bridged bicyclic cycloaliphatic ring, and a 3- to 7-membered heterocyclic ring comprising 1-3 heteroatoms independently selected from nitrogen, oxygen, and sulfur. In some embodiments, R is optionally substituted C₁₋₆ aliphatic. In some embodiments, R is C₁₋₆ aliphatic. In some embodiments, R is hydrogen or C₁₋₆ aliphatic.

As defined above, each of m, n, and p is independently 0-4. In some embodiments, m is 0. In some embodiments, m is 1. In some embodiments, m is 0-1. In some embodiments, m is 2. In some embodiments, m is 3. In some embodiments, m is 4. In some embodiments, m is 1-4. In some embodiments, n is 0. In some embodiments, n is 1. In some embodiments, n is 0-1. In some embodiments, n is 2. In some embodiments, n is 3. In some embodiments, n is 4. In some embodiments, n is 1-4. In some embodiments, p is 0. In some embodiments, p is 1. In some embodiments, p is 2. In some embodiments, p is 3. In some embodiments, p is 4. In some embodiments, p is 1-4. In some embodiments, p is 0-1. In some embodiments, p is 0-2. In some embodiments, p is 0-3.

As defined above, x is 0, 1, or 2. In some embodiments, x is 0. Accordingly, in some embodiments, R² is SR. In some embodiments, x is 1. Accordingly, in some embodiments, R² is S(═O)R. In some embodiments, x is 2. Accordingly, in some embodiments, R² is SO₂R.

In some embodiments, the present disclosure provides a compound of Formula I-a:

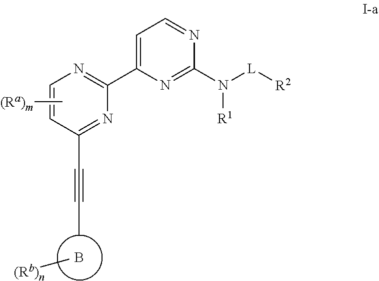

or a pharmaceutically acceptable salt thereof, wherein each of Ring B, R^{a}, R^{b}, L, R¹, R², m, and n is as described above and defined herein.

In some embodiments, the present disclosure provides a compound of Formula I-b:

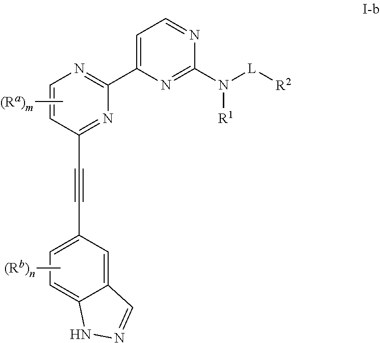

or a pharmaceutically acceptable salt thereof, wherein each of R^{a}, R^{b}, L, R¹, R², m, and n is as described above and defined herein.

In some embodiments, the present disclosure provides a compound of Formula I-c:

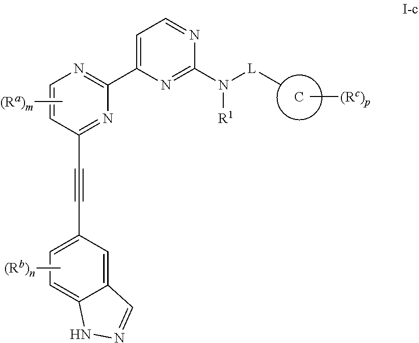

or a pharmaceutically acceptable salt thereof, wherein each of Ring C, R^{a}, R^{b}, R^{c}, L, R¹, m, n, and p is as described above and defined herein.

In some embodiments, the present disclosure provides a compound of Formula I-d:

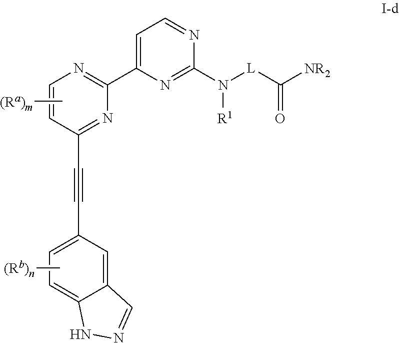

or a pharmaceutically acceptable salt thereof, wherein each of R^{a}, R^{b}, R¹, L, R, m, and n is as described above and defined herein.

In some embodiments, the present disclosure provides a compound of Formula I-a-i:

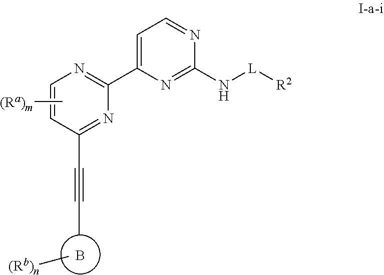

or a pharmaceutically acceptable salt thereof, wherein each of Ring B, R^{a}, R^{b}, L, R², m, and n is as described above and defined herein.

In some embodiments, the present disclosure provides a compound of Formula I-b-i:

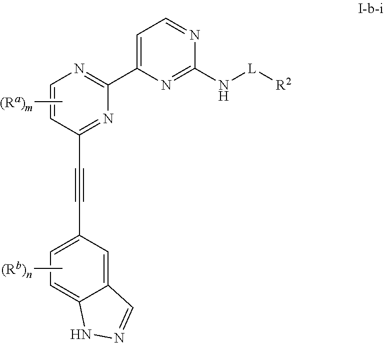

or a pharmaceutically acceptable salt thereof, wherein each of R^{a}, R^{b}, L, R², m, and n is as described above and defined herein.

In some embodiments, the present disclosure provides a compound of Formula I-c-i:

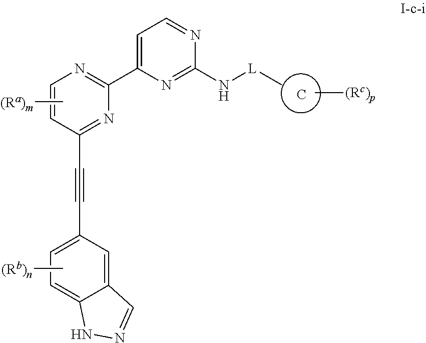

or a pharmaceutically acceptable salt thereof, wherein each of Ring C, R^{a}, R^{b}, R^{c}, L, m, n, and p is as described above and defined herein.

In some embodiments, the present disclosure provides a compound of Formula I-d-i:

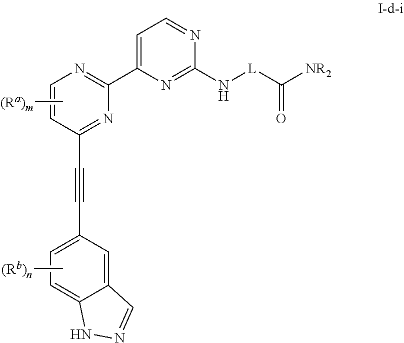

or a pharmaceutically acceptable salt thereof, wherein each of R^{a}, R^{b}, L, R, m, and n is as described above and defined herein.

It will be understood that, unless otherwise specified or prohibited by the foregoing definitions of Formulae I-a, I-a-i, I-b, I-b-i, I-c, I-c-i, I-d, and I-d-i, embodiments of variables as defined above and described in classes and subclasses herein for Formula I also apply to compounds of Formulae I-a, I-a-i, I-b, I-b-i, I-c, I-c-i, I-d, and I-d-i, mutatis mutandis, both singly and in combination.

In some embodiments, the present disclosure provides a compound selected from the group consisting of:

TABLE 1

| | |
|---|---|
| 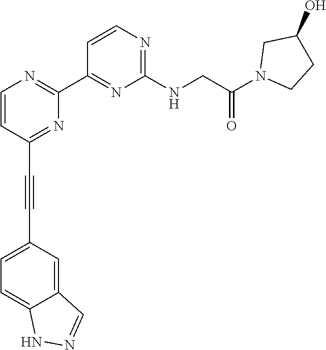 [SMILES: O=C(CNc1nccc(-c2nccc(C#Cc3ccc4[nH]ncc4c3)n2)n1)N1CC[C@H](O)C1] | Example 1 |
| 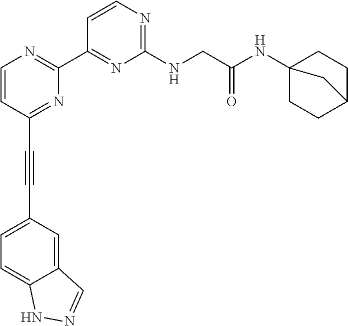 [SMILES: O=C(CNc1nccc(-c2nccc(C#Cc3ccc4[nH]ncc4c3)n2)n1)NC12CCC(CC1)C2] | Example 2 |
| 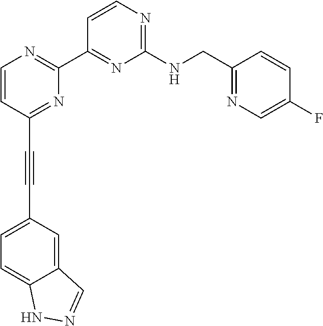 [SMILES: Fc1ccc(CNc2nccc(-c3nccc(C#Cc4ccc5[nH]ncc5c4)n3)n2)nc1] | Example 3 |
| 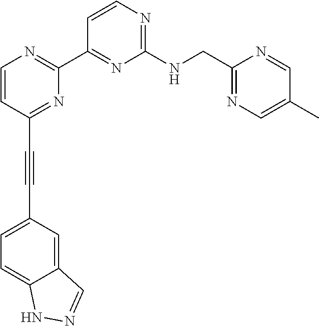 [SMILES: Cc1cnc(CNc2nccc(-c3nccc(C#Cc4ccc5[nH]ncc5c4)n3)n2)nc1] | Example 4 |
| 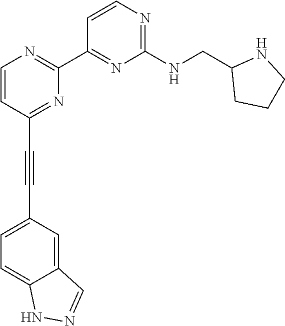 [SMILES: C(#Cc1ccnc(-c2ccnc(NCC3CCCN3)n2)n1)c1ccc2[nH]ncc2c1] | Example 5 |
| 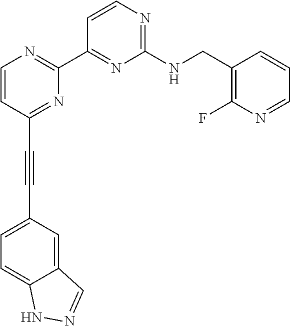 [SMILES: Fc1ncccc1CNc1nccc(-c2nccc(C#Cc3ccc4[nH]ncc4c3)n2)n1] | Example 6 |
| 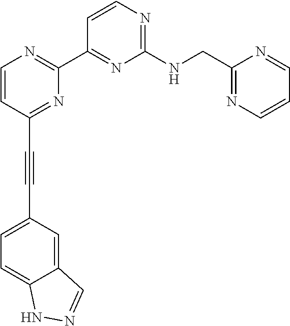 [SMILES: C(#Cc1ccnc(-c2ccnc(NCc3ncccn3)n2)n1)c1ccc2[nH]ncc2c1] | Example 7 |
| 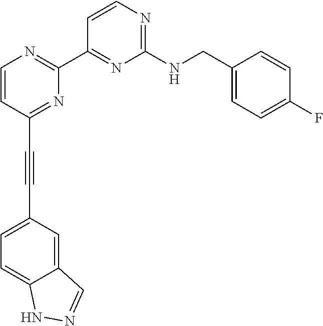 [SMILES: Fc1ccc(CNc2nccc(-c3nccc(C#Cc4ccc5[nH]ncc5c4)n3)n2)cc1] | Example 8 |
| 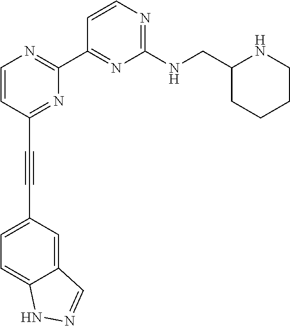 [SMILES: C(#Cc1ccnc(-c2ccnc(NCC3CCCCN3)n2)n1)c1ccc2[nH]ncc2c1] | Example 9 |
| 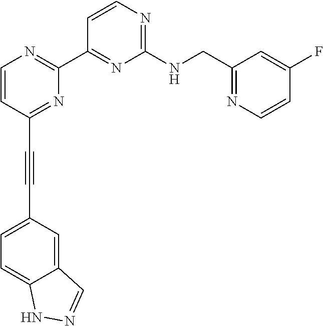 [SMILES: Fc1ccnc(CNc2nccc(-c3nccc(C#Cc4ccc5[nH]ncc5c4)n3)n2)c1] | Example 10 |
| 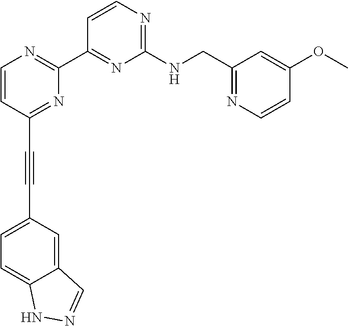 [SMILES: COc1ccnc(CNc2nccc(-c3nccc(C#Cc4ccc5[nH]ncc5c4)n3)n2)c1] | Example 11 |
| 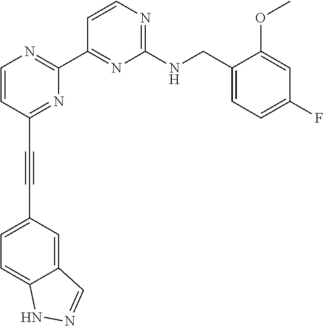 [SMILES: COc1cc(F)ccc1CNc1nccc(-c2nccc(C#Cc3ccc4[nH]ncc4c3)n2)n1] | Example 12 |
| 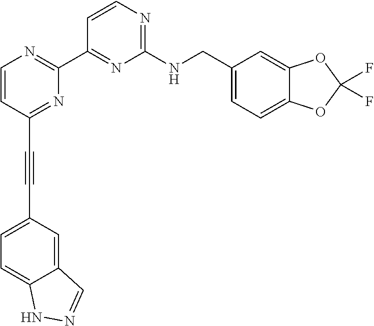 [SMILES: FC1(F)Oc2ccc(CNc3nccc(-c4nccc(C#Cc5ccc6[nH]ncc6c5)n4)n3)cc2O1] | Example 13 |
| 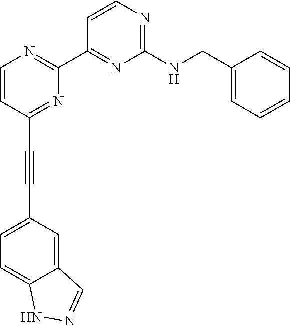 [SMILES: C(#Cc1ccnc(-c2ccnc(NCc3ccccc3)n2)n1)c1ccc2[nH]ncc2c1] | Example 14 |
| 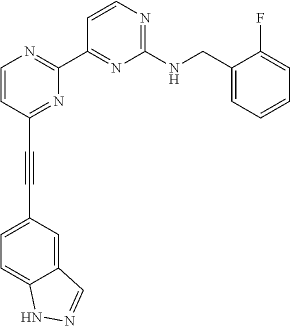 [SMILES: Fc1ccccc1CNc1nccc(-c2nccc(C#Cc3ccc4[nH]ncc4c3)n2)n1] | Example 15 |
| 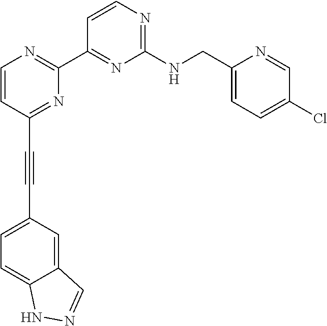 [SMILES: Clc1ccc(CNc2nccc(-c3nccc(C#Cc4ccc5[nH]ncc5c4)n3)n2)nc1] | Example 16 |
| 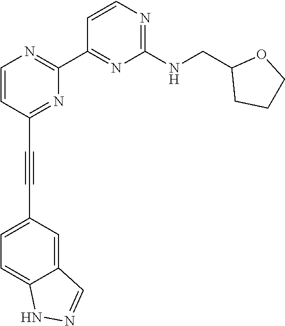 [SMILES: C(#Cc1ccnc(-c2ccnc(NCC3CCCO3)n2)n1)c1ccc2[nH]ncc2c1] | Example 17 |
| 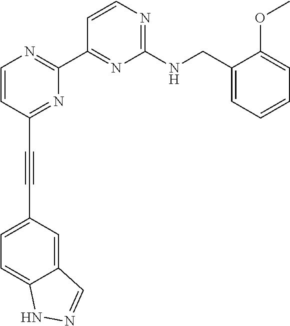 [SMILES: COc1ccccc1CNc1nccc(-c2nccc(C#Cc3ccc4[nH]ncc4c3)n2)n1] | Example 18 |
| 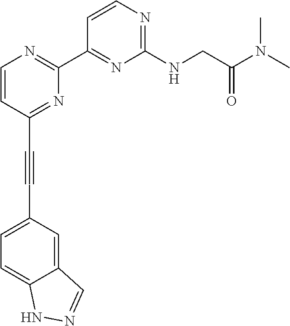 [SMILES: CN(C)C(=O)CNc1nccc(-c2nccc(C#Cc3ccc4[nH]ncc4c3)n2)n1] | Example 19 |
| 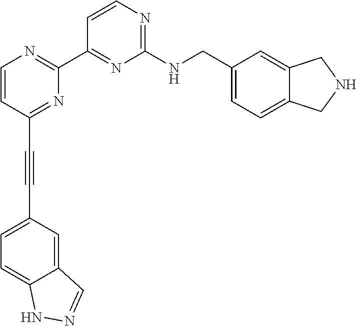 [SMILES: C(#Cc1ccnc(-c2ccnc(NCc3ccc4c(c3)CNC4)n2)n1)c1ccc2[nH]ncc2c1] | Example 20 |
| 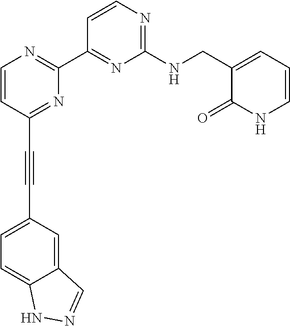 [SMILES: O=c1[nH]cccc1CNc1nccc(-c2nccc(C#Cc3ccc4[nH]ncc4c3)n2)n1] | Example 21 |
| 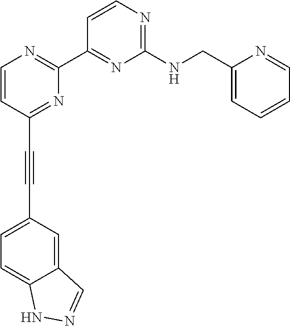 [SMILES: C(#Cc1ccnc(-c2ccnc(NCc3ccccn3)n2)n1)c1ccc2[nH]ncc2c1] | Example 22 |
| 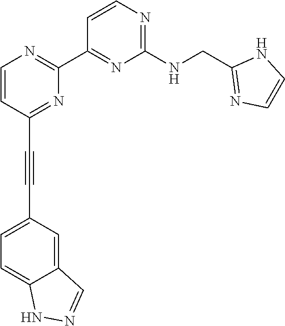 [SMILES: C(#Cc1ccnc(-c2ccnc(NCc3ncc[nH]3)n2)n1)c1ccc2[nH]ncc2c1] | Example 23 |
| 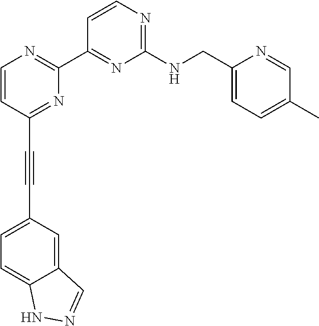 [SMILES: Cc1ccc(CNc2nccc(-c3nccc(C#Cc4ccc5[nH]ncc5c4)n3)n2)nc1] | Example 24 |
| 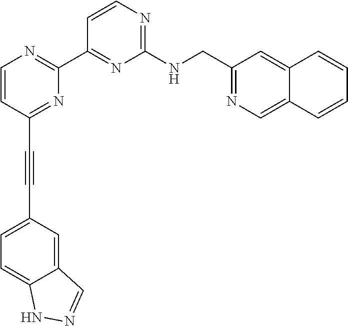 [SMILES: C(#Cc1ccnc(-c2ccnc(NCc3cc4ccccc4cn3)n2)n1)c1ccc2[nH]ncc2c1] | Example 25 |
| 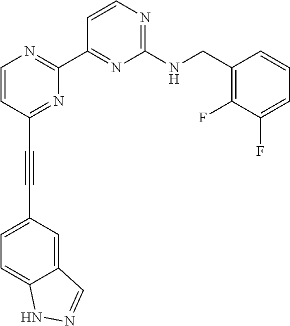 [SMILES: Fc1cccc(CNc2nccc(-c3nccc(C#Cc4ccc5[nH]ncc5c4)n3)n2)c1F] | Example 26 |
| 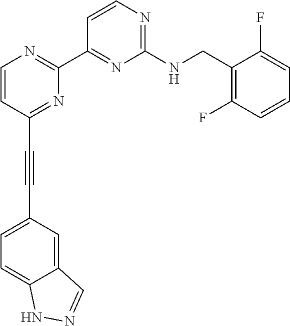 [SMILES: Fc1cccc(F)c1CNc1nccc(-c2nccc(C#Cc3ccc4[nH]ncc4c3)n2)n1] | Example 27 |
| 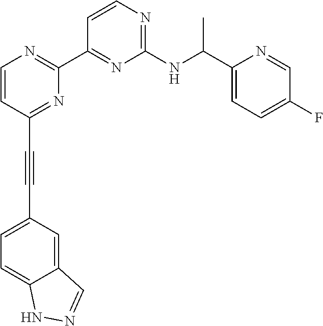 [SMILES: CC(Nc1nccc(-c2nccc(C#Cc3ccc4[nH]ncc4c3)n2)n1)c1ccc(F)cn1] | Example 28 |
| 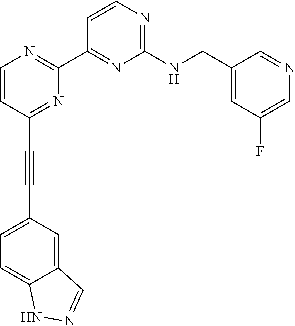 [SMILES: Fc1cncc(CNc2nccc(-c3nccc(C#Cc4ccc5[nH]ncc5c4)n3)n2)c1] | Example 29 |
| 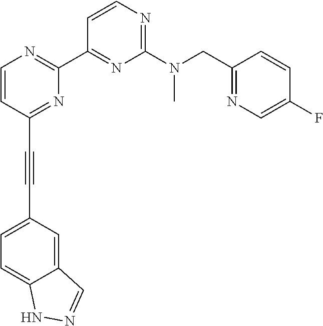 [SMILES: CN(Cc1ccc(F)cn1)c1nccc(-c2nccc(C#Cc3ccc4[nH]ncc4c3)n2)n1] | Example 30 |
| 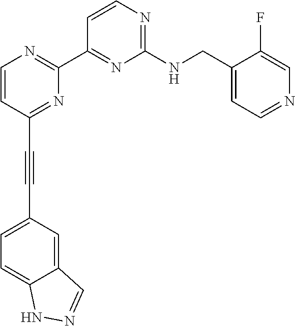 [SMILES: Fc1cnccc1CNc1nccc(-c2nccc(C#Cc3ccc4[nH]ncc4c3)n2)n1] | Example 31 |
| 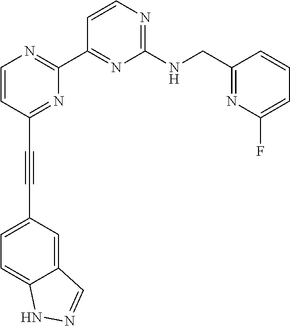 [SMILES: Fc1cccc(CNc2nccc(-c3nccc(C#Cc4ccc5[nH]ncc5c4)n3)n2)n1] | Example 32 |
| 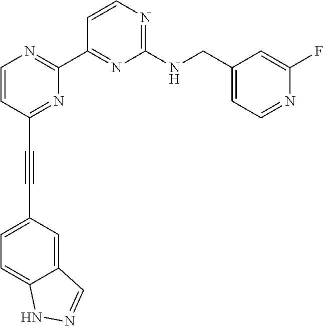 [SMILES: Fc1cc(CNc2nccc(-c3nccc(C#Cc4ccc5[nH]ncc5c4)n3)n2)ccn1] | Example 33 |
| 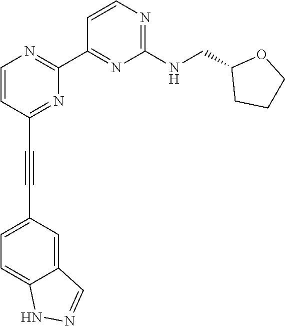 [SMILES: C(#Cc1ccnc(-c2ccnc(NC[C@H]3CCCO3)n2)n1)c1ccc2[nH]ncc2c1] | Example 34 |
| 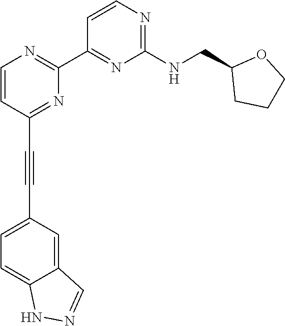 [SMILES: C(#Cc1ccnc(-c2ccnc(NC[C@@H]3CCCO3)n2)n1)c1ccc2[nH]ncc2c1] | Example 35 |
| 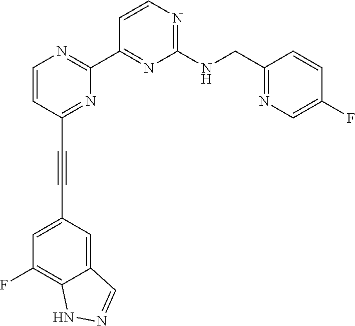 [SMILES: Fc1ccc(CNc2nccc(-c3nccc(C#Cc4cc(F)c5[nH]ncc5c4)n3)n2)nc1] | Example 36 |
| 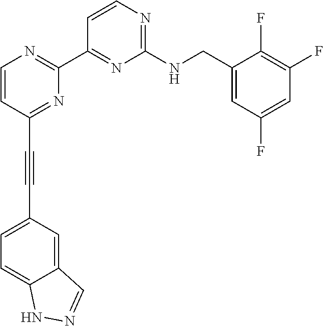 [SMILES: Fc1cc(F)c(F)c(CNc2nccc(-c3nccc(C#Cc4ccc5[nH]ncc5c4)n3)n2)c1] | Example 37 |
| 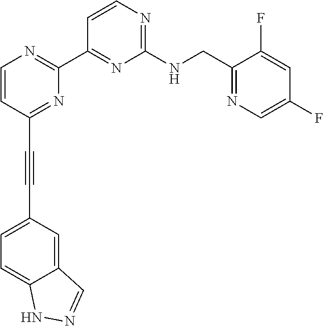 [SMILES: Fc1cnc(CNc2nccc(-c3nccc(C#Cc4ccc5[nH]ncc5c4)n3)n2)c(F)c1] | Example 38 |
| 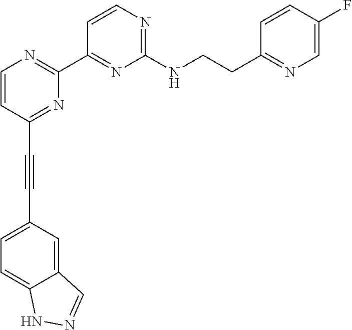 [SMILES: Fc1ccc(CCNc2nccc(-c3nccc(C#Cc4ccc5[nH]ncc5c4)n3)n2)nc1] | Example 39 |
| 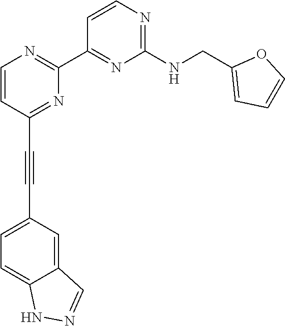 [SMILES: C(#Cc1ccnc(-c2ccnc(NCc3ccco3)n2)n1)c1ccc2[nH]ncc2c1] | Example 40 |
| 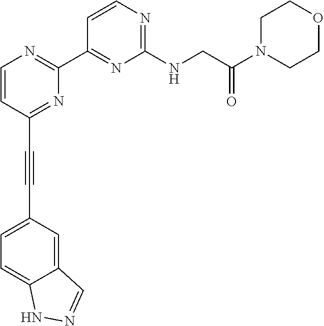 [SMILES: O=C(CNc1nccc(-c2nccc(C#Cc3ccc4[nH]ncc4c3)n2)n1)N1CCOCC1] | Example 41 |
| 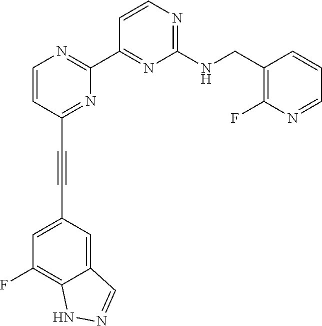 [SMILES: Fc1ncccc1CNc1nccc(-c2nccc(C#Cc3cc(F)c4[nH]ncc4c3)n2)n1] | Example 42 |
| 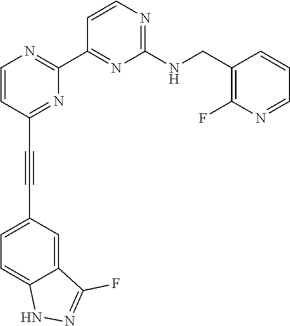 [SMILES: Fc1ncccc1CNc1nccc(-c2nccc(C#Cc3ccc4[nH]nc(F)c4c3)n2)n1] | Example 43 |
| 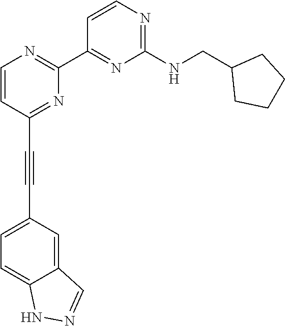 [SMILES: C(#Cc1ccnc(-c2ccnc(NCC3CCCC3)n2)n1)c1ccc2[nH]ncc2c1] | Example 44 |
| 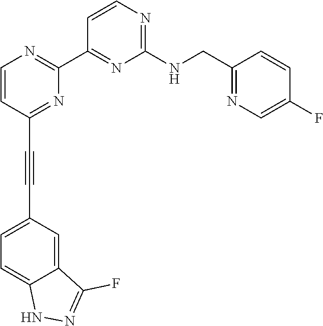 [SMILES: Fc1ccc(CNc2nccc(-c3nccc(C#Cc4ccc5[nH]nc(F)c5c4)n3)n2)nc1] | Example 45 |
| 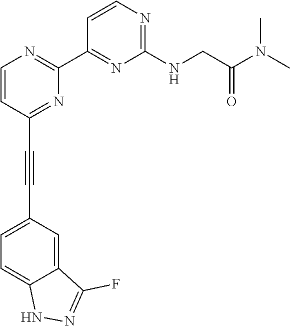 [SMILES: CN(C)C(=O)CNc1nccc(-c2nccc(C#Cc3ccc4[nH]nc(F)c4c3)n2)n1] | Example 46 |
| 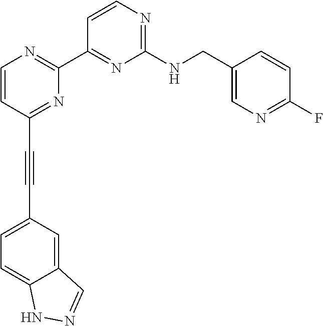 [SMILES: Fc1ccc(CNc2nccc(-c3nccc(C#Cc4ccc5[nH]ncc5c4)n3)n2)cn1] | Example 47 |
| 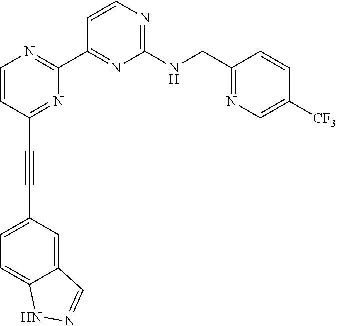 [SMILES: FC(F)(F)c1ccc(CNc2nccc(-c3nccc(C#Cc4ccc5[nH]ncc5c4)n3)n2)nc1] | Example 48 |
| 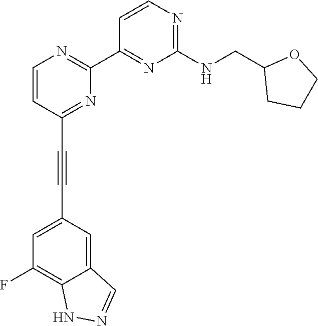 [SMILES: Fc1cc(C#Cc2ccnc(-c3ccnc(NCC4CCCO4)n3)n2)cc2cn[nH]c12] | Example 49 |
| 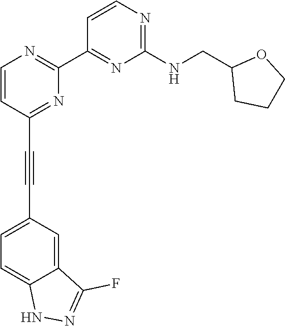 [SMILES: Fc1n[nH]c2ccc(C#Cc3ccnc(-c4ccnc(NCC5CCCO5)n4)n3)cc12] | Example 50 |
| 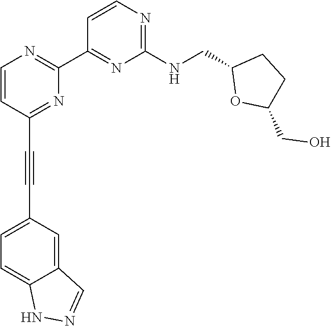 [SMILES: OC[C@H]1CC[C@@H](CNc2nccc(-c3nccc(C#Cc4ccc5[nH]ncc5c4)n3)n2)O1] | Example 51 |
| 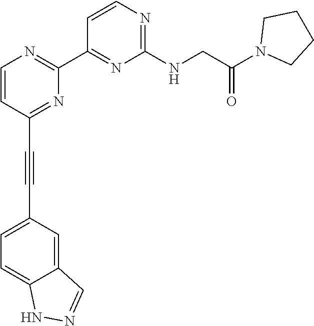 [SMILES: O=C(CNc1nccc(-c2nccc(C#Cc3ccc4[nH]ncc4c3)n2)n1)N1CCCC1] | Example 52 |
| 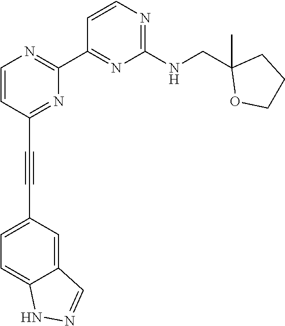 [SMILES: CC1(CNc2nccc(-c3nccc(C#Cc4ccc5[nH]ncc5c4)n3)n2)CCCO1] | Example 53 |
| 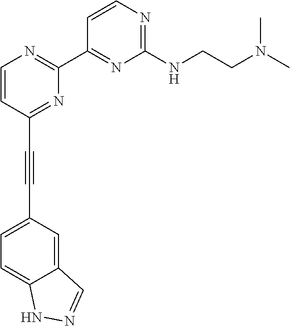 [SMILES: CN(C)CCNc1nccc(-c2nccc(C#Cc3ccc4[nH]ncc4c3)n2)n1] | Example 54 |
| 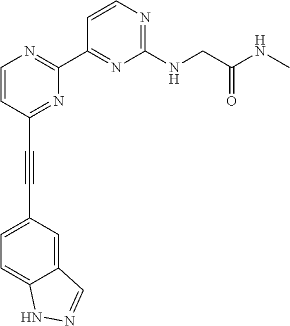 [SMILES: CNC(=O)CNc1nccc(-c2nccc(C#Cc3ccc4[nH]ncc4c3)n2)n1] | Example 55 |
| 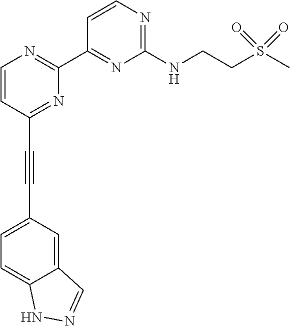 [SMILES: CS(=O)(=O)CCNc1nccc(-c2nccc(C#Cc3ccc4[nH]ncc4c3)n2)n1] | Example 56 |
| 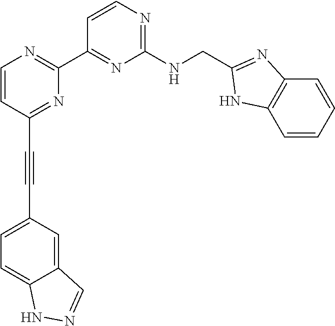 [SMILES: C(#Cc1ccnc(-c2ccnc(NCc3nc4ccccc4[nH]3)n2)n1)c1ccc2[nH]ncc2c1] | Example 57 |
| 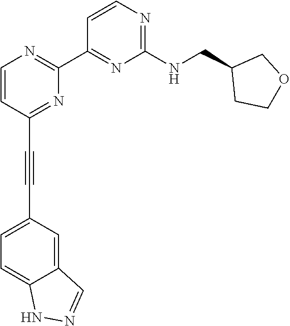 [SMILES: C(#Cc1ccnc(-c2ccnc(NC[C@@H]3CCOC3)n2)n1)c1ccc2[nH]ncc2c1] | Example 58 |
| 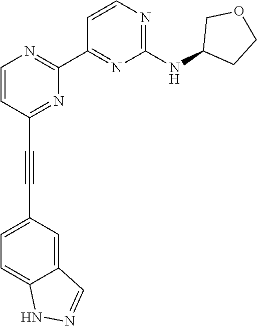 [SMILES: C(#Cc1ccnc(-c2ccnc(N[C@@H]3CCOC3)n2)n1)c1ccc2[nH]ncc2c1] | Example 59 |
| 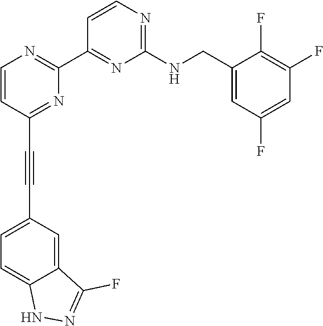 [SMILES: Fc1cc(F)c(F)c(CNc2nccc(-c3nccc(C#Cc4ccc5[nH]nc(F)c5c4)n3)n2)c1] | Example 60 |
| 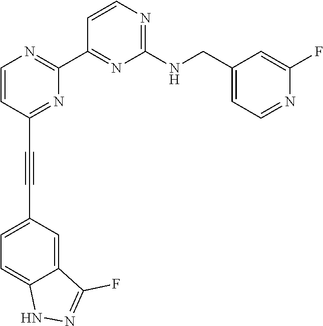 [SMILES: Fc1cc(CNc2nccc(-c3nccc(C#Cc4ccc5[nH]nc(F)c5c4)n3)n2)ccn1] | Example 61 |
| 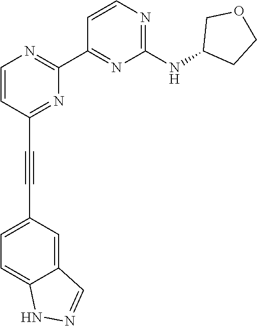 [SMILES: C(#Cc1ccnc(-c2ccnc(N[C@H]3CCOC3)n2)n1)c1ccc2[nH]ncc2c1] | Example 62 |
| 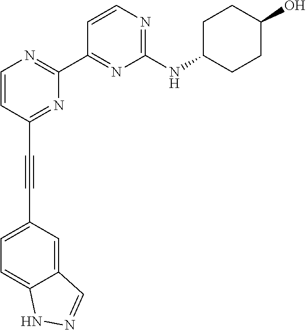 [SMILES: O[C@H]1CC[C@H](Nc2nccc(-c3nccc(C#Cc4ccc5[nH]ncc5c4)n3)n2)CC1] | Example 63 |
| 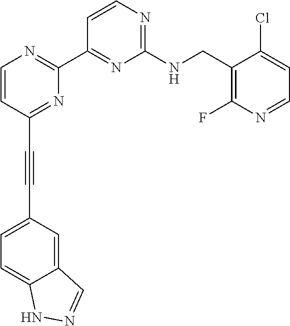 [SMILES: Fc1nccc(Cl)c1CNc1nccc(-c2nccc(C#Cc3ccc4[nH]ncc4c3)n2)n1] | Example 64 |
| 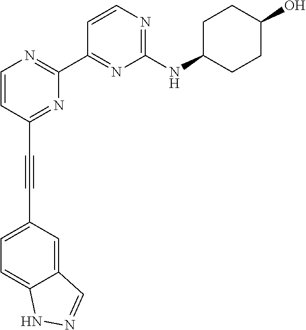 [SMILES: O[C@H]1CC[C@@H](Nc2nccc(-c3nccc(C#Cc4ccc5[nH]ncc5c4)n3)n2)CC1] | Example 65 |
| 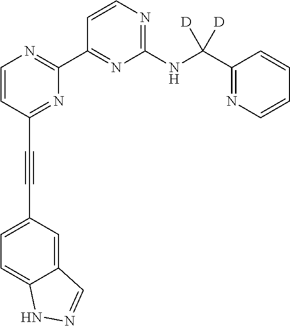 [SMILES: [2H]C([2H])(Nc1nccc(-c2nccc(C#Cc3ccc4[nH]ncc4c3)n2)n1)c1ccccn1] | Example 66 |
| 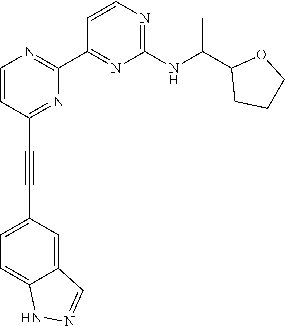 [SMILES: CC(Nc1nccc(-c2nccc(C#Cc3ccc4[nH]ncc4c3)n2)n1)C1CCCO1] | Example 67 |
| 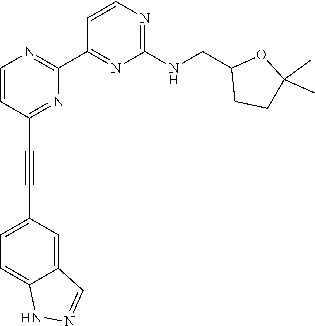 [SMILES: CC1(C)CCC(CNc2nccc(-c3nccc(C#Cc4ccc5[nH]ncc5c4)n3)n2)O1] | Example 68 |
| 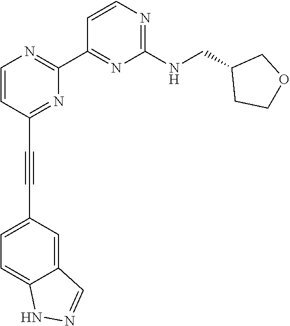 [SMILES: C(#Cc1ccnc(-c2ccnc(NC[C@H]3CCOC3)n2)n1)c1ccc2[nH]ncc2c1] | Example 69 |
| 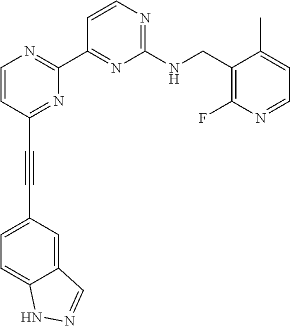 [SMILES: Cc1ccnc(F)c1CNc1nccc(-c2nccc(C#Cc3ccc4[nH]ncc4c3)n2)n1] | Example 70 |
| 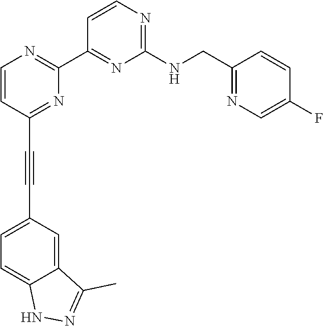 [SMILES: Cc1n[nH]c2ccc(C#Cc3ccnc(-c4ccnc(NCc5ccc(F)cn5)n4)n3)cc12] | Example 71 |
| 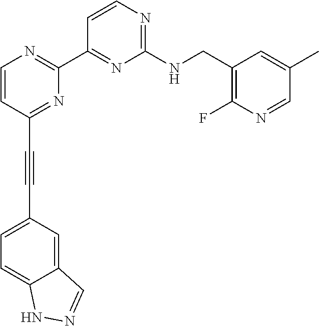 [SMILES: Cc1cnc(F)c(CNc2nccc(-c3nccc(C#Cc4ccc5[nH]ncc5c4)n3)n2)c1] | Example 72 |
| 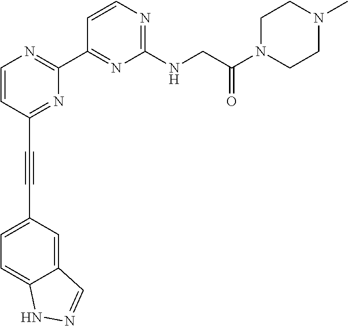 [SMILES: CN1CCN(C(=O)CNc2nccc(-c3nccc(C#Cc4ccc5[nH]ncc5c4)n3)n2)CC1] | Example 73 |
| 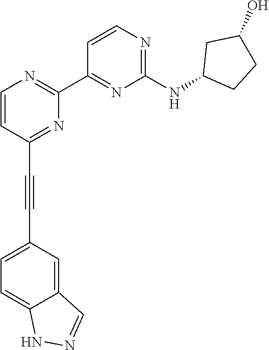 [SMILES: O[C@@H]1CC[C@H](Nc2nccc(-c3nccc(C#Cc4ccc5[nH]ncc5c4)n3)n2)C1] | Example 74 |
| 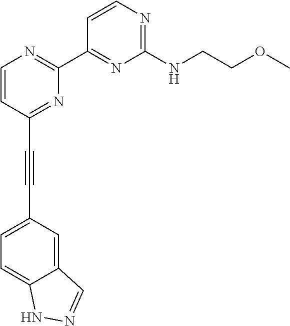 [SMILES: COCCNc1nccc(-c2nccc(C#Cc3ccc4[nH]ncc4c3)n2)n1] | Example 75 |
| 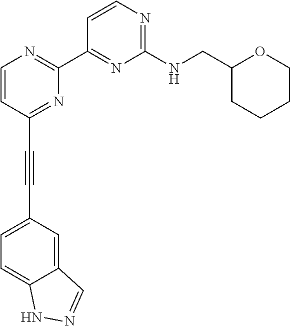 [SMILES: C(#Cc1ccnc(-c2ccnc(NCC3CCCCO3)n2)n1)c1ccc2[nH]ncc2c1] | Example 76 |
| 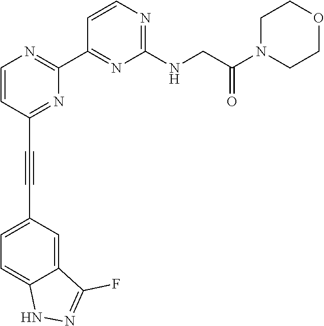 [SMILES: O=C(CNc1nccc(-c2nccc(C#Cc3ccc4[nH]nc(F)c4c3)n2)n1)N1CCOCC1] | Example 77 |
| 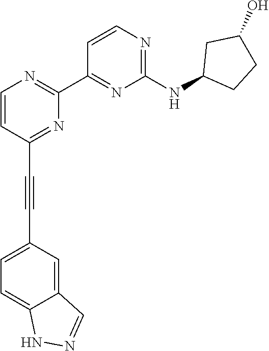 [SMILES: O[C@@H]1CC[C@@H](Nc2nccc(-c3nccc(C#Cc4ccc5[nH]ncc5c4)n3)n2)C1] | Example 78 |
| 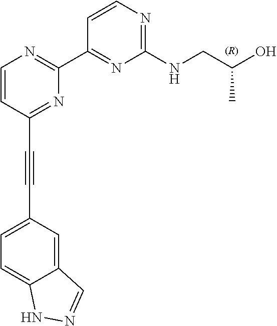 | Example 79 |
| 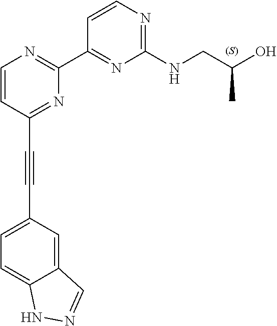 | Example 80 |
| 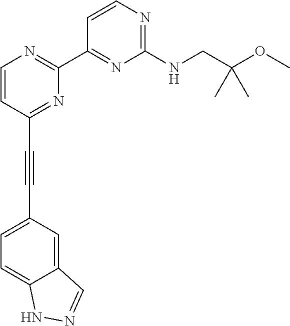 [SMILES: COC(C)(C)CNc1nccc(-c2nccc(C#Cc3ccc4[nH]ncc4c3)n2)n1] | Example 81 |
| 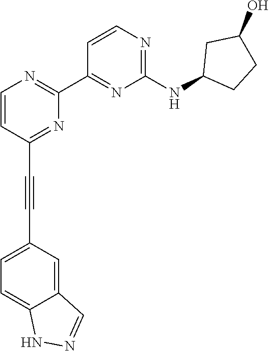 [SMILES: O[C@H]1CC[C@@H](Nc2nccc(-c3nccc(C#Cc4ccc5[nH]ncc5c4)n3)n2)C1] | Example 82 |
| 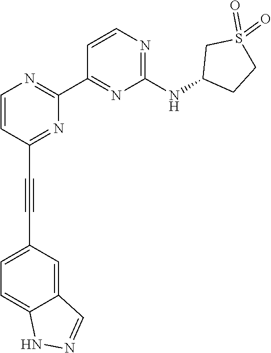 [SMILES: O=S1(=O)CC[C@H](Nc2nccc(-c3nccc(C#Cc4ccc5[nH]ncc5c4)n3)n2)C1] | Example 83 |
| 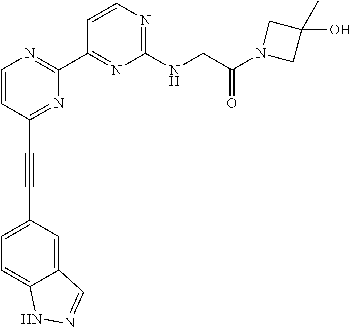 [SMILES: CC1(O)CN(C(=O)CNc2nccc(-c3nccc(C#Cc4ccc5[nH]ncc5c4)n3)n2)C1] | Example 84 |
| 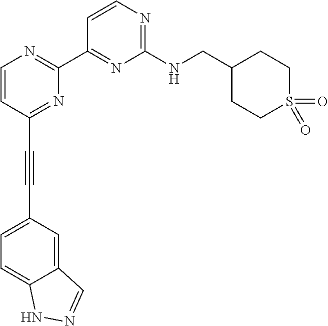 [SMILES: O=S1(=O)CCC(CNc2nccc(-c3nccc(C#Cc4ccc5[nH]ncc5c4)n3)n2)CC1] | Example 85 |
| 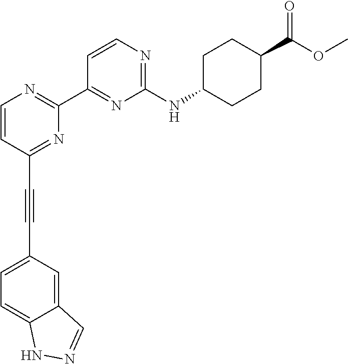 [SMILES: COC(=O)[C@H]1CC[C@H](Nc2nccc(-c3nccc(C#Cc4ccc5[nH]ncc5c4)n3)n2)CC1] | Example 86 |
| 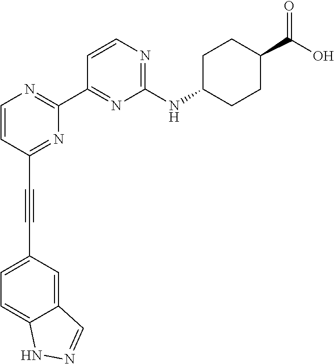 [SMILES: O=C(O)[C@H]1CC[C@H](Nc2nccc(-c3nccc(C#Cc4ccc5[nH]ncc5c4)n3)n2)CC1] | Example 87 |
| 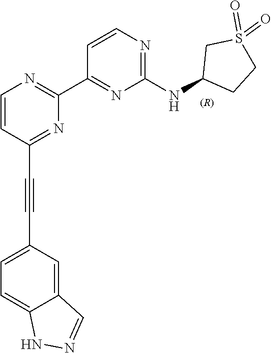 | Example 88 |
| 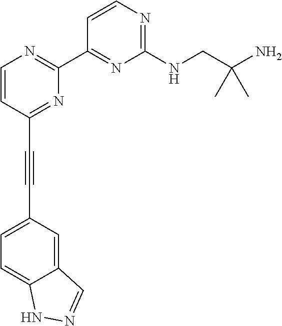 [SMILES: CC(C)(N)CNc1nccc(-c2nccc(C#Cc3ccc4[nH]ncc4c3)n2)n1] | Example 89 |
| 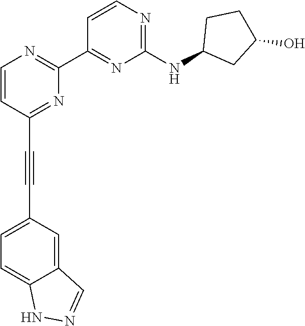 [SMILES: O[C@H]1CC[C@H](Nc2nccc(-c3nccc(C#Cc4ccc5[nH]ncc5c4)n3)n2)C1] | Example 90 |
| 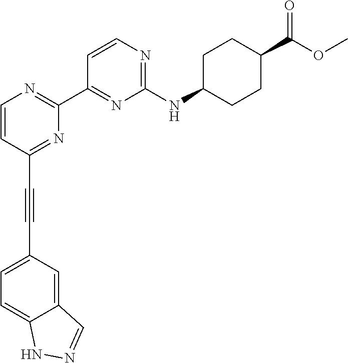 [SMILES: COC(=O)[C@H]1CC[C@@H](Nc2nccc(-c3nccc(C#Cc4ccc5[nH]ncc5c4)n3)n2)CC1] | Example 91 |
| 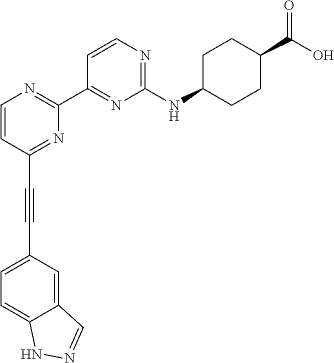 [SMILES: O=C(O)[C@H]1CC[C@@H](Nc2nccc(-c3nccc(C#Cc4ccc5[nH]ncc5c4)n3)n2)CC1] | Example 92 |
| 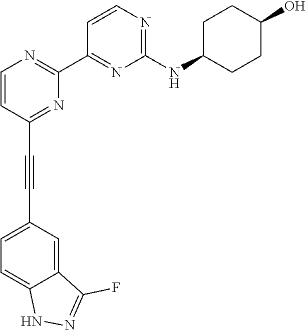 [SMILES: O[C@H]1CC[C@@H](Nc2nccc(-c3nccc(C#Cc4ccc5[nH]nc(F)c5c4)n3)n2)CC1] | Example 93 |
| 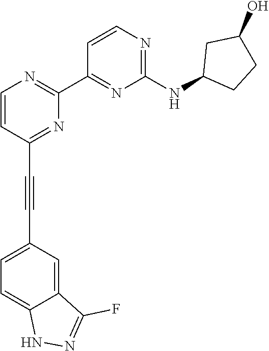 [SMILES: O[C@H]1CC[C@@H](Nc2nccc(-c3nccc(C#Cc4ccc5[nH]nc(F)c5c4)n3)n2)C1] | Example 94 |
| 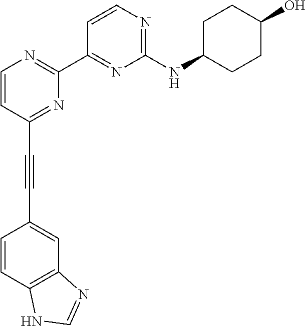 [SMILES: O[C@H]1CC[C@@H](Nc2nccc(-c3nccc(C#Cc4ccc5[nH]cnc5c4)n3)n2)CC1] | Example 95 |
| 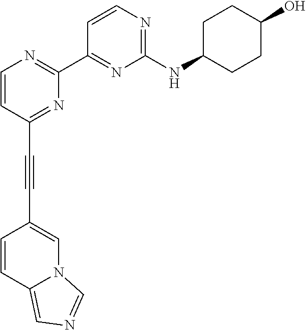 [SMILES: O[C@H]1CC[C@@H](Nc2nccc(-c3nccc(C#Cc4ccc5cncn5c4)n3)n2)CC1] | Example 96 |
| 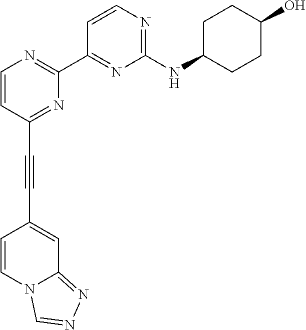 [SMILES: O[C@H]1CC[C@@H](Nc2nccc(-c3nccc(C#Cc4ccn5cnnc5c4)n3)n2)CC1] | Example 97 |

or a pharmaceutically acceptable salt thereof.

It will be appreciated that each of the compounds described herein and each of the subclasses of compounds described above may be substituted as described generally herein, or may be substituted according to any one or more of the subclasses described above and herein.

It will be appreciated that throughout the present disclosure, unless otherwise indicated, reference to a compound of Formula I is intended to also include Formulae I-a, I-a-i, I-b, I-b-i, I-c, I-c-i, I-d, and I-d-i, and compound species of such formulae disclosed herein.

Some of the foregoing compounds can comprise one or more asymmetric centers, and thus can exist in various isomeric forms, e.g., stereoisomers and/or diastereomers. Thus, provided compounds and pharmaceutical compositions thereof may be in the form of an individual enantiomer, diastereomer or geometric isomer, or may be in the form of a mixture of stereoisomers. In certain embodiments, the compounds described herein are enantiopure compounds. In certain other embodiments, mixtures of stereoisomers or diastereomers are provided.

Furthermore, certain compounds, as described herein may have one or more double bonds that can exist as either the Z or E isomer, unless otherwise indicated. The present disclosure additionally encompasses the compounds as individual isomers substantially free of other isomers and alternatively, as mixtures of various isomers, e.g., racemic mixtures of stereoisomers. In addition to the above-mentioned compounds per se, the present disclosure also encompasses pharmaceutically acceptable derivatives of these compounds and compositions comprising one or more compounds described herein and one or more pharmaceutically acceptable excipients or additives. In some embodiments, a compound in the present disclosure or a subgenera thereof is provided as a pharmaceutically acceptable salt.

Provided compounds may be prepared by crystallization of a compound under different conditions and may exist as one or a combination of polymorphs. For example, different polymorphs may be identified and/or prepared using different solvents, or different mixtures of solvents for recrystallization; by performing crystallizations at different temperatures; or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other techniques. Thus, the present disclosure encompasses provided compounds, their derivatives, their tautomeric forms, their stereoisomers, their polymorphs, their pharmaceutically acceptable salts, their pharmaceutically acceptable solvates and pharmaceutically acceptable compositions containing them. Tautomeric forms of compounds of the present disclosure include, for example, the substituted indazolyl compounds in which the proton on the nitrogen can be attached to either of the two nitrogen atoms of any of the aforementioned compounds of general Formula I and related formulae.

Pharmaceutical Compositions

As discussed above, the present disclosure provides novel compounds that have biological properties useful for the treatment of any of a number of conditions or diseases in which inhibiting the activity of ROCK1 and/or ROCK2 has or plays a therapeutically useful role.

Accordingly, in some embodiments, the present disclosure provides pharmaceutical compositions comprising a compound of Formula I as described herein (or a prodrug, pharmaceutically acceptable salt or other pharmaceutically acceptable derivative thereof), and optionally comprise a pharmaceutically acceptable carrier. In certain embodiments, provided compositions optionally further comprise one or more additional therapeutic agents. Alternatively, a compound or composition described herein may be administered to a patient in need thereof in combination with the administration of one or more other therapeutic agents. For example, additional therapeutic agents for conjoint administration or inclusion in a pharmaceutical composition with a compound described herein may be an approved agent to treat the same or related indication, or it may be any one of a number of agents undergoing review and/or approval in the Food and Drug Administration that ultimately obtain approval for the treatment of any disorder described herein. It will also be appreciated that certain provided compounds can exist in free form (e.g., as a free base or free acid), or where appropriate, as a pharmaceutically acceptable derivative thereof. According to the present disclosure, a pharmaceutically acceptable derivative includes, but is not limited to, pharmaceutically acceptable salts, esters, salts of such esters, or a pro-drug or other adduct or derivative of a compound of described herein which, upon administration to a patient in need thereof, is capable of providing, directly or indirectly, a compound as otherwise described herein, or a metabolite or residue thereof.

As used herein with reference to compounds of Formula I and subgenera thereof, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of compounds of Formula I and subgenera thereof, or separately by reacting a free base or free acid functional group with a suitable reagent, as described generally below. For example, a free base functional group can be reacted with a suitable acid. Furthermore, where the compounds of Formula I and subgenera thereof carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of provided compounds which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the disclosure. The term “prodrug” refers to compounds that are rapidly transformed in vivo to yield the parent compound of the above formula, for example by hydrolysis in blood, or N-demethylation of a compound of the disclosure where R¹ is methyl. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As described above, the pharmaceutical compositions of the present disclosure additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutical composition, its use is contemplated to be within the scope of this disclosure. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut (peanut), corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension or crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyethynylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose and starch. Such dosage forms may also comprise, as in normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The present disclosure encompasses pharmaceutically acceptable topical formulations of provided compounds. The term “pharmaceutically acceptable topical formulation”, as used herein, means any formulation which is pharmaceutically acceptable for intradermal administration of a compound of the disclosure by application of the formulation to the epidermis. In certain embodiments of the disclosure, the topical formulation comprises a carrier system. Pharmaceutically effective carriers include, but are not limited to, solvents (e.g., alcohols, poly alcohols, water), creams, lotions, ointments, oils, plasters, liposomes, powders, emulsions, microemulsions, and buffered solutions (e.g., hypotonic or buffered saline) or any other carrier known in the art for topically administering pharmaceuticals. A more complete listing of art-known carriers is provided by reference texts that are standard in the art, for example, Remington's Pharmaceutical Sciences, 16th Edition, 1980 and 17th Edition, 1985, both published by Mack Publishing Company, Easton, Pa., the disclosures of which are incorporated herein by reference in their entireties. In certain other embodiments, the topical formulations described herein may comprise excipients. Any pharmaceutically acceptable excipient known in the art may be used to prepare pharmaceutically acceptable topical formulations. Examples of excipients that can be included in the topical formulations of the disclosure include, but are not limited to, preservatives, antioxidants, moisturizers, emollients, buffering agents, solubilizing agents, other penetration agents, skin protectants, surfactants, and propellants, and/or additional therapeutic agents used in combination with one or more provided compounds. Suitable preservatives include, but are not limited to, alcohols, quaternary amines, organic acids, parabens, and phenols. Suitable antioxidants include, but are not limited to, ascorbic acid and its esters, sodium bisulfite, butylated hydroxytoluene, butylated hydroxyanisole, tocopherols, and chelating agents like EDTA and citric acid. Suitable moisturizers include, but are not limited to, glycerin, sorbitol, polyethylene glycols, urea, and propylene glycol. Suitable buffering agents for use with the disclosure include, but are not limited to, citric, hydrochloric, and lactic acid buffers. Suitable solubilizing agents include, but are not limited to, quaternary ammonium chlorides, cyclodextrins, benzyl benzoate, lecithin, and polysorbates. Suitable skin protectants that can be used in the topical formulations of the disclosure include, but are not limited to, vitamin E oil, allatoin, dimethicone, glycerin, petrolatum, and zinc oxide.

In certain embodiments, the pharmaceutically acceptable topical formulations described herein comprise at least a compound of the disclosure and a penetration enhancing agent. The choice of topical formulation will depend or several factors, including the condition to be treated, the physicochemical characteristics of provided compounds and other excipients present, their stability in the formulation, available manufacturing equipment, and costs constraints. As used herein the term “penetration enhancing agent” means an agent capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). In certain exemplary embodiments, penetration agents for use with the disclosure include, but are not limited to, triglycerides (e.g., soybean oil), aloe compositions (e.g., aloe-vera gel), ethyl alcohol, isopropyl alcohol, octylphenylpolyethylene glycol (e.g., Triton X-100), oleic acid, polyethylene glycol 400, propylene glycol, N-decylmethylsulfoxide, fatty acid esters (e.g., isopropyl myristate, methyl laurate, glycerol monooleate, and propylene glycol monooleate) and N-methyl pyrrolidone.

In certain embodiments, the compositions may be in the form of ointments, pastes, creams, lotions, gels, powders, solutions, sprays, inhalants or patches. In certain exemplary embodiments, formulations of the compositions described herein are creams, which may further contain saturated or unsaturated fatty acids such as stearic acid, palmitic acid, oleic acid, palmito-oleic acid, cetyl or oleyl alcohols, stearic acid being particularly preferred. Creams described herein may also contain a non-ionic surfactant, for example, polyoxy-40-stearate. In certain embodiments, the active component is admixed under sterile conditions with a pharmaceutically acceptable carrier and any needed preservatives or buffers as may be required. Ophthalmic formulation, eardrops, and eye drops are also contemplated as being within the scope of this disclosure. Formulations for intraocular administration are also included. Additionally, the present disclosure contemplates the use of transdermal patches, which have the added advantage of providing controlled delivery of a compound to the body. Such dosage forms are made by dissolving or dispensing the compound in the proper medium. As discussed above, penetration enhancing agents can also be used to increase the flux of the compound across the skin. The rate can be controlled by either providing a rate controlling membrane or by dispersing the compound in a polymer matrix or gel.

It will also be appreciated that the compounds and pharmaceutical compositions described herein can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a provided compound may be administered concurrently with another anti-inflammatory agent), or they may achieve different effects (e.g., control of any adverse effects). In non-limiting examples, one or more compounds described herein may be formulated with at least one cytokine, growth factor or other biological, such as an interferon, e.g., alpha interferon, or with at least another small molecule compound. Non-limiting examples of pharmaceutical agents that may be combined therapeutically with compounds of the present disclosure include: antivirals and antifibrotics such as interferon alpha, combination of interferon alpha and ribavirin, Lamivudine, Adefovir dipivoxil and interferon gamma; anticoagulants such as heparin and warfarin; antiplatelets e.g., aspirin, ticlopidine and clopidogrel; other growth factors involved in regeneration, e.g., VEGF and FGF and mimetics of these growth factors; antiapoptotic agents; and motility and morphogenic agents.

In certain embodiments, the pharmaceutical compositions described herein further comprise one or more additional therapeutically active ingredients (e.g., anti-inflammatory and/or palliative). For purposes of the present disclosure, the term “Palliative” refers to treatment that is focused on the relief of symptoms of a disease and/or side effects of a therapeutic regimen, but is not curative. For example, palliative treatment encompasses painkillers, antinausea medications and anti-sickness drugs.

Research Uses, Clinical Uses, Pharmaceutical Uses and Methods of Treatment

Research Uses

According to the present disclosure, provided compounds may be assayed in any of the available assays known in the art for identifying compounds having the ability to modulate the activity of ROCK1 and/or ROCK2 and in particular to antagonize the activity of ROCK1 and/or ROCK2. For example, the assay may be cellular or non-cellular, in vivo or in vitro, high- or low-throughput format, etc.

Thus, in one aspect, preferred compounds disclosed herein include those which inhibit the activity of ROCK1 and/or ROCK2.

Clinical Uses of Compounds that Inhibit the Activity of ROCK1 and/or ROCK2

Hepatic Disease

Fibrotic Liver Disease: Liver fibrosis is the scarring response of the liver to chronic liver injury; when fibrosis progresses to cirrhosis, morbid complications can develop. In fact, end-stage liver fibrosis or cirrhosis is the seventh leading cause of death in the United States, and afflicts hundreds of millions of people worldwide; deaths from end-stage liver disease in the United States are expected to triple over the next 10-15 years, mainly due to the hepatitis C epidemic. In addition to the hepatitis C virus, many other forms of chronic liver injury also lead to end-stage liver disease and cirrhosis, including other viruses such as hepatitis B and delta hepatitis, chronic alcoholism, non-alcoholic steatohepatitis (NASH), extrahepatic obstructions (stones in the bile duct), cholangiopathies (primary biliary cirrhosis and sclerosing cholangitis), autoimmune liver disease, and inherited metabolic disorders (Wilson's disease, hemochromatosis, and alpha-1 antitrypsin deficiency).

Treatment of liver fibrosis has focused to date on eliminating the primary injury. For extrahepatic obstructions, biliary decompression is the recommended mode of treatment whereas patients with Wilson's disease are treated with zinc acetate. In chronic hepatitis C infection, interferon has been used as antiviral therapies with limited response: ˜20% when used alone or ˜50% response when used in combination with ribavirin. In addition to the low-level of response, treatment with interferon with or without ribavirin is associated with numerous severe side effects including neutropenia, thrombocytopenia, anemia, depression, generalized fatigue and flu-like symptoms, which are sufficiently significant to necessitate cessation of therapy. Treatments for other chronic liver diseases such as hepatitis B, autoimmune hepatitis and Wilson's disease are also associated with many side effects, while primary biliary cirrhosis, primary sclerosing cholangitis and non-alcoholic fatty liver disease have no effective treatment other than liver transplantation.

The advantage of treating fibrosis rather than only the underlying etiology is that antifibrotic therapies should be broadly applicable across the full spectrum of chronic liver diseases. While transplantation is currently the most effective cure for liver fibrosis, mounting evidence indicates that not only fibrosis, but even cirrhosis is reversible. Unfortunately, patients often present with advanced stages of fibrosis and cirrhosis, when many therapies such as antivirals can no longer be safely used due to their side effect profile. Such patients would benefit enormously from effective antifibrotic therapy, because attenuating or reversing fibrosis may prevent many late stage complications such as infection, ascites, and loss of liver function and preclude the need for liver transplantation. The compounds disclosed herein are beneficial for the treatment of the foregoing conditions, and generally are antifibrotic and/or antiapoptotic agents for this and other organ or tissues.

Hepatic Ischemia-Reperfusion Injury: Currently, transplantation is the most effective therapeutic strategy for liver fibrosis. However, in spite of the significant improvement in clinical outcome during the last decade, liver dysfunction or failure is still a significant clinical problem after transplantation surgery. Ischemia-reperfusion (IR) injury to the liver is a major alloantigen-independent component affecting transplantation outcome, causing up to 10% of early organ failure, and leading to the higher incidence of both acute and chronic rejection. Furthermore, given the dramatic organ shortage for transplantation, surgeons are forced to consider cadaveric or steatotic grafts or other marginal livers, which have a higher susceptibility to reperfusion injury. In addition to transplantation surgery, liver IR injury is manifested in clinical situations such as tissue resections (Pringle maneuver), and hemorrhagic shock.

The damage to the postischemic liver represents a continuum of processes that culminate in hepatocellular injury. Ischemia activates Kupffer cells, which are the main sources of vascular reactive oxygen species (ROS) formation during the initial reperfusion period. In addition to Kupffer cell-induced oxidant stress, with increasing length of the ischemic episode, intracellular generation of ROS by xanthine oxidase and in particular mitochondria may also contribute to liver dysfunction and cell injury during reperfusion. Endogenous antioxidant compounds, such as superoxide dismutase, catalase, glutathione, alpha-tocopherol, and beta-carotene, may limit the effects of oxidant injury but these systems can quickly become overwhelmed by large quantities of ROS. Work by Lemasters and colleagues, has indicated that, in addition to formation of ROS, intracellular calcium dyshomeostasis is a key contributor to liver IR injury. Cell death of hepatocytes and endothelial cells in this setting is characterized by swelling of cells and their organelles, release of cell contents, eosinophilia, karyolysis, and induction of inflammation, characteristic of oncotic necrosis. More recent reports indicate that liver cells also die by apoptosis, which is morphologically characterized by cell shrinkage, formation of apoptotic bodies with intact cell organelles and absence of an inflammatory response.

Indeed, minimizing the adverse effects of IR injury could significantly increase the number of patients that may successfully undergo liver transplantation. Pharmacologic interventions that reduce cell death and/or enhance organ regeneration represent a therapeutic approach to improve clinical outcome in liver transplantation, liver surgery with vascular exclusion and trauma and can therefore reduce recipient/patient morbidity and mortality. The compounds disclosed herein are beneficial for the treatment of the foregoing conditions.

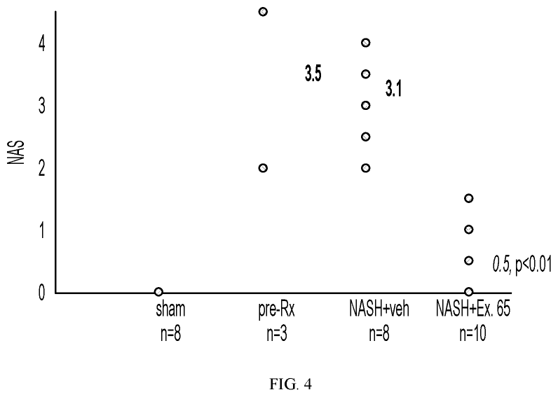

Cirrhotic or Non-Cirrhotic Hepatocellular Carcinoma (HCC): Since 1980, HCC incidence rates have more than tripled and death rates more than doubled in part due to nonalcoholic fatty liver disease (NAFLD) (Global Cancer Facts and Figures 4th Edition; http://www.definedhealth.com/the-dash-to-treat-nash-the-next-big-global-epidemic/; last accessed Oct. 18, 2020; Tesfay M., Goldkamp W J., and Neuschwander-Tetri B. A. Missouri Med. 2018; 115:225-229; Sanyal A. J, et al. Hepatology 2015; 61:1392-1405). With the recent rise in diabetes, obesity, and metabolic syndrome, NAFLD has reached epidemic proportions. Within the United States alone, NAFLD-associated complications are predicted to become the primary cause for liver transplant over the next few decades. The threat associated with NAFLD lies in more than just in its risk for progression to NASH, wherein hepatic inflammation accompanies steatosis. Left untreated, the NAFLD continuum encompasses fibrosis or scarring, cirrhosis, and hepatic decompensation. Cirrhosis, regardless of etiology, is a known risk factor for HCC. Emerging evidence indicates that NASH, with or without fibrosis, can progress to HCC, a phenomenon known as noncirrhotic HCC (Hwang A., et al. PLoS ONE 2018; 13:e0198937; Bissoondial T. L., et al. Diagnostics 2020; 10:784; Liao K., et al. ACS Omega 2020; 5:18465-18471). Multiple clinical studies have reported a subset of NASH subjects presenting with non-cirrhotic HCC (Friedman S. L. J. Hepatol 2014; 60:1-2; Perumpail R. B., et al. Dig. Dis. Sci. 2015; 60:3142-3148; Ertle J., et al. Int. J. Cancer 2011; 128:2436-2443; Kolly P. and Dufour J. F. Diagnostics 2016; 6:22; Cholankeril G., et al. World J. Hepatol. 2017; 9:533-543).

HCC remains a treatment challenge due to late detection and resistance to currently approved drugs. While a small proportion of HCC patients diagnosed at an early stage can be treated by tumor resection, cryoablation or liver transplant, these treatments are not effective or even feasible in the majority of HCC patients diagnosed at an advanced stage of the disease (Kolly P. and Dufour J. F. Diagnostics 2016; 6:22; Cholankeril G., et al. World J. Hepatol. 2017; 9:533-543; Bruix J., Sherman M. Hepatology. 2011; 53(3):1020-1022. pmid:21374666; Heimbach J. K., et al. Hepatology. 2018; 67(1):358-380. pmid:28130846). When diagnosed late, HCC is almost always fatal. The highly vascularized hepatic bed facilitates tumor growth while making tumor resection extremely challenging. In fact, Barcelona Clinic Liver Cancer stages B-D are deemed noncurative. Sorafenib is the most widely used drug in treating such patients (Regan-Fendt K., et al. Cancers (Basel) 2020 Sep. 23; 12(10):2730; Qiu Y., et al. Cell Death Discovery (2019); 5:120; Tang W., et al. Signal Transduction and Targeted Therapy (2020)5:87). However, sorafenib extends median survival in this patient population by less than 3 months. This minimal therapeutic response is attributed to HCC tumors having an intrinsic resistance to the cytostatic effects of sorafenib including expression of micro(mi)RNA-181a. For HCC patients who become resistant to sorafenib, second-line treatment, such as regorafenib and checkpoint blockade anti-PD-1 antibodies, nivolumab and pembrolizumab, may be considered. However, only a subset of such patients respond to this combination therapy. In recent clinical trials in the first-line setting, nivolumab or pembrolizumab could not significantly improve survival of HCC patients compared to sorafenib and best supportive care, respectively. Lenvatinib, recently approved as first-line therapy for advanced HCCs, was not significantly superior to sorafenib in improving overall survival. Thus, there is an urgent need to develop strategies to overcome sorafenib resistance and discover new, more effective therapies for HCC.

The compounds disclosed herein are beneficial for the treatment of the foregoing hepatic diseases and/or conditions.

Cerebral and/or Cerebrovascular Disease

Cerebral Infarction: Stroke and cerebrovascular disease are a leading cause of morbidity and mortality in the US: at least 600,000 Americans develop strokes each year, and about 160,000 of these are fatal. Research on the pathophysiological basis of stroke has produced new paradigms for prevention and treatment, but translation of these approaches into improved clinical outcomes has proved to be painfully slow. Preventive strategies focus primarily on reducing or controlling risk factors such as diabetes, hypertension, cardiovascular disease, and lifestyle; in patients with severe stenosis, carotid endarterectomy may be indicated. Cerebral angioplasty is used investigationally, but the high restenosis rates observed following coronary angioplasty suggest this approach may pose unacceptable risk for many patients. Therapeutic strategies focus primarily on acute treatment to reduce injury in the ischemic penumbra, the region of reversibly damaged tissue surrounding an infarct. Thrombolytic therapy has been shown to improve perfusion to the ischemic penumbra, but it must be administered within three hours of the onset of infarction. Several neuroprotective agents that block specific tissue responses to ischemia are promising, but none have yet been approved for clinical use. While these therapeutic approaches limit damage in the ischemic penumbra, they do not address the underlying problem of inadequate blood supply due to occluded arteries. An alternative strategy is to induce formation of collateral blood vessels in the ischemic region; this occurs naturally in chronic ischemic conditions, but stimulation of vascularization via therapeutic angiogenesis has potential therapeutic benefit.

Recent advances in imaging have confirmed the pathophysiological basis of the clinical observations of evolving stroke. Analysis of impaired cerebral blood flow (CBF) in the region of an arterial occlusion supports the hypothesis that a central region of very low CBF, the ischemic core, is irreversibly damaged, but damage in surrounding or intermixed zones where CBF is of less severely reduced, the ischemic penumbra, can be limited by timely reperfusion. Plate recently reviewed the evidence suggesting that therapeutic angiogenesis may be useful for treatment or prevention of stroke. Analysis of cerebral vasculature in stroke patients showed a strong correlation between blood vessel density and survival and a higher density of microvessels in the ischemic hemisphere compared to the contralateral region.

The compounds disclosed herein are beneficial for the treatment of the foregoing cerebral and/or cerebrovascular diseases and/or conditions.

Cardiac and/or Cardiovascular Disease

Ischemic heart disease: Heart disease, in particular, ischemic heart disease, is a leading cause of morbidity and mortality in the US, afflicting millions of Americans each year at a cost expected to exceed $300 billion/year. Numerous pharmacological and interventional approaches are being developed to improve treatment of ischemic heart disease including reduction of modifiable risk factors, improved revascularization procedures, and therapies to halt progression and/or induce regression of atherosclerosis. One of the most exciting areas of research for the treatment of myocardial ischemia is therapeutic angiogenesis. Recent studies support the concept that administration of angiogenic growth factors, either by gene transfer or as a recombinant protein, augments nutrient perfusion through neovascularization. The newly developed, supplemental collateral blood vessels constitute endogenous bypass conduits around occluded native arteries, improving perfusion to ischemic tissue.

The compounds disclosed herein are beneficial for the treatment of the foregoing cardiac and/or cardiovascular diseases and/or conditions.

Renal Disease

Chronic renal dysfunction: Chronic kidney disease (CKD) (also referred to as chronic renal dysfunction) is a progressive, degenerative disorder that ultimately results in acute renal failure and requires dialysis as an intervention; renal transplantation is the only potential cure. Initiating conditions of renal dysfunction include ischemia, diabetes, underlying cardiovascular disease, or renal toxicity associated with certain chemotherapeutics, antibiotics, and radiocontrast agents. Most end-stage pathological changes include extensive fibrinogenesis, epithelial atrophy, and inflammatory cell infiltration into the kidneys.

Acute kidney injury: Acute renal failure, or acute kidney injury (AKI), is often a complication of diseases including diabetes or renal ischemia, procedures such as heminephrectomy, or as a side effect of therapeutics administered to treat disease. The widely prescribed anti-tumor drug cis-diamminedichloroplatinum (cisplatin), for example, has side effects that include a high incidence of nephrotoxicity and renal dysfunction, mainly in the form of renal tubular damage that leads to impaired glomerular filtration. Administration of gentamicin, an aminoglycoside antibiotic, or cyclosporin A, a potent immunosuppressive compound, causes similar nephrotoxicity. The serious side effects of these effective drugs restrict their use. The development of agents that protect renal function and enhance renal regeneration after administration of nephrotoxic drugs will be of substantial benefit to numerous patients, especially those with malignant tumors, and may allow the maximal therapeutic potentials of these drugs to be realized.

AKI-related CKD: There has been increasing recognition that CKD and AKI are two closely linked and interconnected renal diseases (Hsu R. K., Hsu C.-Y. Semin Nephrol. 2016 July; 36(4):283-92). The severity of CKD (e.g., as measured by levels of glomerular filtration rate (GFR) and proteinuria) has been shown to be associated with the development of AKI. Clinical and experimental evidence also suggests that AKI can initiate the development of and/or accelerate the progression of CKD (Hsu R. K., Hsu C. Y. Semin Nephrol. 2016 July; 36(4):283-292; He L., et al. Kidney Int. 2017 November; 92(5):1071-1083; Sanoff S., Okusa M. D. Contrib. Nephrol. 2011; 171:213-217). AKI is found to have an independent and graded association with progression to end-stage renal disease (ESRD) in patients with acute myocardial infarction (Newsome B. B., et al. Arch. Intern. Med. 2008; 168(6):609-616). Patients with AKI have significantly higher risks of developing CKD and ESRD and higher mortality as compared to patients without AKI. Increased severity of AKI is often associated with a higher risk for CKD or ESRD (Coca S. G., et al. Kidney Int. 2012; 81(5):442-448).

Studies have demonstrated that either a single episode of severe AKI or repeating episodes of less severe AKI can result in CKD (He L., et al. Kidney Int. 2017 November; 92(5):1071-1083; Fu Y., et al. Am. J. Physiol. Renal Physiol. 2018 Oct. 1; 315(4):F1098-F1106; Black L. M., et al. Am. J. Physiol. Renal Physiol. 2018 Oct. 1; 315(4):F1107-F1118). Renal ischemia-reperfusion- or nephrotoxin-related AKI can lead to renal interstitial fibrosis associated with gradual loss of renal function over time, a characteristic of CKD. In rodent studies, markers of CKD (e.g., proteinuria and renal α-smooth muscle actin (αSMA)) appear as early as three weeks after incident of AKI. Without wishing to be bound by any particular theory, the AKI-CKD transition is believed to be a complex interplay between injured or stressed renal tubular cells, interstitial fibroblasts, the vasculature, and the immune system. After AKI, humoral factors from regenerating tubules as well as inflammatory cells (e.g., monocytes, lymphocytes, and dendritic cells) activate interstitial precursor cells that become (myo)fibroblasts, which proliferate and produce connective tissue. The majority of resident precursors, pericytes or fibroblasts, are cells with branching processes that contact capillaries and tubules. Two important effects ensue: (1) platelet-derived growth factor (PDGFR) B-mediated migration and transformation of pericytes or fibroblasts to αSMA-expressing myofibroblasts and (2) dysangiogenic vascular endothelial growth factor (VEGF) signaling, causing loss of endothelial integrity attributable to loss of the nursing function of pericytes that stabilize capillaries. With continued activation, the interstitium becomes widened by proliferating myofibroblasts and connective tissue, and the injured endothelium regresses, causing capillary rarefaction. It has been postulated that tubules undergoing pathologic growth arrest during regeneration after AKI fail to differentiate, signal vicariously through multiple profibrotic pathways, and secrete fibrogenic peptides into the interstitium, instigating fibrosis. Underlying this hypothesis is the activation of signaling pathways required for dedifferentiation, migration, and proliferation after AKI. For this purpose, the surviving epithelium produces and secretes growth factors, cytokines, and autacoids. Although these signaling and secretory events are required for normal regeneration, they should cease when tubules recover. Persistence of proliferative signaling in growth-arrested undifferentiated epithelium undergoing atrophy is inherently abnormal. Atrophic tubules are engaged in pathologically increased signaling through increased expression of profibrotic moieties (e.g., PDGF-B, connective tissue growth factor (CTGF), and transforming growth factor (TGF)β). Without wishing to be bound by any particular theory, AKI is often associated with maladaptive repair such as tubular cell cycle arrest, tubular loss, profibrogenic cytokine production, pericyte-myofibroblast transition and interstitial matrix deposition and long-term dysfunction.

Renal Fibrosis: Several growth factor receptors have been implicated in the development of fibrosis of the kidney (Liu, F., et al. Int. J. Mol. Sci. 2016 Jun. 20; 17(5), PMCID:PMC4926504). Platelet-derived growth factor receptor beta (PDGFRβ) is postulated to play a particularly important role in the development of renal fibrosis (Floege, J., et al. J. Am. Soc. Nephrol. 2008 January; 19(1):12-23; Ostendorf, T., et al. Pediatr. Nephrol. 2012 July; 27(7):1041-50; Ostendorf. T., et al. Kidney Int. Suppl. (2011) 2014 November; 4(1):65-9, PMCID:PMC4536969; Abbound, H. E. Annu. Rev. Physiol. 1995; 57:297-309).

Nephrotic Syndrome (NS): NS is a group of rare renal diseases, including focal and segmental glomerulosclerosis (FSGS), minimal change disease (MCD), and membranous nephropathy. FSGS is a rare disease that attacks the kidney's filtering units (glomeruli) causing serious scarring which leads to permanent kidney damage and even failure (Fogo, A. B. Nat. Rev. Nephrol. 2015 February; 11(2):76-87, PMCID:PMC4772430). It will be appreciated that there are at least three types of FSGS. Primary FSGS is FSGS that has no known cause (also referred to as idiopathic FSGS). Secondary FSGS is caused by one or more factors such as infection, drug toxicity, diseases such as diabetes or sickle cell disease, obesity, or other kidney diseases. Genetic FSGS (also called familial FSGS) is caused by one or more genetic mutations. Primary FSGS is idiopathic in nature. Manifestations of this disease include hypoalbuminemia and edema, lipid abnormalities and nephrotic range proteinuria. More than 5400 patients are diagnosed with FSGS every year (O'Shaughnessy, M. M., et al. Nephrol. Dial. Transplant 2018 Apr. 1; 33(4):661-9). However, this is considered an underestimate because a limited number of biopsies are performed, and the number of FSGS cases is rising more than any other cause of NS. Standard of care for this patient population is steroid therapy. Current treatments for FSGS include corticosteroids, calcineurin inhibitors, mycophenolate mofetil, adrenocorticotropic hormone (ATCH), and rituximab; these are effective in at most 25-40% of patients. A subset of this population is resistant to steroids (steroid-resistant, or SR), and proteinuria, which is toxic to renal tubules, remains uncorrected. Consequently, this subset proceeds relatively rapidly to end-stage renal disease (ESRD). There is therefore an urgent need to develop therapies that reduce proteinuria in primary SR-FSGS (Nourbakhsh, N. and Mak, R. H. Pediatric Health Med. Ther. 2017; 8:29-37, PMCID:PMC5774596).

Minimal Change Disease (MCD): MCD is a kidney disease in which large amounts of protein are lost in the urine. It is one of the most common causes of the nephrotic syndrome worldwide. In children, MCD is usually primary (or idiopathic), but in adults, the disease is usually secondary. Secondary causes for MCD include allergic reactions, use of certain painkillers such as non-steroidal anti-inflammatory drugs (NSAIDs), tumors, or viral infections.

Anti-Neutrophil Cytoplasmic Antibody (ANCA)-Associated Glomerulonephritis: ANCA-associated glomerulonephritis is a rapidly progressive renal disease and includes, e.g., Wegener's granulomatosis, microscopic polyangiitis, and renal limited vasculitis. Wegener's granulomatosis is an organ- and/or life-threatening autoimmune disease of unknown etiology. The classical clinical triad consists of necrotizing granulomatous inflammation of the upper and/or lower respiratory tract, necrotizing glomerulonephritis, and an autoimmune necrotizing systemic vasculitis affecting predominantly small vessels. The detection of anti-neutrophil cytoplasmic antibodies directed against proteinase 3 (PR3-ANCA) is a highly specific indicator for Wegener's granulomatosis. Microscopic polyangiitis is a disorder that causes blood vessel inflammation (vasculitis), which can lead to organ damage. The kidneys, lungs, nerves, skin, and joints are the most commonly affected areas of the body. MPA is diagnosed in people of all ages, all ethnicities, and both genders. The cause of this disorder is unknown. Renal limited vasculitis is a type of anti-neutrophil cytoplasmic antibody (ANCA)-associated vasculitis that presents with only a renal manifestation; no other organs, including lungs, are involved.

Lupus Nephritis: Lupus nephritis is inflammation of the kidney that is caused by an autoimmune disease, systemic lupus erythematous (SLE). With lupus, the body's immune system targets its own body tissues; lupus nephritis occurs when lupus involves the kidneys.

Anti-Globular Basement Membrane (anti-GBM) Nephropathy: Anti-GBM nephropathy is a disease that occurs as a result of injury to small blood vessels (capillaries) in the kidneys and/or lungs. In anti-GBM disease, autoantibodies are targeted to the basement membrane in capillary blood vessels of the kidneys and lung, where they target and damage GBM.

IgA nephropathy: IgA nephropathy, also known as Berger's disease, is a kidney disease that occurs when IgA deposits build up in the kidneys, causing inflammation that damages kidney tissues. IgA nephropathy affects the kidneys by attacking the glomeruli. The buildup of IgA deposits inflames and damages the glomeruli, causing the kidneys to leak blood and protein into the urine. The damage may lead to scarring of the nephrons that progresses slowly over many years. Eventually, IgA nephropathy can lead to end-stage kidney disease.

Alport syndrome (AS): AS is a genetic condition characterized by kidney disease, hearing loss, and eye abnormalities. Most affected individuals experience progressive loss of kidney function, usually resulting in end-stage kidney disease. In 80% of cases, Alport syndrome is inherited in an X-linked manner and is caused by mutation(s) in the COL4A5 gene. In other cases, it can be inherited in either an autosomal recessive, or rarely in an autosomal dominant manner, and is caused by mutation(s) in the COL4A3 and/or COL4A4 genes. Current therapies include hearing aid, hemodialysis, peritoneal dialysis and kidney transplantation.

Polycystic Kidney Disease (e.g., autosomal recessive polycystic kidney disease (ARPKD)-congenital hepatic fibrosis (CHF)): ARPKD-CHF is a highly aggressive fibropolycystic disease that is characterized by the formation and expansion of fluid-filled cysts in the kidneys, enlargement of the kidneys and progressive fibrosis of both the kidney and the liver (Hartung, E. A., and Guay-Woodford, L. M. Pediatrics 2014 September; 134(3):e833-e845; Gunay-Aygun, M., et al. J. Pediatr. 2006 August; 149(2):159-64). Caroli's disease manifests as cystic dilatation of the intrahepatic ducts, often accompanies ARPKD-CHF (Sung, J. M., et al. Clin. Nephrol. 1992 December; 38(6):324-8). In some embodiments, a subject is suffering from, susceptible to, or at risk of Caroli's disease. Afflicted children that survive past two years of age more often than not require renal and/or hepatic transplantation by age ten. The need for transplantation is often driven by both progressive organ dysfunction and by significant enlargement of the diseased organ(s), and is accompanied by severe pain (www.arpkdchf.org).

Renal Cysts: Aberrant signaling by tyrosine kinases, including platelet-derived growth factor (PDGF) and vascular endothelial growth factor (VEGF) and their receptors (R), PDGFR and VEGFR/KDR, respectively, has been implicated in the formation and expansion of renal cysts. A PDGF-driven ciliopathy and/or overexpression of PDGF in the cyst lining and adjacent tubules are thought to, in part, drive renal cystic disease (Torres, V. E., et al. Lancet 2007 Apr. 14; 369(9569):1287-301; Park. J. H. et al. Polycystic Kidney Disease Brisbane; 2015:375-96; Nakamura, T., et al. J. Am. Soc. Nephrol. 1993 January; 3(7):1378-86). Cowley et al. posited that elevated and abnormal c-myc proto-oncogene expression drives ARPKD (Proc. Natl. Acad. Sci. U.S.A. 1987 December:84(23):8394-8); c-myc expression is controlled by PDGF (Frick, K. K., et al. C. J. Biol. Chem. 1988 Feb. 25; 263(6):2948-52).

Collagen Type III Glomerulopathy: Collagen type III glomerulopathy, also known as collagenic or collagenofibrotic glomerulopathy, is characterized by pathological accumulation of collagen type III in glomeruli. Collagen type III glomerulopathy presents either in childhood, often with a family history suggesting autosomal recessive inheritance, or in adults as a sporadic occurrence. Proteinuria is a typical manifestation, with progression to end stage renal disease (ESRD) in approximately 10 years. Although there is markedly elevated serum precursor collagen type III protein in the circulation, the usual manner of diagnosis is with kidney biopsy, which discloses type III collagen in subendothelial aspects of capillary walls and often in the mesangial matrix.

Nail-patella syndrome: Nail-patella syndrome is a multi-organ disorder caused by mutations in the LMX1B gene. Nail-patella syndrome manifests with orthopedic and cutaneous deformities, as well as kidney complications due to development of structural lesions of collagen type III within glomerular basement membranes. Although the structural lesions may be asymptomatic, they are usually accompanied by proteinuria.

The compounds disclosed herein are beneficial for the treatment of the foregoing renal diseases and/or conditions.

Pulmonary Disease

Lung (Pulmonary) Fibrosis: Idiopathic pulmonary fibrosis (IPF) accounts for a majority of chronic interstitial lung diseases, and has an estimated incidence rate of 10.7 cases for 100,000 per year, with an estimated mortality of 50-70%. IPF is characterized by an abnormal deposition of collagen in the lung with an unknown etiology. Although the precise sequence of the pathogenic sequelae is unknown, disease progression involves epithelial injury and activation, formation of distinctive subepithelial fibroblast/myofibroblast foci, and excessive extracellular matrix accumulation. The development of this pathological process is preceded by an inflammatory response, often dominated by macrophages and lymphocytes, which is mediated by the local release of chemoattractant factors and upregulation of cell-surface adhesion molecules. Lung injury leads to vasodilation and leakage of plasma proteins into interstitial and alveolar spaces, as well as activation of the coagulation cascade and deposition of fibrin. Fibroblasts migrate into this provisional fibrin matrix where they synthesize extracellular matrix molecules. In non-pathogenic conditions, excess fibrin is usually degraded by plasmin, a proteinase that also has a role in the activation of matrix metalloproteinases (MMPs). Activated MMPs degrade extracellular matrix and participate in fibrin removal, resulting in the clearance of the alveolar spaces and the ultimate restoration of injured tissues. In pathological conditions, however, these processes can lead to progressive and irreversible changes in lung architecture, resulting in progressive respiratory insufficiency and an almost universally terminal outcome in a relatively short period of time. Fibrosis is the final common pathway of a variety of lung disorders, and in this context, the diagnosis of pulmonary fibrosis implies the recognition of an advanced stage in the evolution of a complex process of abnormal repair. While many studies have focused on inflammatory mechanisms for initiating the fibrotic response, the synthesis and degradation of the extracellular matrix represent the central event of the disease. It is this process that presents a very attractive site of therapeutic intervention.

The course of IPF is characterized by progressive respiratory insufficiency, leading to death within 3 to 8 years from the onset of symptoms. Management of interstitial lung disease in general, and in particular idiopathic pulmonary fibrosis, is difficult, unpredictable and unsatisfactory. Attempts have been made to use anti-inflammatory therapy to reverse inflammation, relief, stop disease progression and prolong survival. Corticosteroids are the most frequently used anti-inflammatory agents and have been the mainstay of therapy for IPF for more than four decades, but the efficacy of this approach is unproven, and toxicities are substantial. No studies have compared differing dosages or duration of corticosteroid treatment in matched patients. Interpretation of therapy efficacy is obscured by several factors including heterogeneous patient populations, inclusion of patients with histologic entities other than usual interstitial pneumonia, lack of objective, validated endpoints, and different criteria for “response.” Cytotoxic drugs such as Azathioprine and cyclophosphamide have also being used in combination with low dose oral corticosteroids. The results of such treatments vary from no improvement to significant prolongation of survival. Overall, currently available treatments for lung fibrosis are sub-optimal. Potential new therapies have emerged from the use of animal models of pulmonary fibrosis and recent advances in the cellular and molecular biology of inflammatory reactions. Such therapies involve the use of cytokines, oxidants and growth factors that are elaborated during the fibrotic reaction. Despite the use of newer strategies for treatment, the overall prognosis for patients with interstitial lung disease has had little quantifiable change, and the population survival remains unchanged for the last 30 years. Interferon gamma (IFN) may be effective in the treatment of IPF in some patients but its role is controversial. Literature indicated that IFN-gamma may be involved in small airway disease in silicotic lung. Others showed that IFN gamma mediates, bleomycin-induced pulmonary inflammation and fibrosis.

The compounds disclosed herein are beneficial for the treatment of the foregoing pulmonary diseases and/or conditions.

Dermal Disease

Scleroderma and/or systemic sclerosis (scleroderma/SSc): Scleroderma, which literally means hard skin, is a chronic fibrotic disorder of unknown etiology that affects the skin and other internal organs (SSc) (www.scleroderma.org). Many patients who suffer from scleroderma/SSc also have loss of lung function. Scleroderma/SSc and related diseases afflict approximately 400,000 to 990,000 people in the USA every year. Mortality and morbidity in scleroderma/SSc are very high and directly related to the extent of fibrosis of the involved organs (Hinchcliff, M. and Varga, J. Am. Fam. Physician 2008 October; 78(8):961-8). A number of international studies suggest that scleroderma/SSc occurs much more frequently in the USA than elsewhere and that it occurs three to four times more frequently among women (Mayes, M. D., et al. Arthritis Rheum. 2003 August; 48(8):2246-55).

According to several studies, the total economic cost of scleroderma/SSc in the USA reaches $1.5 billion annually. Morbidity represents the major cost burden, associated with $820 million (55%) of total cost. The high cost of scleroderma/SSc reflects the burden of chronic disease affecting an early age of disease onset and its high morbidity (Wilson, L. Semin. Arthritis Rheum. 1997 October; 27(2):73-84). Hence, there is a critical need for effective and affordable therapies.

Scleroderma/SSc can be classified in terms of the degree and location of the skin involvement and has been categorized into two major groups—diffuse and limited. The diffuse form of scleroderma/SSc involves symmetric thickening of skin of the extremities, face and trunk. Organs affected include the esophagus, intestines, lungs, heart, and kidneys (Mayes, M. D. Semin. Cutan. Med. Surg. 1998 March; 17(1):22-6; Jacobsen, L. et al. J. Am. Acad. Dermatol. 2003 August; 49(2):323-5). The limited form of scleroderma/SSc tends to be confined to the skin of fingers and face. The limited form of scleroderma/SSc is the CREST_variant of scleroderma/SSc based on the clinical pattern of calcinosis with tiny deposits of calcium in the skin, Raynaud's phenomenon in the fingers, toes, nose, tongue, or ears, poor functioning of muscle of esophagus, sclerodactyly of the skin of the fingers or toes, and telangiectasias on the face, hands and mouth (Winterbauer, R. H. Bull. Johns Hopkins Hospital 1964; 114:361-83; Wollheim, F. A. Classification of systemic sclerosis. Visions and reality. Rheumatology (Oxford) 2005).

Multiple fibrotic pathways are activated in scleroderma/SSc for reasons that are not completely understood. The pathogenesis of fibrosis in scleroderma/SSc involves a complex set of interactions involving immune activation, microvascular damage and the activation of fibroblasts. Scleroderma/SSc is characterized by excessive deposition of collagen in the skin and other involved organs and abnormalities of blood vessels (Jimenez, S. A., et al. Rheum. Dis. Clin. North Am. 1996 November; 22(4):647-74; Sakkas, L. I. Autoimmunity 2005 March; 38(2):113-6). TGFβ1, a multifunctional cytokine, is an indirect mitogen for human fibroblasts, which through upregulating PDGF, is capable of inducing normal fibroblasts into a pathogenic myofibroblast phenotype that mediates ECM (collagen) accumulation (Mauch, C., et al. J. Invest. Dermatol. 1993 January; 100(1):92S-96S; Hummers, L. K., et al. J. Rheumatol. 2009 March; 36(3):576-82). The ubiquitous growth factors TGFβ and PDGF are the most potent proteins involved in fibroblast proliferation, collagen gene expression and connective tissue (collagen) accumulation (Antoniades, H. N. Baillieres Clin. Endocrinol. Metab. 1991 December; 5(4):595-613). Numerous other cytokines including VEGF, as well as cell-matrix interactions, also modify collagen expression and can influence the effects of TGFβ1 and PDGF (Trojanowska, M. Rheumatology (Oxford) 2008 October; 47 Suppl 5:v2-4). Persistent overproduction of collagen and other connective tissue results in excessive accumulation of ECM components leading to the formation of scar tissue (fibrosis) in the skin and other organs and is responsible for the progressive nature of scleroderma/SSc (Mauch, C. Rheum. Dis. Clin. North Am. 1990 February; 16(1):93-107). This leads to thickness and firmness of involved areas. Overall, the pathogenic cascade at different stages of scleroderma/SSc may have autoimmune, inflammatory, fibrotic and vascular components with systemic fibrosis and vasculopathy. Studies indicate that severe fibrosis and abnormal vascular remodeling were detected and the systemic vasculopathy is a hallmark in the pathogenesis of scleroderma/SSc (Yamamoto, T. Autoimmune mechanisms of scleroderma and a role of oxidative stress. 2011 January; 2(1):4-10).

Other findings suggest that the pathology of scleroderma/SSc is driven by PDGF, and elevated expression of PDGF and its receptors have been found in scleroderma skin and lung tissues (Mauch 1993). Studies indicate that abnormal vascular remodeling with significant elevations of VEGF and PDGF in SSc patients and systemic vasculopathy is the most striking feature of SSc (Ou, X. M., et al. Int. Immunopharmacol. 2009 January; 9(1):70-9; Pytel, D., et al. Anticancer Agents Med. Chem. 2009 January; 9(1):66-76). PDGF and VEGF, together with their cognate receptors, have been shown to be upregulated in the skin of SSc patients.

The clinical management of patients with scleroderma/SSc remains a challenge and involves several therapeutic approaches. Methotrexate, cyclophosphamide, calcium channel blockers, ACE inhibitors, prostacyclin analogues and D-penicillamine are the most widely studied treatments for SSc. IV gamma globulins, mycophenolate mophetil, rituximab, fluoxetine, pirfenidone, relaxin, halofuginone, and anti-TGF-beta antibodies await more solid data, and side effects are common (Sapadin, A. N., et al. Arch. Dermatol. 2002 January; 138(1):99-105; Stummvoll G. H. Acta Med. Austriaca 2002; 29(1):14-9; Zandman-Goddard, G., et al. Clin. Dev. Immunol. 2005; 12(3):165-73; Grassegger, A., et al. Clin. Exp. Dermatol. 2004 November; 29(6):584-8; Nash, R. A., et al. Blood 2007; 110(4):1388-96; Gavino, E. S. and Furst D. E. BioDrugs 2001; 15(9):609-14; Au, K., et al. Curr. Rhemuatol. Rep. 2009 April; 11(2):111). A combination of immunosuppressive agents and imatinib was tested in SSc patients for treating SSc-related lung disease (Kay, J. Arthritis Rheum. 2008 August; 58(8):2543-8; Sabnani, I. Rheumatology (Oxford) 2009 January; 58(1):49-52). Overall, the result of current research is mixed, with limited positive reports.

The compounds disclosed herein are beneficial for the treatment of the foregoing dermal diseases and/or conditions.

Gastrointestinal Disease

Inflammatory bowel disease (IBD): IBD is an inflammatory condition that comprises both ulcerative colitis (UC) and Crohn's disease (CD). While UC affects the entire colon, CD typically affects the ileum but can occur to any part of GI tract. IBD can manifest as acute or chronic colitis, characterized by recurrent intestinal inflammation accompanied by diarrhea and abdominal pain (Arivarasu, N., et al. Tissue Barriers 2018; 6(2):e1463897; Ponder, A. and Long, M. D. Clin. Epidemiol. 2013; 5:237-47). Recurring bouts of inflammation can lead to tissue remodeling and is a serious presentation in IBD and a major cause of morbidity, often requiring hospitalization and surgical intervention (Wendelsdorf, K., et al. J. Theor. Biol. 2010 Jun. 21; 264(4):1225-39; Fornaro, R., et al. J. Dig. Dis. 2015 October; 16(10):558-67).

Incidence of IBD is increasing worldwide and is an expanding global health problem (Amosy, E., et al. Clin. Med. Insights Gastroenterol. 2013; 6:33-47). An estimated 2.5-3 million people in Europe are affected by IBD (Burisch, J., et al. J. Crohns Colitis 2013 May; 7(4):322-37). According to the Centers for Disease Control and Prevention (CDC), 3.1 million adults in this country were diagnosed with IBD in 2015, a substantial increase from the ˜1.4 million adults diagnosed per 2008 reports (www.cdc.gov/IBD; www.cdc.gov/ibd/pdf/inflammatory-bowel-disease-an-expensive-disease.pdf). IBD accounts for ˜1,300,000 physician visits and ˜92,000 hospitalizations each year in the United States. Of these, 75% patients diagnosed with CD and 25% patients diagnosed with UC and require surgery. Risk factors associated with IBD include environmental, genetic and immunologic factors (Abegunde, A. T., et al. World J. Gastroenterol. 2016 Jul. 21; 22(27):6296-6317; Frolkis, A., et al. Can. J. Gastroenterol. 2013 March; 27(3):e28-24).

IBD is a major cause of morbidity in patients and is a major consumer of the health care budget. A European study estimated that direct healthcare costs for IBD in Europe are ˜5 billion Euros/year (Bursich 2013). In 2008, CDC reports indicate that direct treatment costs with IBD were estimated ˜$6.3 billion and indirect costs were estimated to cost an additional $5.5 billion (www.cdc.gov/IBD). A study in 2017 indicated that the annual direct and indirect costs related to ulcerative colitis (UC) are estimated to be as high as €12.5-29.1 billion in Europe and US$8.1-14.9 billion in the USA (Ungaro, R., et al. Lancet 2017 Apr. 29; 389(10080):1756-1770). Thus, IBD is an expensive disease without cure.

IBD is an autoimmune disease with excessive activation of the adaptive immune response. Various factors including genetic factors alter the intestinal flora and trigger an inflammatory reaction, activate T cells, B cells, mast cells, macrophages and microglia, smooth muscle cells and fibroblasts in the colon, inducing mucosal disruption (Hildner, K., et al. Dig. Dis. 2016; 34Suppl 1:40-7; Curciarello, R., et al. Front Med. (Lausanne) 2017 Aug. 7; 4:126). Epithelial and endothelial damage release chemotactic factors promoting recruitment and activation of inflammatory cells, and release various cytokines including TNFα, and activate fibroblasts via TGFβ1. Activated fibroblasts, i.e. myofibroblasts, secrete growth factors including platelet derived growth factor (PDGF) and vascular endothelial growth factor (VEGF) (Scaldaferri, et al. Gastroenterology 2009 February; 136(2):585-95.e5). Studies indicate that angiogenesis is also an important part of IBD pathogenesis in the colon of IBD patients. In fact, Alkim, et al. demonstrated enhanced microvessel density in the intestinal tissue of both UC and CD patients, which correlated both the level of local VEGF expression and disease activity (Int. J. Inflam. 2015; 2015:970890).

Anti-inflammatory drugs, including 5-aminosalicylic acid (5-ASA)-based preparations, are often the first line of therapy in IBD (Segars, L. W., et al. Clin. Pharm. 1992 June; 11(6):514-28). Anti-TNFα antibodies such as infliximab and adalimumab are also being used. Nevertheless, patients treated with adalimumab are at increased risk for serious infections and lymphoma (Dulai, P. S., et al. Clin. Gastroenterol. Hepatol. 2014 September; 12(9):1443-51). Corticosteroids, other immuno-suppressants, and antibiotics exhibit multiple side effects with relatively poor treatment responses (Kopylov, U., et al. Adv. Gastroenterol. 2016 July:9(4):513-26; Waljee, A. K., et al. PLoS One 2016 Jun. 23; 11(6):e0158017; Cosnes, J., et al. Gut 2005; 54:237-241).

Studies indicate that PDGF and its receptors are highly expressed in areas of ongoing inflammation and fibrosis in IBD (Zeisberg, M. and Kalluri, R. Am. J. Physiol. Cell Physiol. 2013 Feb. 1; 304(3):C216-C225). PDGF activates fibroblasts and IBD-fibroblasts proliferate more rapidly than normal fibroblasts; collagen secretion from IBD patients' fibroblasts was increased compared to collagen secretion by normal fibroblasts. IBD is also associated with increased circulating PDGF and the level of this growth factor has been reported to correspond with disease severity (Andrae, J., et al. Genes Dev. 2008 May 15; 22(10):1276-1312).

Studies indicate that angiogenesis as a novel component of IBD pathogenesis and angiogenic activity is increased in IBD patients. Serum VEGF levels were significantly higher in IBD patients compared to controls in several studies. Griga et al. demonstrated that sources of increased serum VEGF were from inflamed intestinal tissue of IBD patients (Scand. J. Gastroenterol. 1998 May; 33(5):504-8; Hepatogastroenterology 2002 January-February; 49(43):116-23; Hepatogastroenterology 1999 March-April; 46(26):920-3; Eur. J. Gastroenterol. Hepatol. 1999 February; 11(2):175-9). Furthermore, they found VEGF expression was markedly increased in the inflamed mucosa of both CD and UC patients, when compared with normal mucosa of the same patient. Studies also showed that VEGF expression was increased in colon and was higher across all IBD groups (both CD and UC) when compared with healthy controls. Scaldaferri, et al. (2009) reported that VEGF receptor (VEGFR/KDR) levels were increased in intestinal samples of IBD patients, and in mice with experimental colitis.

The compounds disclosed herein are beneficial for the treatment of the foregoing gastrointestinal diseases and/or conditions.

Exemplary Assays

Efficacy of the compounds disclosed herein on the aforementioned disorders and diseases or the potential to be of benefit for the prophylaxis or treatment thereof may be demonstrated in various studies, ranging from biochemical effects evaluated in vitro and effects on cells in culture, to in-vivo models of disease, wherein direct clinical manifestations of the disease can be observed and measured, or wherein early structural and/or functional events occur that are established to be involved in the initiation or progression of the disease. The positive effects of the compounds disclosed herein have been demonstrated in a variety of such assays and models, for a number of diseases and disorders. One skilled in the art can readily determine following the guidance described herein whether a compound disclosed herein useful for the purposed herein described.

As detailed in the exemplification herein, in assays to determine the ability of compounds to inhibit the activities of ROCK1 and/or ROCK2 measured in vitro, certain provided compounds exhibited IC₅₀ values ≤50 μM. In certain other embodiments, provided compounds exhibit IC₅₀ values ≤40 μM. In certain other embodiments, provided compounds exhibit IC₅₀ values ≤30 μM. In certain other embodiments, provided compounds exhibit IC₅₀ values ≤20 μM. In certain other embodiments, provided compounds exhibit IC₅₀ values ≤10 μM. In certain other embodiments, provided compounds exhibit IC₅₀ values ≤7.5 μM. In certain embodiments, provided compounds exhibit IC₅₀ values ≤5 μM. In certain other embodiments, provided compounds exhibit IC₅₀ values ≤2.5 μM. In certain embodiments, provided compounds exhibit IC₅₀ values ≤1 μM. In certain other embodiments, provided compounds exhibit IC₅₀ values ≤750 nM. In certain other embodiments, provided compounds exhibit IC₅₀ values ≤500 nM. In certain other embodiments, provided compounds exhibit IC₅₀ values ≤250 nM. In certain other embodiments, provided compounds exhibit IC₅₀ values ≤100 nM. In other embodiments, exemplary compounds exhibited IC₅₀ values ≤75 nM. In other embodiments, exemplary compounds exhibited IC₅₀ values ≤50 nM. In other embodiments, exemplary compounds exhibited IC₅₀ values ≤40 nM. In other embodiments, exemplary compounds exhibited IC₅₀ values ≤30 nM. In other embodiments, exemplary compounds exhibited IC₅₀ values ≤20 nM. In other embodiments, exemplary compounds exhibited IC₅₀ values ≤10 nM. In other embodiments, exemplary compounds exhibited IC₅₀ values ≤5 nM.

As detailed in the exemplification herein, in assays to determine the affinity of compounds in binding to ROCK1 and/or ROCK2 measured in vitro, certain provided compounds exhibited equilibrium dissociation constant Kd values ≤50 μM. In certain other embodiments, provided compounds exhibit Kd values ≤40 μM. In certain other embodiments, provided compounds exhibit Kd values ≤30 μM. In certain other embodiments, provided compounds exhibit Kd values ≤20 μM. In certain other embodiments, provided compounds exhibit Kd values ≤10 μM. In certain other embodiments, provided compounds exhibit Kd values ≤7.5 μM. In certain embodiments, provided compounds exhibit Kd values ≤5 μM. In certain other embodiments, provided compounds exhibit Kd values ≤2.5 μM. In certain embodiments, provided compounds exhibit Kd values ≤1 μM. In certain other embodiments, provided compounds exhibit Kd values ≤750 nM. In certain other embodiments, provided compounds exhibit Kd values ≤500 nM. In certain other embodiments, provided compounds exhibit Kd values ≤250 nM. In certain other embodiments, provided compounds exhibit Kd values ≤100 nM. In other embodiments, exemplary compounds exhibited Kd values ≤75 nM. In other embodiments, exemplary compounds exhibited Kd values ≤50 nM. In other embodiments, exemplary compounds exhibited Kd values ≤40 nM. In other embodiments, exemplary compounds exhibited Kd values ≤30 nM. In other embodiments, exemplary compounds exhibited Kd values ≤20 nM. In other embodiments, exemplary compounds exhibited Kd values ≤10 nM. In other embodiments, exemplary compounds exhibited Kd values ≤5 nM.

In certain embodiments, the compounds disclosed herein are selective inhibitors of either ROCK1 or ROCK2. In some embodiments, compounds disclosed herein selectively inhibit ROCK2, and thus, in some embodiments, exhibit less of ability to cause hypotension. In some embodiments, compounds disclosed herein inhibit both ROCK1 and ROCK2 to achieve optimal efficacies.

As used herein, the term “selective inhibition” or “selectively inhibit(s)” means that a provided compound has greater inhibition of ROCK2 in at least one assay described herein (e.g., biochemical or cellular) as compared to ROCK1. In some embodiments, the term “selective inhibition” or “selectively inhibit(s)” means that a provided compound is at least 2 times, at least 3 times, at least 5 times, at least 10 times, at least 15 times, at least 20 times, at least 25 times, at least 30 times, at least 40 times, at least 50 times, at least 60 times, at least 70 times, at least 80 times, at least 90 times, at least 100 times, at least 150 times, at least 200 times, at least 300 times, at least 400 times, at least 500 times, or at least 1000 times more potent as an inhibitor of ROCK2 as compared to inhibition of ROCK1. In some embodiments, the selectivity of a provided compound is determined based on an assay described herein. In some such embodiments, the selectivity of a provided compound is determined based on DiscoverX's KINOMEscan™ KdELECT technology.

Pharmaceutical Uses and Methods of Treatment

As discussed above, certain of the compounds as described herein exhibit activity generally as modulators of the activity of ROCK1 and/or ROCK2. More specifically, compounds disclosed herein demonstrate the ability to inhibit the activity of ROCK1 and/or ROCK2. Thus, in certain embodiments, compounds disclosed herein are useful for the treatment of any of a number of conditions or diseases in which inhibiting the activity of ROCK1 and/or ROCK2 has or plays a therapeutically useful role. Thus, compounds disclosed herein are useful for the treatment of any condition, disease or disorder in which inhibiting the activity of ROCK1 and/or ROCK2 has a beneficial role.

Accordingly, in another aspect, methods for the treatment of ROCK1 and/or ROCK2 related disorders are provided comprising administering a therapeutically effective amount of a compound of Formula I as described herein, to a subject in need thereof. In certain embodiments, a method for the treatment of ROCK1 and/or ROCK2 related disorders is provided comprising administering a therapeutically effective amount of a provided compound, or a pharmaceutical composition comprising a provided compound to a subject in need thereof, in such amounts and for such time as is necessary to achieve the desired result.

In certain embodiments, the method involves the administration of a therapeutically effective amount of the compound or a pharmaceutically acceptable derivative(s) thereof to a subject (including, but not limited to a human or animal) in need of it. Subjects for which the benefits of the compounds disclosed herein are intended for administration include, in addition to humans, livestock, domesticated, zoo and companion animals.

Thus, as described above, in one aspect, the present disclosure provides a method for treating disorders mediated by ROCK1 and/or ROCK2, the method comprising administering a therapeutically effective amount of a compound of Formula I as described herein, to a subject in need thereof. In certain embodiments, provided methods are useful for the treatment of one or more ROCK1 and/or ROCK2 mediated disorders selected from hepatic diseases, renal diseases, cerebral and/or cerebrovascular diseases, cardiac and/or cardiovascular diseases, pulmonary diseases, dermal diseases, gastrointestinal diseases, stroke, myocardial infarction and other ischemic or fibrotic diseases. It will be appreciated that the compounds and compositions, according to the methods disclosed herein, may be administered using any amount and any route of administration effective for the treatment of conditions or diseases mediated by ROCK1 and/or ROCK2. Thus, the expression “effective amount” as used herein, refers to a sufficient amount of agent to inhibit the activity of ROCK1 and/or ROCK2 that exhibits a therapeutic effect. The exact amount required will vary from subject to subject, depending on the species, age, and general condition of the subject, the severity of the infection, the particular therapeutic agent, its mode and/or route of administration, and the like. The compounds disclosed herein are preferably formulated in unit dosage form for ease of administration and uniformity of dosage. The expression “unit dosage form” as used herein refers to a physically discrete unit of therapeutic agent appropriate for the patient to be treated. It will be understood, however, that the total daily dosage of compounds and compositions disclosed herein will be decided by the attending physician within the scope of sound medical judgment. The specific therapeutically effective dose for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, the present disclosure provides a method of inhibiting ROCK1 and/or ROCK2 in a patient or in a biological sample. In some embodiments, the present disclosure provides a method of inhibiting ROCK1 and/or ROCK2, the method comprising contacting a biological sample with a compound of Formula I, or a pharmaceutically acceptable salt thereof.

In some embodiments, the present disclosure provides a method of inhibiting ROCK2 selectively as compared to ROCK1 in a biological sample or in a patient.

In some embodiments, the present disclosure provides a method of treating or lessening the severity of one or more diseases or disorders associated with or mediated by ROCK1 and/or ROCK2. In some embodiments, a disease or disorder associated with or mediated by ROCK1 and/or ROCK2 is a disease or disorder as described herein. In some embodiments, a method of treating or lessening the severity of one or more diseases or disorders associated with or mediated by ROCK1 and/or ROCK2 includes the step of administering to a patient in need thereof a compound of Formula I, or a pharmaceutically acceptable salt thereof. In some embodiments, a patient in need thereof comprises a subject, or a population of subjects, who is/are suffering from, has/have been diagnosed with, or is/are suspected of having a disease or disorder associated with or mediated by ROCK1 and/or ROCK2.

Furthermore, after formulation with an appropriate pharmaceutically acceptable carrier in a desired dosage, the pharmaceutical compositions disclosed herein can be administered to humans and other animals orally, rectally, parenterally, intracisternally, intravaginally, intraperitoneally, subcutaneously, intradermally, intra-ocularly, topically (as by powders, ointments, or drops), buccally, as an oral or nasal spray, or the like, depending on the severity of the disease or disorder being treated. In certain embodiments, the compounds disclosed herein may be administered at dosage levels of about 0.001 mg/kg to about 50 mg/kg, preferably from about 0.1 mg/kg to about 10 mg/kg for parenteral administration, or preferably from about 1 mg/kg to about 50 mg/kg, more preferably from about 10 mg/kg to about 50 mg/kg for oral administration, of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. In certain embodiments, compounds are administered orally or parenterally.

Moreover, pharmaceutical compositions comprising one or more compounds disclosed herein may also contain other compounds or agents for which co-administration with the compound(s) disclosed herein is therapeutically advantageous. As many pharmaceutical agents are used in the treatment of the diseases and disorders for which the compounds disclosed herein are also beneficial, any may be formulated together for administration. Synergistic formulations are also embraced herein, where the combination of at least one compound disclosed herein and at least one other compound act more beneficially than when each is given alone.

Biomarkers

In some embodiments, the present disclosure provides certain biomarkers that can distinguish subjects (e.g., subjects suffering from or at risk of a hepatic, renal, cerebral and/or cerebrovascular, cardiac and/or cardiovascular, pulmonary disease, dermal, or gastrointestinal disorder or condition described above and herein) who are more likely than others to respond to therapy with a compound of Formula I as described herein. The present disclosure provides the insight that certain biomarkers can distinguish patients who are likely to respond to therapy, for example, because the drivers of their hepatic, renal, cerebral and/or cerebrovascular, cardiac and/or cardiovascular, pulmonary disease, dermal, or gastrointestinal disorder or condition correspond and/or associated with, and/or secondary and/or related to the mechanism of action of a compound of Formula I as described herein. For example, in some embodiments, an altered level (e.g., an expression level) of one or more gene products or proteins that are part of the mechanism of action of a compound of Formula I as described herein (e.g., down- or up-regulated by a compound of Formula I as described herein) is indicative of patients who are likely to respond to therapy comprising a compound of Formula I as described herein. In some embodiments, a patient with an altered level of one or more biomarkers may have an improved response to treatment with a compound of Formula I as described herein relative to a patient that does not have a level of the biomarker that meets the threshold criteria.

Generally, as used herein, a biomarker is a component of a biological sample that may be detected and/or quantified when present in the biological sample. A biomarker may include one or more of a peptide, protein, nucleic acid (e.g., polynucleotide, DNA, RNA, etc.), polysaccharide (e.g., lectins or sugars), lipid, enzyme, small molecule, ligand, receptor, antigen, or antibody. In some embodiments, a biomarker comprises a protein. In some embodiments, a biomarker comprises a nucleic acid (e.g., mRNA, miRNA, siRNA, etc.). In some embodiments, a biomarker comprises an oncogene (e.g., oncogenic miRNA). In some embodiments, a level of a biomarker corresponds to a level of gene expression (e.g., RNA expression, e.g., mRNA expression, miRNA expression, siRNA expression, etc.). In some embodiments, a level of a biomarker corresponds to a level of oncogene expression (e.g., oncogenic miRNA expression). In some embodiments, a level of a biomarker corresponds to a level of protein expression, including any fragment or degradation product thereof.

In some embodiments, a biomarker is detected and/or quantified in a tissue sample (e.g., from a biopsy, such as a liver, brain, heart, kidney, lung, skin, or gastrointestinal tract biopsy) and/or in a biological fluid (e.g., blood, urine, etc.). In some embodiments, a biomarker (e.g., a level of mRNA, miRNA, siRNA, etc.) is detected and/or quantified in a liver, brain, heart, kidney, lung, skin, or gastrointestinal tract tissue sample, e.g., obtained from a liver, brain, heart, kidney, lung, skin, or gastrointestinal tract biopsy. In some embodiments, a biomarker (e.g., a level of a protein or protein fragment) is detected and/or quantified in a urine sample. In some embodiments, a biomarker (e.g., a level of a protein or protein fragment) is detected and/or quantified in a blood sample.

In some embodiments, one biomarker is used to characterize subjects. In some embodiments, more than one biomarker (e.g., two, three, etc.) is used to characterize subjects.

It will be appreciated that biomarkers (e.g., genes and/or proteins) identified using non-human animal models can be predictive of biomarkers relevant to treatment of human subjects (e.g., according to methods described herein). For example, a corresponding human analog of a biomarker (e.g., genes and/or proteins) identified using a non-human animal model can be determined; in some embodiments, such corresponding human analogs are useful in the treatment of human subjects as described herein. In some embodiments, a rodent (e.g., rat or mouse) model is used to identify biomarkers expected to be relevant to treatment of human subjects (e.g., according to methods described herein).

In some embodiments, one or more biomarkers are differentially present in a sample taken from a subject of one status as compared with a subject of another status (e.g., more responsive to therapy with a compound of Formula I as described herein vs less responsive to therapy with a compound of Formula I as described herein). In some embodiments, one or more biomarkers are differentially present in a sample taken from the same subject at two or more different time points, i.e., when the status of the subject has changed from one time point to another.

In some embodiments, detection of levels of one or more biomarkers are used to select and/or characterize patients who may be responsive to therapy with a compound of Formula I as described herein. In some embodiments, levels of one or more biomarkers in a sample obtained from a subject are compared to a threshold level. In some embodiments, a biomarker is considered altered if the level is altered relative to a threshold level (e.g., altered by at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold, or more). In some embodiments, an altered biomarker is elevated relative to a threshold level (e.g., elevated by at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold, or more). In some embodiments, an altered biomarker is reduced relative to a threshold level (e.g., reduced by at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold, or more). In some embodiments, a biomarker is considered altered if the level is altered relative to a threshold level (e.g., altered by at least 0.5, about 1.0, about 1.5, about 2.0, about 2.5, about 3.0, about 3.5, about 4.0, about 4.5, or about 5.0, or more standard deviations). In some embodiments, an altered biomarker is elevated relative to a threshold level (e.g., elevated by at least about 0.5, about 1.0, about 1.5, about 2.0, about 2.5, about 3.0, about 3.5, about 4.0, about 4.5, or about 5.0, or more standard deviations). In some embodiments, an altered biomarker is reduced relative to a threshold level (e.g., reduced by at least about 0.5, about 1.0, about 1.5, about 2.0, about 2.5, about 3.0, about 3.5, about 4.0, about 4.5, or about 5.0, or more standard deviations). In some embodiments, a threshold level is determined from a population of healthy volunteers (e.g., a mean or median level from a population of healthy volunteers).

Any suitable means can be used to determine levels of one or more biomarkers in accordance with the present disclosure. In some embodiments, a method includes an in vitro method for determining a level of a biomarker. For example, in vitro methods for determining a level of a biomarker include, but are not limited to, a chemiluminescence assay, enzymatic assay, enzyme immunoassay, multiplex immunoassay, ELISA, chromatographic immunoassay, electrophoresis assay, radioimmunoassay, colorimetric assay, chromatography/mass spectrometry (e.g., GC/MS, LC/MS, LC/MS/MS, etc.), High Performance Liquid Chromatography (“HPLC”), and/or PCR (e.g., real-time PCR). In some embodiments, a method for detecting a level of a biomarker includes chromatographic and/or MS methods. Exemplary methods include, but are not limited to, gas chromatography (GC), liquid chromatography/mass spectroscopy (LC-MS), gas chromatography/mass spectroscopy (GC-MS), nuclear magnetic resonance (NMR), magnetic resonance imaging (MRI), Fourier Transform InfraRed (FT-IR), and inductively coupled plasma mass spectrometry (ICP-MS).

In some embodiments, a level of a biomarker corresponds to a level of gene expression (e.g., RNA, e.g., mRNA, miRNA, siRNA, etc.) and is quantified using methods known in the art. In some embodiments, a method of determining a level of expression of a biomarker gene (e.g., RNA, e.g., mRNA, miRNA, siRNA, etc.) can be or include a chemiluminescence assay, UV spectroscopy, hybridization assay (e.g., Fluorescent in Situ Hybridization (FISH), e.g., RNA-FISH), enzymatic assay, enzyme immunoassay (e.g., ELISA), multiplex assay, electrophoresis assay, radioassay, colorimetric assay, chromatography/mass spectrometry (e.g., GC/MS, LC/MS, LC/MS/MS, etc.), High Performance Liquid Chromatography (“HPLC”), and/or PCR (e.g., quantitative PCR and/or real-time PCR).

In some embodiments, a level of a biomarker corresponds to a level of protein, including any fragment or degradation product thereof, and is quantified using methods known in the art. In some embodiments, a method of determining a level of expression of a biomarker protein can be or include a chemiluminescence assay, enzymatic assay, enzyme immunoassay, multiplex immunoassay, ELISA, chromatographic immunoassay, electrophoresis assay, radioimmunoassay, colorimetric assay, UV spectroscopy, chromatography/mass spectrometry (e.g., GC/MS, LC/MS, LC/MS/MS, etc.), or High Performance Liquid Chromatography (“HPLC”).

In some embodiments, a biomarker useful in methods provided herein is a miRNA selected from Table 2, or a human analog thereof. In some embodiments, a level of a biomarker selected from Table 2 is a level of gene expression of a miRNA selected from Table 2, or a human analog thereof.

TABLE 2

| | | | |
|---|---|---|---|
| miR-99b-5p | miR-125a-3p | miR-181a-5p | miR-191-5p |
| miR-143-3p | miR-106a-5p | miR-27a-5p | miR-93-5p |
| miR-93-3p | miR-322-3p | miR-339-3p | miR-351-5p |
| miR-17-3p | miR-320-3p | miR-425-5p | miR-542-5p |
| miR-802-5p | miR-501-3p | miR-871-3p | miR-5123 |
| let-7k | miR-7115-5p | | |

In some embodiments, a biomarker useful in methods provided herein is a miRNA selected from the group consisting of miR-181a-5p and miR-425-5p. In some embodiments, a level of a biomarker selected from the group consisting of miR-181a-5p and miR-425-5p is a level of gene expression of a miRNA selected from the group consisting of miR-181a-5p and miR-425-5p. In some embodiments, a biomarker useful in methods provided herein is miR-181a-5p. In some embodiments, a level of a biomarker is a level of gene expression of miR-181a-5p. In some embodiments, a biomarker useful in methods provided herein is miR-425-5p. In some embodiments, a level of a biomarker is a level of gene expression of miR-425-5p.

In some embodiments, a biomarker useful in methods provided herein is a human miRNA selected from the group consisting of hsa-miR-181a-5p and hsa-miR-425-5p. In some embodiments, a level of a biomarker selected from the group consisting of hsa-miR-181a-5p and hsa-miR-425-5p is a level of gene expression of a human miRNA selected from the group consisting of hsa-miR-181a-5p and hsa-miR-425-5p. In some embodiments, a biomarker useful in methods provided herein is hsa-miR-181a-5p. In some embodiments, a level of a biomarker is a level of gene expression of hsa-miR-181a-5p. In some embodiments, a biomarker useful in methods provided herein is hsa-miR-425-5p. In some embodiments, a level of a biomarker is a level of gene expression of hsa-miR-425-5p.

In some embodiments, a biomarker useful in methods provided herein is a hepatic miRNA selected from Table 2, or a human analog thereof. In some embodiments, a level of a biomarker selected from Table 2 is a level of gene expression of a hepatic miRNA selected from Table 2, or a human analog thereof.

In some embodiments, a biomarker useful in methods provided herein is an oncogenic miRNA selected from Table 2, or a human analog thereof. In some embodiments, a level of a biomarker selected from Table 2 is a level of gene expression of an oncogenic miRNA selected from Table 2, or a human analog thereof.

In some embodiments, a biomarker useful in methods provided herein is a miRNA selected from Table 2, or a human analog thereof, with a change in mean expression for sham animals relative to animals submitted to a fast-food diet (FFD animals) of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold. In some embodiments, a biomarker useful in methods provided herein is a miRNA selected from Table 2, or a human analog thereof, with a change in mean expression for sham animals relative to FFD animals of at least about 0.5, about 1.0, about 1.5, about 2.0, about 2.5, about 3.0, about 3.5, about 4.0, about 4.5, or about 5.0 standard deviations. In some embodiments, a biomarker useful in methods provided herein is a miRNA selected from Table 2, or a human analog thereof, with a change in mean expression for FFD animals relative to animals administered FFD+a compound of Formula I as described herein of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold. In some embodiments, a biomarker useful in methods provided herein is a miRNA selected from Table 2, or a human analog thereof, with a change in mean expression for FFD animals relative to animals administered FFD+a compound of Formula I as described herein of at least about 0.5, about 1.0, about 1.5, about 2.0, about 2.5, about 3.0, about 3.5, about 4.0, about 4.5, or about 5.0 standard deviations. In some embodiments, a biomarker useful in methods provided herein is a miRNA selected from Table 2, or a human analog thereof, with a change in mean expression for sham animals relative to animals administered FFD+a compound of Formula I as described herein of less than about 100%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, or about 5%. In some embodiments, a biomarker useful in methods provided herein is a miRNA selected from Table 2, or a human analog thereof, with a change in mean expression for sham animals relative to animals administered FFD+a compound of Formula I as described herein of less than about 0.5, about 1.0, about 1.5, about 2.0, about 2.5, about 3.0, about 3.5, about 4.0, about 4.5, or about 5.0 standard deviations.

In some embodiments, a biomarker useful in methods provided herein is a miRNA selected from Table 2, or a human analog thereof, with:
a change in mean expression for sham animals relative to FFD animals of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold; and/or
a change in mean expression for FFD animals relative to animals administered FFD+a compound of Formula I as described herein of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold; and/or
a change in mean expression for sham animals relative to animals administered FFD+a compound of Formula I as described herein of less than about 100%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, or about 5%.

In some embodiments, a biomarker useful in methods provided herein is a miRNA selected from Table 2, or a human analog thereof, with:
a change in mean expression for sham animals relative to FFD animals of at least about 0.5, about 1.0, about 1.5, about 2.0, about 2.5, about 3.0, about 3.5, about 4.0, about 4.5, or about 5.0 standard deviations; and/or
a change in mean expression for FFD animals relative to animals administered FFD+a compound of Formula I as described herein of at least about 0.5, about 1.0, about 1.5, about 2.0, about 2.5, about 3.0, about 3.5, about 4.0, about 4.5, or about 5.0 standard deviations; and/or
a change in mean expression for sham animals relative to animals administered FFD+a compound of Formula I as described herein of less than about 0.5, about 1.0, about 1.5, about 2.0, about 2.5, about 3.0, about 3.5, about 4.0, about 4.5, or about 5.0 standard deviations.

In some embodiments, the FFD animal is subject to FFD+CCl₄+glucose.

In some embodiments, a biomarker useful in methods provided herein is a protein, a fragment thereof, or a human analog thereof. In some embodiments, a biomarker useful in methods provided herein is selected from Table 3, a fragment thereof, or a human analog thereof. In some embodiments, a level of a biomarker selected from Table 3 is a level of expression of a biomarker selected from Table 3, a fragment thereof, or a human analog thereof.

TABLE 3

| α-smooth muscle | | | |
|---|---|---|---|
| actin (α-SMA) | collagen I | collagen III | N-cadherin |
| TIMP-1 | plasminogen activator | metalloproteinase | tissue plasminogen |
| | inhibitor (PAI-1) | (e.g. MMP-1, MMP-7 | activator (tPA) |
| | | and MMP-9) | |
| urokinase | soluble VEGF | epidermal growth | plasma soluble TNF |
| plasminogen activator | | factor (EGF) | receptor 2 (sTNFR2) |
| (uPA) | | | |
| kidney injury | neutrophil gelatinase- | urinary fibronectin, | monocyte chemotactic |
| molecule-1 | associated lipocalin | high molecular weight | protein-1 (MCP-1) |
| (KIM-1) | (NGAL) | type IV collagen | |
| | | (HMW collagen IV) | |

In some embodiments, a biomarker useful in methods provided herein is α-smooth muscle actin (α-SMA), a fragment thereof, or a human analog thereof. In some embodiments, a level of a biomarker is a level of expression of α-SMA, a fragment thereof, or a human analog thereof. In some embodiments, a biomarker useful in methods provided herein is collagen I, a fragment thereof, or a human analog thereof. In some embodiments, a level of a biomarker is a level of expression of collagen I, a fragment thereof, or a human analog thereof. In some embodiments, a biomarker useful in methods provided herein is collagen III, a fragment thereof, or a human analog thereof. In some embodiments, a level of a biomarker is a level of expression of collagen III, a fragment thereof, or a human analog thereof.

In some embodiments, a biomarker useful in methods provided herein is renal α-SMA, a fragment thereof, or a human analog thereof. In some embodiments, a level of a biomarker is a level of expression of renal α-SMA, a fragment thereof, or a human analog thereof. In some embodiments, a biomarker useful in methods provided herein is renal collagen I, a fragment thereof, or a human analog thereof. In some embodiments, a level of a biomarker is a level of expression of renal collagen I, a fragment thereof, or a human analog thereof. In some embodiments, a biomarker useful in methods provided herein is renal collagen III, a fragment thereof, or a human analog thereof. In some embodiments, a level of a biomarker is a level of expression of renal collagen III, a fragment thereof, or a human analog thereof.

In some embodiments, a biomarker useful in methods provided herein is a biomarker selected from Table 3, a fragment thereof, or a human analog thereof, with a change in mean expression for sham animals relative to animals submitted to unilateral ureteral obstruction (UUO animals) of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold. In some embodiments, a biomarker useful in methods provided herein is a biomarker selected from Table 3, a fragment thereof, or a human analog thereof, with a change in mean expression for sham animals relative to UUO animals of at least about 0.5, about 1.0, about 1.5, about 2.0, about 2.5, about 3.0, about 3.5, about 4.0, about 4.5, or about 5.0 standard deviations. In some embodiments, a biomarker useful in methods provided herein is a biomarker selected from Table 3, a fragment thereof, or a human analog thereof, with a change in mean expression for UUO animals relative to animals treated with UUO+a compound of Formula I as described herein of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold. In some embodiments, a biomarker useful in methods provided herein is a biomarker selected from Table 3, a fragment thereof, or a human analog thereof, with a change in mean expression for UUO animals relative to animals treated with UUO+a compound of Formula I as described herein of at least about 0.5, about 1.0, about 1.5, about 2.0, about 2.5, about 3.0, about 3.5, about 4.0, about 4.5, or about 5.0 standard deviations. In some embodiments, a biomarker useful in methods provided herein is a biomarker selected from Table 3, a fragment thereof, or a human analog thereof, with a change in mean expression for sham animals relative to animals treated with UUO+a compound of Formula I as described herein of less than about 100%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, or about 5%. In some embodiments, a biomarker useful in methods provided herein is a biomarker selected from Table 3, a fragment thereof, or a human analog thereof, with a change in mean expression for sham animals relative to animals treated with UUO+a compound of Formula I as described herein of less than about 0.5, about 1.0, about 1.5, about 2.0, about 2.5, about 3.0, about 3.5, about 4.0, about 4.5, or about 5.0 standard deviations.

In some embodiments, a biomarker useful in methods provided herein is a biomarker selected from Table 3, a fragment thereof, or a human analog thereof, with:
a change in mean expression for sham animals relative to UUO animals of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold; and/or
a change in mean expression for UUO animals relative to animals treated with UUO+a compound of Formula I as described herein of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold; and/or
a change in mean expression for sham animals relative to animals treated with UUO+a compound of Formula I as described herein of less than about 100%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, or about 5%.

In some embodiments, a biomarker useful in methods provided herein is a biomarker selected from Table 3, a fragment thereof, or a human analog thereof, with:
a change in mean expression for sham animals relative to UUO animals of at least about 0.5, about 1.0, about 1.5, about 2.0, about 2.5, about 3.0, about 3.5, about 4.0, about 4.5, or about 5.0 standard deviations; and/or
a change in mean expression for UUO animals relative to animals treated with UUO+a compound of Formula I as described herein of at least about 0.5, about 1.0, about 1.5, about 2.0, about 2.5, about 3.0, about 3.5, about 4.0, about 4.5, or about 5.0 standard deviations; and/or
a change in mean expression for sham animals relative to animals treated with UUO+a compound of Formula I as described herein of less than about 0.5, about 1.0, about 1.5, about 2.0, about 2.5, about 3.0, about 3.5, about 4.0, about 4.5, or about 5.0 standard deviations.

In some embodiments, the present disclosure provides insights that altered levels of one or more biomarkers described above and herein, a fragment thereof, or a human analog thereof, may be useful in selecting and/or characterizing patients for therapy comprising a compound of Formula I as described herein.

In some embodiments, patients are selected and/or characterized based on the percentage of altered levels of biomarkers observed in a biological sample obtained from the patient. For example, in some embodiments, a patient has been determined to have an altered level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the biomarkers described above and herein, a fragment thereof, or a human analog thereof. In some embodiments, a patient has been determined to have an elevated level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the biomarkers described above and herein, a fragment thereof, or a human analog thereof. In some embodiments, a patient has been determined to have a reduced level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the biomarkers described above and herein, a fragment thereof, or a human analog thereof.

In some embodiments, a patient has been determined to have an altered level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the biomarkers in Table 2, or a human analog thereof, with a change in mean expression for sham animals relative to FFD animals of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold. In some embodiments, a patient has been determined to have an altered level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the biomarkers in Table 2, or a human analog thereof, with a change in mean expression for sham animals relative to FFD animals of at least about 0.5, about 1.0, about 1.5, about 2.0, about 2.5, about 3.0, about 3.5, about 4.0, about 4.5, or about 5.0 standard deviations. In some embodiments, a patient has been determined to have an altered level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the biomarkers in Table 2, or a human analog thereof, with a change in mean expression for FFD animals relative to animals administered with FFD+a compound of Formula I as described herein of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold. In some embodiments, a patient has been determined to have an altered level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the biomarkers in Table 2, or a human analog thereof, with a change in mean expression for FFD animals relative to animals administered FFD+a compound of Formula I as described herein of at least about 0.5, about 1.0, about 1.5, about 2.0, about 2.5, about 3.0, about 3.5, about 4.0, about 4.5, or about 5.0 standard deviations. In some embodiments, a patient has been determined to have an altered level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the biomarkers in Table 2, or a human analog thereof, with a change in mean expression for sham animals relative to animals administered FFD+a compound of Formula I as described herein of less than about 100%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, or about 5%. In some embodiments, a patient has been determined to have an altered level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the biomarkers in Table 2, or a human analog thereof, with a change in mean expression for sham animals relative to animals administered FFD+a compound of Formula I as described herein of less than about 0.5, about 1.0, about 1.5, about 2.0, about 2.5, about 3.0, about 3.5, about 4.0, about 4.5, or about 5.0 standard deviations.

In some embodiments, a patient has been determined to have an altered level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the biomarkers in Table 2, or a human analog thereof, with:
a change in mean expression for sham animals relative to FFD animals of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold; and/or
a change in mean expression for FFD animals relative to animals administered FFD+a compound of Formula I as described herein of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold; and/or
a change in mean expression for sham animals relative to animals administered FFD+a compound of Formula I as described herein of less than about 100%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, or about 5%.

In some embodiments, a patient has been determined to have an altered level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the biomarkers in Table 2, or a human analog thereof, with:
a change in mean expression for sham animals relative to FFD animals of at least about 0.5, about 1.0, about 1.5, about 2.0, about 2.5, about 3.0, about 3.5, about 4.0, about 4.5, or about 5.0 standard deviations; and/or
a change in mean expression for FFD animals relative to animals administered FFD+a compound of Formula I as described herein of at least about 0.5, about 1.0, about 1.5, about 2.0, about 2.5, about 3.0, about 3.5, about 4.0, about 4.5, or about 5.0 standard deviations; and/or
a change in mean expression for sham animals relative to animals administered FFD+a compound of Formula I as described herein of less than about 0.5, about 1.0, about 1.5, about 2.0, about 2.5, about 3.0, about 3.5, about 4.0, about 4.5, or about 5.0 standard deviations.

In some embodiments, a patient has been determined to have an altered level of at least one biomarker selected from Table 2, or a human analog thereof, with a change in mean expression for sham animals relative to FFD animals of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, or about 3-fold and at least one biomarker selected from Table 2, or a human analog thereof, with a change in mean expression for sham animals relative to FFD animals of at least about 4-fold, about 5-fold, about 10-fold, or about 20-fold. In some embodiments, a patient has been determined to have an altered level of at least one biomarker selected from Table 2, or a human analog thereof, with a change in mean expression for sham animals relative to FFD animals of at least about 0.5, about 1.0, about 1.5, about 2.0, or about 2.5 standard deviations and at least one biomarker selected from Table 2, or a human analog thereof, with a change in mean expression for sham animals relative to FFD animals of at least about 3.0, about 3.5, about 4.0, about 4.5, or about 5.0 standard deviations.

In some embodiments, a patient has been determined to have an altered level of at least one biomarker selected from Table 2, or a human analog thereof, with a change in mean expression for FFD animals relative to animals administered FFD+a compound of Formula I as described herein of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, or about 3-fold and at least one biomarker in Table 2, or a human analog thereof, with a change in mean expression for FFD animals relative to animals administered FFD+a compound of Formula I as described herein of at least about 4-fold, about 5-fold, about 10-fold, or about 20-fold. In some embodiments, a patient has been determined to have an altered level of at least one biomarker selected from Table 2, or a human analog thereof, with a change in mean expression for FFD animals relative to animals administered FFD+a compound of Formula I as described herein of at least about 0.5, about 1.0, about 1.5, about 2.0, or about 2.5 standard deviations and at least one biomarker selected from Table 2, or a human analog thereof, with a change in mean expression for FFD animals relative to animals administered FFD+a compound of Formula I as described herein of at least about 3.0, about 3.5, about 4.0, about 4.5, or about 5.0 standard deviations.

In some embodiments, a patient has been determined to have an altered level of at least one biomarker selected from Table 2, or a human analog thereof, with a change in mean expression for sham animals relative to animals administered FFD+a compound of Formula I as described herein of less than about 100%, about 95%, about 90%, about 85%, about 80%, about 75%, about 70%, about 65%, or about 60% and at least one biomarker selected from Table 2, or a human analog thereof, with a change in mean expression for sham animals relative to animals administered FFD+a compound of Formula I as described herein of less than about 55%, about 50%, about 45%, about 40%, about 35%, about 30%, about 25%, about 20%, about 15%, about 10%, or about 5%. In some embodiments, a patient has been determined to have an altered level of at least one biomarker selected from Table 2, or a human analog thereof, a change in mean expression for sham animals relative to animals administered FFD+a compound of Formula I as described herein of at least about 0.5, about 1.0, about 1.5, about 2.0, or about 2.5 standard deviations and at least one biomarker selected from Table 2, or a human analog thereof, with a change in mean expression for sham animals relative to animals administered FFD+a compound of Formula I as described herein of at least about 3.0, about 3.5, about 4.0, about 4.5, or about 5.0 standard deviations.

In some embodiments, a patient has been determined to have an altered level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of a biomarker selected from Table 3, a fragment thereof, or a human analog thereof, with a change in mean expression for sham animals relative to UUO animals of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold. In some embodiments, a patient has been determined to have an altered level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of a biomarker selected from Table 3, a fragment thereof, or a human analog thereof, with a change in mean expression for sham animals relative to UUO animals of at least about 0.5, about 1.0, about 1.5, about 2.0, about 2.5, about 3.0, about 3.5, about 4.0, about 4.5, or about 5.0 standard deviations. In some embodiments, a patient has been determined to have an altered level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of a biomarker selected from Table 3, a fragment thereof, or a human analog thereof, with a change in mean expression for UUO animals relative to animals treated with UUO+a compound of Formula I as described herein of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold. In some embodiments, a patient has been determined to have an altered level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of a biomarker selected from Table 3, a fragment thereof, or a human analog thereof, with a change in mean expression for UUO animals relative to animals treated with UUO+a compound of Formula I as described herein of at least about 0.5, about 1.0, about 1.5, about 2.0, about 2.5, about 3.0, about 3.5, about 4.0, about 4.5, or about 5.0 standard deviations. In some embodiments, a patient has been determined to have an altered level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of a biomarker selected from Table 3, a fragment thereof, or a human analog thereof, with a change in mean expression for sham animals relative to animals treated with UUO+a compound of Formula I as described herein of less than about 100%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, or about 5%. In some embodiments, a patient has been determined to have an altered level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of a biomarker selected from Table 3, a fragment thereof, or a human analog thereof, with a change in mean expression for sham animals relative to animals treated with UUO+a compound of Formula I as described herein of less than about 0.5, about 1.0, about 1.5, about 2.0, about 2.5, about 3.0, about 3.5, about 4.0, about 4.5, or about 5.0 standard deviations.

In some embodiments, a patient has been determined to have an altered level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of a biomarker selected from Table 3, a fragment thereof, or a human analog thereof, with:
a change in mean expression for sham animals relative to UUO animals of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold; and/or
a change in mean expression for UUO animals relative to animals treated with UUO+a compound of Formula I as described herein of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold; and/or
a change in mean expression for sham animals relative to animals treated with UUO+a compound of Formula I as described herein of less than about 100%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, or about 5%.

In some embodiments, a patient has been determined to have an altered level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of a biomarker selected from Table 3, a fragment thereof, or a human analog thereof, with:
a change in mean expression for sham animals relative to UUO animals of at least about 0.5, about 1.0, about 1.5, about 2.0, about 2.5, about 3.0, about 3.5, about 4.0, about 4.5, or about 5.0 standard deviations; and/or
a change in mean expression for UUO animals relative to animals treated with UUO+a compound of Formula I as described herein of at least about 0.5, about 1.0, about 1.5, about 2.0, about 2.5, about 3.0, about 3.5, about 4.0, about 4.5, or about 5.0 standard deviations; and/or
a change in mean expression for sham animals relative to animals treated with UUO+a compound of Formula I as described herein of less than about 0.5, about 1.0, about 1.5, about 2.0, about 2.5, about 3.0, about 3.5, about 4.0, about 4.5, or about 5.0 standard deviations.

It will be appreciated that a “change in mean expression” between, e.g., sham animals relative to FFD animals or UUO animals; or FFD animals relative to animals administered FFD+a compound of Formula I as described herein, or UUO animals relative to animals treated with UUO+a compound of Formula I as described herein; or sham animals relative to animals administered FFD+a compound of Formula I as described herein or animals treated with UUO+a compound of Formula I as described herein, refers to a comparison of a mean expression value with another mean expression value. For example, a biomarker with a change in mean expression for sham animals relative to FFD animals or UUO animals of at least about 2-fold refers to a biomarker with mean expression for FFD animals or UUO animals that is at least about 2-fold higher or lower than mean expression for sham animals. As another example, a biomarker with a change in mean expression for sham animals relative to animals administered FFD+a compound of Formula I as described herein or animals treated with UUO+a compound of Formula I as described herein of less than about 50% refers to a biomarker with a mean expression for animals administered FFD+a compound of Formula I as described herein or animals treated with UUO+a compound of Formula I as described herein that is less than 50% higher or lower than (i.e., within 50% of) mean expression for sham animals.

In some embodiments, the present disclosure encompasses the recognition that levels of one or more urinary and/or circulating biomarkers may be indicative of and/or correlated with levels of one or more biomarkers described herein. In some embodiments, such urinary and/or circulating biomarkers may be used in provided methods, e.g., to select and/or characterize patients for therapy comprising a compound of Formula I as described herein.

In some embodiments, a patient is determined to have an altered level of a biomarker when the level of the biomarker is above or below a threshold level (e.g., a predetermined median or mean level). In some embodiments, a patient is determined to have an altered level of a biomarker when the level of the biomarker is different from a threshold level by at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold, or more. In some embodiments, a patient is determined to have an altered level of a biomarker when the level of the biomarker is different from a threshold level by at least about 0.5, about 1.0, about 1.5, about 2.0, about 2.5, about 3.0, about 3.5, about 4.0, about 4.5, or about 5.0, or more standard deviations.

In some embodiments, a patient is determined to have an elevated level of a biomarker when the level of the biomarker is above a threshold level (e.g., a predetermined median or mean level). In some embodiments, a patient is determined to have an elevated level of a biomarker when the level of the biomarker is above a threshold level by at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold, or more. In some embodiments, a patient is determined to have an elevated level of a biomarker when the level of the biomarker is above a threshold level by at least about 0.5, about 1.0, about 1.5, about 2.0, about 2.5, about 3.0, about 3.5, about 4.0, about 4.5, or about 5.0, or more standard deviations.

In some embodiments, a patient is determined to have a reduced level of a biomarker when the level of the biomarker is below a threshold level (e.g., a predetermined median or mean level). In some embodiments, a patient is determined to have a reduced level of a biomarker when the level of the biomarker is below a threshold level by at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold, or more. In some embodiments, a patient is determined to have a reduced level of a biomarker when the level of the biomarker is below a threshold level by at least about 0.5, about 1.0, about 1.5, about 2.0, about 2.5, about 3.0, about 3.5, about 4.0, about 4.5, or about 5.0, or more standard deviations.

In some embodiments, the present disclosure provides methods of identifying biomarkers useful for selecting, identifying, and/or characterizing patients likely to benefit from a treatment with a compound of Formula I as described herein. For example, in some embodiments, biomarkers are identified based on a mean change across a population of subjects administered a compound of Formula I as described herein relative to a comparable reference population. In some embodiments, biomarkers useful in methods provided herein are biomarkers that have been established to have a mean increase or decrease of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold, or more in a population of subjects administered a compound of Formula I as described herein relative to a comparable reference population. In some embodiments, biomarkers useful in methods provided herein are biomarkers that have been established to have a mean increase or decrease of at least about 0.5, about 1.0, about 1.5, about 2.0, about 2.5, about 3.0, about 3.5, about 4.0, about 4.5, or about 5.0, or more standard deviations in a population of subjects administered a compound of Formula I as described herein relative to a comparable reference population. In some embodiments, a population of subjects is a population of human subjects. In some embodiments, a population of subjects is a population of non-human animal subjects (e.g., rodent subjects). In some embodiments, a reference population has not received a compound of Formula I as described herein. In some embodiments, a reference population has received an otherwise comparable composition that does not provide a compound of Formula I as described herein (e.g., a placebo).

Alternatively or additionally, in some embodiments, biomarkers are identified based on a mean change across a population of subjects with confirmed hepatic disease(s), renal disease(s), cerebral and/or cerebrovascular disease(s), cardiac and/or cardiovascular disease(s), pulmonary disease(s), dermal disease(s), or gastrointestinal disease(s) described herein relative to a population of healthy volunteers. In some embodiments, biomarkers useful in methods provided herein are biomarkers that have been established to have a mean increase or decrease of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold, or more in a population of subjects with confirmed hepatic disease(s), renal disease(s), cerebral and/or cerebrovascular disease(s), cardiac and/or cardiovascular disease(s), pulmonary disease(s), dermal disease(s), or gastrointestinal disease(s) described herein relative to a population of healthy volunteers. In some embodiments, biomarkers useful in methods provided herein are biomarkers that have been established to have a mean increase or decrease of at least about 0.5, about 1.0, about 1.5, about 2.0, about 2.5, about 3.0, about 3.5, about 4.0, about 4.5, or about 5.0, or more standard deviations in a population of subjects with confirmed hepatic disease(s), renal disease(s), cerebral and/or cerebrovascular disease(s), cardiac and/or cardiovascular disease(s), pulmonary disease(s), dermal disease(s), or gastrointestinal disease(s) described herein relative to a population of healthy volunteers. In some embodiments, a population of subjects is a population of human subjects. In some embodiments, a population of subjects is a population of non-human animal subjects (e.g., rodent subjects).

Provided Methods

Provided herein are methods of treating a subject or a population of subjects comprising administering a compound of Formula I as described herein (e.g., by administering a composition that comprises and/or delivers a compound of Formula I as described herein) to the subject(s) in need thereof.

In some embodiments, such administering is achieved by administering a composition that delivers a compound of Formula I as described herein (e.g., in some embodiments, a composition that is or comprises a compound of Formula I as described herein, or a composition that otherwise delivers a compound of Formula I as described herein—e.g., that is or comprises a prodrug of a compound of Formula I as described herein, a complex or other entity that releases a compound of Formula I as described herein upon administration, etc.).

In some embodiments, provided are methods related to treatment of hepatic disease(s) and selecting, identifying, and/or characterizing patients likely to benefit from a treatment with a compound of Formula I as described herein. In some embodiments, provided methods are useful for treating a hepatic disease selected from those described above and herein in a subject in need thereof. In some such embodiments, a hepatic disease to be treated by provided methods of the present disclosure is selected from the group consisting of hepatitis B, hepatitis C, delta hepatitis, chronic alcoholism, extrahepatic obstructions (stones in the bile duct), cholangiopathies (primary biliary cirrhosis and sclerosing cholangitis), autoimmune liver disease, inherited metabolic disorders (Wilson's disease, hemochromatosis, alpha-1 antitrypsin deficiency, liver steatosis, NASH, liver fibrosis, liver cirrhosis, liver IR injury, and HCC. In some embodiments, a hepatic disease to be treated by provided methods of the present disclosure is liver steatosis. In some embodiments, a hepatic disease to be treated by provided methods of the present disclosure is NASH. In some embodiments, a hepatic disease to be treated by provided methods of the present disclosure is hepatic fibrosis (e.g., fibrotic liver disease). It will be appreciated that provided methods may be suitable for treating hepatic diseases, disorders, and conditions in which fibrosis is the sole or a predominant component, as well as those in which fibrosis is a secondary component (e.g., a symptom and/or result of an underlying disease, disorder, or condition). In some embodiments, a hepatic disease to be treated by provided methods of the present disclosure is liver fibrosis. In some embodiments, a hepatic disease to be treated by provided methods of the present disclosure is liver fibrosis secondary to, or otherwise associated with, an underlying indication. In some embodiments, a hepatic disease to be treated by provided methods of the present disclosure is liver cirrhosis. In some embodiments, a hepatic disease to be treated by provided methods of the present disclosure is liver cirrhosis secondary to, or otherwise associated with, an underlying indication. In some embodiments, a hepatic disease to be treated by provided methods of the present disclosure is liver IR injury. In some embodiments, a hepatic disease to be treated by provided methods of the present disclosure is HCC. In some embodiments, a hepatic disease to be treated by provided methods of the present disclosure is cirrhotic or non-cirrhotic HCC. In some embodiments, a hepatic disease to be treated by provided methods of the present disclosure is cirrhotic HCC. In some embodiments, a hepatic disease to be treated by provided methods of the present disclosure is non-cirrhotic HCC. In some embodiments, a hepatic disease to be treated by provided methods of the present disclosure is sorafenib-resistant HCC.

In some embodiments, provided are methods related to treatment of renal disease(s) and selecting, identifying, and/or characterizing patients likely to benefit from a treatment with a compound of Formula I as described herein. In some embodiments, provided methods are useful for treating a renal disease selected from those described above and herein in a subject in need thereof. In some embodiments, a renal disease to be treated by provided methods of the present disclosure is selected from the group consisting of CKD, AKI, AKI-related CKD, renal fibrosis, renal fibrosis secondary to, or otherwise associated with, an underlying indication, NS, MCD, ANCA-associated glomerulonephritis, lupus nephritis, anti-GBM nephropathy, IgA nephropathy, also known as Berger's disease, AS, polycystic kidney disease, ARPKD-CHF, renal cysts, collagen type III glomerulopathy, and nail-patella syndrome. In some embodiments, a renal disease to be treated by provided methods of the present disclosure is CKD. In some embodiments, a renal disease to be treated by provided methods of the present disclosure is AKI. In some embodiments, a renal disease to be treated by provided methods of the present disclosure is AKI-related CKD.

In some embodiments, provided methods are useful for reducing fibrosis of the kidney in a subject in need thereof. In some embodiments, provided methods are useful for treating a kidney disease, disorder, or condition characterized by or otherwise associated with fibrosis. The present disclosure encompasses the recognition that treating fibrosis (e.g., using provided methods) instead of the underlying etiology may allow for broadly applicable antifibrotic kidney therapies. It will be appreciated that provided methods may be suitable for treating kidney diseases, disorders, and conditions in which fibrosis is the sole or a predominant component, as well as those in which fibrosis is a secondary component (e.g., a symptom and/or result of an underlying disease, disorder, or condition). In some embodiments, provided methods are useful for treating fibrosis associated with an acute injury, such as that incurred from trauma and/or surgery. In some embodiments, provided methods are useful for treating damaged and/or ischemic organs, transplants, or grafts, as well as ischemia/reperfusion injury or post-surgical scarring.

In some embodiments, a renal disease to be treated by provided methods of the present disclosure is renal fibrosis. In some embodiments, a renal disease to be treated by provided methods of the present disclosure is renal fibrosis secondary to, or otherwise associated with, an underlying indication. In some embodiments, a renal disease to be treated by provided methods of the present disclosure is renal fibrosis associated with renal failure, renal obstruction, renal trauma, renal transplantation, CKD, diabetes, hypertension, radiocontrast nephropathy, immune-mediated glomerulonephritides (e.g., lupus nephritis, ANCA-associated glomerulonephritides (e.g., Wegener's granulomatosis, microscopic polyangiitis, or renal limited vasculitis), anti-GBM nephropathy, IgA nephropathy, membranous glomerulonephritis, or focal and segmental glomerulosclerosis), non-immune-mediated glomerulonephritides (e.g., autosomal dominant polycystic kidney disease, collagen type III glomerulopathy, nail-patella syndrome, or Alport syndrome), minimal change disease, or nephrotic syndrome (e.g., steroid-resistant nephrotic syndrome). In some embodiments, a renal disease to be treated by provided methods of the present disclosure is nephrotic syndrome and/or diseases, disorders, or conditions associated with nephrotic syndrome (e.g., focal and segmental glomerulosclerosis, minimal change disease, and membranous nephropathy). In some embodiments, a renal disease to be treated by provided methods of the present disclosure is a fibrotic disease of the kidney that is or comprises: focal segmental glomerulosclerosis (FSGS), steroid resistant nephrotic syndrome (SRNS), proteinuria, lupus nephritis, minimal change disease, ANCA-associated glomerulonephritis, Alport syndrome, anti-GBM nephropathy, IgA nephropathy, membranous glomerulonephritis (MG), autosomal dominant polycystic kidney disease (ADPKD), or CKD. In some embodiments, a renal disease to be treated by provided methods of the present disclosure is a fibrotic disease of the kidney that is or comprises ANCA-associated glomerulonephritis. In some embodiments, a renal disease to be treated by provided methods of the present disclosure is ANCA-associated glomerulonephritis selected from Wegener's granulomatosis, microscopic polyangiitis (MPA), and renal limited vasculitis. In some embodiments, a renal disease to be treated by provided methods of the present disclosure is focal and segmental glomerulosclerosis. In some embodiments, a renal disease to be treated by provided methods of the present disclosure is Alport syndrome. In some embodiments, a renal disease to be treated by provided methods of the present disclosure is polycystic kidney disease (e.g., autosomal dominant polycystic kidney disease or autosomal recessive polycystic kidney disease).

In some embodiments, a renal disease to be treated by provided methods of the present disclosure is NS. In some embodiments, a renal disease to be treated by provided methods of the present disclosure is MCD. In some embodiments, a renal disease to be treated by provided methods of the present disclosure is ANCA-associated glomerulonephritis. In some embodiments, a renal disease to be treated by provided methods of the present disclosure is lupus nephritis. In some embodiments, a renal disease to be treated by provided methods of the present disclosure is anti-GBM nephropathy. In some embodiments, a renal disease to be treated by provided methods of the present disclosure is IgA nephropathy, also known as Berger's disease. In some embodiments, a renal disease to be treated by provided methods of the present disclosure is AS. In some embodiments, a renal disease to be treated by provided methods of the present disclosure is polycystic kidney disease. In some embodiments, a renal disease to be treated by provided methods of the present disclosure is ARPKD-CHF. In some embodiments, a renal disease to be treated by provided methods of the present disclosure is or comprises renal cysts. In some embodiments, a renal disease to be treated by provided methods of the present disclosure is collagen type III glomerulopathy. In some embodiments, a renal disease to be treated by provided methods of the present disclosure is nail-patella syndrome.

In some embodiments, provided are methods related to treatment of cerebral and/or cerebrovascular disease(s) and selecting, identifying, and/or characterizing patients likely to benefit from a treatment with a compound of Formula I as described herein. In some embodiments, provided methods are useful for treating a cerebral and/or cerebrovascular disease selected from those described above and herein in a subject in need thereof. In some embodiments, a cerebral and/or cerebrovascular disease to be treated by provided methods of the present disclosure is stroke. In some embodiments, a cerebral and/or cerebrovascular disease to be treated by provided methods of the present disclosure is cerebral infarction.

In some embodiments, provided are methods related to treatment of cardiac and/or cardiovascular disease(s) and selecting, identifying, and/or characterizing patients likely to benefit from a treatment with a compound of Formula I as described herein. In some embodiments, provided methods are useful for treating a cardiac and/or cardiovascular disease selected from those described above and herein in a subject in need thereof. In some embodiments, a cardiac and/or cardiovascular disease to be treated by provided methods of the present disclosure is cardiac fibrosis and/or fibrosis associated with cardiovascular system. In some embodiments, a cardiac and/or cardiovascular disease to be treated by provided methods of the present disclosure is cardiac fibrosis secondary to, or otherwise associated with, an underlying indication. In some embodiments, a cardiac and/or cardiovascular disease to be treated by provided methods of the present disclosure is cardiac and/or cardiovascular fibrosis associated with ischemic heart disease, myocardial ischemia, atherosclerosis, myocardial perfusion (e.g., as a consequence of chronic cardiac ischemia or myocardial infarction), vascular occlusion, or restenosis. In some embodiments, a cardiac and/or cardiovascular disease to be treated by provided methods of the present disclosure is ischemic heart disease, myocardial ischemia, atherosclerosis, myocardial perfusion (e.g., as a consequence of chronic cardiac ischemia or myocardial infarction), vascular occlusion, or restenosis.

In some embodiments, provided are methods related to treatment of pulmonary disease(s) and selecting, identifying, and/or characterizing patients likely to benefit from a treatment with a compound of Formula I as described herein. In some embodiments, provided methods are useful for treating a pulmonary disease selected from those described above and herein in a subject in need thereof. In some embodiments, a pulmonary disease to be treated by provided methods of the present disclosure is pulmonary fibrosis. In some embodiments, a pulmonary disease to be treated by provided methods of the present disclosure is pulmonary fibrosis secondary to, or otherwise associated with, an underlying indication. In some embodiments, a pulmonary disease to be treated by provided methods of the present disclosure is interstitial lung diseases (e.g., fibrosing interstitial lung diseases). In some embodiments, a pulmonary disease to be treated by provided methods of the present disclosure is pneumonias (e.g., idiopathic interstitial pneumonias). In some embodiments, a pulmonary disease to be treated by provided methods of the present disclosure is IPF. In some embodiments, a pulmonary disease to be treated by provided methods of the present disclosure is pulmonary fibrosis associated with an infection (e.g., a bacterial, viral, or fungal infection). In some embodiments, a pulmonary disease to be treated by provided methods of the present disclosure is pulmonary fibrosis associated with a viral infection (e.g., an influenza or coronavirus infection, such as COVID-19).

In some embodiments, provided are methods related to treatment of dermal disease(s) and selecting, identifying, and/or characterizing patients likely to benefit from a treatment with a compound of Formula I as described herein. In some embodiments, provided methods are useful for treating a dermal disease selected from those described above and herein in a subject in need thereof. In some embodiments, a dermal disease to be treated by provided methods of the present disclosure is dermal fibrosis. In some embodiments, a dermal disease to be treated by provided methods of the present disclosure is dermal fibrosis secondary to, or otherwise associated with, an underlying indication. In some embodiments, a dermal disease to be treated by provided methods of the present disclosure is scleroderma and/or systemic sclerosis (e.g., diffuse systemic sclerosis or limited systemic sclerosis).

In some embodiments, provided are methods related to treatment of gastrointestinal disease(s) and selecting, identifying, and/or characterizing patients likely to benefit from a treatment with a compound of Formula I as described herein. In some embodiments, provided methods are useful for treating a gastrointestinal disease selected from those described above and herein in a subject in need thereof. In some embodiments, a gastrointestinal disease to be treated by provided methods of the present disclosure is gastrointestinal fibrosis (e.g., fibrosis of esophagus, stomach, intestines, and/or colon). In some embodiments, a gastrointestinal disease to be treated by provided methods of the present disclosure is gastrointestinal fibrosis secondary to, or otherwise associated with, an underlying indication. In some embodiments, a gastrointestinal disease to be treated by provided methods of the present disclosure is IBD. In some embodiments, a gastrointestinal disease to be treated by provided methods of the present disclosure is IBD (e.g., ulcerative colitis or Crohn's disease), e.g., treating gastrointestinal fibrosis associated with IBD.

The present disclosure is based in part on the recognition that certain biomarkers can distinguish patients who are likely to respond to therapy, for example because the drivers of their hepatic, renal, cerebral and/or cerebrovascular, cardiac and/or cardiovascular, pulmonary disease, dermal, or gastrointestinal disorder or condition correspond and/or associated with, and/or secondary and/or related to the mechanism of action of a compound of Formula I as described herein. In some embodiments, a patient to be treated with a method of the present disclosure has an altered level of one or more gene products or proteins that are part of the mechanism of action of a compound of Formula I as described herein.

In some embodiments, the present disclosure provides a method of treating a patient diagnosed with, suspected of having, or at risk of a hepatic disease, a renal disease, a cerebral and/or cerebrovascular disease, a cardiac and/or cardiovascular disease, a pulmonary disease, a dermal disease, a gastrointestinal disease, comprising administering an effective amount of a compound of Formula I as described herein to a patient that has been determined to have an altered level of one or more biomarkers described herein.

In some embodiments, the present disclosure provides a method of treating a hepatic disease, a renal disease, a cerebral and/or cerebrovascular disease, a cardiac and/or cardiovascular disease, a pulmonary disease, a dermal disease, a gastrointestinal disease described above and here in a patient characterized by an altered level of one or more biomarkers described above and herein, comprising administering an effective amount of a compound of Formula I as described herein to the patient.

In some embodiments, the present disclosure provides a method comprising administering an effective amount of a compound of Formula I as described herein to a patient that has been determined to have (i) at least one symptom selected from proteinuria and/or hypoalbuminemia and/or hyperlipidemia and/or hyperglycemia and/or edema; and (ii) an altered level of one or more biomarkers described herein.

In some embodiments, the present disclosure provides a method comprising administering an effective amount of a compound of Formula I as described herein to a patient that has been determined to have (i) nephrotic syndrome; and (ii) an altered level of one or more biomarkers described herein.

In some embodiments, the present disclosure provides a method comprising administering an effective amount of a compound of Formula I as described herein to a patient that has been determined to have (i) liver steatosis and/or NASH; and (ii) an altered level of one or more biomarkers described herein.

In some embodiments, the present disclosure provides a method comprising administering an effective amount of a compound of Formula I as described herein to a patient in need thereof, wherein the patient has been determined to have an altered level of one or more biomarkers described above and herein.

In some embodiments, one or more biomarkers are described above and herein including any classes and subclasses thereof, both singly and in combination. In some embodiments, a patient has been determined to have an altered level of at least at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the biomarkers described above and herein, a fragment thereof, or a human analog thereof, including any classes and subclasses thereof, both singly and in combination. In some embodiments, an altered level of a biomarker is a level that is different from (e.g., at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold, or more, above or below) a corresponding threshold level.

In some embodiments, one or more biomarkers are selected from biomarkers identified using a method described herein. In some embodiments, one or more biomarkers are selected from biomarkers whose levels have been established to have a mean increase or decrease of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold, or more in a population of subjects administered a compound of Formula I as described herein relative to a comparable reference population. In some embodiments, one or more biomarkers are selected from biomarkers whose levels have been established to have a mean increase or decrease of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold, or more in a population of subjects with confirmed hepatic disease(s), renal disease(s), cerebral and/or cerebrovascular disease(s), cardiac and/or cardiovascular disease(s), pulmonary disease(s), dermal disease(s), or gastrointestinal disease(s) described above and herein relative to a population of healthy volunteers.

In some embodiments, the present disclosure provides a method of treating a patient diagnosed with, suspected of having, or at risk for a hepatic disease, a renal disease, a cerebral and/or cerebrovascular disease, a cardiac and/or cardiovascular disease, a pulmonary disease, a dermal disease, or a gastrointestinal disease described above and herein, comprising (i) obtaining or determining a level of one or more biomarkers described above and herein in a biological sample obtained from the patient; and (ii) comparing the determined level(s) to a corresponding threshold level. In some embodiments, a method further comprises performing an assay on a biological sample obtained from the patient to determine level(s) of one or more biomarkers. In some embodiments, if the level of one or more biomarkers is different from the corresponding threshold level, then a compound of Formula I as described herein is administered to the patient. In some embodiments, if the level of one or more biomarkers is comparable to (e.g., is not different from) the corresponding threshold level, then a compound of Formula I as described herein is not administered to the subject.

In some embodiments, the present disclosure provides methods of administering a compound of Formula I as described herein to a subject or population of subjects described herein, according to a regimen established to achieve one or more desirable outcomes. In some embodiments, the hepatic disease, renal disease, cerebral and/or cerebrovascular disease, cardiac and/or cardiovascular disease, pulmonary disease, dermal disease, or gastrointestinal disease is stabilized (i.e., does not worsen) and/or is ameliorated (i.e., one or more symptoms improve) in a patient treated with a compound of Formula I as described herein. In some embodiments, treatment of a patient with a compound of Formula I as described herein increases or decreases a level of one or more biomarkers (i.e., such that the level of the one or more biomarkers is less different from a threshold level than prior to treatment with a compound of Formula I as described herein). In some embodiments, treatment of a patient with a compound of Formula I as described herein decreases a level of one or more biomarkers that was elevated prior to treatment with a compound of Formula I as described herein (e.g., one or more biomarkers described above and herein). In some embodiments, treatment of a patient with a compound of Formula I as described herein increases a level of one or more biomarkers that was reduced prior to treatment with a compound of Formula I as described herein (e.g., one or more biomarkers described above and herein).

In some embodiments, a regimen has been established to achieve one or more desirable outcomes, relative to that observed for a comparable reference population that has not received a compound of Formula I as described herein (e.g., that has received a placebo). A placebo as used herein is a dosage form that matches that of an active study compound, but does not deliver the active study compound (e.g., a compound of Formula I as described herein). For example, a placebo can be a capsule that visually matches an active study drug and is composed of the same capsule shell but is filled with the pharmaceutical excipient (and lacking the active study drug), e.g., silicified microcrystalline cellulose. In some embodiments, in methods provided herein, a reference composition may be or may have been administered at the same intervals and/or in the same amounts as a composition providing a compound of Formula I as described herein.

In some embodiments, provided methods are useful for monitoring subjects (e.g., monitoring status of subjects over time and/or monitoring therapy). In some embodiments, the present disclosure provides methods comprising (i) administering an effective amount of a compound of Formula I as described herein to a patient characterized by an altered level of one or more biomarkers described above and herein; and (ii) monitoring levels of the one or more biomarkers, e.g., over a period of time. In some embodiments, if the level of one or more biomarkers remains altered in the patient, then therapy with a compound of Formula I as described herein is discontinued. In some embodiments, if the level of one or more biomarkers remains altered in the patient, then the dose and/or dosing frequency of a compound of Formula I as described herein is increased.

In some embodiments, the present disclosure provides a method comprising determining levels of one or more biomarkers described herein in each of a plurality of biological samples obtained at different time points from a single patient; and comparing the determined levels from a first time point with those from at least one later time point. In some embodiments, the present disclosure provides a method comprising determining levels of one or more biomarkers described herein from a biological sample obtained from a subject for whom levels of the one or more biomarkers have previously been obtained at least once; and comparing the determined levels with the previously obtained levels. In some embodiments, a first time point and one or more later time points are separated from one another by a reasonably consistent interval. In some embodiments, such methods further comprise administering therapy comprising a compound of Formula I as described herein to a subject (e.g., a subject who has been determined to have moved from a non-responsive to a responsive state).

In some embodiments, a meaningful change in a determined level over time indicates a change in the subject's status. In some embodiments, a meaningful change in a determined level over time is a change (e.g., an increase or a decrease) of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold, or more compared to a threshold level. In some embodiments, a meaningful change in a determined level over time is a change of more than about 0.5, about 1.0, about 1.5, or about 2.0, or more standard deviations away from a threshold level.

In some embodiments, provided methods are useful for monitoring therapy (e.g., efficacy and/or other indicators of response). In some embodiments, a sample from a first time point is or was obtained from a subject prior to administration of a compound of Formula I as described herein, and a sample from a second time point is or was obtained from the subject after administration of a compound of Formula I as described herein. In some such embodiments, if the levels of one or more biomarkers are no longer altered and/or are altered to a lesser degree in a later sample compared to a first sample, then therapy comprising a compound of Formula I as described herein is continued. In some such embodiments, if the levels of one or more biomarkers remain altered in a later sample compared to a first sample, then therapy comprising a compound of Formula I as described herein is discontinued, or dosage amount and/or dosing frequency of therapy comprising a compound of Formula I as described herein is increased.

In some embodiments, the present disclosure provides a method for treatment with a compound of Formula I as described herein that includes: (i) receiving a report listing the level of one or more biomarkers (e.g., one or more biomarkers described above and herein) for a patient with a hepatic disease, a renal disease, a cerebral and/or cerebrovascular disease, a cardiac and/or cardiovascular disease, a pulmonary disease, a dermal disease, a gastrointestinal disease; (ii) receiving a request for reimbursement of the screening and/or of a particular therapeutic regimen; and (iii) approving payment and/or reimbursement for treatment with therapy comprising a compound of Formula I as described herein if the report indicates the level of one or more biomarkers is above a threshold level.

Subjects to Be Treated

In some embodiments, one or more subjects or populations are selected to receive a compound of Formula I as described herein based on one or more markers and/or characteristics such as, for example, one or more risk factors of a hepatic, renal, cerebral and/or cerebrovascular, cardiac and/or cardiovascular, pulmonary disease, dermal, or gastrointestinal disorder or condition described above and herein and/or an altered level of one or more biomarkers described above and herein, etc.

In some embodiments, a subject or population thereof is selected to receive a compound of Formula I as described herein using technologies provided herein (e.g., based on assessment of one or more markers and/or characteristics, such as an assessment of one or more biomarkers described above and herein). In some embodiments, such technologies are used to inform or determine one or more features of a therapeutic regimen (e.g., selection of subject(s) to receive a particular therapy (e.g., therapy comprising a compound of Formula I as described herein and/or dose thereof and/or timing of administration of such therapy).

In some embodiments, assessment of one or more markers and/or characteristics is performed with respect to the same subject at a plurality of different time points. In some embodiments, assessment of one or more markers and/or characteristics is performed with respect to a particular patient prior to initiation of a particular therapeutic regimen (e.g., a therapeutic regimen comprising a compound of Formula I as described herein) and/or prior to administration of a particular dose of therapy (e.g., therapy comprising a compound of Formula I as described herein) in accordance with such therapeutic regimen.

In some embodiments, a subject or population thereof is suffering from or is susceptible to a hepatic, renal, cerebral and/or cerebrovascular, cardiac and/or cardiovascular, pulmonary disease, dermal, or gastrointestinal disease as described above and herein. In some embodiments, a subject or population thereof is suffering from or is susceptible to a disease, disorder, or condition characterized by or otherwise associated with a hepatic, renal, cerebral and/or cerebrovascular, cardiac and/or cardiovascular, pulmonary disease, dermal, or gastrointestinal disease as described above and herein.

In some embodiments, a subject or population thereof is suffering from or is susceptible to fibrosis. In some embodiments, a subject or population thereof is suffering from or is susceptible to a disease, disorder, or condition characterized by or otherwise associated with fibrosis. In some embodiments, a subject or population thereof is suffering from or is susceptible to fibrosis of liver, kidney, brain, heart, lung, skin, and/or gastrointestinal tract.

In some embodiments, a subject or population thereof is suffering from or is susceptible to an acute injury (e.g., an acute organ injury, such as acute lung injury, acute liver injury, or acute kidney injury). In some embodiments, a subject or population thereof is suffering from or is susceptible to a chronic injury (e.g., a chronic organ injury, such as chronic lung injury, chronic liver injury, or chronic kidney injury). In some embodiments, a subject or population thereof is suffering from a traumatic injury. In some embodiments, a subject or population thereof has undergone, is undergoing, or will undergo an organ transplantation. In some embodiments, a subject or population thereof is suffering from or susceptible to a damaged and/or ischemic organ, transplant, or graft. In some embodiments, a subject or population thereof is suffering from or susceptible to ischemia/reperfusion injury. In some embodiments, a subject or population thereof is suffering from or susceptible to post-surgical scarring.

In some embodiments, a subject or population thereof is suffering from or is susceptible to hepatitis B, hepatitis C, delta hepatitis, chronic alcoholism, extrahepatic obstructions (stones in the bile duct), cholangiopathies (primary biliary cirrhosis and sclerosing cholangitis), autoimmune liver disease, inherited metabolic disorders (Wilson's disease, hemochromatosis, alpha-1 antitrypsin deficiency, liver steatosis, NASH, liver fibrosis, liver cirrhosis, liver IR injury, or HCC. In some embodiments, a subject or population thereof is suffering from or is susceptible to liver steatosis. In some embodiments, a subject or population thereof is suffering from or is susceptible to NASH. In some embodiments, a subject or population thereof is suffering from or is susceptible to hepatic fibrosis (e.g., fibrotic liver disease). In some embodiments, a subject or population thereof is suffering from or is susceptible to liver fibrosis. In some embodiments, a subject or population thereof is suffering from or is susceptible to liver fibrosis secondary to, or otherwise associated with, an underlying indication. In some embodiments, a subject or population thereof is suffering from or is susceptible to liver cirrhosis. In some embodiments, a subject or population thereof is suffering from or is susceptible to liver cirrhosis secondary to, or otherwise associated with, an underlying indication. In some embodiments, a subject or population thereof is suffering from or is susceptible to liver IR injury. In some embodiments, a subject or population thereof is suffering from or is susceptible to HCC. In some embodiments, a subject or population thereof is suffering from or is susceptible to cirrhotic or non-cirrhotic HCC. In some embodiments, a subject or population thereof is suffering from or is susceptible to cirrhotic HCC. In some embodiments, a subject or population thereof is suffering from or is susceptible to non-cirrhotic HCC. In some embodiments, a subject or population thereof is suffering from or is susceptible to sorafenib-resistant HCC.

In some embodiments, a subject or population thereof is suffering from or is susceptible to CKD, AKI, AKI-related CKD, renal fibrosis, renal fibrosis secondary to, or otherwise associated with, an underlying indication, NS, MCD, ANCA-associated glomerulonephritis, lupus nephritis, anti-GBM nephropathy, IgA nephropathy, also known as Berger's disease, AS, polycystic kidney disease, ARPKD-CHF, renal cysts, collagen type III glomerulopathy, or nail-patella syndrome. In some embodiments, a subject or population thereof is suffering from or is susceptible to CKD. In some embodiments, a subject or population thereof is suffering from or is susceptible to AKI. In some embodiments, a subject or population thereof is suffering from or is susceptible to AKI-related CKD.

In some embodiments, a subject or population thereof is suffering from or is susceptible to renal fibrosis. In some embodiments, a subject or population thereof is suffering from or is susceptible to renal fibrosis secondary to, or otherwise associated with, an underlying indication. In some embodiments, a subject or population thereof is suffering from or is susceptible to renal fibrosis associated with renal failure, renal obstruction, renal trauma, renal transplantation, CKD, diabetes, hypertension, radiocontrast nephropathy, immune-mediated glomerulonephritides (e.g., lupus nephritis, ANCA-associated glomerulonephritides (e.g., Wegener's granulomatosis, microscopic polyangiitis, or renal limited vasculitis), anti-GBM nephropathy, IgA nephropathy, membranous glomerulonephritis, or focal and segmental glomerulosclerosis), non-immune-mediated glomerulonephritides (e.g., autosomal dominant polycystic kidney disease, collagen type III glomerulopathy, nail-patella syndrome, or Alport syndrome), minimal change disease, or nephrotic syndrome (e.g., steroid-resistant nephrotic syndrome). In some embodiments, a subject or population thereof is suffering from or is susceptible to nephrotic syndrome and/or diseases, disorders, or conditions associated with nephrotic syndrome (e.g., focal and segmental glomerulosclerosis, minimal change disease, and membranous nephropathy). In some embodiments, a subject or population thereof is suffering from or is susceptible to a fibrotic disease of the kidney that is or comprises: focal segmental glomerulosclerosis (FSGS), steroid resistant nephrotic syndrome (SRNS), proteinuria, lupus nephritis, minimal change disease, ANCA-associated glomerulonephritis, Alport syndrome, anti-GBM nephropathy, IgA nephropathy, membranous glomerulonephritis (MG), autosomal dominant polycystic kidney disease (ADPKD), or CKD. In some embodiments, a subject or population thereof is suffering from or is susceptible to a fibrotic disease of the kidney that is or comprises ANCA-associated glomerulonephritis. In some embodiments, a subject or population thereof is suffering from or is susceptible to ANCA-associated glomerulonephritis selected from Wegener's granulomatosis, microscopic polyangiitis (MPA), and renal limited vasculitis. In some embodiments, a subject or population thereof is suffering from or is susceptible to focal and segmental glomerulosclerosis. In some embodiments, a subject or population thereof is suffering from or is susceptible to Alport syndrome. In some embodiments, a subject or population thereof is suffering from or is susceptible to polycystic kidney disease (e.g., autosomal dominant polycystic kidney disease or autosomal recessive polycystic kidney disease).

In some embodiments, a subject or population thereof is suffering from or is susceptible to NS. In some embodiments, a subject or population thereof is suffering from or is susceptible to MCD. In some embodiments, a subject or population thereof is suffering from or is susceptible to ANCA-associated glomerulonephritis. In some embodiments, a subject or population thereof is suffering from or is susceptible to lupus nephritis. In some embodiments, a subject or population thereof is suffering from or is susceptible to anti-GBM nephropathy. In some embodiments, a subject or population thereof is suffering from or is susceptible to IgA nephropathy, also known as Berger's disease. In some embodiments, a subject or population thereof is suffering from or is susceptible to AS. In some embodiments, a subject or population thereof is suffering from or is susceptible to polycystic kidney disease. In some embodiments, a subject or population thereof is suffering from or is susceptible to ARPKD-CHF. In some embodiments, a subject or population thereof is suffering from or is susceptible to renal cysts. In some embodiments, a subject or population thereof is suffering from or is susceptible to collagen type III glomerulopathy. In some embodiments, a subject or population thereof is suffering from or is susceptible to nail-patella syndrome.

In some embodiments, a subject or population thereof is suffering from or is susceptible to stroke. In some embodiments, a subject or population thereof is suffering from or is susceptible to cerebral infarction.

In some embodiments, a subject or population thereof is suffering from or is susceptible to cardiac fibrosis and/or fibrosis associated with cardiovascular system. In some embodiments, a subject or population thereof is suffering from or is susceptible to cardiac fibrosis secondary to, or otherwise associated with, an underlying indication. In some embodiments, a subject or population thereof is suffering from or is susceptible to cardiac and/or cardiovascular fibrosis associated with ischemic heart disease, myocardial ischemia, atherosclerosis, myocardial perfusion (e.g., as a consequence of chronic cardiac ischemia or myocardial infarction), vascular occlusion, or restenosis. In some embodiments, a subject or population thereof is suffering from or is susceptible to ischemic heart disease, myocardial ischemia, atherosclerosis, myocardial perfusion (e.g., as a consequence of chronic cardiac ischemia or myocardial infarction), vascular occlusion, or restenosis.

In some embodiments, a subject or population thereof is suffering from or is susceptible to pulmonary fibrosis. In some embodiments, a subject or population thereof is suffering from or is susceptible to pulmonary fibrosis secondary to, or otherwise associated with, an underlying indication. In some embodiments, a subject or population thereof is suffering from or is susceptible to interstitial lung diseases (e.g., fibrosing interstitial lung diseases). In some embodiments, a subject or population thereof is suffering from or is susceptible to pneumonias (e.g., idiopathic interstitial pneumonias). In some embodiments, a subject or population thereof is suffering from or is susceptible to IPF. In some embodiments, a subject or population thereof is suffering from or is susceptible to pulmonary fibrosis associated with an infection (e.g., a bacterial, viral, or fungal infection). In some embodiments, a subject or population thereof is suffering from or is susceptible to pulmonary fibrosis associated with a viral infection (e.g., an influenza or coronavirus infection, such as COVID-19).

In some embodiments, a subject or population thereof is suffering from or is susceptible to dermal fibrosis. In some embodiments, a subject or population thereof is suffering from or is susceptible to dermal fibrosis secondary to, or otherwise associated with, an underlying indication. In some embodiments, a subject or population thereof is suffering from or is susceptible to scleroderma and/or systemic sclerosis (e.g., diffuse systemic sclerosis or limited systemic sclerosis).

In some embodiments, a subject or population thereof is suffering from or is susceptible to gastrointestinal fibrosis (e.g., fibrosis of esophagus, stomach, intestines, and/or colon). In some embodiments, a subject or population thereof is suffering from or is susceptible to gastrointestinal fibrosis secondary to, or otherwise associated with, an underlying indication. In some embodiments, a subject or population thereof is suffering from or is susceptible to IBD. In some embodiments, a subject or population thereof is suffering from or is susceptible to IBD (e.g., ulcerative colitis or Crohn's disease), e.g., treating gastrointestinal fibrosis associated with IBD.

In some embodiments, a subject or population thereof has one or more symptoms selected from proteinuria, hypoalbuminemia, hyperlipidemia, hyperglycemia, and edema. In some embodiments, a subject or population thereof has proteinuria. In some embodiments, a subject or population thereof has hypoalbuminemia. In some embodiments, a subject or population thereof has hyperlipidemia. In some embodiments, a subject or population thereof has hyperglycemia. In some embodiments, a subject or population thereof has edema.

In some embodiments, a subject or population thereof has liver steatosis. In some embodiments, a subject or population thereof has NASH.

Administration

A composition providing a compound of Formula I as described herein can be administered to subjects in accordance with methods provided herein.

In some embodiments, a composition providing a compound of Formula I as described herein is a composition comprising a compound of Formula I as described herein (in a pharmaceutically acceptable form as described herein), formulated together with one or more pharmaceutically acceptable carriers. In some embodiments, a composition providing a compound of Formula I as described herein is or comprises a compound of Formula I as described herein present in a unit dose amount appropriate for administration in a therapeutic regimen that shows a statistically significant probability of achieving a predetermined therapeutic effect when administered to a relevant population. In some embodiments, compositions providing a compound of Formula I as described herein may be specially formulated for administration in solid or liquid form, including those adapted for the following: oral administration, for example, drenches (aqueous or non-aqueous solutions or suspensions), capsules, tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

In some embodiments, as described herein, composition providing a compound of Formula I as described herein is formulated for oral administration (e.g., in a capsule form). In some embodiments, a composition providing a compound of Formula I as described herein is administered orally.

In some embodiments, a composition providing a compound of Formula I as described herein is administered as one or more unit dosage forms. In some embodiments, a composition providing a compound of Formula I as described herein is administered as one or more solid unit dosage forms (e.g., one or more capsules or tablets). In some embodiments, a compound of Formula I as described herein is administered as one or more oral unit dosage forms. In some embodiments, a composition providing a compound of Formula I as described herein is an immediate release solid unit dosage form.

In some embodiments, a composition providing a compound of Formula I as described herein is a capsule. In some embodiments, a composition providing a compound of Formula I as described herein is a tablet.

In some embodiments, a compound of Formula I as described herein is administered as a capsule comprising 10 mg, 50 mg, or 250 mg of a compound of Formula I as described herein. In some embodiments, a compound of Formula I as described herein is administered as a capsule comprising 10 mg, 50 mg, or 250 mg of a compound of Formula I as described herein with no excipients.

It will be appreciated that a suitable number of unit dosage forms (e.g., tablets or capsules) are administered in order to provide a suitable dose as described herein. For example, in some embodiments, one unit dosage form (e.g., tablet or capsule) is administered in order to provide a suitable dose (e.g., a dose of about 50 mg, about 100 mg, about 200 mg, about 250 mg, about 400 mg, about 500 mg, or about 600 mg); in some embodiments, more than one (e.g., 2, 3, 4, 5, etc.) unit dosage forms (e.g., tablets or capsules) are administered in order to provide a suitable dose (e.g., a dose of about 50 mg, about 100 mg, about 200 mg, about 250 mg, about 400 mg, about 500 mg, or about 600 mg). In some embodiments, when multiple unit dosage forms are administered, each unit dosage form contains the same amount of a compound of Formula I as described herein, in order to provide a suitable dose as described herein; in some embodiments, when multiple unit dosage forms are administered, each unit dosage form contains different amounts of a compound of Formula I as described herein, in order to provide a suitable dose as described herein.

In some embodiments, a composition providing a compound of Formula I as described herein is administered as a single dose. In some embodiments, a composition providing a compound of Formula I as described herein is administered at regular intervals. Administration at an “interval,” as used herein, indicates that the therapeutically effective amount is administered periodically (as distinguished from a one-time dose). In some embodiments, a composition providing a compound of Formula I as described herein is administered bimonthly (Q2M), monthly (QM), twice monthly (BIM), triweekly (Q3W), biweekly (Q2W), weekly (QW), twice weekly (BIW), thrice weekly (TIW), daily (QD), twice daily (BID), thrice daily (TID), or four times a day (QID) in accordance with methods provided herein. In some embodiments, a composition providing a compound of Formula I as described herein is administered twice daily (BID). In some embodiments, a composition providing a compound of Formula I as described herein is administered once daily (QD).

In some embodiments, a compound of Formula I as described herein is administered in a daily dose of from about 50 mg to about 600 mg, from about 100 mg to about 600 mg, from about 200 mg to about 600 mg, from about 400 mg to about 600 mg, from about 50 mg to about 500 mg, from about 100 mg to about 500 mg, from about 250 mg to about 500 mg, from about 50 mg to about 250 mg, from about 100 mg to about 1000 mg, from about 200 mg to about 1000 mg, from about 500 mg to about 1000 mg, or from about 200 mg to about 500 mg. In some embodiments, a compound of Formula I as described herein is administered in a dose of about 50 mg, about 100 mg, about 200 mg, about 250 mg, about 400 mg, about 500 mg, or about 600 mg. In some embodiments, a compound of Formula I as described herein is administered in a daily dose of about 50 mg, about 100 mg, about 200 mg, about 400 mg, about 500 mg, or about 600 mg. In some embodiments, a compound of Formula I as described herein is administered in a twice daily dose of about 50 mg, about 100 mg, about 250 mg, or about 500 mg.

In some embodiments, a composition providing a compound of Formula I as described herein is administered at regular intervals indefinitely. In some embodiments, a composition providing a compound of Formula I as described herein is administered at regular intervals for a defined period of time.

In some embodiments, a composition providing a compound of Formula I as described herein is administered to a subject in a fed state (e.g., after a meal, such as within 1 hour, 45 minutes, 30 minutes, or 15 minutes of a meal). In some embodiments, a compound of Formula I as described herein is administered to a subject in a fasted state (e.g., after a fast of at least 4 hours, at least 6 hours, at least 8 hours, at least 10 hours, at least 12 hours, or at least 16 hours).

### EXEMPLARY EMBODIMENTS

The following number embodiments, while non-limiting, are exemplary of certain aspects of the present disclosure.

1. A compound of Formula I:

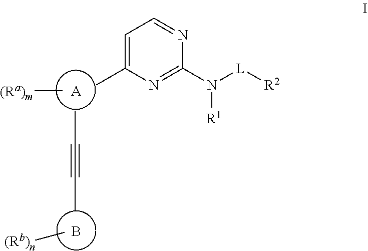

or a pharmaceutically acceptable salt thereof, wherein:
Ring A is selected from phenyl and a 6-membered heteroaryl ring comprising 1-3 nitrogen atoms;
Ring B is selected from phenyl, a 5- to 6-membered heteroaryl ring comprising 1-3 heteroatoms independently selected from nitrogen, oxygen, and sulfur, and a 9- to 10-membered heteroaryl ring comprising 1-4 heteroatoms independently selected from nitrogen, oxygen, and sulfur;
each R^{a} is independently selected from halogen, CN, CO₂R, C(O)NR₂, NR₂, OR, SR, and optionally substituted C₁₋₆ aliphatic;
each R^{b} is independently selected from halogen, CN, CO₂R, C(O)NR₂, NR₂, OR, SR, oxo and optionally substituted C₁₋₆ aliphatic;
R¹ is hydrogen or optionally substituted C₁₋₆ aliphatic;
L is a covalent bond or a bivalent C₁₋₆ straight or branched hydrocarbon chain;
R² is

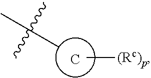

C(O)NR₂, NR₂, OR, or S(═O)ₓR;
Ring C is selected from a 3- to 7-membered cycloaliphatic ring, phenyl, a 3- to 7-membered heterocyclic ring comprising 1-3 heteroatoms independently selected from nitrogen, oxygen, and sulfur, a 5- to 6-membered heteroaryl ring comprising 1-4 heteroatoms independently selected from nitrogen, oxygen, and sulfur, and a 9- to 10-membered heteroaryl ring comprising 1-5 heteroatoms independently selected from nitrogen, oxygen, and sulfur
each R^{c} is independently selected from halogen, oxo, OR, CO₂R, C(O)N(R)₂, and optionally substituted C₁₋₆ aliphatic, or
   two independent occurrences of R^{c}, taken together with their intervening atom(s), form an optionally substituted 5-to 8-membered heterocyclic ring comprising 1-4 heteroatoms independently selected from nitrogen, oxygen, and sulfur;
each R is independently selected from hydrogen and an optionally substituted group selected from C₁₋₆ aliphatic, phenyl, a 7- to 9-membered bridged bicyclic cycloaliphatic ring, and a 3- to 7-membered heterocyclic ring comprising 1-3 heteroatoms independently selected from nitrogen, oxygen, and sulfur; or:
   two independent occurrences of R, taken together with the nitrogen atom to which they are attached, form an optionally substituted 3- to 7-membered heterocyclic ring comprising 0-3 additional heteroatoms independently selected from nitrogen, oxygen, and sulfur;
x is 0, 1, or 2; and
each of m, n, and p is independently 0-4.
   2. The compound according to embodiment 1, wherein the compound is of formula I-a:

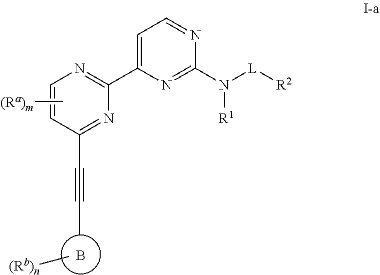

or a pharmaceutically acceptable salt thereof.
3. The compound according to embodiment 1 or 2, wherein the compound is of formula I-b:

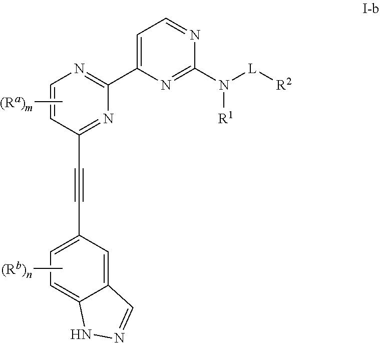

or a pharmaceutically acceptable salt thereof.
4. The compound according to any one of embodiments 1-3, wherein the compound is of formula I-c:

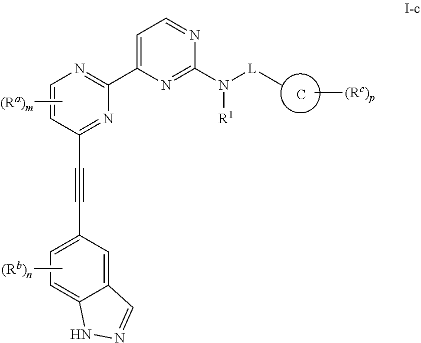

or a pharmaceutically acceptable salt thereof.
5. The compound according to any one of embodiments 1-3, wherein the compound is of formula I-d:

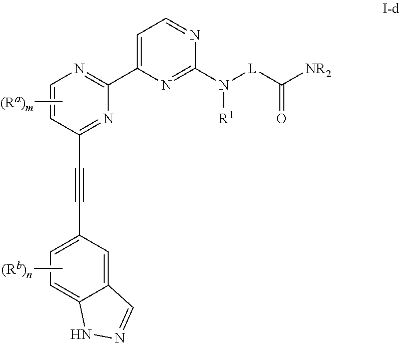

or a pharmaceutically acceptable salt thereof.
6. The compound according to embodiment 1, wherein Ring A is a 6-membered heteroaryl ring comprising 1-3 nitrogen atoms.
7. The compound according to embodiment 6, wherein Ring A is pyrimidinyl.
8. The compound according to embodiment 6 or 7, wherein Ring A is

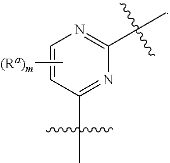

9. The compound according to embodiment 1 or 2, wherein Ring B is a 9- to 10-membered heteroaryl ring having 1-4 heteroatoms independently selected from nitrogen, oxygen, and sulfur.
10. The compound according to embodiment 9, wherein Ring B is indazolyl.
11. The compound according to embodiment 9 or 10, wherein Ring B is

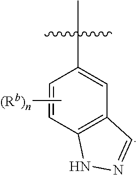

12. The compound according to any one of the preceding embodiments, each R^{b} is independently selected from halogen and optionally substituted C₁₋₆ aliphatic.
13. The compound according to embodiment 12, each R^{b} is halogen.
14. The compound according to embodiment 12 or 13, each R^{b} is fluoro.
15. The compound according to any one of the preceding embodiments, wherein R¹ is hydrogen.
16. The compound according to any one of embodiments 1-14, wherein R¹ is optionally substituted C₁₋₆ aliphatic.
17. The compound according to any one of the preceding embodiments, wherein L is a covalent bond.
18. The compound according to any one of embodiments 1-16, wherein L is a bivalent C₁₋₆ straight or branched hydrocarbon chain.
19. The compound according to embodiment 18, wherein L is —CH₂—.
20. The compound according to any one of embodiments 1-3 and 6-19, wherein R² is

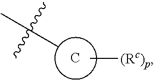

wherein Ring C is selected from a 3- to 7-membered cycloaliphatic ring, phenyl, a 3- to 7-membered heterocyclic ring comprising 1-3 heteroatoms independently selected from nitrogen, oxygen, and sulfur, a 5- to 6-membered heteroaryl ring comprising 1-4 heteroatoms independently selected from nitrogen, oxygen, and sulfur, and a 9- to 10-membered heteroaryl ring comprising 1-5 heteroatoms independently selected from nitrogen, oxygen, and sulfur.
   21. The compound according to embodiment 20, wherein Ring C is a 3- to 7-membered cycloaliphatic ring.
   22. The compound according to embodiment 21, wherein Ring C is cyclopentyl.
   23. The compound according to embodiment 21 or 22, wherein Ring C is selected from

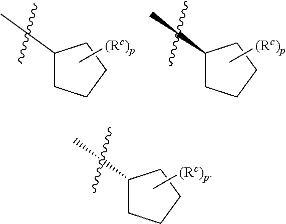

24. The compound according to embodiment 21, wherein Ring C is cyclohexyl.
25. The compound according to embodiment 21 or 24, wherein Ring C is selected from

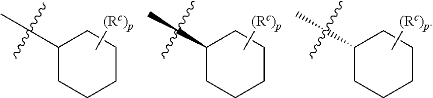

26. The compound according to embodiment 20, wherein Ring C is phenyl.
27. The compound according to embodiment 20, wherein Ring C is a 3- to 7-membered heterocyclic ring comprising 1-3 heteroatoms independently selected from nitrogen, oxygen, and sulfur.
28. The compound according to embodiment 27, wherein Ring C is a 5-membered heterocyclic ring comprising 1 heteroatom selected from nitrogen, oxygen, and sulfur.
29. The compound according to embodiment 27 or 28, wherein Ring C is tetrahydrofuranyl.
30. The compound according to any one of embodiments 27-29, wherein Ring C is selected from

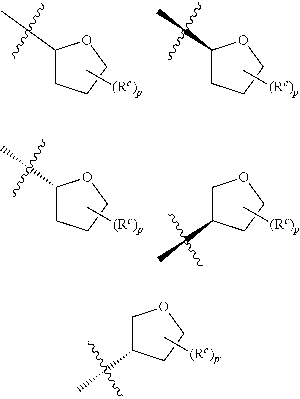

31. The compound according to embodiment 20, wherein Ring C is a 5- to 6-membered heteroaryl ring comprising 1-3 heteroatoms independently selected from nitrogen, oxygen, and sulfur.
32. The compound according to embodiment 20, wherein Ring C is a 9- to 10-membered heteroaryl ring comprising 1-5 heteroatoms independently selected from nitrogen, oxygen, and sulfur.
33. The compound according to any one of embodiments 1-3 and 6-19, wherein R² is C(O)NR₂,
wherein each R is independently selected from hydrogen or an optionally substituted group selected from C₁₋₆ aliphatic and a 7- to 9-membered bridged bicyclic cycloaliphatic ring, or two occurrences of R, taken together with the nitrogen atom to which they are attached, form an optionally substituted 3- to 7-membered heterocyclic ring comprising 0-3 additional heteroatoms independently selected from nitrogen, oxygen, and sulfur.
   34. The compound according to any one of embodiments 1-3 and 6-19, wherein R² is selected from —OCH₃, —OH, —NH₂,

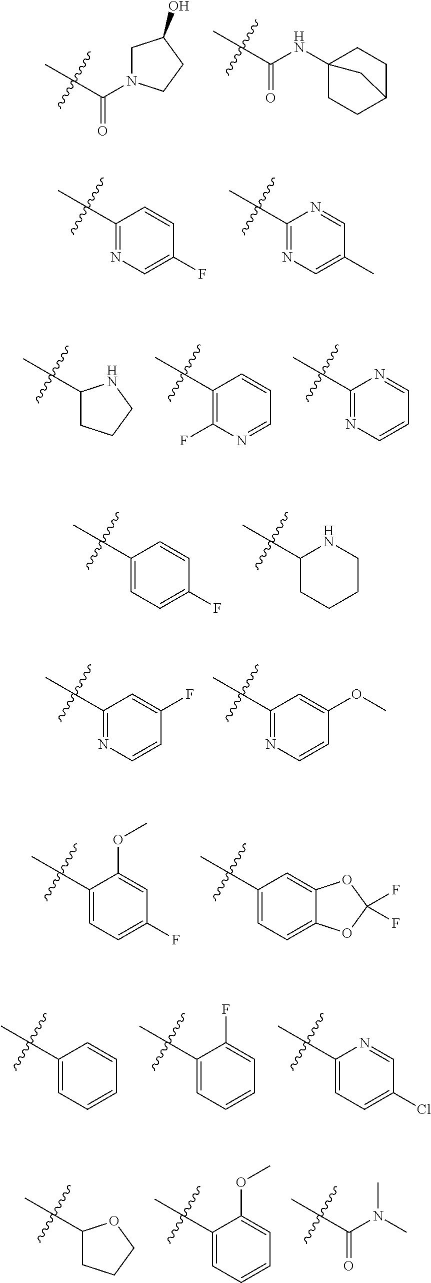

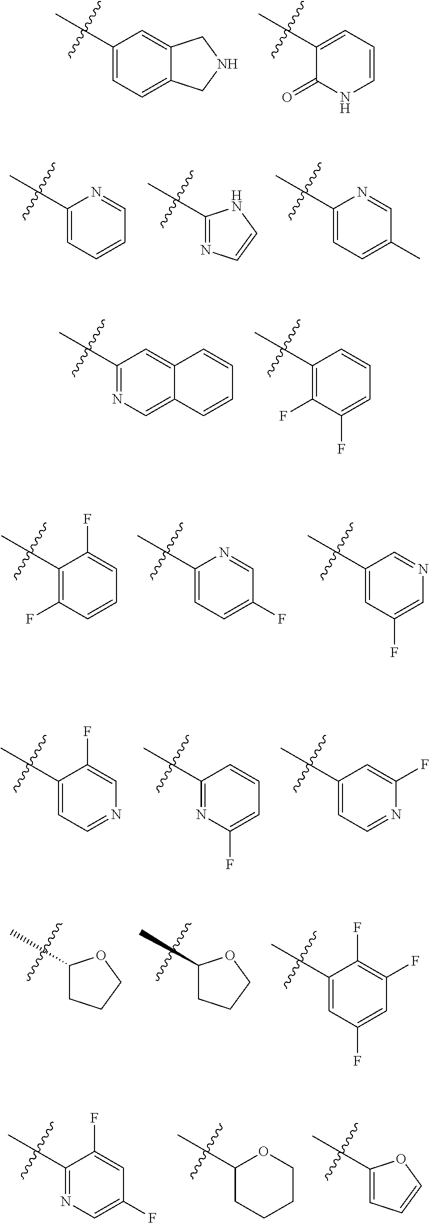

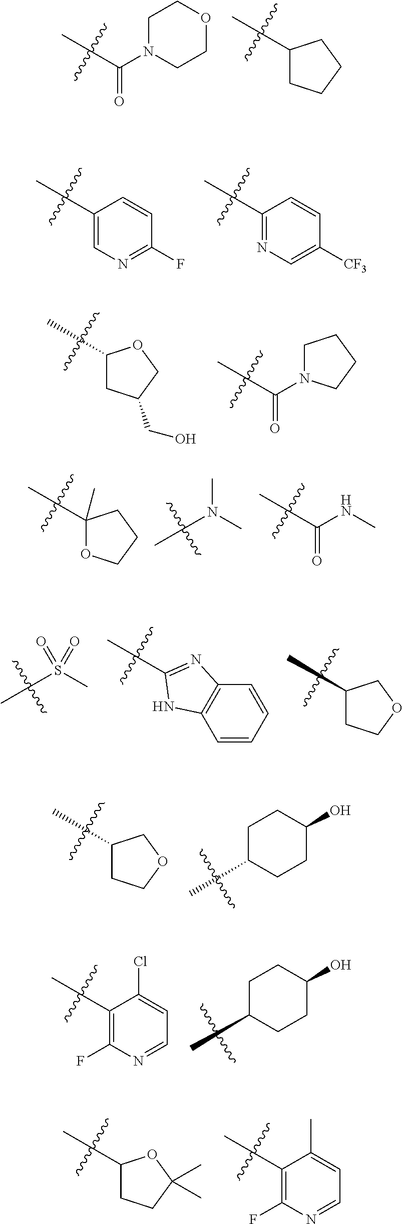

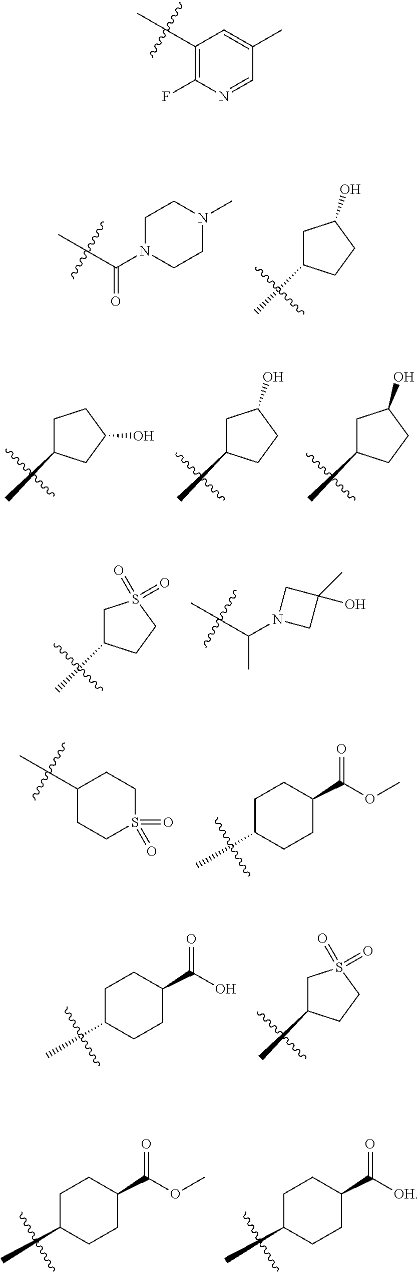

35. The compound according to any one of embodiments 1-3, wherein the compound is selected from those listed in Table 1, or a pharmaceutically acceptable salt thereof.
36. A pharmaceutical composition comprising a compound according to any one of embodiments 1-35, or a pharmaceutically acceptable salt thereof, and a pharmaceutically acceptable carrier.
37. A method of inhibiting ROCK1 and/or ROCK2, the method comprising contacting a biological sample with a compound according to any one of embodiments 1-35, or a pharmaceutically acceptable salt thereof.
38. The method according to embodiment 37, wherein the compound is selective for ROCK2.
39. A method of treating or lessening the severity of a disease or disorder associated with or mediated by Rho-associated coiled-coil kinase (ROCK), the method comprising administering to a patient in need thereof a compound according to any one of embodiments 1-35, or a pharmaceutically acceptable salt thereof.
40. The method according to embodiment 39, wherein the compound is selective for ROCK2.
41. The method according to embodiment 39 or 40, wherein the disease or disorder is selected from a hepatic disease, a renal disease, a cerebral and/or cerebrovascular disease, a cardiac and/or cardiovascular disease, a pulmonary disease, a dermal disease, a gastrointestinal disease, an ischemic disease, and a fibrotic disease described above and herein.
42. The method according to embodiment 41, wherein the fibrotic disease is fibrosis of gastrointestinal tract, heart, kidney, lung, liver, or skin.
43. The method according to embodiment 41, wherein the hepatic disease is selected from the group consisting of hepatitis B, hepatitis C, delta hepatitis, chronic alcoholism, extrahepatic obstructions (stones in the bile duct), cholangiopathies (primary biliary cirrhosis and sclerosing cholangitis), autoimmune liver disease, inherited metabolic disorders (Wilson's disease, hemochromatosis, alpha-1 antitrypsin deficiency, liver steatosis, non-alcoholic steatohepatitis (NASH), liver fibrosis, liver cirrhosis, liver ischemia-reperfusion (IR) injury, and hepatocellular carcinoma (HCC).
44. The method according to embodiment 41, wherein the renal disease is selected from the group consisting of chronic kidney disease (CKD), acute kidney injury (AKI), acute kidney injury (AKI)-related chronic kidney disease (CKD), renal fibrosis, renal fibrosis secondary to, or otherwise associated with, an underlying indication, nephrotic syndrome (NS), minimal change disease (MCD), anti-neutrophil cytoplasmic antibody (ANCA)-associated glomerulonephritis, lupus nephritis, anti-globular basement membrane (anti-GBM) nephropathy, IgA nephropathy, also known as Berger's disease, Alport syndrome (AS), polycystic kidney disease, polycystic kidney disease (e.g., autosomal recessive polycystic kidney disease (ARPKD)-congenital hepatic fibrosis (CHF)), renal cysts, collagen type III glomerulopathy, and nail-patella syndrome.
45. The method according to embodiment 41, wherein the cerebral and/or cerebrovascular disease is selected from the group consisting of stroke and cerebral infarction.
46. The method according to embodiment 41, wherein the cardiac and/or cardiovascular disease is selected from the group consisting of ischemic heart disease, myocardial ischemia, atherosclerosis, myocardial perfusion (e.g., as a consequence of chronic cardiac ischemia or myocardial infarction), vascular occlusion, and restenosis.
47. The method according to embodiment 41, wherein the disease or disorder is scleroderma or systemic sclerosis or inflammatory bowel disease.
48. A method comprising administering an effective amount of a compound according to any one of embodiments 1-35 to a patient that has been determined to have an altered level of one or more biomarkers described above and herein or a human analog thereof.
49. A method of treating a hepatic disease, a renal disease, a cerebral and/or cerebrovascular disease, a cardiac and/or cardiovascular disease, a pulmonary disease, a dermal disease, or a gastrointestinal disease described above and herein, the method comprising administering an effective amount of a compound according to any one of embodiments 1-35 to a patient that has been determined to have an altered level of one or more biomarkers described above and herein or a human analog thereof.
50. A method comprising administering an effective amount of a compound according to any one of embodiments 1-35 to a patient that has been determined to have (i) proteinuria and/or hypoalbuminemia and/or hyperlipidemia and/or hyperglycemia and/or edema; and (ii) an altered level of one or more biomarkers described above and herein or a human analog thereof.
51. A method comprising administering an effective amount of a compound according to any one of embodiments 1-35 to a patient that has been determined to have (i) liver steatosis and/or non-alcoholic steatohepatitis (NASH); and (ii) an altered level of one or more biomarkers described above and herein or a human analog thereof.
52. The method of any one of embodiment 48-51, wherein the patient has been determined to have an altered level of one or more biomarkers described above and herein, or a human analog thereof, with:
a change in mean expression for sham animals relative to FFD animals of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold; and/or
a change in mean expression for FFD animals relative to animals administered FFD+a compound according to any one of embodiments 1-35 of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold; and/or
a change in mean expression for sham animals relative to animals administered FFD+a compound according to any one of embodiments 1-35 of less than about 100%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, or about 5%.
   53. The method of any one of embodiment 48-51, wherein the patient has been determined to have an altered level of one or more biomarkers described above and herein, or a human analog thereof, with:
a change in mean expression for sham animals relative to UUO animals of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold; and/or
a change in mean expression for UUO animals relative to animals treated with UUO+a compound according to any one of embodiments 1-35 of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold; and/or
a change in mean expression for sham animals relative to animals treated with UUO+a compound according to any one of embodiments 1-35 of less than about 100%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, or about 5%.
   54. The method of any one of embodiments 48-53, wherein the patient has been determined to have an altered level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the biomarkers.
   55. The method of any one of embodiments 48-54, wherein the level of the one or more biomarkers is at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold different from that of a corresponding threshold level.
   56. The method of embodiment 55, wherein the threshold level corresponds to a predetermined mean or median level of the biomarker in a population of healthy subjects.
   57. The method of any one of embodiments 48-56, wherein the altered level of one or more biomarkers was obtained from or determined in a biological sample obtained from the patient.
   58. The method of any one of embodiments 48-57, wherein the method further comprises obtaining or determining a level of one or more biomarkers in a biological sample obtained from the patient.
   59. A method comprising:
(i) obtaining or determining a level of one or more biomarkers in a biological sample obtained from the patient, wherein the one or more biomarkers are described above and herein or a human analog thereof, and
(ii) comparing the level of the one or more biomarkers with that of a corresponding threshold level.
   60. The method of embodiment 59, wherein if the level of one or more of the biomarkers is different from the corresponding threshold level, then administering to the patient an effective amount of a compound according to any one of embodiments 1-35.
   61. The method of embodiment 59 or 60, wherein if the level of one or more of the biomarkers is comparable to the corresponding threshold level, then the patient is not administered a compound according to any one of embodiments 1-35.
   62. The method of any one of embodiments 59-61, wherein the one or more biomarkers are described above and herein, or a human analog thereof, with:
a change in mean expression for sham animals relative to FFD animals of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold; and/or
a change in mean expression for FFD animals relative to animals administered FFD+a compound according to any one of embodiments 1-35 of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold; and/or
a change in mean expression for sham animals relative to animals administered FFD+a compound according to any one of embodiments 1-35 of less than about 100%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, or about 5%.
   63. The method of any one of embodiments 59-61, wherein the one or more biomarkers are described above and herein, or a human analog thereof, with:
a change in mean expression for sham animals relative to UUO animals of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold; and/or
a change in mean expression for UUO animals relative to animals treated with UUO+a compound according to any one of embodiments 1-35 of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold; and/or
a change in mean expression for sham animals relative to animals treated with UUO+a compound according to any one of embodiments 1-35 of less than about 100%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, or about 5%.
   64. The method of any one of embodiments 59-63, wherein if the level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the biomarkers is different from the corresponding threshold level, then administering to the patient an effective amount of a compound according to any one of embodiments 1-35.
   65. The method of any one of embodiments 59-63, wherein if the level of the one or more biomarkers is at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold different from the corresponding threshold level, then administering to the patient an effective amount of a compound according to any one of embodiments 1-35.
   66. A method of treating a patient diagnosed with, suspected of having, or at risk of a hepatic disease, a renal disease, a cerebral and/or cerebrovascular disease, a cardiac and/or cardiovascular disease, a pulmonary disease, a dermal disease, or a gastrointestinal disease described above and herein, wherein the patient is characterized by an altered level of one or more biomarkers described above and herein or a human analog thereof, the method comprising:
(i) administering an effective amount of a compound according to any one of embodiments 1-35; and
(ii) monitoring levels of the one or more biomarkers.
   67. The method of embodiment 66, wherein if the levels of one or more biomarkers remain altered in the patient, discontinuing further therapy with a compound according to any one of embodiments 1-35.
   68. The method of embodiment 67, wherein if the levels of at least at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the one or more biomarkers remain altered in the patient, discontinuing further therapy with a compound according to any one of embodiments 1-35.
   69. The method of embodiment 66, wherein if the levels of the one or more biomarkers remain altered in the patient, increasing the dose and/or dosing frequency of a compound according to any one of embodiments 1-35 administered to the patient.
   70. The method of embodiment 69, wherein if the levels of at least at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the one or more biomarkers remain altered in the patient, increasing the dose and/or dosing frequency of a compound according to any one of embodiments 1-35 administered to the patient.
   71. The method of any one of embodiments 66-70, wherein the one or more biomarkers are described above and herein, or a human analog thereof, with:
a change in mean expression for sham animals relative to FFD animals of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold; and/or
a change in mean expression for FFD animals relative to animals administered FFD+a compound according to any one of embodiments 1-35 of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold; and/or
a change in mean expression for sham animals relative to animals administered FFD+a compound according to any one of embodiments 1-35 of less than about 100%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, or about 5%.
   72. The method of any one of embodiments 66-70, wherein the one or more biomarkers are described above and herein, or a human analog thereof, with:
a change in mean expression for sham animals relative to UUO animals of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold; and/or
a change in mean expression for UUO animals relative to animals treated with UUO+a compound according to any one of embodiments 1-35 of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold; and/or
a change in mean expression for sham animals relative to animals treated with UUO+a compound according to any one of embodiments 1-35 of less than about 100%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, or about 5%.
   73. A method of treating a hepatic disease, a renal disease, a cerebral and/or cerebrovascular disease, a cardiac and/or cardiovascular disease, a pulmonary disease, a dermal disease, or a gastrointestinal disease described above and herein, the method comprising administering an effective amount of a compound according to any one of embodiments 1-35 to a patient that has been determined to have an altered level of one or more biomarkers,
wherein the one or more biomarkers are described above and herein, or human analogs thereof, whose levels have been established to have:
a mean change in a population of subjects administered a compound according to any one of embodiments 1-35 relative to a comparable reference population; and/or
a mean change in a population of subjects with confirmed hepatic disease, renal disease, cerebral and/or cerebrovascular disease, cardiac and/or cardiovascular disease, pulmonary disease, dermal disease, or gastrointestinal disease relative to a population of healthy volunteers.
   74. The method of embodiment 73, wherein the one or more biomarkers are described above and herein or human analogs thereof whose levels have been established to have:
a mean change of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold in a population of subjects administered a compound according to any one of embodiments 1-35 relative to a comparable reference population; and/or
a mean change of at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold in a population of subjects with confirmed hepatic disease, renal disease, cerebral and/or cerebrovascular disease, cardiac and/or cardiovascular disease, pulmonary disease, dermal disease, or gastrointestinal disease relative to a population of healthy volunteers.
   75. The method of embodiment 73 or 74, wherein the population of subjects is a population of rodent subjects.
   76. The method of embodiment 73 or 74, wherein the population of subjects is a population of human subjects.
   77. The method of any one of embodiments 73-76, wherein the patient has been determined to have a level of the one or more biomarkers that is at least about 1.05-fold, about 1.1-fold, about 1.15-fold, about 1.2-fold, about 1.25-fold, about 1.3-fold, about 1.35-fold, about 1.4-fold, about 1.45-fold, about 1.5-fold, about 1.55-fold, about 1.6-fold, about 1.65-fold, about 1.7-fold, about 1.75-fold, about 1.8-fold, about 1.85-fold, about 1.9-fold, about 1.95-fold, about 2-fold, about 3-fold, about 4-fold, about 5-fold, about 10-fold, or about 20-fold different from that of a corresponding threshold level.
   78. The method of embodiment 77, wherein the threshold level corresponds to a predetermined mean or median level of the biomarker in a population of healthy subjects.
   79. The method of any one of embodiments 73-78, wherein the patient has been determined to have an altered level of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the biomarkers.
   80. The method of any one of embodiments 73-79, wherein the altered level of one or more biomarkers was obtained from or determined in a biological sample obtained from the patient.
   81. The method of any one of embodiments 73-80, wherein the method further comprises obtaining or determining a level of one or more biomarkers in a biological sample obtained from the patient.
   82. The method of any one of embodiments 48-81, wherein the level of one or more biomarkers is a level of expression of one or more gene products or proteins.
   83. The method of any one of embodiments 48-82, wherein the biological sample is a liver biopsy sample.
   84. The method of any one of embodiments 48-82, wherein the biological sample is a renal biopsy sample.
   85. The method of any one of embodiments 48-84, wherein the biological sample is a blood sample.
   86. The method of any one of embodiments 48-84, wherein the biological sample is a urine sample.
   87. The method of any one of embodiments 48-86, wherein the patient is diagnosed with, suspected of having, or at risk of a hepatic disease, a renal disease, a cerebral and/or cerebrovascular disease, a cardiac and/or cardiovascular disease, a pulmonary disease, a dermal disease, or a gastrointestinal disease described above and herein.
   88. The method of any one of embodiments 48-87, wherein the patient is diagnosed with, suspected of having, or at risk of a hepatic disease selected from the group consisting of hepatitis B, hepatitis C, delta hepatitis, chronic alcoholism, extrahepatic obstructions (stones in the bile duct), cholangiopathies (primary biliary cirrhosis and sclerosing cholangitis), autoimmune liver disease, inherited metabolic disorders (Wilson's disease, hemochromatosis, alpha-1 antitrypsin deficiency, liver steatosis, non-alcoholic steatohepatitis (NASH), liver fibrosis, liver cirrhosis, liver ischemia-reperfusion (IR) injury, and hepatocellular carcinoma (HCC).
   89. The method of any one of embodiments 48-87, wherein the patient is diagnosed with, suspected of having, or at risk of a renal disease selected from the group consisting of chronic kidney disease (CKD), acute kidney injury (AKI), acute kidney injury (AKI)-related chronic kidney disease (CKD), renal fibrosis, renal fibrosis secondary to, or otherwise associated with, an underlying indication, nephrotic syndrome (NS), minimal change disease (MCD), anti-neutrophil cytoplasmic antibody (ANCA)-associated glomerulonephritis, lupus nephritis, anti-globular basement membrane (anti-GBM) nephropathy, IgA nephropathy, also known as Berger's disease, Alport syndrome (AS), polycystic kidney disease, polycystic kidney disease (e.g., autosomal recessive polycystic kidney disease (ARPKD)-congenital hepatic fibrosis (CHF)), renal cysts, collagen type III glomerulopathy, and nail-patella syndrome.
   90. The method of any one of embodiments 48-87, wherein the patient is diagnosed with, suspected of having, or at risk of a cerebral and/or cerebrovascular disease selected from the group consisting of stroke and cerebral infarction.
   91. The method of any one of embodiments 48-87, wherein the patient is diagnosed with, suspected of having, or at risk of fibrosis of gastrointestinal tract, heart, kidney, lung, liver, or skin.
   92. The method of embodiment 91, wherein the patient is diagnosed with, suspected of having, or at risk of cardiac fibrosis and/or fibrosis associated with cardiovascular system.
   93. The method of any one of embodiments 48-87, wherein the patient is diagnosed with, suspected of having, or at risk of ischemic heart disease, myocardial ischemia, atherosclerosis, myocardial perfusion (e.g., as a consequence of chronic cardiac ischemia or myocardial infarction), vascular occlusion, or restenosis.
   94. The method of embodiment 91, wherein the patient is diagnosed with, suspected of having, or at risk of pulmonary fibrosis.
   95. The method of embodiment 91 or 94, wherein the patient is diagnosed with, suspected of having, or at risk of idiopathic pulmonary fibrosis.
   96. The method of embodiment 91, wherein the patient is diagnosed with, suspected of having, or at risk of dermal fibrosis.
   97. The method of any one of embodiments 48-87, wherein the patient is diagnosed with, suspected of having, or at risk of scleroderma or systemic sclerosis.
   98. The method of embodiment 91, wherein the patient is diagnosed with, suspected of having, or at risk of gastrointestinal fibrosis.
   99. The method of any one of embodiments 48-87, wherein the patient is diagnosed with, suspected of having, or at risk of inflammatory bowel disease.
   100. The method of any one of embodiments 48-99, wherein the compound according to any one of embodiments 1-35 is administered as a pharmaceutical composition.
   101. The method of any one of embodiments 48-100, wherein the compound according to any one of embodiments 1-35 is administered orally.
   102. The method of any one of embodiments 48-101, wherein the compound according to any one of embodiments 1-35 is administered as a pharmaceutically acceptable salt form.

### Treatment Kit

In other embodiments, the present disclosure relates to a kit for conveniently and effectively carrying out the methods in accordance with the present disclosure. In general, the pharmaceutical pack or kit comprises one or more containers filled with one or more of the ingredients of the pharmaceutical compositions described herein. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the dosages of the pharmaceutical compositions, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

### EQUIVALENTS

The representative examples that follow are intended to help illustrate the compounds, compositions, and methods described herein, and are not intended to, nor should they be construed to, limit the scope of the embodiments described. Indeed, various modifications of embodiments described herein and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this disclosure in its various embodiments and the equivalents thereof.

### EXAMPLIFICATION

The compounds of this disclosure and their preparation can be understood further by the examples that illustrate some of the processes by which these compounds are prepared or used. It will be appreciated, however, that these examples do not limit the disclosure. Variations of the disclosure, now known or further developed, are considered to fall within the scope of the present disclosure as described herein and as hereinafter claimed.

1) General Description of Synthetic Methods:

The practitioner has a well-established literature of small molecule chemistry to draw upon, in combination with the information contained herein, for guidance on synthetic strategies, protecting groups, and other materials and methods useful for the synthesis of the compounds of this disclosure.

The various references cited herein provide helpful background information on preparing compounds similar to the provided compounds described herein or relevant intermediates, as well as information on formulation, uses, and administration of such compounds which may be of interest.

Moreover, the practitioner is directed to the specific guidance and examples provided in this document relating to various exemplary compounds and intermediates thereof.

The compounds of this disclosure and their preparation can be understood further by the examples that illustrate some of the processes by which these compounds are prepared or used. It will be appreciated, however, that these examples do not limit the disclosure. Variations of the disclosure, now known or further developed, are considered to fall within the scope of the present disclosure as described herein and as hereinafter claimed.

According to the present disclosure, any available techniques can be used to make or prepare the provided compounds or compositions including them. For example, a variety of solution phase synthetic methods such as those discussed in detail below may be used. Alternatively or additionally, the provided compounds may be prepared using any of a variety combinatorial techniques, parallel synthesis and/or solid phase synthetic methods known in the art.

It will be appreciated as described below, that a variety of provided compounds can be synthesized according to the methods described herein. The starting materials and reagents used in preparing these compounds are either available from commercial suppliers such as Aldrich Chemical Company (Milwaukee, WI), Bachem (Torrance, CA), Sigma (St. Louis, MO), or are prepared by methods well known to a person of ordinary skill in the art following procedures described in such references as Fieser and Fieser 1991, “Reagents for Organic Synthesis”, vols 1-17, John Wiley and Sons, New York, NY, 1991; Rodd 1989 “Chemistry of Carbon Compounds”, vols. 1-5 and supps, Elsevier Science Publishers, 1989; “Organic Reactions”, vols 1-40, John Wiley and Sons, New York, NY, 1991; March 2001, “Advanced Organic Chemistry”, 5th ed. John Wiley and Sons, New York, NY; and Larock 1990, “Comprehensive Organic Transformations: A Guide to Functional Group Preparations”, 2^{nd} ed. VCH Publishers. These schemes are merely illustrative of some methods by which the compounds of this disclosure can be synthesized, and various modifications to these schemes can be made and will be suggested to a person of ordinary skill in the art having regard to this disclosure.

The starting materials, intermediates, and compounds of this disclosure may be isolated and purified using conventional techniques, including filtration, distillation, crystallization, chromatography, and the like. They may be characterized using conventional methods, including physical constants and spectral data.

### General Reaction Procedures:

Unless mentioned specifically, reaction mixtures were stirred using a magnetically driven stirrer bar. An inert atmosphere refers to either dry argon or dry nitrogen. Reactions were monitored either by thin layer chromatography, by proton nuclear magnetic resonance (NMR) or by high-pressure liquid chromatography (HPLC), of a suitably worked up sample of the reaction mixture.

### General Work Up Procedures:

Unless mentioned specifically, reaction mixtures were cooled to room temperature or below then quenched, when necessary, with either water or a saturated aqueous solution of ammonium chloride. Desired products were extracted by partitioning between water and a suitable water-immiscible solvent (e.g. ethyl acetate, dichloromethane, diethyl ether). The desired product-containing extracts were washed appropriately with water followed by a saturated solution of brine. On occasions where the product containing extract was deemed to contain residual oxidants, the extract was washed with a 10% solution of sodium sulfite in saturated aqueous sodium bicarbonate solution, prior to the aforementioned washing procedure. On occasions where the product containing extract was deemed to contain residual acids, the extract was washed with saturated aqueous sodium bicarbonate solution, prior to the aforementioned washing procedure (except in those cases where the desired product itself had acidic character). On occasions where the product containing extract was deemed to contain residual bases, the extract was washed with 10% aqueous citric acid solution, prior to the aforementioned washing procedure (except in those cases where the desired product itself had basic character). Post washing, the desired product containing extracts were dried over anhydrous magnesium sulfate, and then filtered. The crude products were then isolated by removal of solvent(s) by rotary evaporation under reduced pressure, at an appropriate temperature (generally less than 45° C.).

### General Purification Procedures:

Unless mentioned specifically, chromatographic purification refers to flash column chromatography on silica and/or preparative thin layer chromatography (TLC) plates, using a single solvent or mixed solvent as eluent. Suitably purified desired product containing elutes were combined and concentrated under reduced pressure at an appropriate temperature (generally less than 45° C.) to constant mass. Final compounds were dissolved in 50% aqueous acetonitrile, filtered and transferred to vials, then freeze-dried under high vacuum before submission for biological testing.

The following represents non-limiting examples of the synthetic methods.

### Intermediate 1. 5-Ethynyl-3-fluoro-1H-indazole (Int-1)

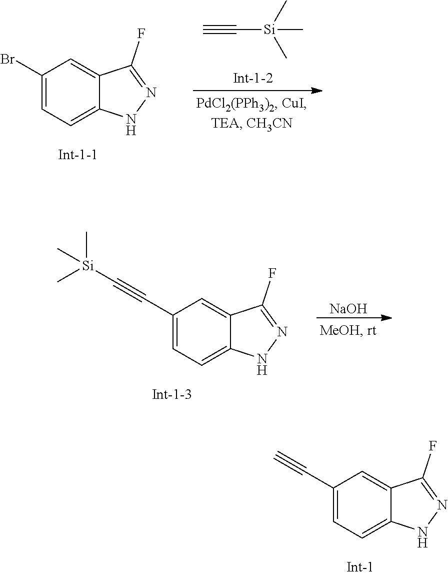

Step 1: 3-Fluoro-5-((trimethylsilyl)ethynyl)-1H-indazole (Int-1-3): Under nitrogen, to a mixture of 5-bromo-3-fluoro-1H-indazole (Int-1-1, CAS #1211537-09-5, commercially available or can be easily prepared according to WO 2019/225552, 6.0 g, 27.9 mmol), trimethylsilylacetylene (Int-1-2, 5.48 g, 55.8 mmol), Cu(I) iodide (57 mg, 0.3 mmol), PdCl₂(PPh₃)₂ (210.6 mg, 0.3 mmol), and Et₃N (8.0 mL) was added acetonitrile (30 mL). The resulting mixture was stirred at 70° C. for 2 h. LC-MS showed the reaction was complete. After cooling to room temperature, the reaction mixture was filtered, concentrated and washed with water (3×30 mL). The solids were used directly in the next step without purification.

Step 2: 5-Ethynyl-3-fluoro-1H-indazole (Int-1): To a solution of the residue from Step 1 in methanol (50 mL) was added NaOH (2.232 g, 55.8 mmol). The reaction mixture was stirred at room temperature for 2 h. LC-MS showed the reaction was complete. The reaction mixture was diluted with water (50 mL) and filtered. The aqueous layer was collected and extracted with DCM (3×100 mL). The organic layers were combined, dried over MgSO₄, filtered, and concentrated in vacuo to give 5-ethynyl-3-fluoro-1H-indazole (Int-1) as an off-white solid, which was used directly in the next step without purification.

### Intermediate 2. tert-Butyl 5-ethynyl-1H-indazole-1-carboxylate (Int-2)

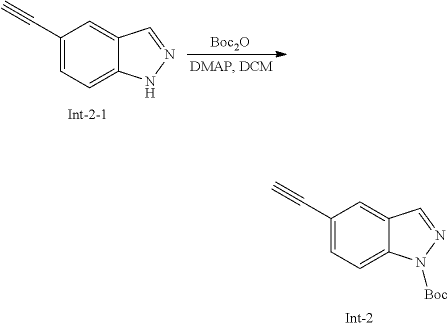 [SMILES: "Boc2O" ; C#Cc1ccc2[nH]ncc2c1 ; C#Cc1ccc2c(cnn2C(=O)OC(C)(C)C)c1]

To a suspension of the compound 5-ethynyl-1H-indazole (Int-2-1, CAS #1207351-15-2, commercially available or can be easily prepared according to X. Ren et al. J. Med. Chem. 2013, 56, 879-894, 0.4 g, 2.03 mmol) in DCM (25.0 mL) was added 4-dimethylaminopyridine (DMAP, 0.25 g, 2.03 mmol) and di-tert-butyl dicarbonate (Boc₂O, 0.58 g, 2.64 mmol). The resulting mixture was stirred at room temperature for 1 h. LC-MS showed the reaction was complete. The reaction mixture was partitioned between DCM (25 mL) and water (25 mL). The organic layer was collected and the aqueous layer was extracted with DCM (50 mL). The combined organic layers were washed with brine, dried over Na₂SO₄, filtered, and concentrated under reduced pressure. The residue was purified by column chromatography (ISCO) on neutral alumina to afford tert-butyl 5-ethynyl-1H-indazole-1-carboxylate (Int-2, 250 mg, yield: 51%) as a pale yellow gum. MS (ESI⁺): m/z: 243.2 (M+H)⁺.

### Intermediate 3. tert-Butyl 5-ethynyl-3-fluoro-1H-indazole-1-carboxylate (Int-3)

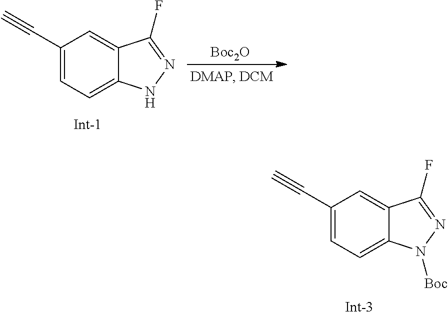 [SMILES: "Boc2O" ; C#Cc1ccc2[nH]nc(F)c2c1 ; C#Cc1ccc2c(c1)c(F)nn2C(=O)OC(C)(C)C]

Int-3 was prepared in a manner analogous to the procedure described for Int-2 to provide the compound as a yellow oil. MS (ESI⁺): m/z: 261.10 (M+H)⁺.

Intermediate 4. 2′,4-Dichloro-2,4′-bipyrimidine (Int-4):

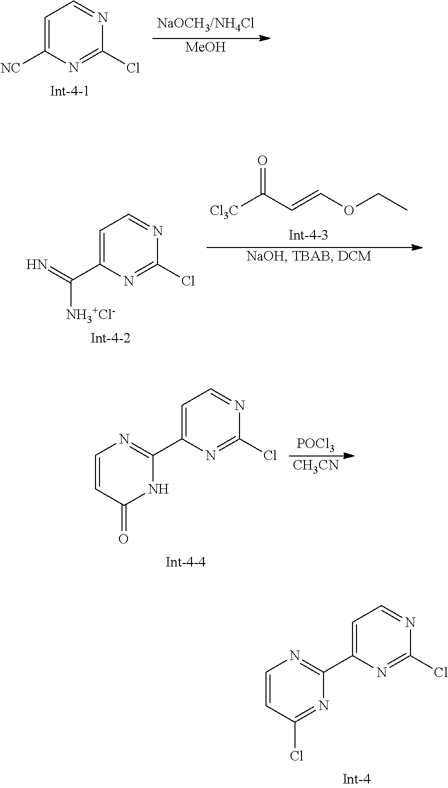

Step 1: 2-Chloropyrimidine-4-carboximidamide HCl salt (Int-4-2): To a solution of 2-chloropyrimidine-4-carbonitrile (Int-4-1, 20 g, 143.3 mmol) in MeOH (200 mL) was added NaOCH₃ (5.42 g, 100.3 mmol) at room temperature. The resulting mixture was stirred at rt for 40 min. NH₄Cl (15.3 g, 286.6 mmol) was added and the reaction mixture was stirred at 50° C. for 2.5 h. After cooling to rt, the solvent was removed in vacuo to give chloropyrimidine-4-carboximidamide HCl salt (Int-4-2) as a brownish solid, which was used directly in the nest step without further purification.

Step 2: 2′-Chloro-[2,4′-bipyrimidin]-4(3H)-one (Int-4-4): A solution of (E)-1,1,1-trichloro-4-ethoxybut-3-en-2-one (Int-4-3, 31.2 g, 143 mmol) in DCM (300 mL) was added to a vigorously stirred mixture of chloropyrimidine-4-carboximidamide HCl salt (Int-4-2, 27.6 g, 143 mmol) in aq. 2M solution of NaOH (286 mL) and tetrabutylammonium bromide (TBAB, cat. 0.6 g). The resulting mixture was stirred at rt for 7 h. The aqueous layer was collected, acidified with conc. HCl to pH≈2, and extracted with DCM (3×100 mL). The combined organic layer was dried over MgSO₄, filtered and concentrated to dryness to give 2′-chloro-[2,4′-bipyrimidin]-4(3H)-one (Int-4-4, 12.08 g) as a yellow solid, which was used directly in the next step without further purification.

Step 3: 2′,4-Dichloro-2,4′-bipyrimidine (Int-4): Under N₂, to a suspension of 2′-chloro-[2,4′-bipyrimidin]-4(3H)-one (Int-4-4) from Step 2 in anhydrous acetonitrile was added POCl₃ dropwise. The resulting mixture was stirred at 65° C. for 40 min. LC-MS showed the reaction was complete. Excess POCl₃ was removed completely under reduced pressure and the residue was partitioned between sat. NaHCO₃ and DCM (pH ≥8). The product was extracted with DCM (3×100 mL). The combined organic layer was dried over MgSO₄, filtered and concentrated. The crude product was purified by column chromatography (ISCO) (DCM:EA=10:1) to afford the desired product 2′,4-dichloro-2,4′-bipyrimidine (Int-4) as a white solid (yield: 68%). ¹H-NMR (300 MHz, DMSO-d₆): δ (ppm): 9.04 (d, J=5.34 Hz, 1H), 9.03 (d, J=5.1 Hz, 1H), 8.34 (d, J=5.05 Hz, 1H), 7.94 (d, J=5.35 Hz, 1H). MS (ESI⁺): m/z 226.97 (M+H)⁺.

### Intermediate 5. tert-Butyl 5-((2′-chloro-[2,4′-bipyrimidin]-4-yl)ethynyl)-1H-indazole-1-carboxylate (Int-5)

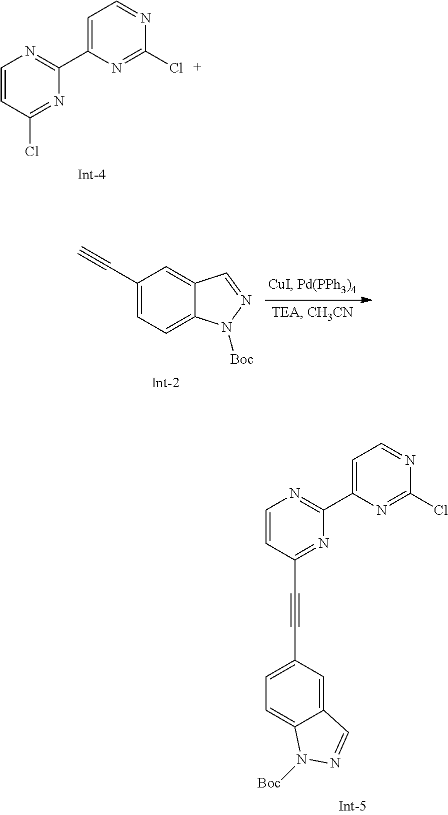 [SMILES: Clc1ccnc(-c2ccnc(Cl)n2)n1 ; C#Cc1ccc2c(cnn2C(=O)OC(C)(C)C)c1 ; CC(C)(C)OC(=O)n1ncc2cc(C#Cc3ccnc(-c4ccnc(Cl)n4)n3)ccc21]

Under N₂, to a mixture of 2′,4-dichloro-2,4′-bipyrimidine (Int-4, 1.0 g, 4.44 mmol), tert-butyl 5-ethynyl-1H-indazole-1-carboxylate (Int-2, 1.18 g, 4.88 mmol), CuI (85.5 mg, 0.45 mmol), and Pd(PPh₃)₄ (1.025 g, 0.9 mmol) was added NEt₃ (2.4 mL) followed by MeCN (30 mL). The resulting mixture was degassed for 10 min with N₂ and then stirred at 70-72° C. for 6 h. After cooling to rt, the reaction mixture was left at rt overnight. The precipitates were collected by filtration and washed with diethyl ether to afford the desired product tert-butyl 5-((2′-chloro-[2,4′-bipyrimidin]-4-yl)ethynyl)-1H-indazole-1-carboxylate (Int-5, yield, 70%). ¹H NMR (300 MHz, CDCl₃) δ 8.97 (d, J=5.1 Hz, 1H), 8.85 (d, J=5.1 Hz, 1H), 8.45 (d, J=4.8 Hz 1H), 8.26-8.21 (m, 2H), 8.08 (s, 1H), 8.79 (m, 1H), 8.56 (m, 1H), 1.73 (s, 9H), MS (ESI⁺): m/z: 433.18 (³⁵Cl, M+H)⁺, 435.18 (³⁷Cl, M+H)⁺.

### Intermediate 6. tert-Butyl 5-((2′-chloro-[2,4′-bipyrimidin]-4-yl)ethynyl)-3-fluoro-1H-indazole-1-carboxylate (Int-6)

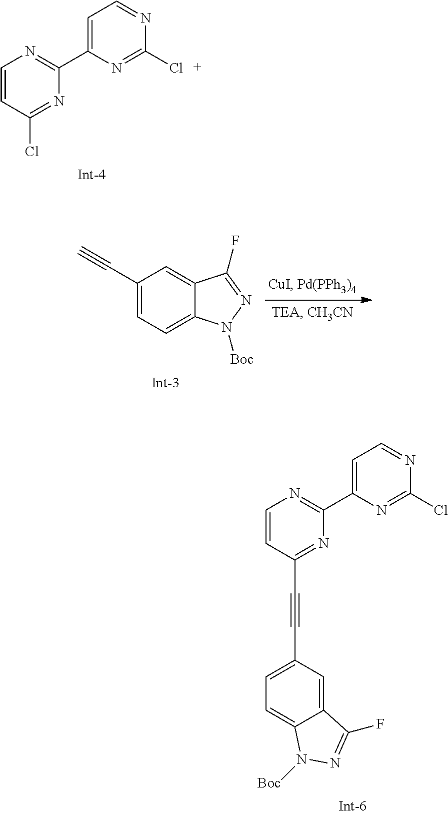 [SMILES: Clc1ccnc(-c2ccnc(Cl)n2)n1 ; C#Cc1ccc2c(c1)c(F)nn2C(=O)OC(C)(C)C ; CC(C)(C)OC(=O)n1nc(F)c2cc(C#Cc3ccnc(-c4ccnc(Cl)n4)n3)ccc21]

Int-6 was prepared in a manner analogous to the procedure described for Intermediate 5 to provide the compound in 27% yield. MS (ESI⁺): m/z: 451.10 (³⁵Cl, M+H)⁺, 453.10 (³⁷Cl, M+H)⁺.

### Intermediate 7. tert-Butyl 5-ethynyl-7-fluoro-1H-indazole-1-carboxylate (Int-7)

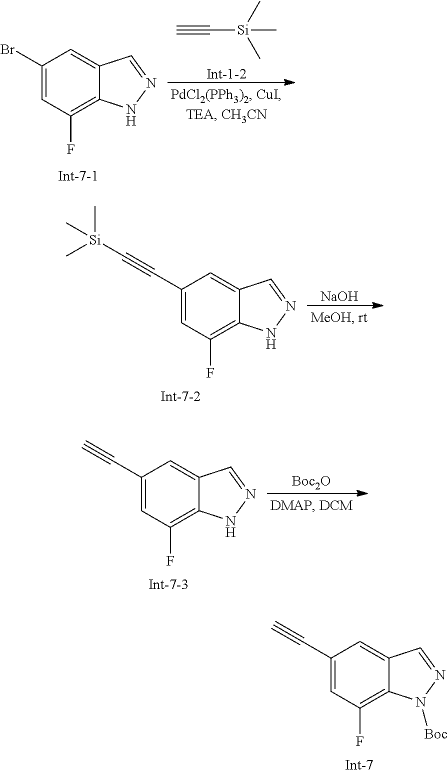

Int-7 was prepared in a manner analogous to the procedures described for Intermediate 1 and Intermediate 2. MS (ESI⁺): m/z: MS (ESI⁺): m/z: 261.2 (M+H)⁺.

### Intermediate 8. tert-Butyl 5-((2′-chloro-[2,4′-bipyrimidin]-4-yl)ethynyl)-7-fluoro-1H-indazole-1-carboxylate (Int-8)

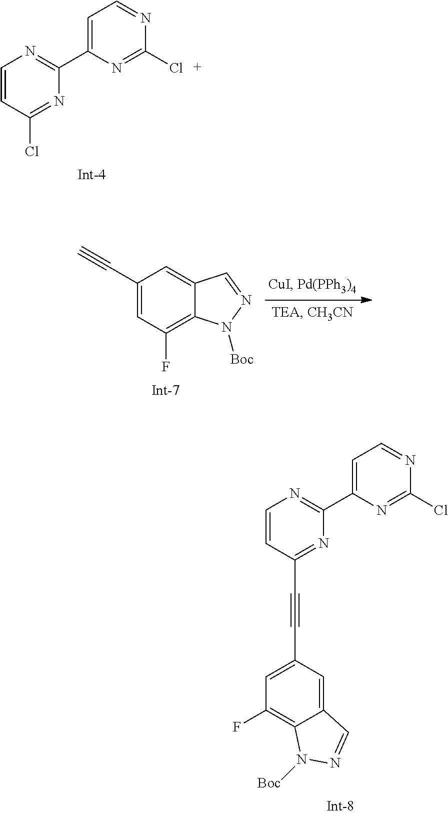 [SMILES: Clc1ccnc(-c2ccnc(Cl)n2)n1 ; C#Cc1cc(F)c2c(cnn2C(=O)OC(C)(C)C)c1 ; CC(C)(C)OC(=O)n1ncc2cc(C#Cc3ccnc(-c4ccnc(Cl)n4)n3)cc(F)c21]

Int-8 was prepared in a manner analogous to the procedure described for Intermediate 5 to provide the compound in 27% yield. MS (ESI⁺): m/z: 451.19 (³⁵Cl, M+H), 453.19 (³⁷Cl, M+H)⁺.

### Intermediate 9. tert-Butyl 5-((2′-chloro-[2,4′-bipyrimidin]-4-yl)ethynyl)-3-methyl-1H-indazole-1-carboxylate (Int-9)

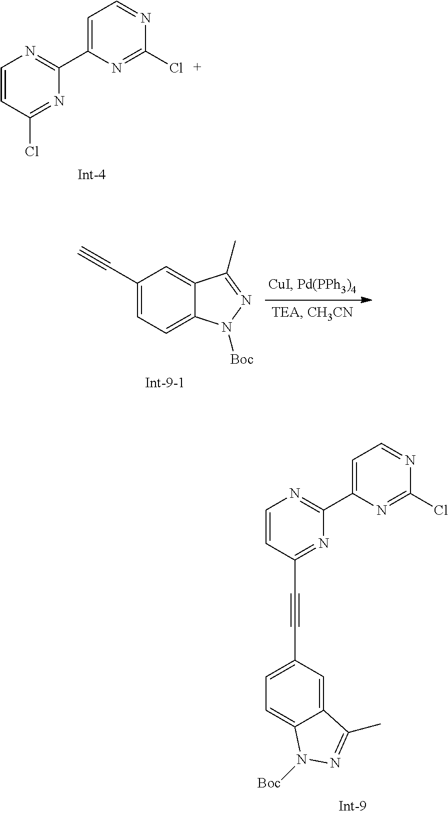 [SMILES: Clc1ccnc(-c2ccnc(Cl)n2)n1 ; C#Cc1ccc2c(c1)c(C)nn2C(=O)OC(C)(C)C ; Cc1nn(C(=O)OC(C)(C)C)c2ccc(C#Cc3ccnc(-c4ccnc(Cl)n4)n3)cc12]

Int-9-1 was prepared from commercially available 5-ethynyl-3-methyl-1H-indazole (CAS #1093307-29-9) and Boc₂O following the procedure described for Intermediate 2. Int-9 was prepared in a manner analogous to the procedure described for Intermediate 5. MS (ESI⁺): m/z: 446.2 (³⁵Cl, M+H)⁺, 448.2 (³⁷Cl, M+H)⁺.

### Example 1. (S)-2-((4-((1H-Indazol-5-yl)ethynyl)-[2,4′-bipyrimidin]-2′-yl)amino)-1-(3-hydroxypyrrolidin-1-yl)ethanone (Ex. 1)

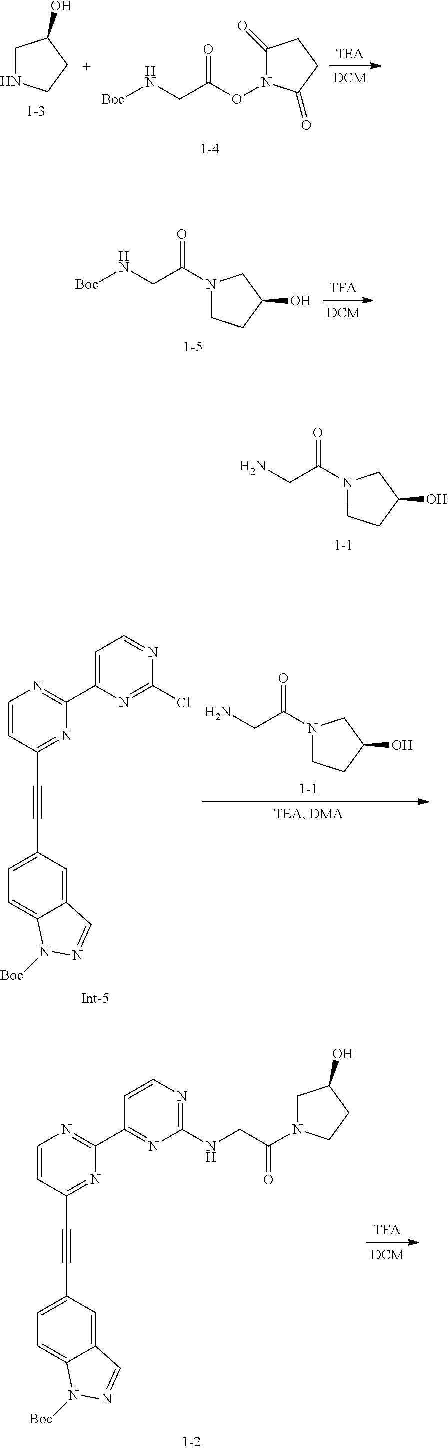

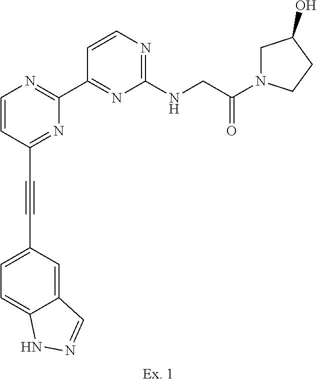 [SMILES: O=C(CNc1nccc(-c2nccc(C#Cc3ccc4[nH]ncc4c3)n2)n1)N1CC[C@H](O)C1]

Step 1: tert-Butyl (S)-(2-(3-hydroxypyrrolidin-1-yl)-2-oxoethyl)carbamate (1-5): To a stirred mixture of (S)-3-hydroxypyrrolidine hydrochloride (1-3, 200 mg, 1.62 mmol) and Boc-Gly-Osu (1-4, 529 mg, 1.94 mmol) in anhydrous dimethylacetamide (4 mL) was added dropwise triethylamine (0.45 mL, 3.24 mmol) at room temperature. The resulting mixture was stirred at room temperature for 2 h, and then diluted with water. The DCM layer was collected, dried over Na₂SO₄, filtered, and the filtrate concentrated to give the crude product 1-5, which was used directly in the next step without purification. MS (ESI⁺): m/z: 245.25 (M+H)⁺.

Step 2: (S)-2-Amino-1-(3-hydroxypyrrolidin-1-yl)ethan-1-one (1-1): To a stirred solution of tert-butyl (S)-(2-(3-hydroxypyrrolidin-1-yl)-2-oxoethyl)carbamate (1-5, from Step 1) in DCM (5 mL) was added dropwise trifluoroacetic acid (2 mL) at room temperature. The reaction mixture was stirred at room temperature for 1 h, then concentrated in vacuo to dryness. The crude product was used directly in the next step without purification. MS (ESI⁺): m/z: 145.17 (M+H)⁺.

Step 3: (S)-tert-Butyl 5-((2′-((2-(3-hydroxypyrrolidin-1-yl)-2-oxoethyl)amino)-[2,4′-bipyrimidin]-4-yl)ethynyl)-1H-indazole-1-carboxylate (1-2): Under N₂, to a stirred mixture of tert-butyl 5-((2′-chloro-[2,4′-bipyrimidin]-4-yl)ethynyl)-1H-indazole-1-carboxylate (Int-5, 50 mg, 0.116 mmol) and (S)-2-amino-1-(3-hydroxypyrrolidin-1-yl)ethan-1-one (1-1, 33.4 mg, 0.232 mmol) in anhydrous dimethylacetamide (DMA, 1.0 mL) was added dropwise triethylamine (TEA, 0.1 mL, 0.717 mmol) at room temperature. The resulting mixture was stirred at 70° C. for 20 h. LC-MS showed the reaction was complete. The reaction mixture was cooled to rt and diluted with H₂O (5 mL). The layers were separated and the aqueous phase was extracted with DCM (3×5 mL). The organic layers were combined, dried over Na₂SO₄, filtered, and the filtrate concentrated in vacuo. The crude product was purified by silica gel flash chromatography (ISCO) to afford the desired product (S)-tert-butyl 5-((2′-((2-(3-hydroxypyrrolidin-1-yl)-2-oxoethyl)amino)-[2,4′-bipyrimidin]-4-yl)ethynyl)-1H-indazole-1-carboxylate (1-2, 9.5 mg, yield: 15%) as a light yellow oil. MS (ESI⁺): m/z: 541.42 (M+H)⁺.

Step 4: (S)-2-((4-((1H-Indazol-5-yl)ethynyl)-[2,4′-bipyrimidin]-2′-yl)amino)-1-(3-hydroxypyrrolidin-1-yl)ethan-1-one (Ex. 1): To a stirred solution of tert-butyl (S)-5-((2′-((2-(3-hydroxypyrrolidin-1-yl)-2-oxoethyl)amino)-[2,4′-bipyrimidin]-4-yl)ethynyl)-1H-indazole-1-carboxylate (1-2, 9.5 mg, 0.02 mmol) in DCM (1.0 mL) was added dropwise trifluoroacetic acid (0.5 mL) at room temperature. The resulting mixture was stirred at room temperature for 1 h. LC-MS showed the reaction was complete. The reaction mixture was concentrated in vacuo and neutralized with sat. NaHCO₃ solution, which was then extracted with ethyl acetate, dried over Na₂SO₄, filtered, and concentrated to dryness. The crude product was purified by silica gel flash chromatography (ISCO) to afford (S)-2-((4-((1H-indazol-5-yl)ethynyl)-[2,4′-bipyrimidin]-2′-yl)amino)-1-(3-hydroxypyrrolidin-1-yl)ethan-1-one (Ex. 1, 5.6 mg, yield: 72%). ¹H-NMR (300 MHz, CD₃OD): δ (ppm): 8.94 (d, J=5.1 Hz, 1H), 8.53 (d, J=5.1 Hz, 1H), 8.19 (br s, 1H), 8.16 (s, 1H), 7.71 (dd, J=5.9, 0.7 Hz, 1H), 7.69 (d, J=6.7 Hz, 1H), 7.67-7.60 (m, 2H), 4.55-4.22 (m, 3H), 3.85-3.48 (m, 4H), 2.21-1.89 (m, 2H). MS (ESI⁺): m/z: 441.34 (M+H)⁺.

### Example 2. 2-((4-((1H-Indazol-5-yl)ethynyl)-[2,4′-bipyrimidin]-2′-yl)amino)-N-(bicyclo[2.2.1]heptan-1-yl)acetamide (Ex. 2)

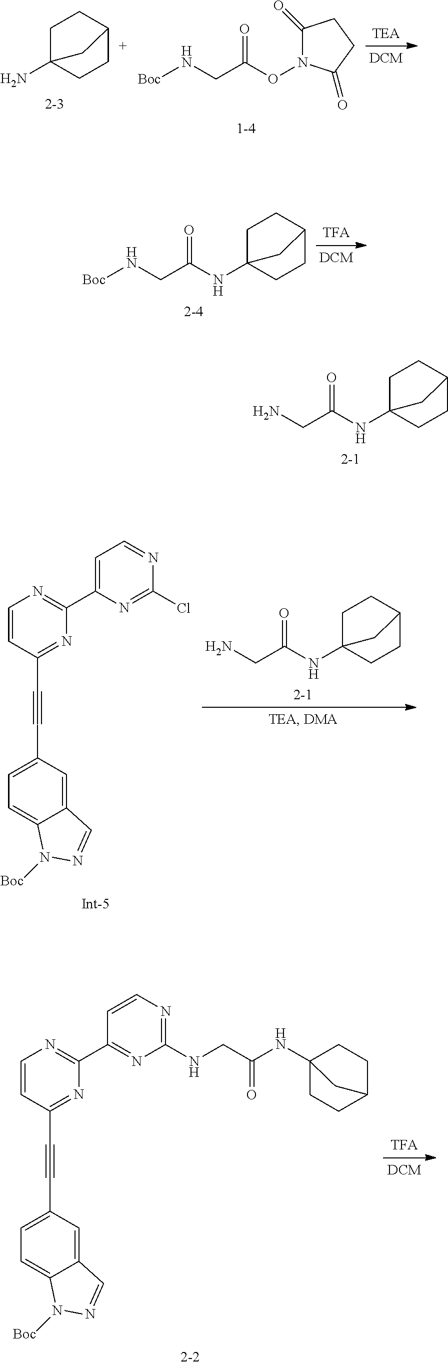

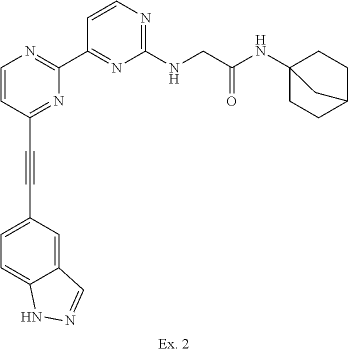 [SMILES: O=C(CNc1nccc(-c2nccc(C#Cc3ccc4[nH]ncc4c3)n2)n1)NC12CCC(CC1)C2]

Ex. 2 was prepared in a manner analogous to the procedure described for Example 1 from bicyclo[2.2.1]heptan-1-amine and tert-butyl 5-((2′-chloro-[2,4′-bipyrimidin]-4-yl)ethynyl)-1H-indazole-1-carboxylate (Int-5). ¹H-NMR (300 MHz, CDCl₃): δ (ppm): 8.92 (d, J=5.1 Hz, 1H), 8.60 (d, J=5.0 Hz, 1H), 8.16 (s, 1H), 8.00 (br s, 1H), 7.82 (dd, J=5.1 Hz, 1H), 7.55 (d, J=8.8 Hz, 1H), 7.53 (d, J=5.0 Hz, 1H), 7.26 (d, J=8.5 Hz, 1H), 4.16 (d, J=6.0 Hz, 2H), 2.13 (m, 1H), 1.80-1.61 (m, 8H), 1.40-1.23 (m, 2H). MS (ESI⁺): m/z: 465.39 (M+H)⁺.

### Example 3. 4-((1H-Indazol-5-yl)ethynyl)-N-((5-fluoropyridin-2-yl)methyl)-[2,4′-bipyrimidin]-2′-amine (Ex. 3)

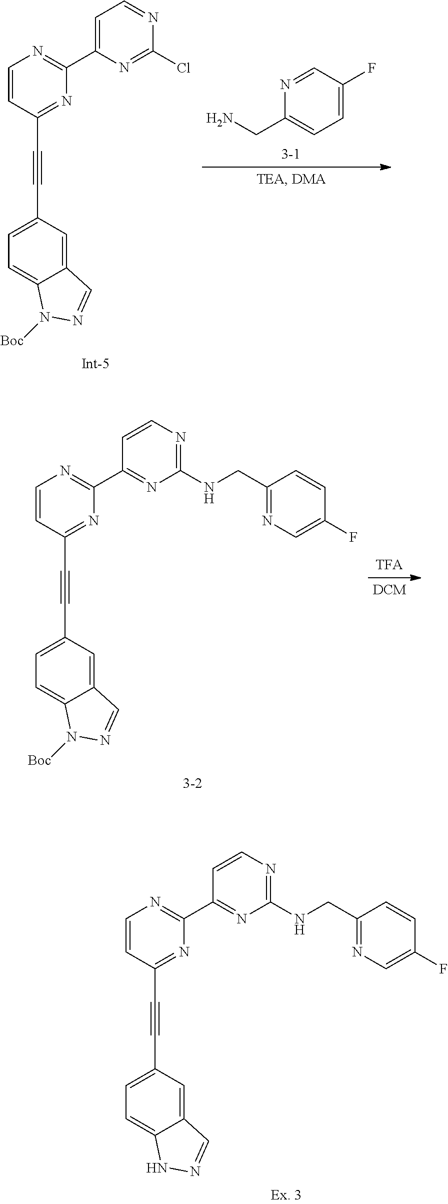 [SMILES: CC(C)(C)OC(=O)n1ncc2cc(C#Cc3ccnc(-c4ccnc(Cl)n4)n3)ccc21 ; NCc1ccc(F)cn1 ; CC(C)(C)OC(=O)n1ncc2cc(C#Cc3ccnc(-c4ccnc(NCc5ccc(F)cn5)n4)n3)ccc21 ; Fc1ccc(CNc2nccc(-c3nccc(C#Cc4ccc5[nH]ncc5c4)n3)n2)nc1]

Ex. 3 was prepared from (5-fluoropyridin-2-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (400 MHz, CD₃OD) δ 8.92 (d, J=6.8 Hz, 1H), 8.50 (d, J=6.8 Hz, 1H), 8.38 (s, 1H), 8.16 (s, 1H), 8.12 (s, 1H), 7.66 (m, 3H), 7.61 (m, 2H), 7.52 (m, 2H), 4.62 (s, 2H). MS (ESI⁺): m/z: 423.23 (M+H)⁺.

### Example 4. 4-((1H-Indazol-5-yl)ethynyl)-N-((5-methylpyrimidin-2-yl)methyl)-[2,4′-bipyrimidin]-2′-amine (Ex. 4)

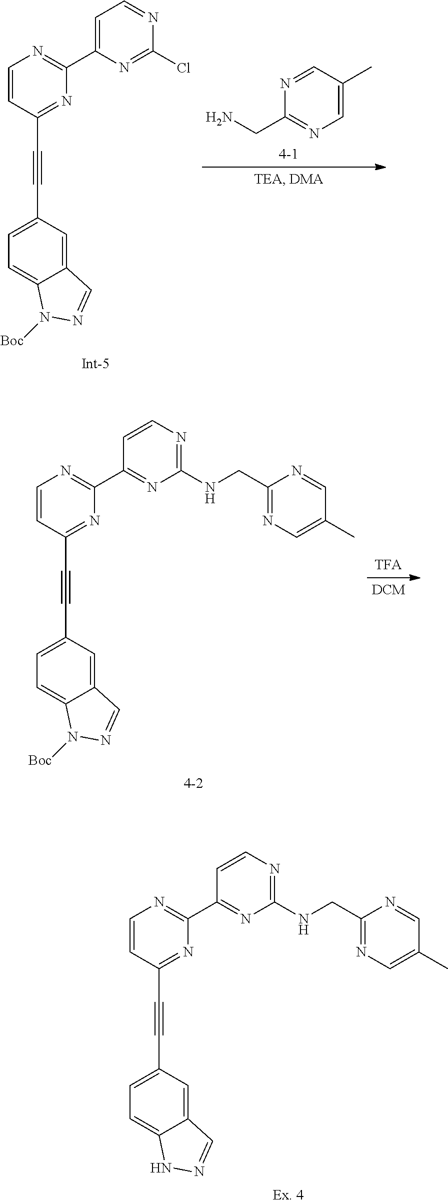

Ex. 4 was prepared from (5-methylpyrimidin-2-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD-CDCl₃): δ=8.93 (s, 1H), 8.75 (s, 1H), 8.59 (s, 2H), 8.52-8.50 (m, 1H), 8.21-8.12 (m, 2H), 7.75-7.61 (m, 3H), 4.94 (s, 2H), 2.32 (s, 3H). MS (ESI⁺): m/z: 420.29 (M+H)⁺.

### Example 5. 4-((1H-Indazol-5-yl)ethynyl)-N-(pyrrolidin-2-ylmethyl)-[2,4′-bipyrimidin]-2′-amine (Ex. 5)

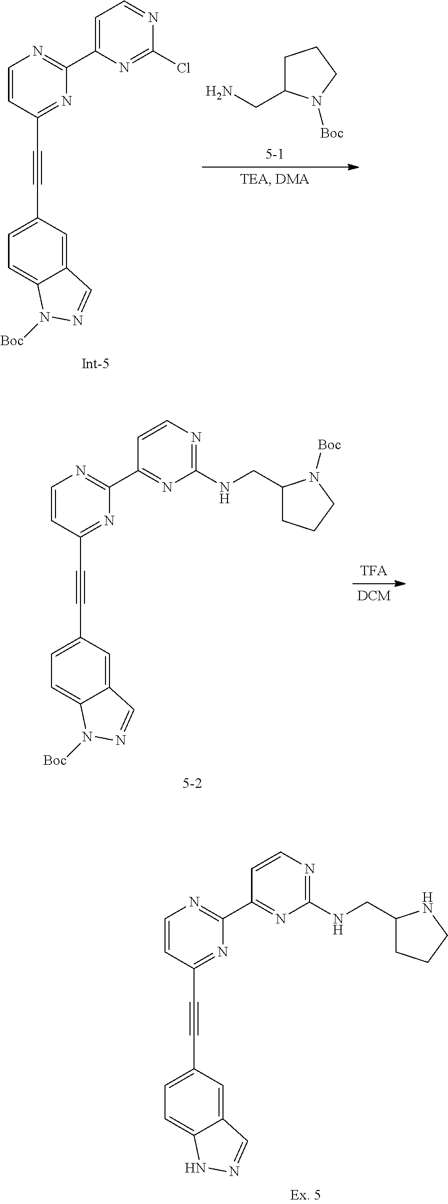 [SMILES: CC(C)(C)OC(=O)n1ncc2cc(C#Cc3ccnc(-c4ccnc(Cl)n4)n3)ccc21 ; CC(C)(C)OC(=O)N1CCCC1CN ; CC(C)(C)OC(=O)N1CCCC1CNc1nccc(-c2nccc(C#Cc3ccc4c(cnn4C(=O)OC(C)(C)C)c3)n2)n1 ; C(#Cc1ccnc(-c2ccnc(NCC3CCCN3)n2)n1)c1ccc2[nH]ncc2c1]

Ex. 5 was prepared from tert-butyl 2-(aminomethyl)pyrrolidine-1-carboxylate and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD-CDCl₃): δ=8.91 (d, J=5.1 Hz, 1H), 8.58 (d, J=5.1 Hz, 1H), 8.14 (s, 1H), 8.09 (s, 1H), 7.80 (d, J=4.8 Hz, 1H), 7.64-7.59 (m, 3H), 3.86-3.67 (m, 3H), 3.37 (s, 1H), 3.24 (q, J=1.5 Hz, 1H), 2.22-1.98 (m, 4H). MS (ESI⁺): m/z: 397.32 (M+H)⁺.

### Example 6. 4-((1H-Indazol-5-yl)ethynyl)-N-((2-fluoropyridin-3-yl)methyl)-[2,4′-bipyrimidin]-2′-amine (Ex. 6)

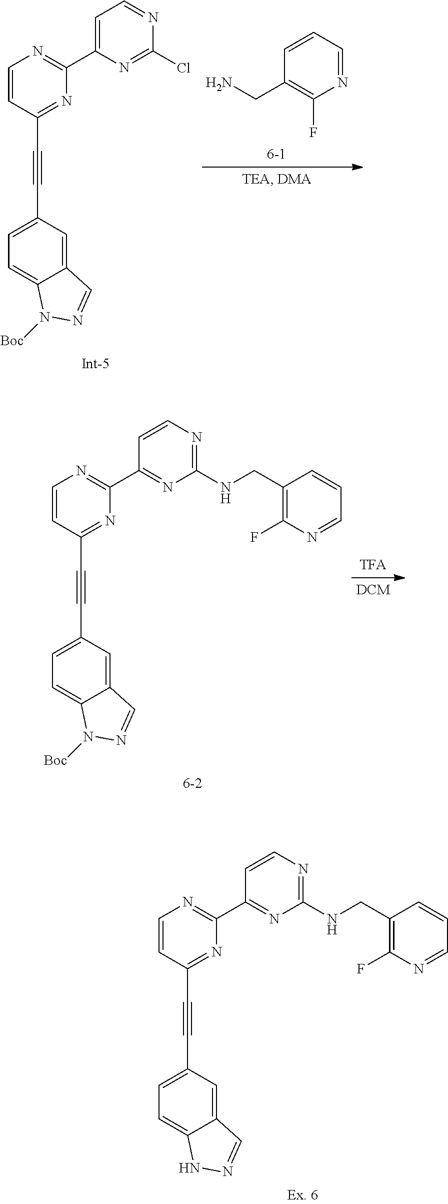 [SMILES: CC(C)(C)OC(=O)n1ncc2cc(C#Cc3ccnc(-c4ccnc(Cl)n4)n3)ccc21 ; NCc1cccnc1F ; CC(C)(C)OC(=O)n1ncc2cc(C#Cc3ccnc(-c4ccnc(NCc5cccnc5F)n4)n3)ccc21 ; Fc1ncccc1CNc1nccc(-c2nccc(C#Cc3ccc4[nH]ncc4c3)n2)n1]

Ex. 6 was prepared from (2-fluoropyridin-3-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H NMR (400 MHz, CD₃OD) δ 8.75 (d, J=6.8 Hz, 1H), 8.37 (d, J=6.8 Hz, 1H), 7.95-7.91 (m, 3H), 7.58 (d, J=6 Hz, 1H), 7.46-6.99 (m, 5H), 4.62 (s, 2H). MS (ESI⁺): m/z: 423.23 (M+H)⁺.

### Example 7. 4-((1H-indazol-5-yl)ethynyl)-N-(pyrimidin-2-ylmethyl)-[2,4′-bipyrimidin]-2′-amine (Ex. 7)

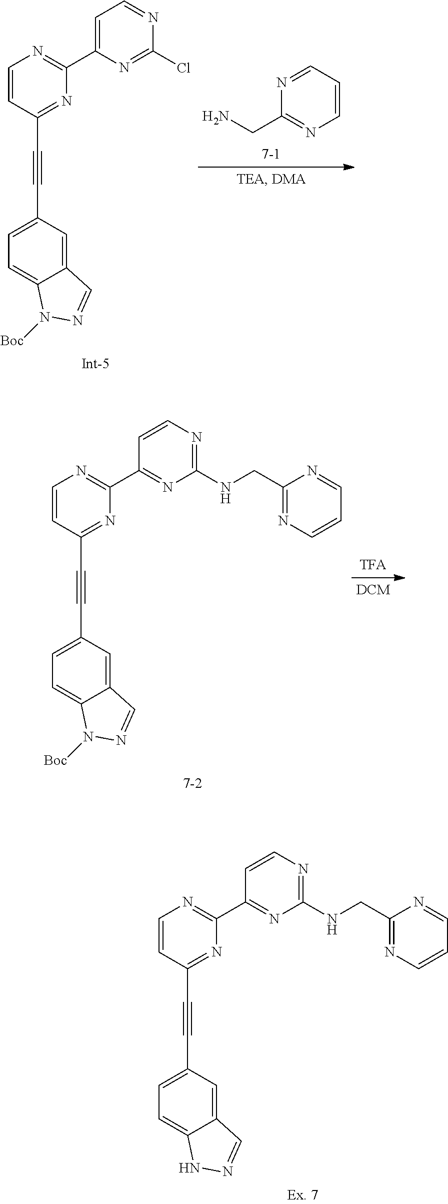 [SMILES: CC(C)(C)OC(=O)n1ncc2cc(C#Cc3ccnc(-c4ccnc(Cl)n4)n3)ccc21 ; NCc1ncccn1 ; CC(C)(C)OC(=O)n1ncc2cc(C#Cc3ccnc(-c4ccnc(NCc5ncccn5)n4)n3)ccc21 ; C(#Cc1ccnc(-c2ccnc(NCc3ncccn3)n2)n1)c1ccc2[nH]ncc2c1]

Ex. 7 was prepared from pyrimidin-2-ylmethanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H NMR (400 MHz, CD₃OD) δ 8.94 (d, J=6.8 Hz, 1H), 8.76 (m, 2H), 8.52 (d, J=6.8 Hz, 1H), 8.20 (s, 1H), 8.16 (s, 1H), 7.71-7.64 (m, 4H), 8.37 (t, J=6.4 Hz, 1H), 4.01 (s, 2H). MS (ESI⁺): m/z: 406.28 (M+H)⁺.

### Example 8. 4-((1H-Indazol-5-yl)ethynyl)-N-(4-fluorobenzyl)-[2,4′-bipyrimidin]-2′-amine (Ex. 8)

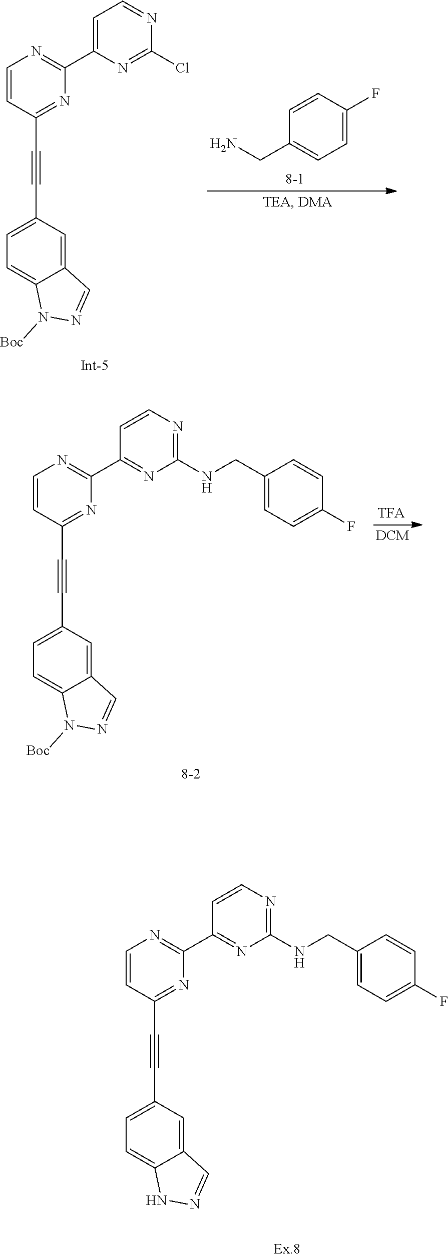 [SMILES: CC(C)(C)OC(=O)n1ncc2cc(C#Cc3ccnc(-c4ccnc(Cl)n4)n3)ccc21 ; NCc1ccc(F)cc1 ; CC(C)(C)OC(=O)n1ncc2cc(C#Cc3ccnc(-c4ccnc(NCc5ccc(F)cc5)n4)n3)ccc21 ; Fc1ccc(CNc2nccc(-c3nccc(C#Cc4ccc5[nH]ncc5c4)n3)n2)cc1]

Ex. 8 was prepared from (4-fluorophenyl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H NMR (400 MHz, CD₃OD) δ 8.92 (d, J=6.8 Hz, 1H), 8.55 (d, J=6.8 Hz, 1H), 8.11 (s, 1H), 8.05 (s, 1H), 7.73 (d, J=6.8 Hz, 1H), 7.52 (m, 2H), 7.38-7.26 (m, 2H), 7.01 (m, 3H), 4.72 (d, 2H). MS (ESI⁺): m/z: 422.25 (M+H)⁺.

### Example 9. 4-((1H-Indazol-5-yl)ethynyl)-N-(piperidin-2-ylmethyl)-[2,4′-bipyrimidin]-2′-amine (Ex. 9)

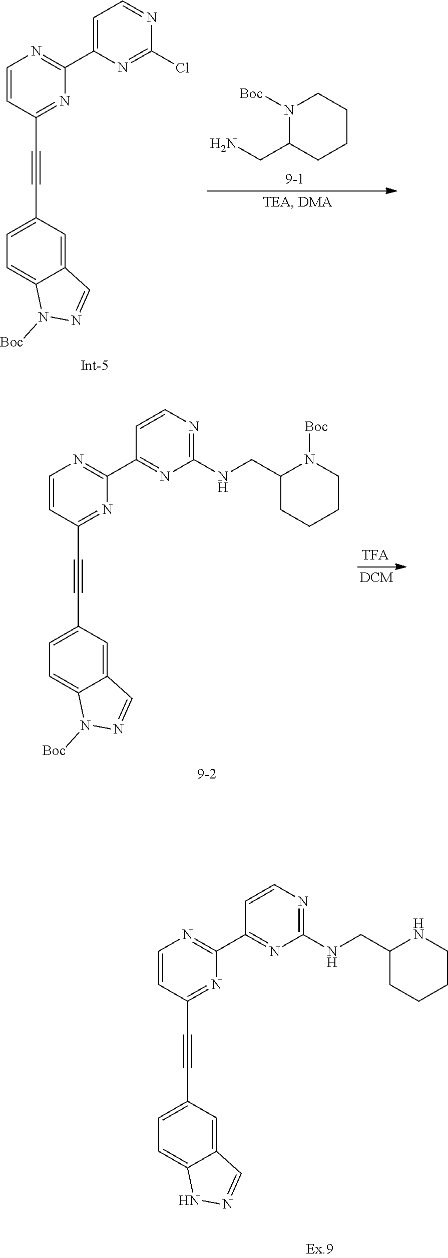 [SMILES: CC(C)(C)OC(=O)n1ncc2cc(C#Cc3ccnc(-c4ccnc(Cl)n4)n3)ccc21 ; CC(C)(C)OC(=O)N1CCCCC1CN ; CC(C)(C)OC(=O)N1CCCCC1CNc1nccc(-c2nccc(C#Cc3ccc4c(cnn4C(=O)OC(C)(C)C)c3)n2)n1 ; C(#Cc1ccnc(-c2ccnc(NCC3CCCCN3)n2)n1)c1ccc2[nH]ncc2c1]

Ex. 9 was prepared from tert-butyl 2-(aminomethyl)piperidine-1-carboxylate and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, DMSO-d₆): δ (ppm): 8.99 (d, J=5.1 Hz, 1H), 8.61 (d, J=5.1 Hz, 1H), 8.20-8.16 (m, 2H), 7.81 (m, 1H), 7.75 (m, 1H), 7.64 (m, 2H), 3.36 (m, 2H), 1.96-0.83 (m, 9H). MS (ESI⁺): m/z: 411.33 (M+H)⁺.

### Example 10. 4-((1H-Indazol-5-yl)ethynyl)-N-((4-fluoropyridin-2-yl)methyl)-[2,4′-bipyrimidin]-2′-amine (Ex. 10)

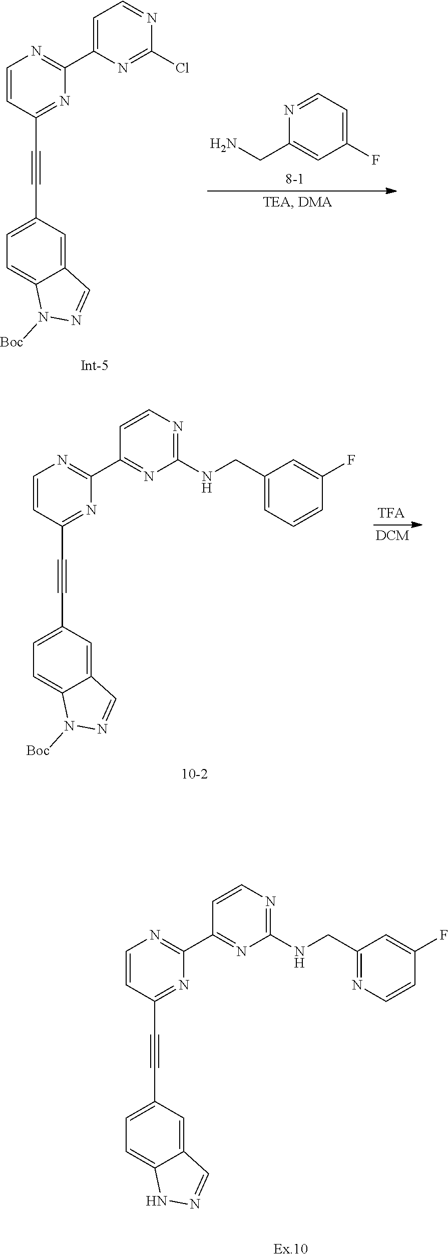 [SMILES: CC(C)(C)OC(=O)n1ncc2cc(C#Cc3ccnc(-c4ccnc(Cl)n4)n3)ccc21 ; NCc1cc(F)ccn1 ; CC(C)(C)OC(=O)n1ncc2cc(C#Cc3ccnc(-c4ccnc(NCc5cccc(F)c5)n4)n3)ccc21 ; Fc1ccnc(CNc2nccc(-c3nccc(C#Cc4ccc5[nH]ncc5c4)n3)n2)c1]

Ex. 10 was prepared from (4-fluoropyridin-2-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, DMSO-d₆): δ (ppm): 8.92 (d, J=5.1 Hz, 1H), 8.56 (m, 2H), 8.43-8.08 (m, 2H), 7.81 (d, J=5.1 Hz, 1H), 7.54-7.53 (m, 4H), 7.40 (m, 1H), 4.92 (s, 2H). MS (ESI⁺): m/z: 423.23 (M+H)⁺.

### Example 11. 4-((1H-Indazol-5-yl)ethynyl)-N-((4-methoxypyridin-2-yl)methyl)-[2,4′-bipyrimidin]-2′-amine (Ex. 11)

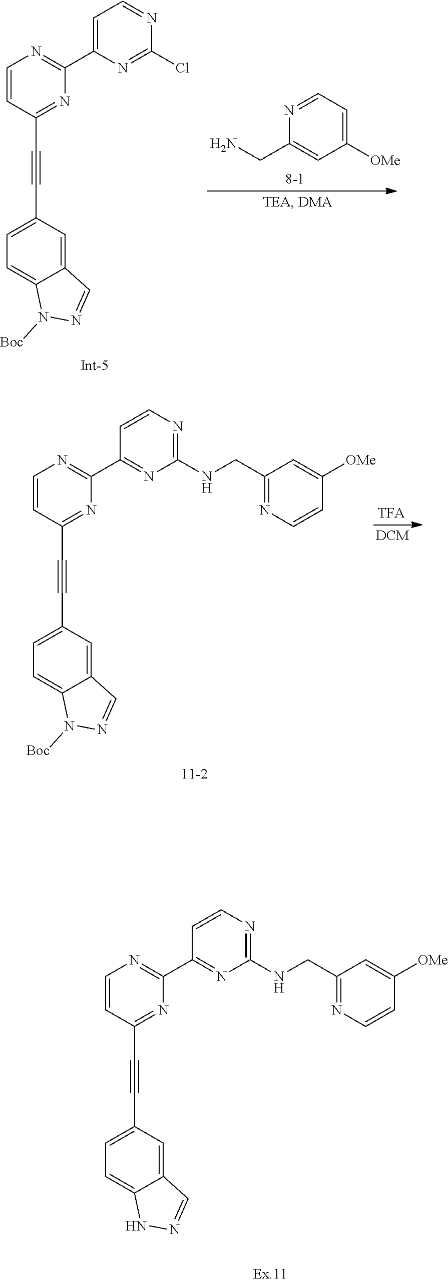 [SMILES: CC(C)(C)OC(=O)n1ncc2cc(C#Cc3ccnc(-c4ccnc(Cl)n4)n3)ccc21 ; COc1ccnc(CN)c1 ; COc1ccnc(CNc2nccc(-c3nccc(C#Cc4ccc5c(cnn5C(=O)OC(C)(C)C)c4)n3)n2)c1 ; COc1ccnc(CNc2nccc(-c3nccc(C#Cc4ccc5[nH]ncc5c4)n3)n2)c1]

Ex. 11 was prepared from (4-methoxypyridin-2-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H NMR (400 MHz, CDCl₃) δ 8.92 (d, J=6.8 Hz, 1H), 8.56 (d, J=6.8 Hz, 1H), 8.36 (d, J=7.6 Hz, 1H), 8.12 (s, 1H), 8.01 (s, 1H), 7.74 (d, J=6.8 Hz, 1H), 7.35-7.25 (m, 3H), 6.93 (d, 1H), 6.70 (dd, J₁=8 Hz, J₁=3.2 Hz, 1H), 4.85 (d, 2H), 3.78 (s, 3H). MS (ESI⁺): m/z: 435.23 (M+H)⁺.

### Example 12. 4-((1H-Indazol-5-yl)ethynyl)-N-(4-fluoro-2-methoxybenzyl)-[2,4′-bipyrimidin]-2′-amine (Ex. 12)

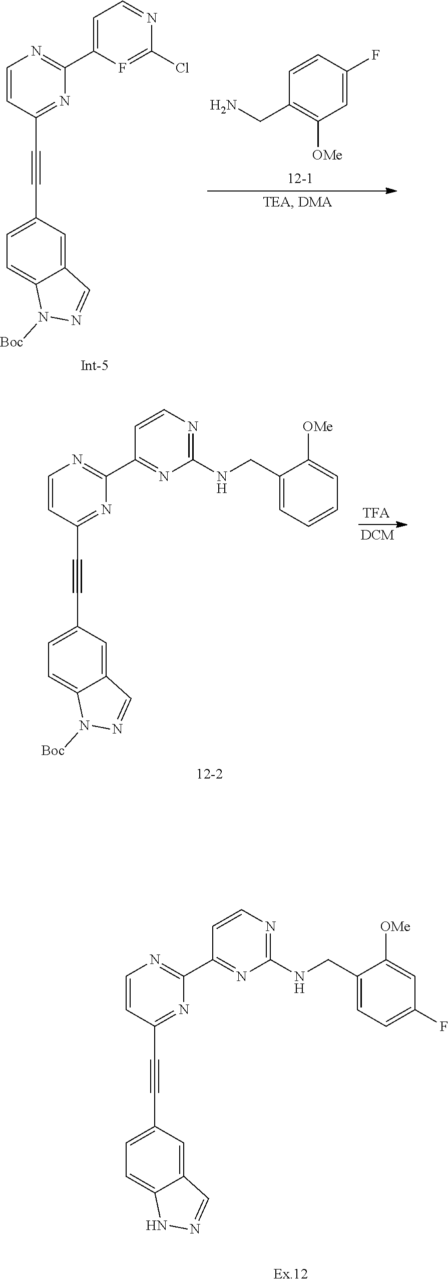

Ex. 12 was prepared from (4-fluoro-2-methoxyphenyl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): δ (ppm): 8.92 (d, J=5.1 Hz, 1H), 8.56 (m, 1H), 8.12 (s, 1H), 8.08 (s, 1H), 7.69 (d, J=5.1 Hz, 1H), 7.53 (m, 2H), 7.50 (d, J=5.0 Hz, 1H), 7.34 (m, 1H), 6.60 (m, 2H), 4.68 (d, J=5.9 Hz, 2H), 3.83 (s, 3H). MS (ESI⁺): m/z: 452.22 (M+H)⁺.

### Example 13. 4-((1H-Indazol-5-yl)ethynyl)-N-((2,2-difluorobenzo[d][1,3]dioxol-5-yl)methyl)-[2,4′-bipyrimidin]-2′-amine (Ex. 13)

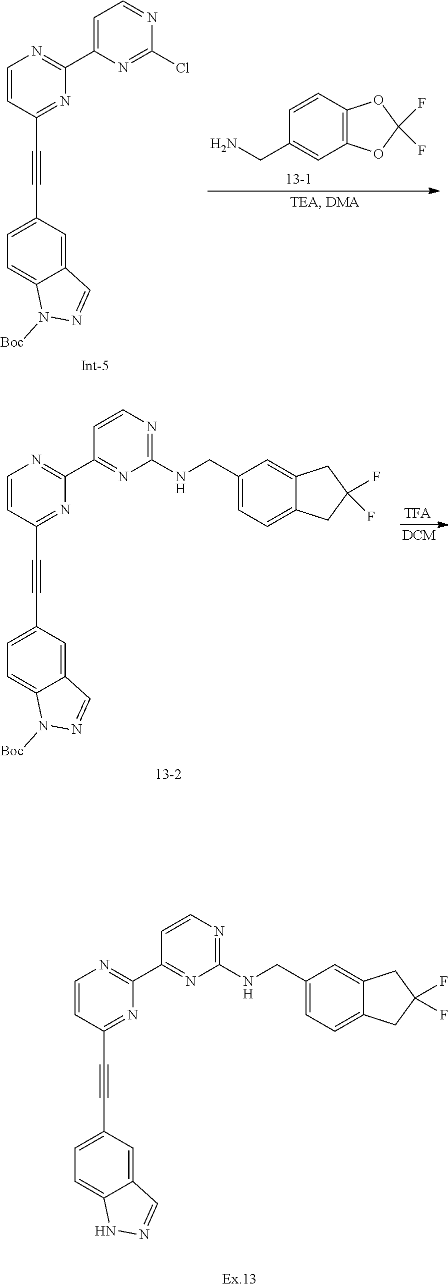 [SMILES: CC(C)(C)OC(=O)n1ncc2cc(C#Cc3ccnc(-c4ccnc(Cl)n4)n3)ccc21 ; NCc1ccc2c(c1)OC(F)(F)O2 ; CC(C)(C)OC(=O)n1ncc2cc(C#Cc3ccnc(-c4ccnc(NCc5ccc6c(c5)CC(F)(F)C6)n4)n3)ccc21 ; FC1(F)Cc2ccc(CNc3nccc(-c4nccc(C#Cc5ccc6[nH]ncc6c5)n4)n3)cc2C1]

Ex. 13 was prepared from (2,2-difluorobenzo[d][1,3]dioxol-5-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, DMSO-d₆): δ (ppm): 9.03 (d, J=5.1 Hz, 1H), 8.52 (d, J=5.0 Hz, 1H), 8.20 (m, 3H), 7.81 (d, J=5.1 Hz, 1H), 7.70 (d, J=9.1 Hz, 1H), 7.60 (dd, J=8.6, 1.5 Hz, 1H), 7.50 (d, J=5.0 Hz, 1H), 7.34 (d, J=8.3 Hz, 1H), 7.24 (m, 1H), 4.58 (d, J=6.1 Hz, 2H). MS (ESI⁺): m/z: 484.19 (M+H)⁺.

### Example 14. 4-((1H-Indazol-5-yl)ethynyl)-N-benzyl-[2,4′-bipyrimidin]-2′-amine (Ex. 14)

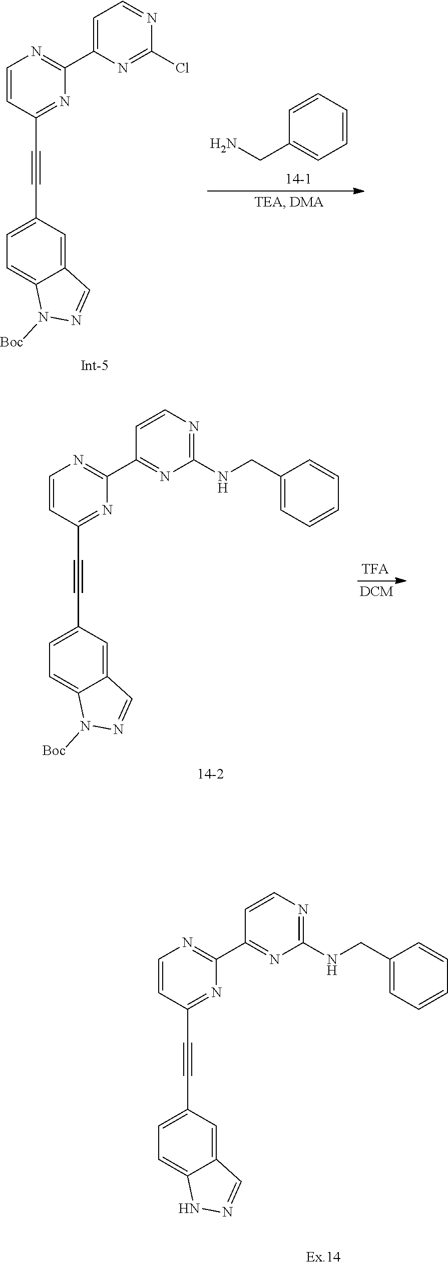 [SMILES: CC(C)(C)OC(=O)n1ncc2cc(C#Cc3ccnc(-c4ccnc(Cl)n4)n3)ccc21 ; NCc1ccccc1 ; CC(C)(C)OC(=O)n1ncc2cc(C#Cc3ccnc(-c4ccnc(NCc5ccccc5)n4)n3)ccc21 ; C(#Cc1ccnc(-c2ccnc(NCc3ccccc3)n2)n1)c1ccc2[nH]ncc2c1]

Ex. 14 was prepared from phenylmethanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, DMSO-d₆): δ (ppm): 9.00 (d, J=5.1 Hz, 1H), 8.49 (d, J=4.8 Hz, 1H), 8.19 (m, 2H), 7.78 (d, J=5.1 Hz, 1H), 7.62 (dd, J₁=18.6 Hz, J₂=6.3 Hz, 2H), 7.45 (d, J=4.8 Hz, 1H). 7.29 (m, 5H), 4.59 (br, 2H). MS (ESI⁺): m/z: 404.28 (M+H)⁺.

### Example 15. 4-((1H-Indazol-5-yl)ethynyl)-N-(2-fluorobenzyl)-[2,4′-bipyrimidin]-2′-amine (Ex. 15)

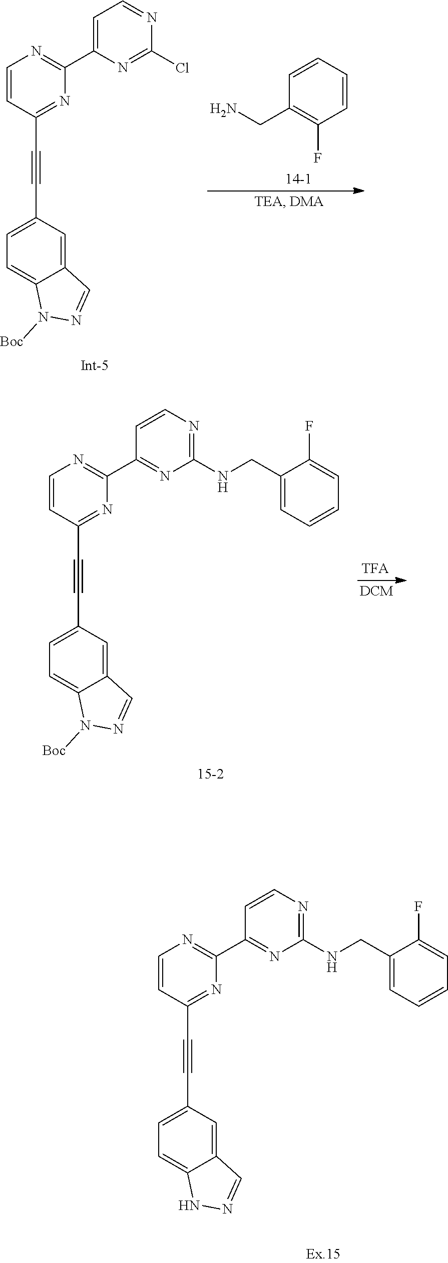 [SMILES: CC(C)(C)OC(=O)n1ncc2cc(C#Cc3ccnc(-c4ccnc(Cl)n4)n3)ccc21 ; NCc1ccccc1F ; CC(C)(C)OC(=O)n1ncc2cc(C#Cc3ccnc(-c4ccnc(NCc5ccccc5F)n4)n3)ccc21 ; Fc1ccccc1CNc1nccc(-c2nccc(C#Cc3ccc4[nH]ncc4c3)n2)n1]

Ex. 15 was prepared from (2-fluorophenyl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, DMSO-d₆): δ (ppm): 9.00 (d, J=5.1 Hz, 1H), 8.50 (d, J=5.1 Hz, 1H), 8.19 (d, J=4.5 Hz, 1H), 8.12 (br, 1H), 7.79 (d, J=5.0 Hz, 1H), 7.61 (dd, J₁=12.0 Hz, J₂=6.0 Hz, 2H), 7.47 (d, J=4.8 Hz, 1H), 7.26 (m, 1H), 7.13 (dd, J₁=18.9 Hz, J₂=11.4 Hz, 2H), 4.62 (br, 2H). MS (ESI⁺): m/z: 422.25 (M+H)⁺.

### Example 16. 4-((1H-Indazol-5-yl)ethynyl)-N-((5-chloropyridin-2-yl)methyl)-[2,4′-bipyrimidin]-2′-amine (Ex. 16)

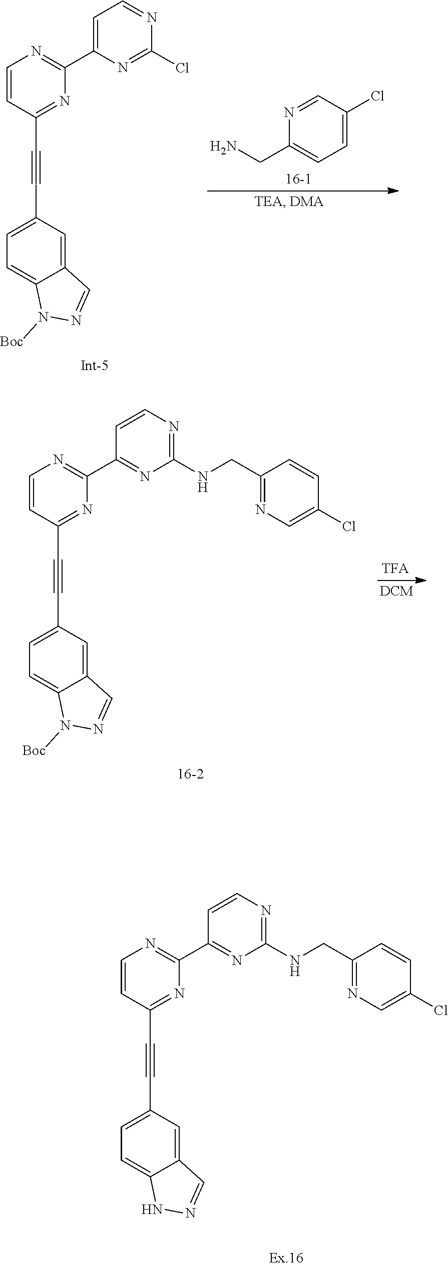 [SMILES: CC(C)(C)OC(=O)n1ncc2cc(C#Cc3ccnc(-c4ccnc(Cl)n4)n3)ccc21 ; NCc1ccc(Cl)cn1 ; CC(C)(C)OC(=O)n1ncc2cc(C#Cc3ccnc(-c4ccnc(NCc5ccc(Cl)cn5)n4)n3)ccc21 ; Clc1ccc(CNc2nccc(-c3nccc(C#Cc4ccc5[nH]ncc5c4)n3)n2)nc1]

Ex. 16 was prepared from (5-chloropyridin-2-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD-CDCl₃): δ=8.91 (d, J=5.1 Hz, 1H), 8.51-8.46 (m, 2H), 8.12 (s, 1H), 8.08 (s, 1H), 7.71-7.70 (m, 2H), 7.62-7.58 (m, 3H), 7.46-7.43 (m, 1H), 4.81 (s, 2H). MS (ESI⁺): m/z: 439.20 (M+H)⁺.

### Example 17. 4-((1H-Indazol-5-yl)ethynyl)-N-((tetrahydrofuran-2-yl)methyl)-[2,4′-bipyrimidin]-2′-amine (Ex. 17)

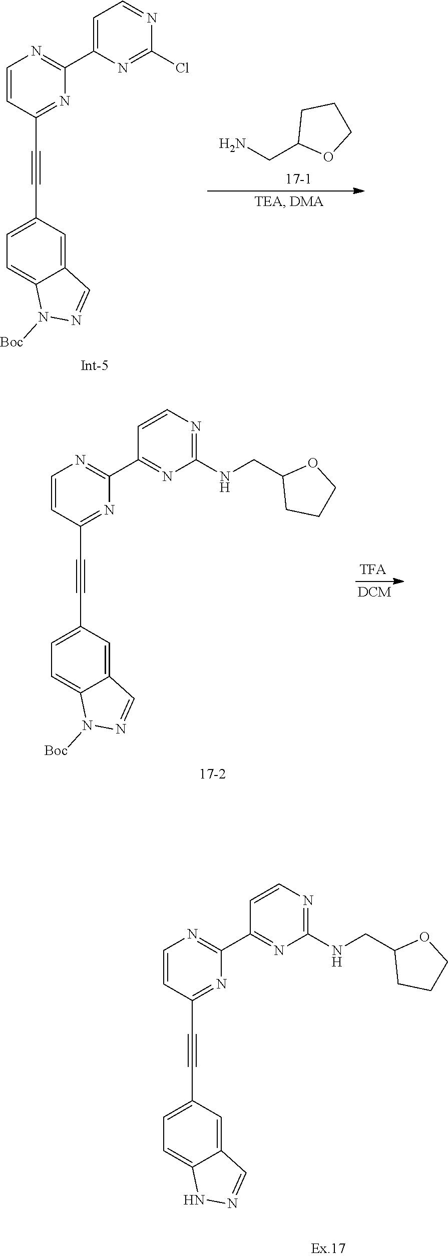 [SMILES: CC(C)(C)OC(=O)n1ncc2cc(C#Cc3ccnc(-c4ccnc(Cl)n4)n3)ccc21 ; NCC1CCCO1 ; CC(C)(C)OC(=O)n1ncc2cc(C#Cc3ccnc(-c4ccnc(NCC5CCCO5)n4)n3)ccc21 ; C(#Cc1ccnc(-c2ccnc(NCC3CCCO3)n2)n1)c1ccc2[nH]ncc2c1]

Ex. 17 was prepared from (tetrahydrofuran-2-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD-CDCl₃): δ=8.91 (d, J=4.8 Hz, 1H), 8.12 (s, 1H), 8.08 (s, 1H), 7.78-7.77 (m, 1H), 7.65-7.58 (m, 3H), 4.18-4.15 (m, 1H), 3.97-3.95 (m, 1H), 3.84-3.80 (m, 2H), 3.63-3.56 (m, 1H), 2.13-1.88 (m, 3H), 1.74-1.66 (m, 1H). MS (ESI⁺): m/z: 398.21 (M+H)⁺.

### Example 18. 4-((1H-Indazol-5-yl)ethynyl)-N-(2-methoxybenzyl)-[2,4′-bipyrimidin]-2′-amine (Ex. 18)

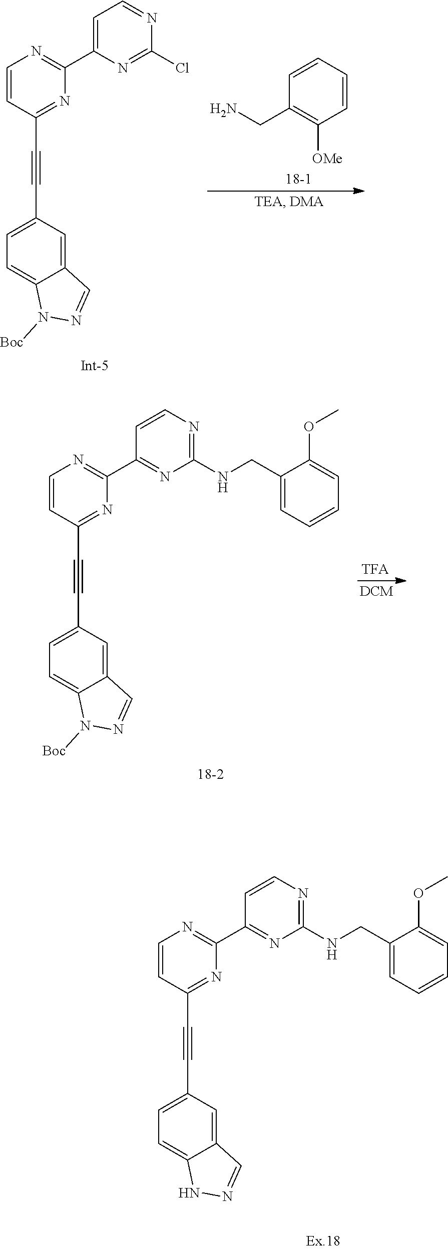 [SMILES: CC(C)(C)OC(=O)n1ncc2cc(C#Cc3ccnc(-c4ccnc(Cl)n4)n3)ccc21 ; COc1ccccc1CN ; COc1ccccc1CNc1nccc(-c2nccc(C#Cc3ccc4c(cnn4C(=O)OC(C)(C)C)c3)n2)n1 ; COc1ccccc1CNc1nccc(-c2nccc(C#Cc3ccc4[nH]ncc4c3)n2)n1]

Ex. 18 was prepared from (2-methoxyphenyl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD-CDCl₃): δ=8.88 (d, J=5.1 Hz, 1H), 8.51 (s, 1H), 8.12-8.07 (m, 2H), 7.66-7.57 (m, 4H), 7.35-7.20 (m, 2H), 6.91-6.87 (m, 2H), 4.69 (s, 2H), 3.87 (s, 3H). MS (ESI⁺): m/z: 434.29 (M+H)⁺.

### Example 19. 2-((4-((1H-Indazol-5-yl)ethynyl)-[2,4′-bipyrimidin]-2′-yl)amino)-N,N-dimethylacetamide (Ex. 19)

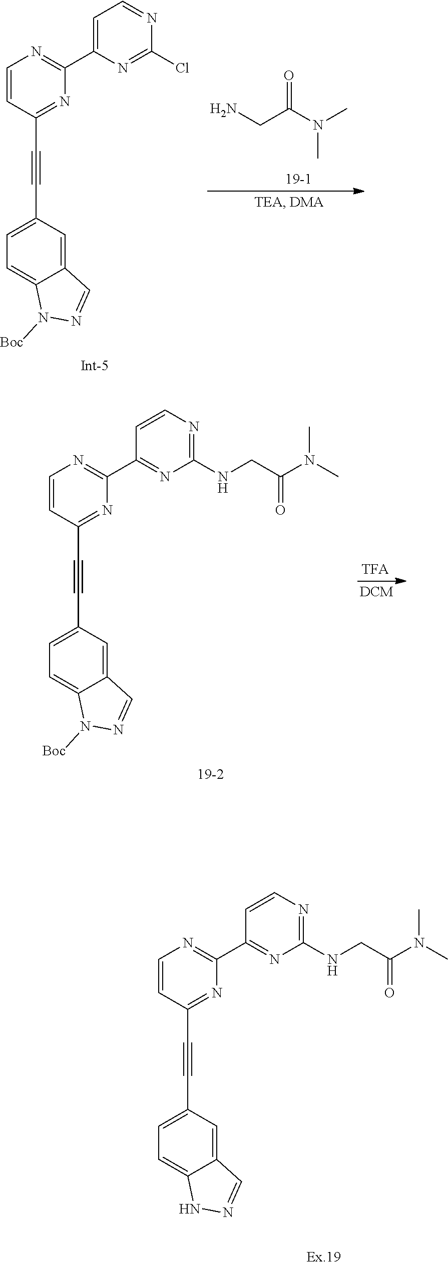

Ex. 19 was prepared from 2-amino-N,N-dimethylacetamide and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD-CDCl₃): δ=8.94-8.91 (m, 1H), 8.59 (s, 1H), 8.13-8.09 (m, 2H), 7.80-7.78 (m, 1H), 7.64-7.55 (m, 3H), 4.39 (s, 2H), 3.12 (s, 3H), 3.00 (s, 3H). MS (ESI⁺): m/z: 399.26 (M+H)⁺.

### Example 20. 4-((1H-Indazol-5-yl)ethynyl)-N-(isoindolin-5-ylmethyl)-[2,4′-bipyrimidin]-2′-amine trifluoroacetate salt (Ex. 20)

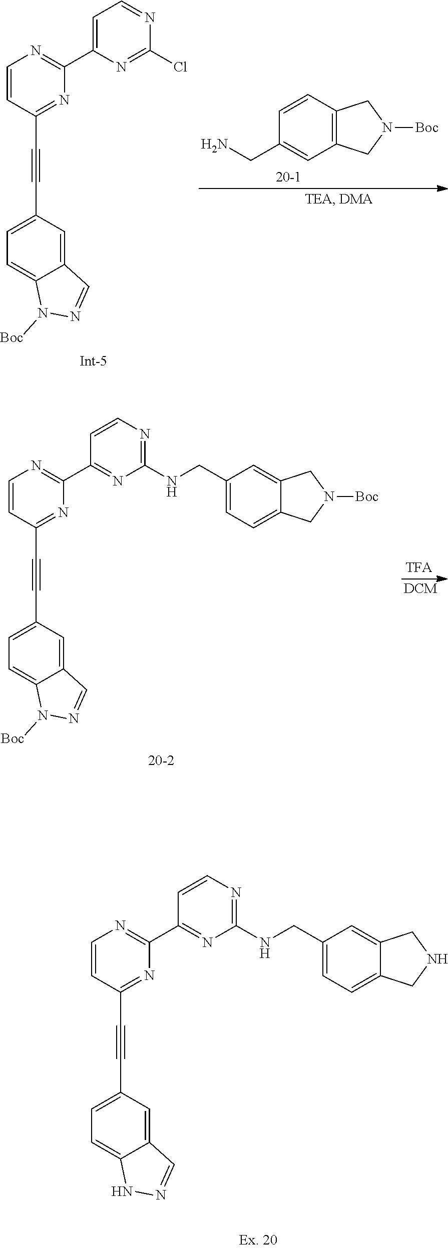 [SMILES: CC(C)(C)OC(=O)n1ncc2cc(C#Cc3ccnc(-c4ccnc(Cl)n4)n3)ccc21 ; CC(C)(C)OC(=O)N1Cc2ccc(CN)cc2C1 ; CC(C)(C)OC(=O)N1Cc2ccc(CNc3nccc(-c4nccc(C#Cc5ccc6c(cnn6C(=O)OC(C)(C)C)c5)n4)n3)cc2C1 ; C(#Cc1ccnc(-c2ccnc(NCc3ccc4c(c3)CNC4)n2)n1)c1ccc2[nH]ncc2c1]

Ex. 20 was prepared from tert-butyl 5-(aminomethyl)isoindoline-2-carboxylate and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (400 MHz, CD₃OD) δ 8.87 (d, J=6.8 Hz, 1H), 8.53 (s, 1H), 8.03 (m, 2H), 7.68 (d, J=6.8 Hz, 1H), 7.64 (d, J=6.8 Hz, 1H), 7.51-7.40 (m, 4H), 7.29 (d, J=10 Hz, 1H), 4.83 (s, 2H), 4.49 (s, 4H). MS (ESI⁺): m/z: 445.5 (M+H)⁺.

### Example 21. 3-(((4-((1H-Indazol-5-yl)ethynyl)-[2,4′-bipyrimidin]-2′-yl)amino)methyl)pyridin-2(1H)-one (Ex. 21)

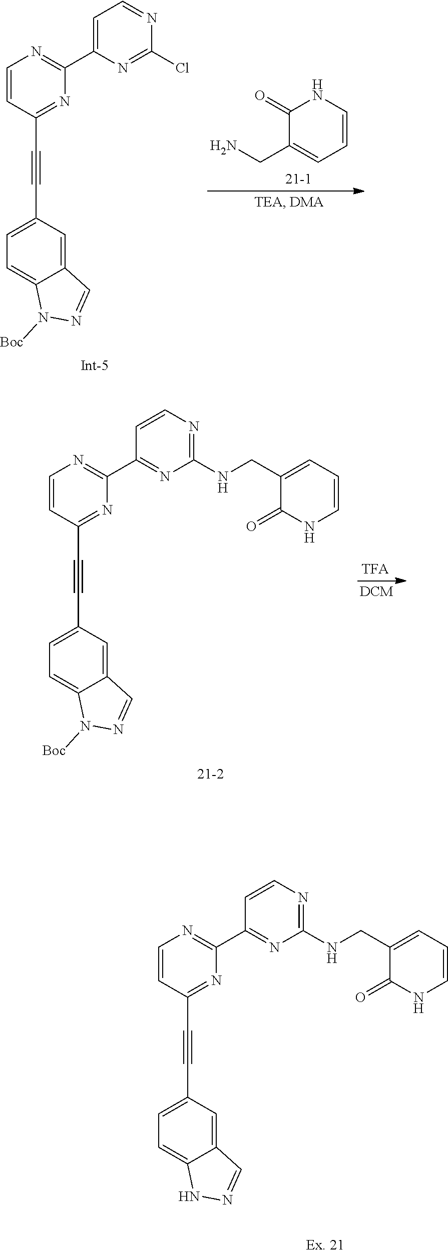 [SMILES: CC(C)(C)OC(=O)n1ncc2cc(C#Cc3ccnc(-c4ccnc(Cl)n4)n3)ccc21 ; NCc1ccc[nH]c1=O ; CC(C)(C)OC(=O)n1ncc2cc(C#Cc3ccnc(-c4ccnc(NCc5ccc[nH]c5=O)n4)n3)ccc21 ; O=c1[nH]cccc1CNc1nccc(-c2nccc(C#Cc3ccc4[nH]ncc4c3)n2)n1]

Ex. 21 was prepared from 3-(aminomethyl)pyridin-2(1H)-one and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (400 MHz, DMSO-d₆) δ 9.05 (d, J=6.8 Hz, 1H), 8.57 (d, J=6.8 Hz, 1H), 8.21 (m, 2H), 7.84 (d, J=6.8 Hz, 1H), 7.69-7.53 (m, 4H), 7.29 (m, 1H), 6.19 (m, 1H), 4.39 (s, 2H). MS (ESI⁺): m/z: 421.27 (M+H)⁺.

### Example 22. 4-((1H-Indazol-5-yl)ethynyl)-N-(pyridin-2-ylmethyl)-[2,4′-bipyrimidin]-2′-amine (Ex. 22)

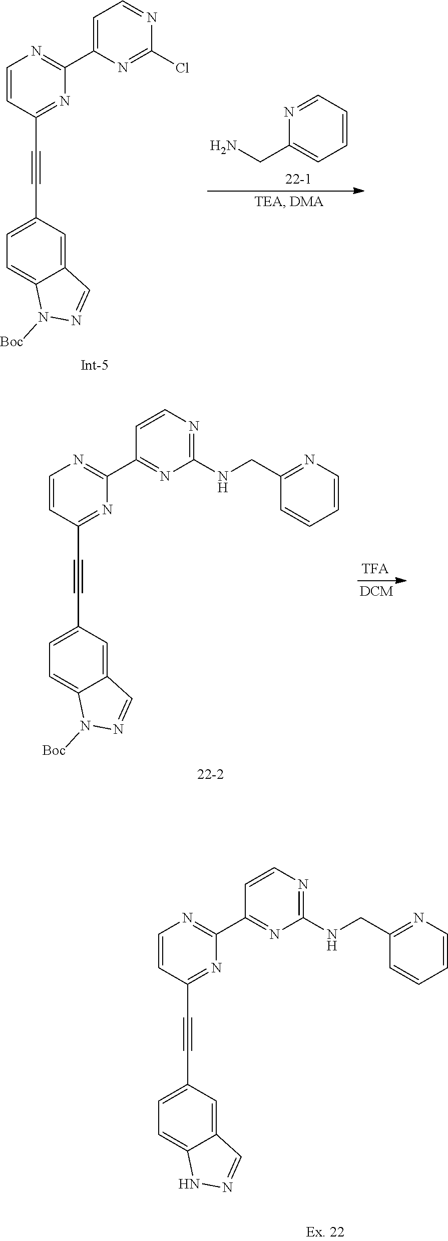 [SMILES: CC(C)(C)OC(=O)n1ncc2cc(C#Cc3ccnc(-c4ccnc(Cl)n4)n3)ccc21 ; NCc1ccccn1 ; CC(C)(C)OC(=O)n1ncc2cc(C#Cc3ccnc(-c4ccnc(NCc5ccccn5)n4)n3)ccc21 ; C(#Cc1ccnc(-c2ccnc(NCc3ccccn3)n2)n1)c1ccc2[nH]ncc2c1]

Ex. 22 was prepared from pyridin-2-ylmethanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD): δ (ppm): 8.95 (d, J=5.1 Hz, 1H), 8.50 (m, 2H), 8.18 (d, J=11.1 Hz, 1H), 7.77 (td, J₁=7.8 Hz, J₂=1.8 Hz, 1H), 7.68 (dd, J₁=4.8 Hz, J₂=1.8 Hz, 2H), 7.63 (s, 2H), 7.49 (d, J=7.5 Hz, 1H), 7.29 (t, J=5.1 Hz, 1H), 4.86 (br, 2H). MS (ESI⁺): m/z: 405.26 (M+H)⁺.

### Example 23. N-((1H-Imidazol-2-yl)methyl)-4-((1H-indazol-5-yl)ethynyl)-[2,4′-bipyrimidin]-2′-amine (Ex. 23)

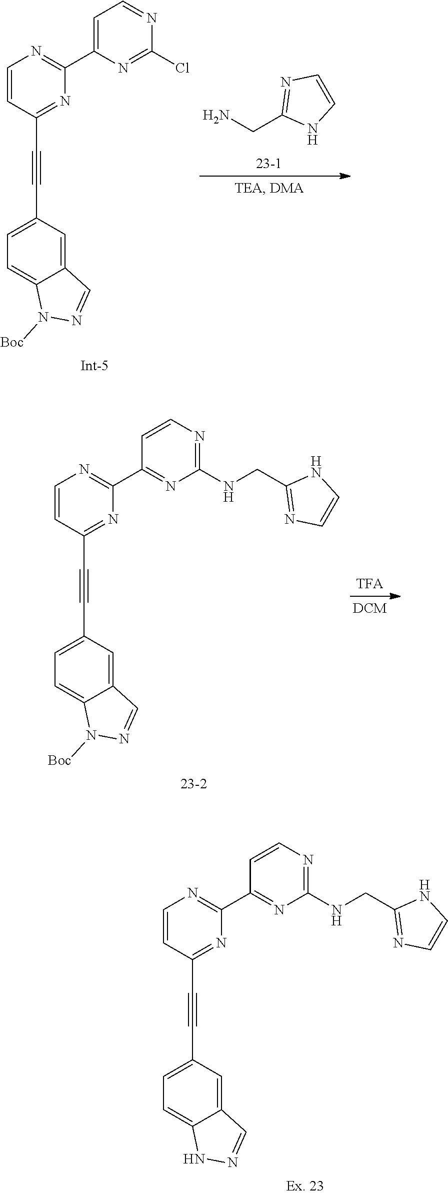 [SMILES: CC(C)(C)OC(=O)n1ncc2cc(C#Cc3ccnc(-c4ccnc(Cl)n4)n3)ccc21 ; NCc1ncc[nH]1 ; CC(C)(C)OC(=O)n1ncc2cc(C#Cc3ccnc(-c4ccnc(NCc5ncc[nH]5)n4)n3)ccc21 ; C(#Cc1ccnc(-c2ccnc(NCc3ncc[nH]3)n2)n1)c1ccc2[nH]ncc2c1]

Ex. 23 was prepared from (1H-imidazol-2-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD): δ (ppm): 9.04 (d, J=5.1 Hz, 1H), 8.63 (d, J=5.0 Hz, 1H), 8.19 (t, J=1.2 Hz, 1H), 8.16 (s, 1H), 7.83 (d, J=5.0 Hz, 1H), 7.78 (d, J=5.1 Hz, 1H), 7.64 (m, 2H), 7.53 (br s, 2H), 4.91 (s, 2H). MS (ESI⁺): m/z: 394.24 (M+H)⁺.

### Example 24. 4-((1H-Indazol-5-yl)ethynyl)-N-((5-methylpyridin-2-yl)methyl)-[2,4′-bipyrimidin]-2′-amine (Ex. 24)

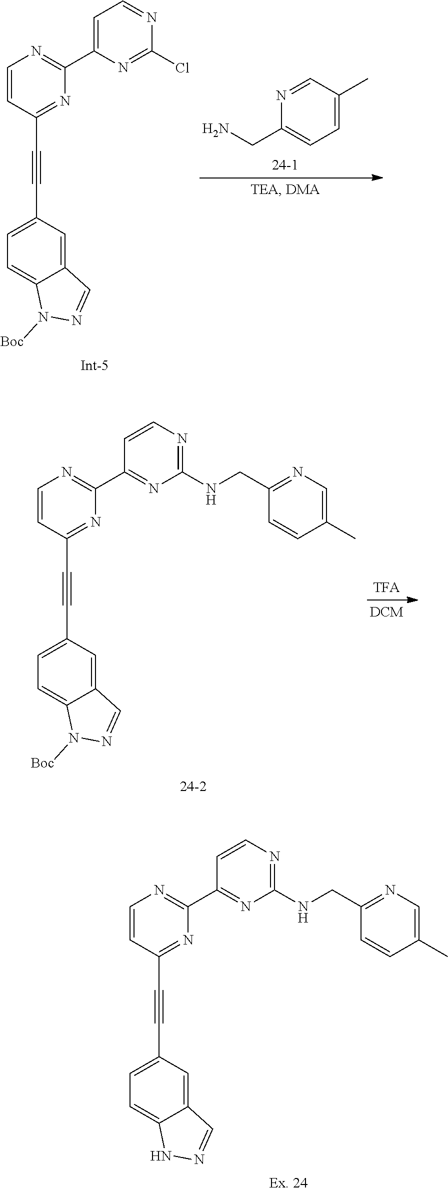

Ex. 24 was prepared from (5-methylpyridin-2-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): δ (ppm): 8.93 (d, J=5.1 Hz, 1H), 8.57 (d, J=5.0 Hz, 1H), 8.39 (m, 1H), 8.14 (s, 1H), 8.07 (s, 1H), 7.72 (d, J=5.1 Hz, 1H), 7.56-7.43 (m, 4H), 7.26 (m, 1H), 4.84 (d, J=5.5 Hz, 2H), 2.32 (s, 3H). MS (ESI⁺): m/z: 419.26 (M+H)⁺.

### Example 25. 4-((1H-Indazol-5-yl)ethynyl)-N-(isoquinolin-3-ylmethyl)-[2,4′-bipyrimidin]-2′-amine (Ex. 25)

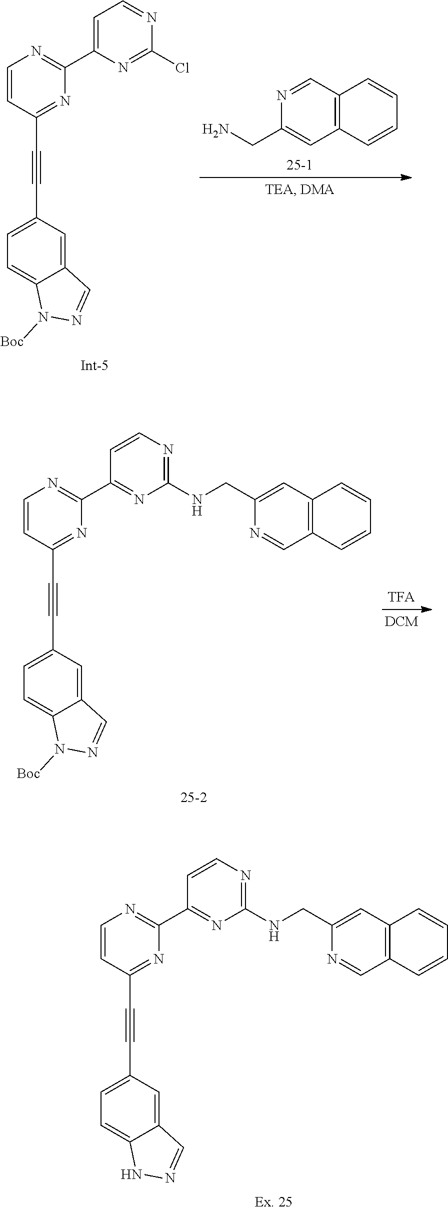 [SMILES: CC(C)(C)OC(=O)n1ncc2cc(C#Cc3ccnc(-c4ccnc(Cl)n4)n3)ccc21 ; NCc1cc2ccccc2cn1 ; CC(C)(C)OC(=O)n1ncc2cc(C#Cc3ccnc(-c4ccnc(NCc5cc6ccccc6cn5)n4)n3)ccc21 ; C(#Cc1ccnc(-c2ccnc(NCc3cc4ccccc4cn3)n2)n1)c1ccc2[nH]ncc2c1]

Ex. 25 was prepared from isoquinolin-3-ylmethanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): δ (ppm): 9.24 (m, 1H), 8.94 (d, J=5.1 Hz, 1H), 8.58 (d, J=5.1 Hz, 1H), 8.13 (m, 1H), 8.06 (m, 1H), 7.96 (d, J=8.1 Hz, 1H), 7.78-7.73 (m, 3H), 7.65 (ddd, J=8.1, 6.8, 1.3 Hz, 1H), 7.57 (m, 1H), 7.52 (m, 2H), 7.47 (d, J=8.1 Hz, 1H), 5.06 (d, J=5.9 Hz, 2H). MS (ESI⁺): m/z: 455.25 (M+H)⁺.

### Example 26. 4-((1H-Indazol-5-yl)ethynyl)-N-(2,3-difluorobenzyl)-[2,4′-bipyrimidin]-2′-amine (Ex. 26)

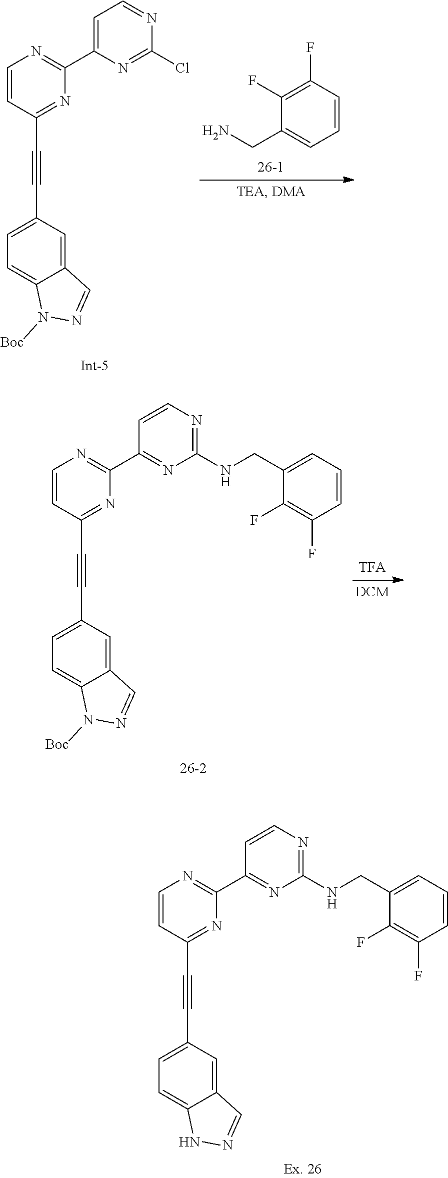 [SMILES: CC(C)(C)OC(=O)n1ncc2cc(C#Cc3ccnc(-c4ccnc(Cl)n4)n3)ccc21 ; NCc1cccc(F)c1F ; CC(C)(C)OC(=O)n1ncc2cc(C#Cc3ccnc(-c4ccnc(NCc5cccc(F)c5F)n4)n3)ccc21 ; Fc1cccc(CNc2nccc(-c3nccc(C#Cc4ccc5[nH]ncc5c4)n3)n2)c1F]

Ex. 26 was prepared from (2,3-difluorophenyl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD-CDCl₃): δ=8.94 (s, 1H), 8.63 (s, 1H), 8.22-8.13 (m, 2H), 7.80-7.58 (m, 4H), 7.30-7.06 (m, 3H), 4.85 (s, 2H). MS (ESI⁺): m/z: 440.2 (M+H)⁺.

### Example 27. 4-((1H-Indazol-5-yl)ethynyl)-N-(2,6-difluorobenzyl)-[2,4′-bipyrimidin]-2′-amine (Ex. 27)

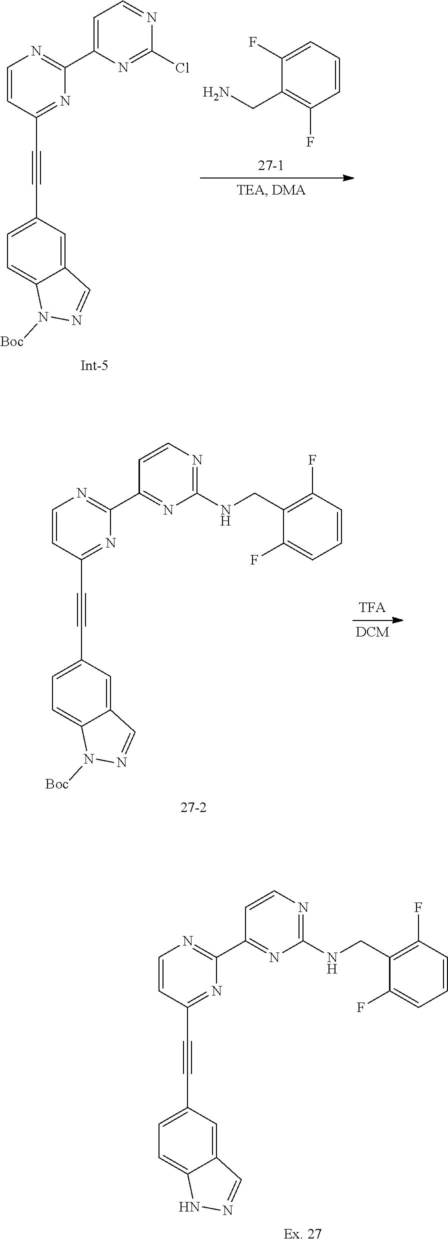 [SMILES: CC(C)(C)OC(=O)n1ncc2cc(C#Cc3ccnc(-c4ccnc(Cl)n4)n3)ccc21 ; NCc1c(F)cccc1F ; CC(C)(C)OC(=O)n1ncc2cc(C#Cc3ccnc(-c4ccnc(NCc5c(F)cccc5F)n4)n3)ccc21 ; Fc1cccc(F)c1CNc1nccc(-c2nccc(C#Cc3ccc4[nH]ncc4c3)n2)n1]

Ex. 27 was prepared from (2,6-difluorophenyl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD-CDCl₃): δ=8.89 (s, 1H), 8.59 (s, 1H), 8.12-8.07 (m, 2H), 7.74-7.56 (m, 4H), 7.31-7.26 (m, 1H), 6.98-6.86 (m, 2H), 4.82 (s, 2H). MS (ESI⁺): m/z: 440.2 (M+H)⁺.

### Example 28. 4-((1H-Indazol-5-yl)ethynyl)-N-(1-(5-fluoropyridin-2-yl)ethyl)-[2,4′-bipyrimidin]-2′-amine (Ex. 28)

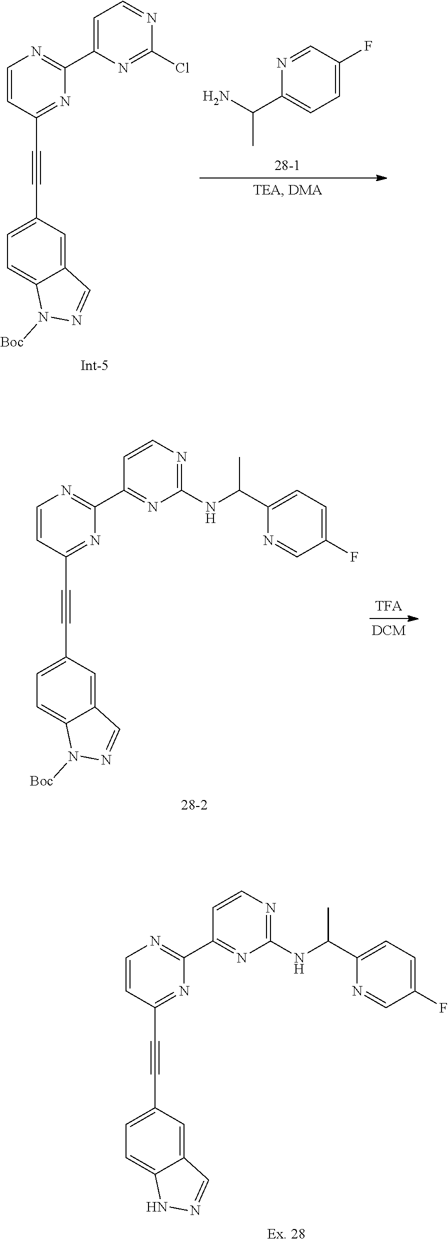

Ex. 28 was prepared from 1-(5-fluoropyridin-2-yl)ethan-1-amine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): δ (ppm): 8.82 (d, J=5.0 Hz, 1H), 8.38 (d, J=5.1 Hz, 1H), 8.31 (d, J=2.7 Hz, 1H), 8.04 (m, 1H), 8.01 (m, 1H), 7.58 (d, J=5.1 Hz, 1H), 7.54 (dd, J=8.8, 1.4 Hz, 1H), 7.48 (d, J=8.8 Hz, 1H), 7.46 (d, J=5.1 Hz, 1H), 7.37 (dd, J=8.8, 4.9 Hz, 1H), 7.30 (dd, J=8.1, 2.9 Hz, 1H), 5.25 (m, 1H), 1.52 (d, J=6.8 Hz, 3H). MS (ESI⁺): m/z: 437.23 (M+H)⁺.

### Example 29. 4-((1H-Indazol-5-yl)ethynyl)-N-((5-fluoropyridin-3-yl)methyl)-[2,4′-bipyrimidin]-2′-amine Ex. 29)

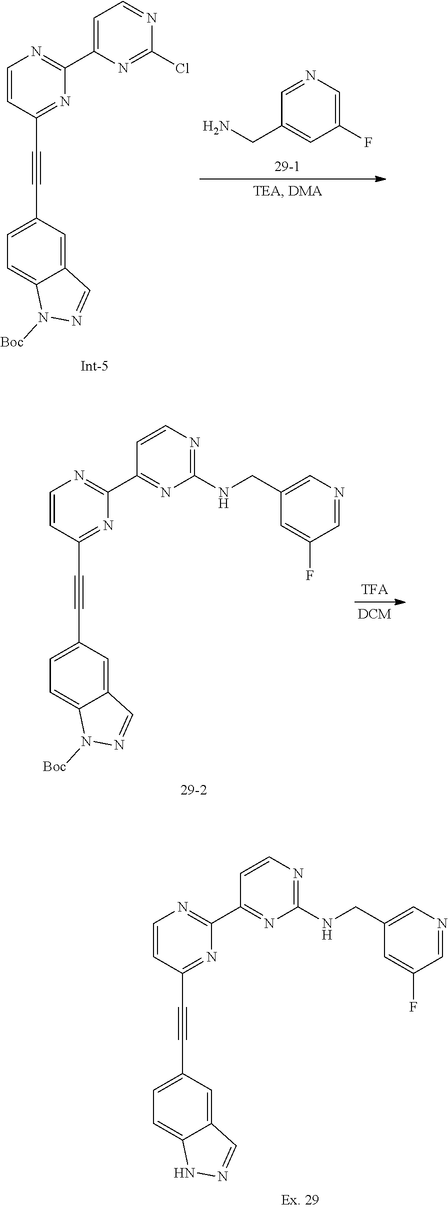 [SMILES: CC(C)(C)OC(=O)n1ncc2cc(C#Cc3ccnc(-c4ccnc(Cl)n4)n3)ccc21 ; NCc1cncc(F)c1 ; CC(C)(C)OC(=O)n1ncc2cc(C#Cc3ccnc(-c4ccnc(NCc5cncc(F)c5)n4)n3)ccc21 ; Fc1cncc(CNc2nccc(-c3nccc(C#Cc4ccc5[nH]ncc5c4)n3)n2)c1]

Ex. 29 was prepared from (5-fluoropyridin-3-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD): δ (ppm): 8.96 (d, J=5.1 Hz, 1H), 8.56-8.49 (m, 2H), 8.38 (m, 1H), 8.32 (m, 1H), 8.19 (s, 1H), 8.16 (s, 1H), 7.71 (s, 1H), 7.70 (s, 1H), 7.64 (m, 2H), 4.81 (m, 2H). MS (ESI⁺): m/z: 423.23 (M+H)⁺.

### Example 30. 4-((1H-Indazol-5-yl)ethynyl)-N-((5-fluoropyridin-2-yl)methyl)-N-methyl-[2,4′-bipyrimidin]-2′-amine (Ex. 30)

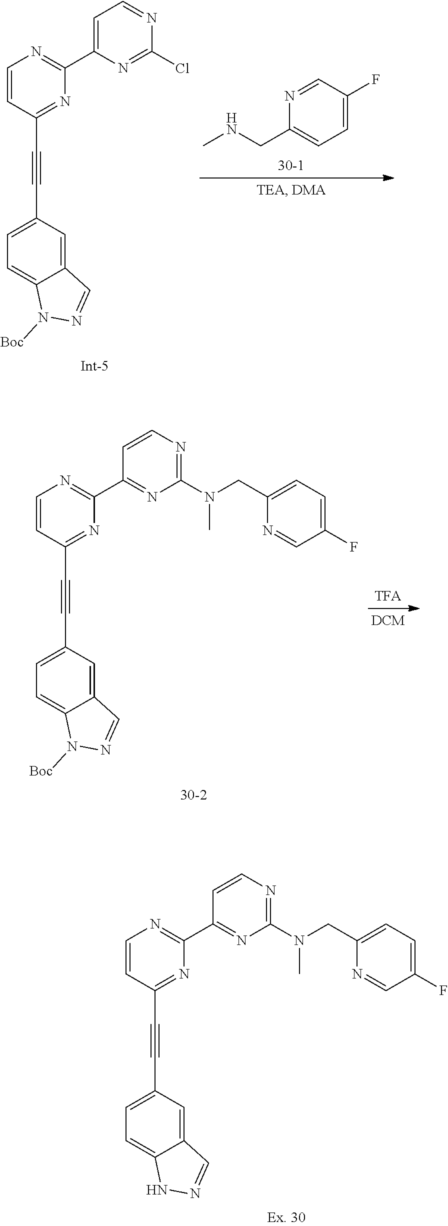

Ex. 30 was prepared from 1-(5-fluoropyridin-2-yl)-N-methylmethanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (400 MHz, CDCl₃) δ 8.90 (d, J=6.8 Hz, 1H), 8.54 (d, J=6.8 Hz, 1H), 8.39 (s, 1H), 8.10 (s, 1H), 8.05 (s, 1H), 7.62 (d, J=6.8 Hz, 1H), 7.56-7.46 (m, 4H), 7.32-7.25 (m, 2H), 5.10 (s, 2H), 3.32 (s, 3H). MS (ESI⁺): m/z: 437.4 (M+H)⁺.

### Example 31. 4-((1H-Indazol-5-yl)ethynyl)-N-((3-fluoropyridin-4-yl)methyl)-[2,4′-bipyrimidin]-2′-amine (Ex. 31)

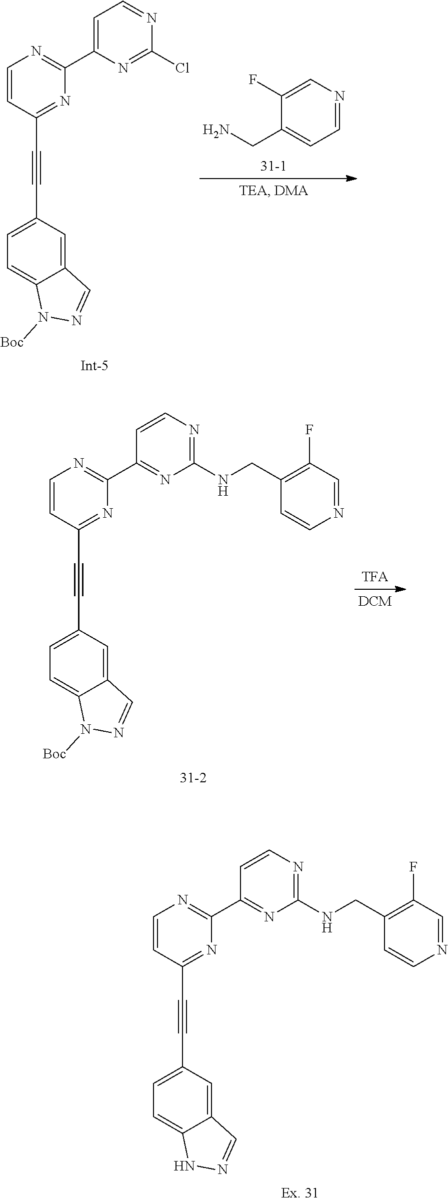 [SMILES: CC(C)(C)OC(=O)n1ncc2cc(C#Cc3ccnc(-c4ccnc(Cl)n4)n3)ccc21 ; NCc1ccncc1F ; CC(C)(C)OC(=O)n1ncc2cc(C#Cc3ccnc(-c4ccnc(NCc5ccncc5F)n4)n3)ccc21 ; Fc1cnccc1CNc1nccc(-c2nccc(C#Cc3ccc4[nH]ncc4c3)n2)n1]

Ex. 31 was prepared from (3-fluoropyridin-4-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD): δ (ppm): 8.93 (d, J=5.1 Hz, 1H), 8.54-8.48 (m, 2H), 8.40 (m, 1H), 8.33 (m, 1H), 8.20 (s, 1H), 8.16 (m, 1H), 7.76 (m, 2H), 7.64 (m, 2H), 4.82 (s, 2H). MS (ESI⁺): m/z: 423.23 (M+H)⁺.

### Example 32. 4-((1H-Indazol-5-yl)ethynyl)-N-((6-fluoropyridin-2-yl)methyl)-[2,4′-bipyrimidin]-2′-amine (Ex. 32)

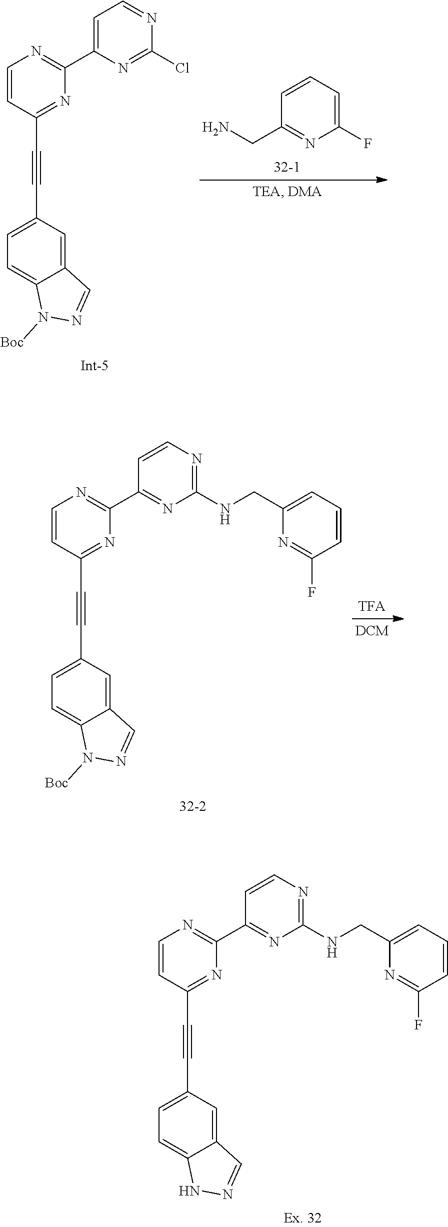 [SMILES: CC(C)(C)OC(=O)n1ncc2cc(C#Cc3ccnc(-c4ccnc(Cl)n4)n3)ccc21 ; NCc1cccc(F)n1 ; CC(C)(C)OC(=O)n1ncc2cc(C#Cc3ccnc(-c4ccnc(NCc5cccc(F)n5)n4)n3)ccc21 ; Fc1cccc(CNc2nccc(-c3nccc(C#Cc4ccc5[nH]ncc5c4)n3)n2)n1]

Ex. 32 was prepared from (6-fluoropyridin-2-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD): δ (ppm): 8.93 (d, J=5.1 Hz, 1H), 8.54-8.48 (m, 2H), 8.40 (m, 1H), 8.33 (m, 1H), 8.20 (s, 1H), 8.16 (m, 1H), 7.76 (m, 2H), 7.64 (m, 2H), 4.82 (s, 2H). MS (ESI⁺): m/z: 423.23 (M+H)⁺.

### Example 33. 4-((1H-Indazol-5-yl)ethynyl)-N-((2-fluoropyridin-4-yl)methyl)-[2,4′-bipyrimidin]-2′-amine (Ex. 33)

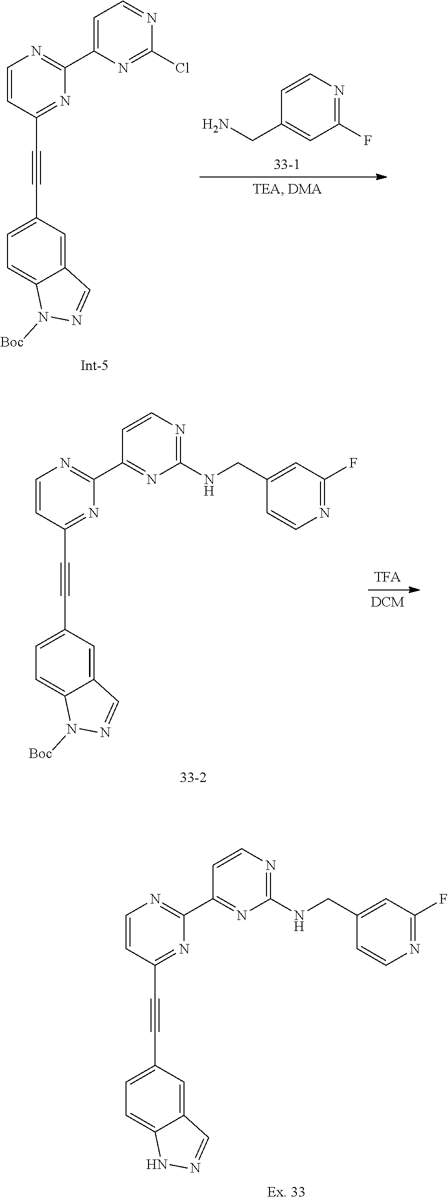 [SMILES: CC(C)(C)OC(=O)n1ncc2cc(C#Cc3ccnc(-c4ccnc(Cl)n4)n3)ccc21 ; NCc1ccnc(F)c1 ; CC(C)(C)OC(=O)n1ncc2cc(C#Cc3ccnc(-c4ccnc(NCc5ccnc(F)c5)n4)n3)ccc21 ; Fc1cc(CNc2nccc(-c3nccc(C#Cc4ccc5[nH]ncc5c4)n3)n2)ccn1]

Ex. 33 was prepared from (2-fluoropyridin-4-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (400 MHz, CD₃OD) δ 8.92 (d, J=6.8 Hz, 1H), 8.50 (d, J=6.8 Hz, 1H), 8.86-8.08 (m, 3H), 7.72 (d, J=6.4 Hz, 1H), 7.63-7.59 (m, 4H), 7.33 (d, J=6.4 Hz, 1H), 7.09 (m, 1H), 4.80 (s, 2H). MS (ESI⁺): m/z: 423.23 (M+H)⁺.

### Example 34. (R)-4-((1H-Indazol-5-yl)ethynyl)-N-((tetrahydrofuran-2-yl)methyl)-[2,4′-bipyrimidin]-2′-amine (Ex. 34)

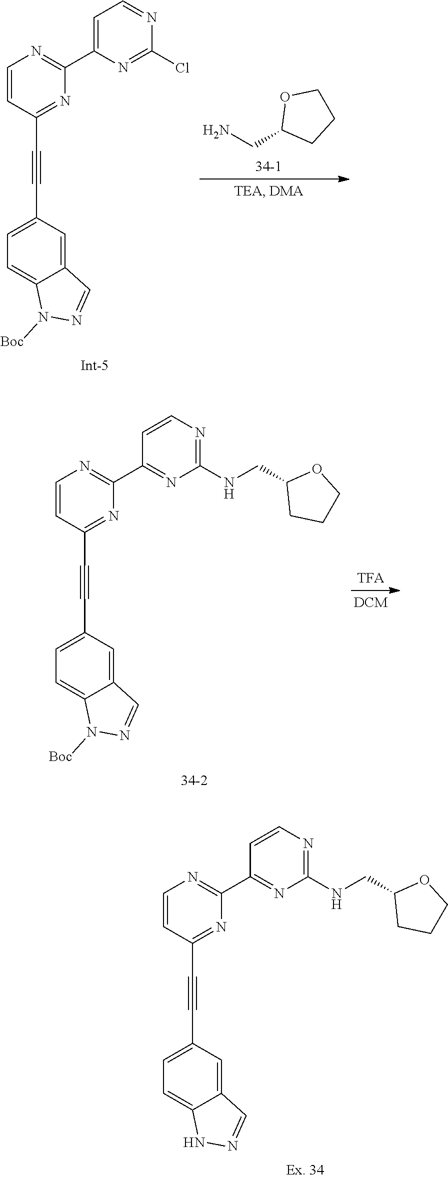 [SMILES: CC(C)(C)OC(=O)n1ncc2cc(C#Cc3ccnc(-c4ccnc(Cl)n4)n3)ccc21 ; NC[C@H]1CCCO1 ; CC(C)(C)OC(=O)n1ncc2cc(C#Cc3ccnc(-c4ccnc(NC[C@H]5CCCO5)n4)n3)ccc21 ; C(#Cc1ccnc(-c2ccnc(NC[C@H]3CCCO3)n2)n1)c1ccc2[nH]ncc2c1]

Ex. 34 was prepared from (R)-(tetrahydrofuran-2-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD-CDCl₃): δ=8.89 (d, J=5.1 Hz, 1H), 8.47 (d, J=4.8 Hz, 1H), 8.13 (s, 1H), 8.09 (s, 1H), 7.65-7.58 (m, 4H), 4.18-4.12 (m, 1H), 3.94-3.73 (m, 3H), 3.59-3.52 (m, 1H), 2.05-1.90 (m, 3H), 1.72-1.65 (m, 1H). MS (ESI⁺): m/z: 398.3 (M+H)⁺.

### Example 35. (S)-4-((1H-Indazol-5-yl)ethynyl)-N-((tetrahydrofuran-2-yl)methyl)-[2,4′-bipyrimidin]-2′-amine (Ex. 35)

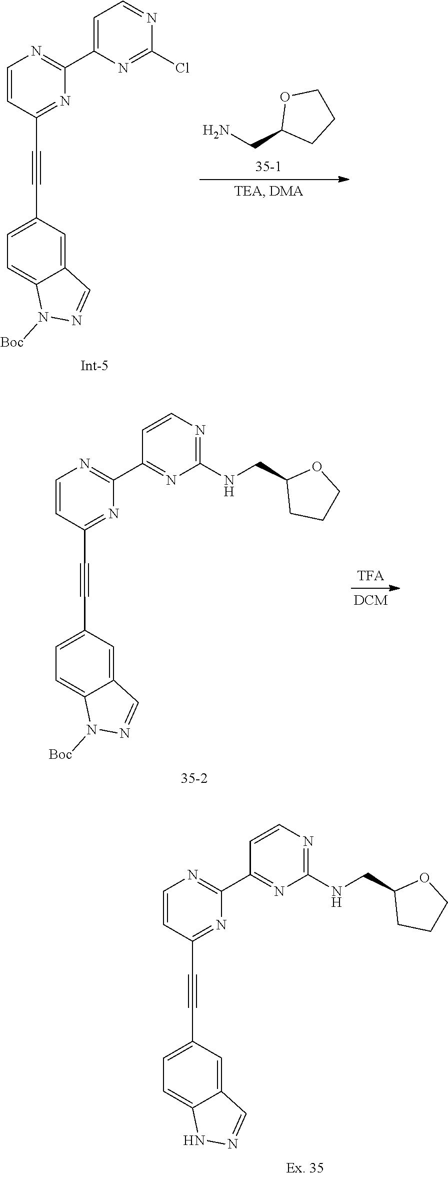 [SMILES: CC(C)(C)OC(=O)n1ncc2cc(C#Cc3ccnc(-c4ccnc(Cl)n4)n3)ccc21 ; NC[C@@H]1CCCO1 ; CC(C)(C)OC(=O)n1ncc2cc(C#Cc3ccnc(-c4ccnc(NC[C@@H]5CCCO5)n4)n3)ccc21 ; C(#Cc1ccnc(-c2ccnc(NC[C@@H]3CCCO3)n2)n1)c1ccc2[nH]ncc2c1]

Ex. 35 was prepared from (S)-(tetrahydrofuran-2-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD-CDCl₃): δ=8.89 (d, J=5.1 Hz, 1H), 8.47 (d, J=4.8 Hz, 1H), 8.12 (s, 1H), 8.08 (s, 1H), 7.65-7.58 (m, 4H), 4.18-4.10 (m, 1H), 3.94-3.77 (m, 2H), 3.80-3.77 (m, 1H), 3.59-3.52 (m, 1H), 2.07-1.88 (m, 3H), 1.75-1.66 (m, 1H). MS (ESI⁺): m/z: 398.3 (M+H)⁺.

### Example 36. 4-((7-Fluoro-1H-indazol-5-yl)ethynyl)-N-((5-fluoropyridin-2-yl)methyl)-[2,4′-bipyrimidin]-2′-amine (Ex. 36)

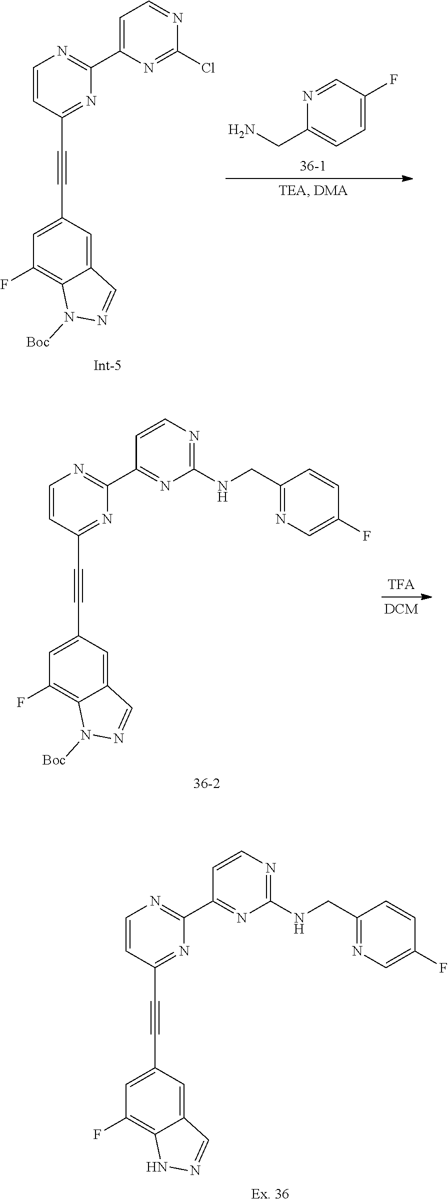 [SMILES: CC(C)(C)OC(=O)n1ncc2cc(C#Cc3ccnc(-c4ccnc(Cl)n4)n3)cc(F)c21 ; NCc1ccc(F)cn1 ; CC(C)(C)OC(=O)n1ncc2cc(C#Cc3ccnc(-c4ccnc(NCc5ccc(F)cn5)n4)n3)cc(F)c21 ; Fc1ccc(CNc2nccc(-c3nccc(C#Cc4cc(F)c5[nH]ncc5c4)n3)n2)nc1]

Ex. 36 was prepared from (5-fluoropyridin-2-yl)methanamine and tert-butyl 5-((2′-chloro-[2,4′-bipyrimidin]-4-yl)ethynyl)-7-fluoro-1H-indazole-1-carboxylate (Int-8) in a manner analogous to the procedures described for Step 3 and Step 4 of Example 1. ¹H-NMR (300 MHz, CDCl₃): δ (ppm): 8.88 (d, J=5.1 Hz, 1H), 8.49 (d, J=5.1 Hz, 1H), 8.36 (m, 1H), 8.09 (d, J=3.2 Hz, 1H), 7.87 (d, J=1.1 Hz, 1H), 7.67 (d, J=5.1 Hz, 1H), 7.50 (d, J=5.0 Hz, 1H), 7.39 (m, 1H), 7.35 (dd, J=7.9, 2.7 Hz, 1H), 7.27 (dd, J=10.8, 1.1 Hz, 1H), 4.80 (s, 2H). MS (ESI⁺): m/z: 441.20 (M+H)⁺.

### Example 37. 4-((1H-Indazol-5-yl)ethynyl)-N-(2,3,5-trifluorobenzyl)-[2,4′-bipyrimidin]-2′-amine (Ex. 37)

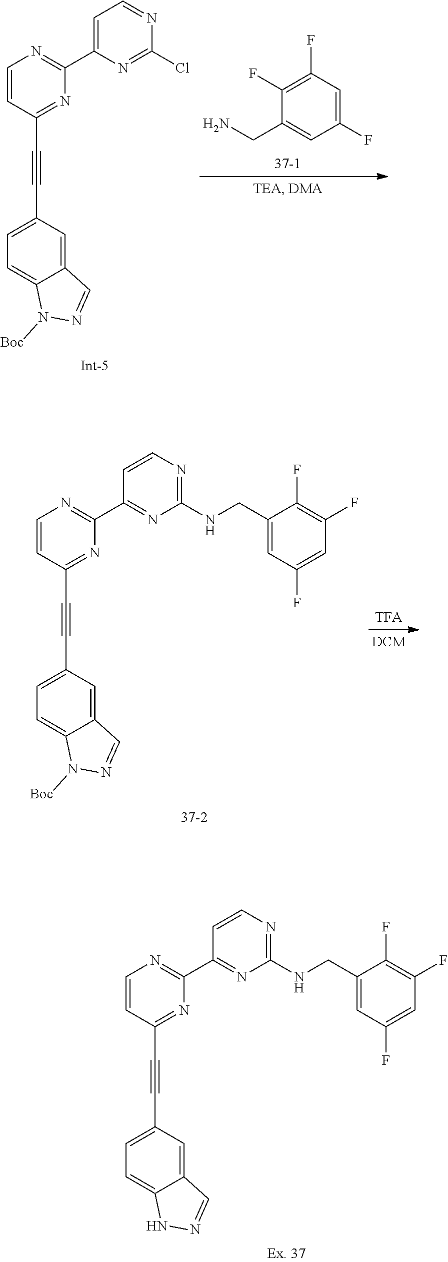 [SMILES: CC(C)(C)OC(=O)n1ncc2cc(C#Cc3ccnc(-c4ccnc(Cl)n4)n3)ccc21 ; NCc1cc(F)cc(F)c1F ; CC(C)(C)OC(=O)n1ncc2cc(C#Cc3ccnc(-c4ccnc(NCc5cc(F)cc(F)c5F)n4)n3)ccc21 ; Fc1cc(F)c(F)c(CNc2nccc(-c3nccc(C#Cc4ccc5[nH]ncc5c4)n3)n2)c1]

Ex. 37 was prepared from (2,3,5-trifluorophenyl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (400 MHz, CD₃OD) δ 8.91 (d, J=6.8 Hz, 1H), 8.53 (d, J=6.8 Hz, 1H), 8.13 (s, 1H), 8.09 (s, 1H), 7.72 (d, J=6.4 Hz, 1H), 7.63-7.61 (m, 3H), 7.25 (m, 1H), 6.95 (m, 1H), 4.76 (s, 2H). MS (ESI⁺): m/z: 458.42 (M+H)⁺.

### Example 38. 4-((1H-Indazol-5-yl)ethynyl)-N-((3,5-difluoropyridin-2-yl)methyl)-[2,4′-bipyrimidin]-2′-amine (Ex. 38)

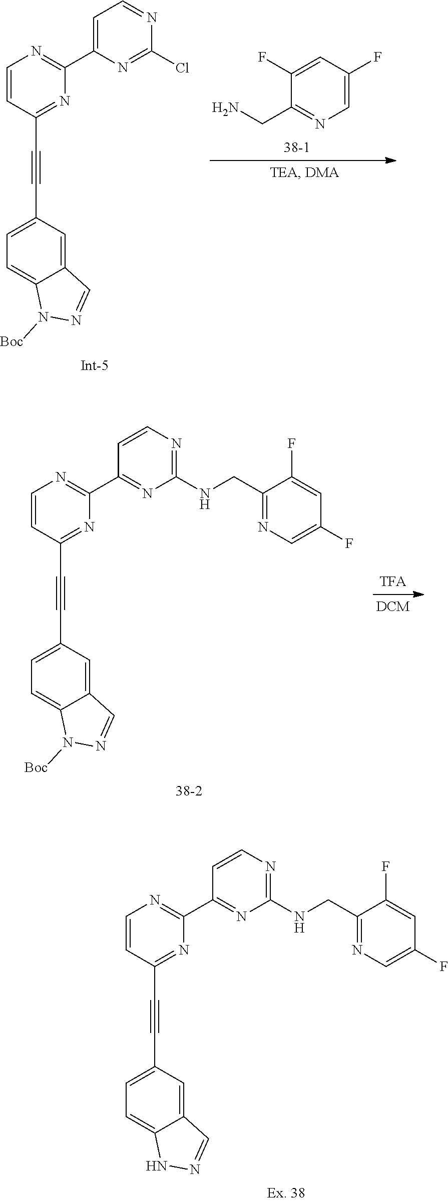 [SMILES: CC(C)(C)OC(=O)n1ncc2cc(C#Cc3ccnc(-c4ccnc(Cl)n4)n3)ccc21 ; NCc1ncc(F)cc1F ; CC(C)(C)OC(=O)n1ncc2cc(C#Cc3ccnc(-c4ccnc(NCc5ncc(F)cc5F)n4)n3)ccc21 ; Fc1cnc(CNc2nccc(-c3nccc(C#Cc4ccc5[nH]ncc5c4)n3)n2)c(F)c1]

Ex. 38 was prepared from (3,5-difluoropyridin-2-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (400 MHz, CD₃OD) δ 8.95 (s, 1H), 8.54 (s, 1H), 8.33 (s, 1H), 8.19-8.15 (m, 2H), 7.70-7.6 (m, 5H), 4.82 (s, 2H). MS (ESI⁺): m/z: 441.23 (M+H)⁺.

### Example 39. 4-((1H-Indazol-5-yl)ethynyl)-N-(2-(5-fluoropyridin-2-yl)ethyl)-[2,4′-bipyrimidin]-2′-amine (Ex. 39)

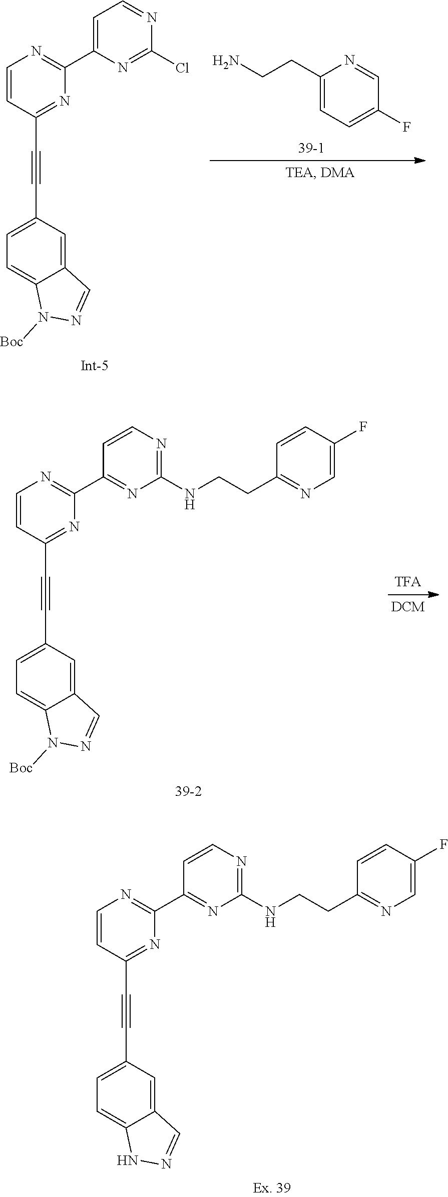 [SMILES: CC(C)(C)OC(=O)n1ncc2cc(C#Cc3ccnc(-c4ccnc(Cl)n4)n3)ccc21 ; NCCc1ccc(F)cn1 ; CC(C)(C)OC(=O)n1ncc2cc(C#Cc3ccnc(-c4ccnc(NCCc5ccc(F)cn5)n4)n3)ccc21 ; Fc1ccc(CCNc2nccc(-c3nccc(C#Cc4ccc5[nH]ncc5c4)n3)n2)nc1]

Ex. 39 was prepared from 2-(5-fluoropyridin-2-yl)ethan-1-amine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H NMR (400 MHz, CD₃OD) δ 8.93 (d, J=6.8 Hz, 1H), 8.53 (d, J=6.8 Hz, 1H), 8.38 (s, 1H), 8.14 (m, 2H), 7.68-7.51 (m, 4H), 7.45-7.44 (m, 2H), 3.91 (m, 2H), 3.16 (m, 2H). MS (ESI⁺): m/z: 437.32 (M+H)⁺.

### Example 40. 4-((1H-Indazol-5-yl)ethynyl)-N-(furan-2-ylmethyl)-[2,4′-bipyrimidin]-2′-amine (Ex. 40)

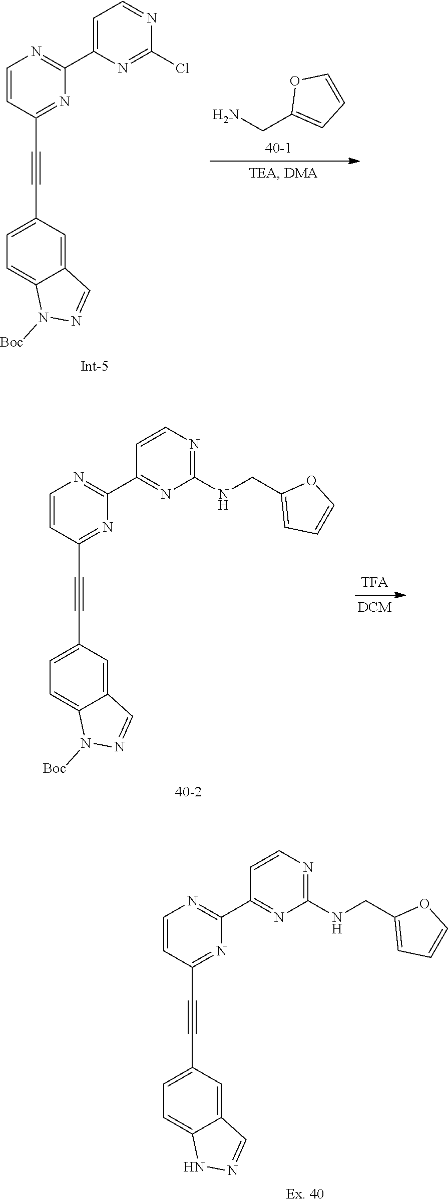 [SMILES: CC(C)(C)OC(=O)n1ncc2cc(C#Cc3ccnc(-c4ccnc(Cl)n4)n3)ccc21 ; NCc1ccco1 ; CC(C)(C)OC(=O)n1ncc2cc(C#Cc3ccnc(-c4ccnc(NCc5ccco5)n4)n3)ccc21 ; C(#Cc1ccnc(-c2ccnc(NCc3ccco3)n2)n1)c1ccc2[nH]ncc2c1]

Ex. 40 was prepared from furan-2-ylmethanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃/DMSO-d₆ (3:1): δ (ppm): 8.92 (d, J=5.1 Hz, 1H), 8.06 (s, 1H), 8.02 (s, 1H), 7.88-7.86 (m, 2H), 7.57 (d, J=5.1 Hz, 1H), 7.50-7.47 (m, 2H), 7.32-7.30 (m, 1H), 7.27-7.25 (m, 2H), 4.81 (s, 2H). MS (ESI⁺): m/z: 394.19 (M+H)⁺.

### Example 41. 2-((4-((1H-Indazol-5-yl)ethynyl)-[2,4′-bipyrimidin]-2′-yl)amino)-1-morpholinoethanone (Ex. 41)

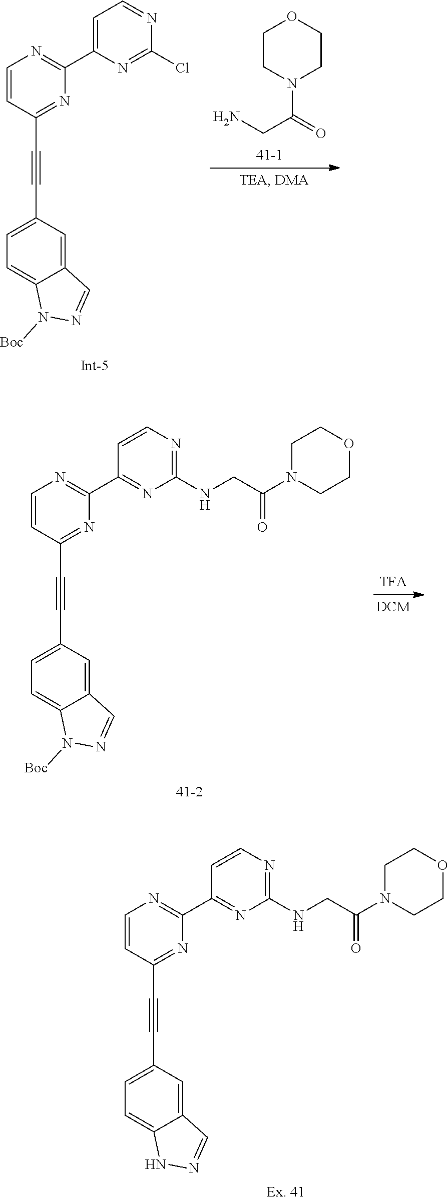 [SMILES: CC(C)(C)OC(=O)n1ncc2cc(C#Cc3ccnc(-c4ccnc(Cl)n4)n3)ccc21 ; NCC(=O)N1CCOCC1 ; CC(C)(C)OC(=O)n1ncc2cc(C#Cc3ccnc(-c4ccnc(NCC(=O)N5CCOCC5)n4)n3)ccc21 ; O=C(CNc1nccc(-c2nccc(C#Cc3ccc4[nH]ncc4c3)n2)n1)N1CCOCC1]

Ex. 41 was prepared from 2-amino-1-morpholinoethan-1-one and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): δ (ppm): 8.91 (d, J=5.1 Hz, 1H), 8.55 (d, J=5.0 Hz, 1H), 8.14 (d, J=0.9 Hz, 1H), 7.97 (m, 1H), 7.74 (d, J=5.1 Hz, 1H), 7.52 (dt, J=8.6, 0.9 Hz, 1H), 7.50 (d, J=5.1 Hz, 1H), 7.39 (dd, J=8.6, 1.4 Hz, 1H), 4.32 (d, J=4.5 Hz, 2H), 3.69 (m, 8H). MS (ESI⁺): m/z: 441.25 (M+H)⁺.

### Example 42. 4-((7-Fluoro-1H-indazol-5-yl)ethynyl)-N-((2-fluoropyridin-3-yl)methyl)-[2,4′-bipyrimidin]-2′-amine (Ex. 42)

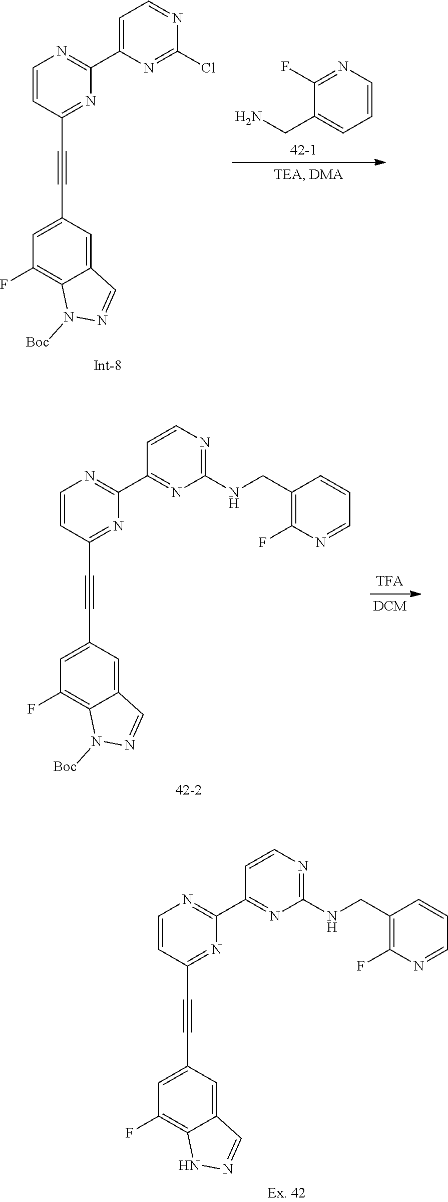 [SMILES: CC(C)(C)OC(=O)n1ncc2cc(C#Cc3ccnc(-c4ccnc(Cl)n4)n3)cc(F)c21 ; NCc1cccnc1F ; CC(C)(C)OC(=O)n1ncc2cc(C#Cc3ccnc(-c4ccnc(NCc5cccnc5F)n4)n3)cc(F)c21 ; Fc1ncccc1CNc1nccc(-c2nccc(C#Cc3cc(F)c4[nH]ncc4c3)n2)n1]

Ex. 42 was prepared from (2-fluoropyridin-3-yl)methanamine and tert-butyl 5-((2′-chloro-[2,4′-bipyrimidin]-4-yl)ethynyl)-7-fluoro-1H-indazole-1-carboxylate (Int-8) in a manner analogous to the procedures described for Step 3 and Step 4 of Example 1. ¹H-NMR (300 MHz, CDCl₃): δ (ppm): 8.87 (d, J=5.1 Hz, 1H), 8.50 (d, J=5.1 Hz, 1H), 8.33 (m, 1H), 8.11 (m, 1H), 7.87 (d, 1H), 7.64 (d, J=5.0 Hz, 1H), 7.56 (d, J=5.0 Hz, 1H), 7.39 (m, 1H), 7.35 (m, 1H), 7.27 (m, 1H), 4.79 (s, 2H). MS (ESI⁺): m/z: 441.26 (M+H)⁺.

### Example 43. 4-((3-Fluoro-1H-indazol-5-yl)ethynyl)-N-((2-fluoropyridin-3-yl)methyl)-[2,4′-bipyrimidin]-2′-amine (Ex. 43)

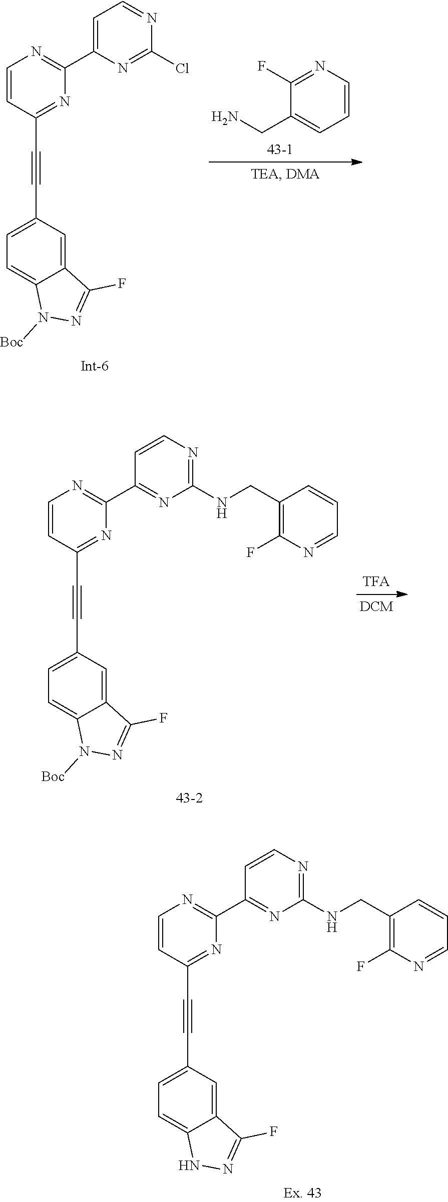 [SMILES: CC(C)(C)OC(=O)n1nc(F)c2cc(C#Cc3ccnc(-c4ccnc(Cl)n4)n3)ccc21 ; NCc1cccnc1F ; CC(C)(C)OC(=O)n1nc(F)c2cc(C#Cc3ccnc(-c4ccnc(NCc5cccnc5F)n4)n3)ccc21 ; Fc1ncccc1CNc1nccc(-c2nccc(C#Cc3ccc4[nH]nc(F)c4c3)n2)n1]

Ex. 43 was prepared (2-fluoropyridin-3-yl)methanamine and tert-butyl 5-((2′-chloro-[2,4′-bipyrimidin]-4-yl)ethynyl)-3-fluoro-1H-indazole-1-carboxylate (Int-6) in a manner analogous to the procedures described for Step 3 and Step 4 of Example 1 from. MS (ESI⁺): m/z: 441.16 (M+H)⁺.

### Example 44. 4-((1H-Indazol-5-yl)ethynyl)-N-(cyclopentylmethyl)-[2,4′-bipyrimidin]-2′-amine (Ex. 44)

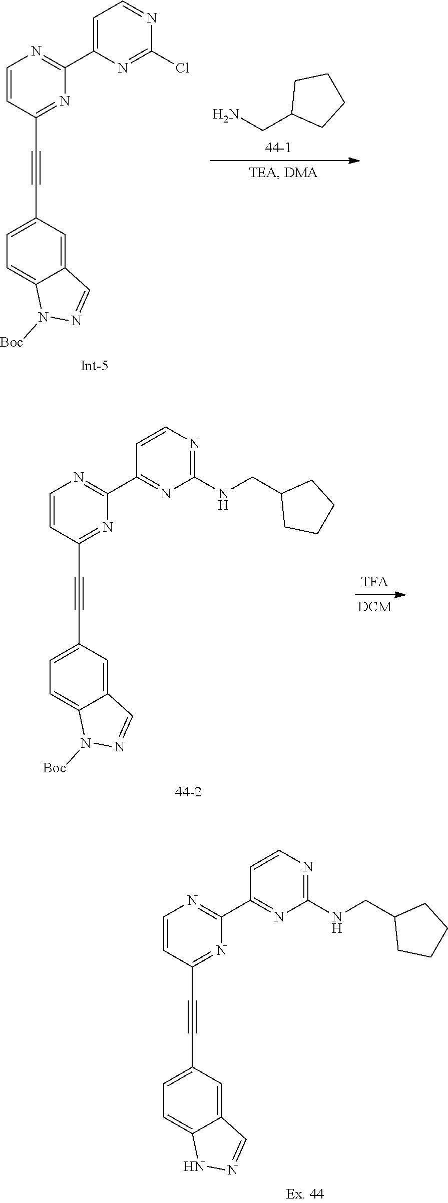 [SMILES: CC(C)(C)OC(=O)n1ncc2cc(C#Cc3ccnc(-c4ccnc(Cl)n4)n3)ccc21 ; NCC1CCCC1 ; CC(C)(C)OC(=O)n1ncc2cc(C#Cc3ccnc(-c4ccnc(NCC5CCCC5)n4)n3)ccc21 ; C(#Cc1ccnc(-c2ccnc(NCC3CCCC3)n2)n1)c1ccc2[nH]ncc2c1]

Ex. 44 was prepared from cyclopentylmethanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, DMSO-d₆): δ (ppm): 8.99 (d, J=5.1 Hz, 1H), 8.63 (d, J=5.1 Hz, 1H), 8.19-8.15 (m, 2H), 7.75-7.73 (m, 2H), 7.64 (m, 2H), 3.36 (m, 2H), 2.26-1.30 (m, 11H). MS (ESI⁺): m/z: 396.25 (M+H)⁺.

### Example 45. 4-((3-Fluoro-1H-indazol-5-yl)ethynyl)-N-((5-fluoropyridin-2-yl)methyl)-[2,4′-bipyrimidin]-2′-amine (Ex. 45)

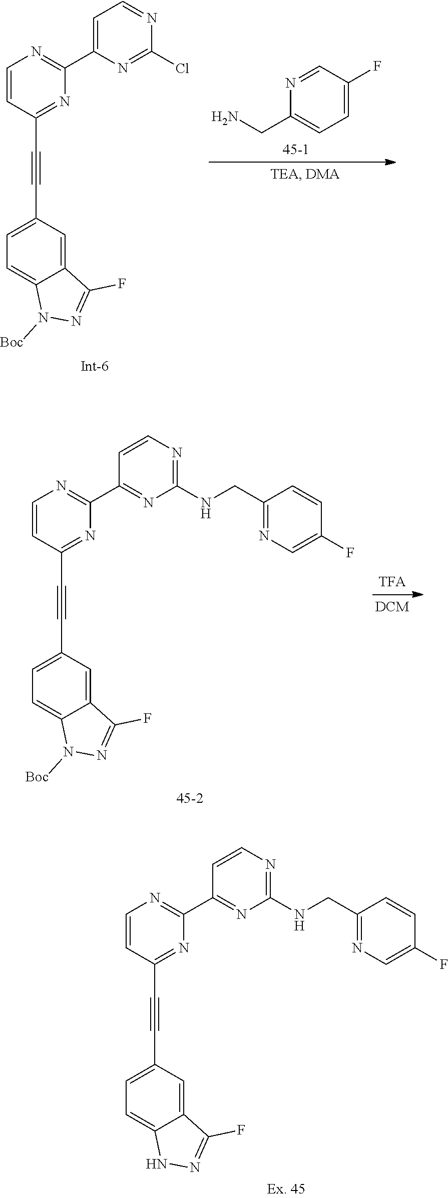 [SMILES: CC(C)(C)OC(=O)n1nc(F)c2cc(C#Cc3ccnc(-c4ccnc(Cl)n4)n3)ccc21 ; NCc1ccc(F)cn1 ; CC(C)(C)OC(=O)n1nc(F)c2cc(C#Cc3ccnc(-c4ccnc(NCc5ccc(F)cn5)n4)n3)ccc21 ; Fc1ccc(CNc2nccc(-c3nccc(C#Cc4ccc5[nH]nc(F)c5c4)n3)n2)nc1]

Ex. 45 was prepared from (5-fluoropyridin-2-yl)methanamine and tert-butyl 5-((2′-chloro-[2,4′-bipyrimidin]-4-yl)ethynyl)-3-fluoro-1H-indazole-1-carboxylate (Int-6) in a manner analogous to the procedures described for Step 3 and Step 4 of Example 1. ¹H-NMR (400 MHz, CD₃OD) δ 8.92 (d, J=6.8 Hz, 1H), 8.51 (d, J=6.8 Hz, 1H), 8.37 (s, 1H), 8.03 (s, 1H), 7.72 (d, J=6.8 Hz, 1H), 7.62-7.58 (m, 2H), 7.47-7.43 (m, 3H), 4.82 (s, 2H). MS (ESI⁺): m/z: 441.23 (M+H)⁺.

### Example 46. 2-((4-((3-Fluoro-1H-indazol-5-yl)ethynyl)-[2,4′-bipyrimidin]-2′-yl)amino)-N,N-dimethylacetamide (Ex. 46)

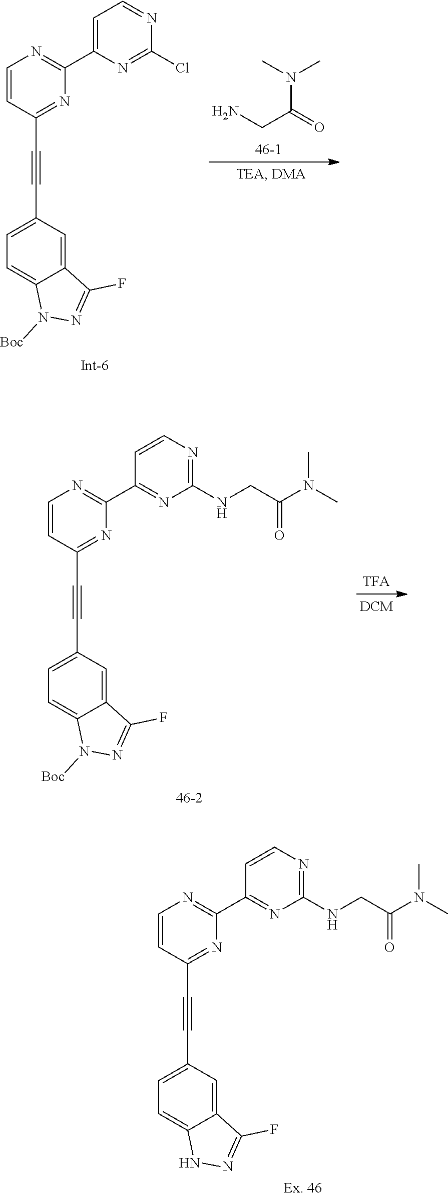

Ex. 46 was prepared from 2-amino-N,N-dimethylacetamide and tert-butyl 5-((2′-chloro-[2,4′-bipyrimidin]-4-yl)ethynyl)-3-fluoro-1H-indazole-1-carboxylate (Int-6) in a manner analogous to the procedures described for Step 3 and Step 4 of Example 1. ¹H-NMR (400 MHz, CDCl₃) δ 8.92 (d, J=6.8 Hz, 1H), 8.55 (d, J=6.8 Hz, 1H), 7.97 (s, 1H), 7.72 (d, J=6.8 Hz, 1H), 7.55-7.25 (m, 3H), 4.31 (d, 2H), 3.05 (s, 3H), 3.03 (s, 3H). MS (ESI⁺): m/z: 417.43 (M+H)⁺.

### Example 47. 4-((1H-Indazol-5-yl)ethynyl)-N-((6-fluoropyridin-3-yl)methyl)-[2,4′-bipyrimidin]-2′-amine (Ex. 47)

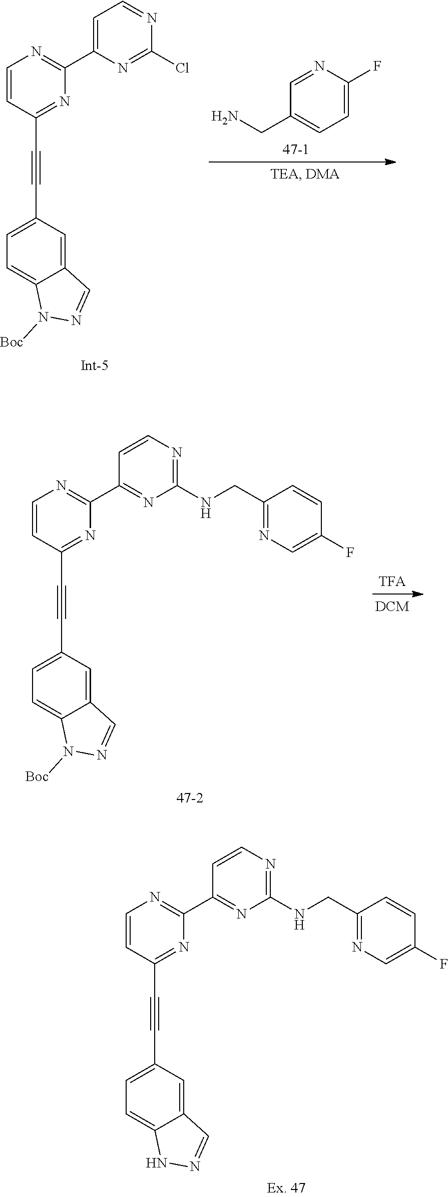 [SMILES: CC(C)(C)OC(=O)n1ncc2cc(C#Cc3ccnc(-c4ccnc(Cl)n4)n3)ccc21 ; NCc1ccc(F)nc1 ; CC(C)(C)OC(=O)n1ncc2cc(C#Cc3ccnc(-c4ccnc(NCc5ccc(F)cn5)n4)n3)ccc21 ; Fc1ccc(CNc2nccc(-c3nccc(C#Cc4ccc5[nH]ncc5c4)n3)n2)nc1]

Ex. 47 was prepared from (6-fluoropyridin-3-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (400 MHz, CDCl₃) δ 8.92 (d, J=6.8 Hz, 1H), 8.56 (d, J=6.8 Hz, 1H), 8.27 (s, 1H), 8.11 (s, 1H), 8.05 (s, 1H), 7.85 (m, 1H), 7.78 (d, J=6.8 Hz, 1H), 7.53-7.49 (m, 5H), 6.89 (m, 1H), 4.74 (d, 2H). MS (ESI⁺): m/z: 423.34 (M+H)⁺.

### Example 48. 4-((1H-Indazol-5-yl)ethynyl)-N-((5-(trifluoromethyl)pyridin-2-yl)methyl)-[2,4′-bipyrimidin]-2′-amine (Ex. 48)

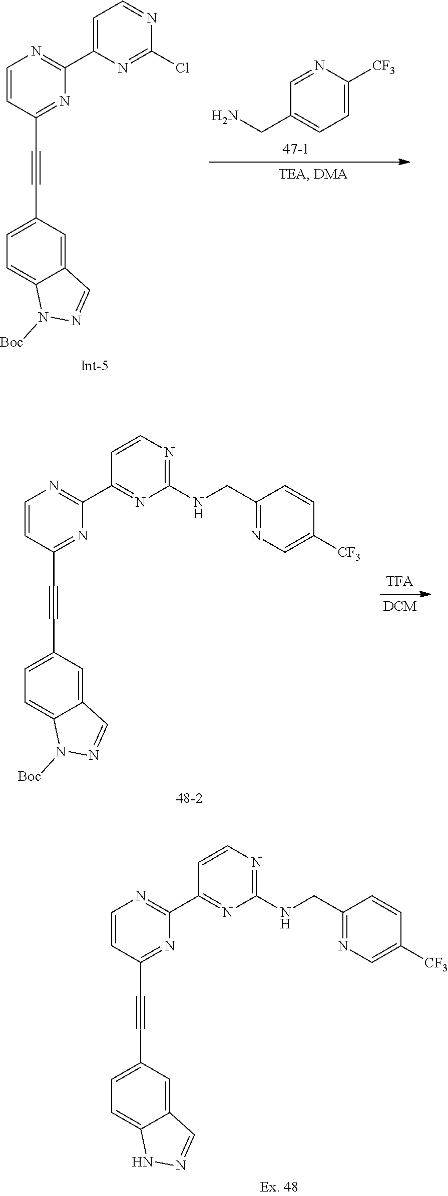 [SMILES: CC(C)(C)OC(=O)n1ncc2cc(C#Cc3ccnc(-c4ccnc(Cl)n4)n3)ccc21 ; NCc1ccc(C(F)(F)F)nc1 ; CC(C)(C)OC(=O)n1ncc2cc(C#Cc3ccnc(-c4ccnc(NCc5ccc(C(F)(F)F)cn5)n4)n3)ccc21 ; FC(F)(F)c1ccc(CNc2nccc(-c3nccc(C#Cc4ccc5[nH]ncc5c4)n3)n2)nc1]

Ex. 48 was prepared from (5-(trifluoromethyl)pyridin-2-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (400 MHz, CDCl₃) δ 8.96 (d, J=7.2 Hz, 1H), 8.84 (s, 1H), 8.60 (d, J=6.8 Hz, 1H), 8.16 (s, 1H), 8.14 (s, 1H), 8.09 (m, 1H), 7.77-7.62 (m, 5H), 4.96 (s, 2H). MS (ESI⁺): m/z: 473.23 (M+H)⁺.

### Example 49. 4-((7-Fluoro-1H-indazol-5-yl)ethynyl)-N-((tetrahydrofuran-2-yl)methyl)-[2,4′-bipyrimidin]-2′-amine (Ex. 49)

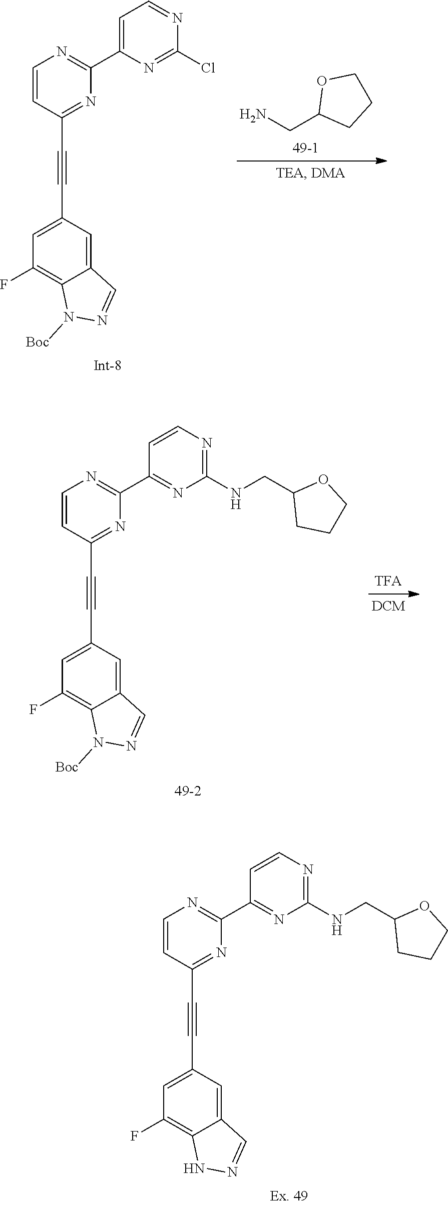 [SMILES: CC(C)(C)OC(=O)n1ncc2cc(C#Cc3ccnc(-c4ccnc(Cl)n4)n3)cc(F)c21 ; NCC1CCCO1 ; CC(C)(C)OC(=O)n1ncc2cc(C#Cc3ccnc(-c4ccnc(NCC5CCCO5)n4)n3)cc(F)c21 ; Fc1cc(C#Cc2ccnc(-c3ccnc(NCC4CCCO4)n3)n2)cc2cn[nH]c12]

Ex. 49 was prepared from (tetrahydrofuran-2-yl)methanamine and tert-butyl 5-((2′-chloro-[2,4′-bipyrimidin]-4-yl)ethynyl)-7-fluoro-1H-indazole-1-carboxylate (Int-8) in a manner analogous to the procedures described for Step 3 and Step 4 of Example 1. MS (ESI⁺): m/z: 416.23 (M+H)⁺.

### Example 50. 4-((3-Fluoro-1H-indazol-5-yl)ethynyl)-N-((tetrahydrofuran-2-yl)methyl)-[2,4′-bipyrimidin]-2′-amine (Ex. 50)

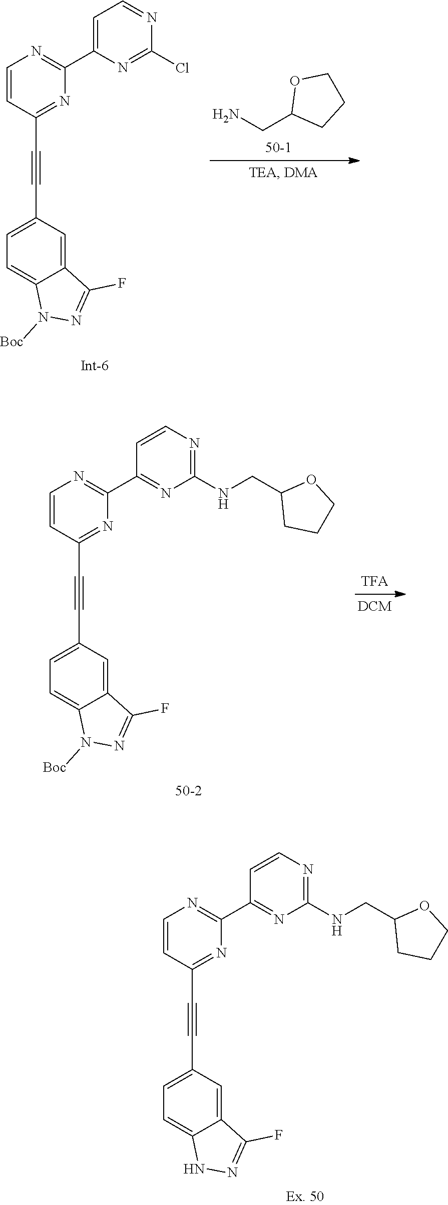 [SMILES: CC(C)(C)OC(=O)n1nc(F)c2cc(C#Cc3ccnc(-c4ccnc(Cl)n4)n3)ccc21 ; NCC1CCCO1 ; CC(C)(C)OC(=O)n1nc(F)c2cc(C#Cc3ccnc(-c4ccnc(NCC5CCCO5)n4)n3)ccc21 ; Fc1n[nH]c2ccc(C#Cc3ccnc(-c4ccnc(NCC5CCCO5)n4)n3)cc12]

Ex. 50 was prepared from (tetrahydrofuran-2-yl)methanamine and tert-butyl 5-((2′-chloro-[2,4′-bipyrimidin]-4-yl)ethynyl)-3-fluoro-1H-indazole-1-carboxylate (Int-6) in a manner analogous to the procedures described for Step 3 and Step 4 of Example 1. MS (ESI⁺): m/z: 416.23 (M+H)⁺.

### Example 51. ((2R,5S)-5-(((4-((1H-Indazol-5-yl)ethynyl)-[2,4′-bipyrimidin]-2′-yl)amino)methyl)tetrahydrofuran-2-yl)methanol (Ex. 51)

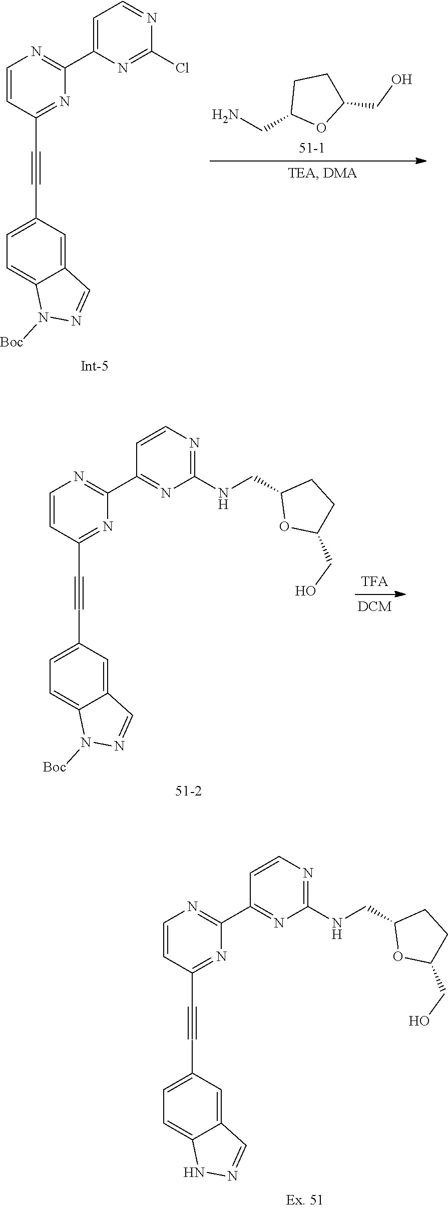 [SMILES: CC(C)(C)OC(=O)n1ncc2cc(C#Cc3ccnc(-c4ccnc(Cl)n4)n3)ccc21 ; NC[C@@H]1CC[C@H](CO)O1 ; CC(C)(C)OC(=O)n1ncc2cc(C#Cc3ccnc(-c4ccnc(NC[C@@H]5CC[C@H](CO)O5)n4)n3)ccc21 ; OC[C@H]1CC[C@@H](CNc2nccc(-c3nccc(C#Cc4ccc5[nH]ncc5c4)n3)n2)O1]

Ex. 51 was prepared from ((2R,5S)-5-(aminomethyl)tetrahydrofuran-2-yl)methanol and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): δ (ppm): 8.88 (d, J=5.1 Hz, 1H), 8.52 (d, J=5.1 Hz, 1H), 8.13 (d, J=0.7 Hz, 1H), 7.84 (br s, 1H), 7.69 (d, J=5.1 Hz, 1H), 7.50-7.46 (m, 2H), 7.13 (d, J=8.9 Hz, 1H), 4.22 (m, 1H), 4.10 (m, 1H), 3.79 (dd, J=12.1, 2.7 Hz, 1H), 3.8 (m, 1H), 3.53 (m, 1H), 3.51 (dd, J=12.0, 4.5 Hz, 1H), 2.10-1.87 (m, 3H), 1.78 (m, 1H). MS (ESI⁺): m/z: 428.27 (M+H)⁺.

### Example 52. 2-((4-((1H-Indazol-5-yl)ethynyl)-[2,4′-bipyrimidin]-2′-yl)amino)-1-(pyrrolidin-1-yl)ethanone (Ex. 52)

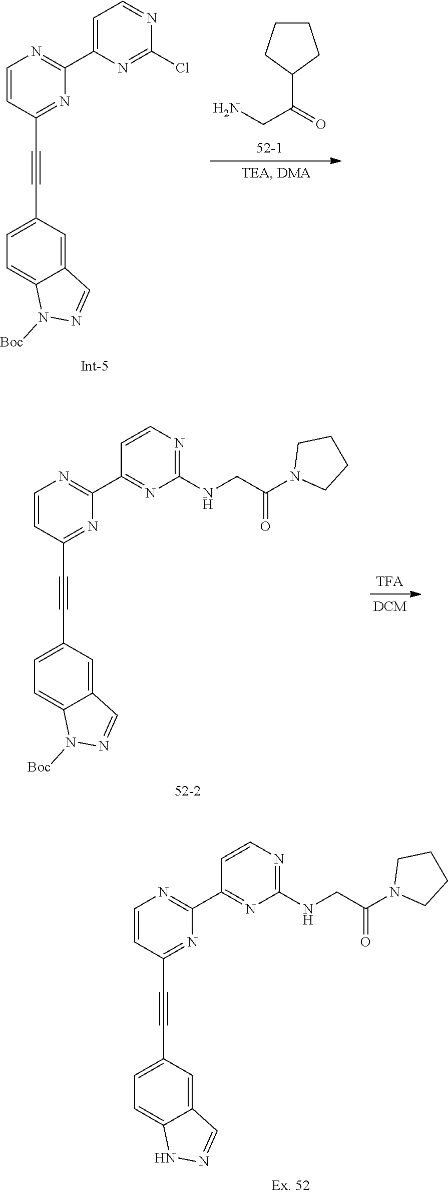 [SMILES: CC(C)(C)OC(=O)n1ncc2cc(C#Cc3ccnc(-c4ccnc(Cl)n4)n3)ccc21 ; NCC(=O)C1CCCC1 ; CC(C)(C)OC(=O)n1ncc2cc(C#Cc3ccnc(-c4ccnc(NCC(=O)N5CCCC5)n4)n3)ccc21 ; O=C(CNc1nccc(-c2nccc(C#Cc3ccc4[nH]ncc4c3)n2)n1)N1CCCC1]

Ex. 52 was prepared from 2-amino-1-(pyrrolidin-1-yl)ethan-1-one and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): δ (ppm): 8.90 (d, J=5.0 Hz, 1H), 8.55 (d, J=5.3 Hz, 1H), 8.13 (d, J=0.6 Hz, 1H), 8.02 (t, J=1.2 Hz, 1H), 7.71 (d, J=5.0 Hz, 1H), 7.54 (dt, J=8.8. 0.9 Hz, 1H), 7.50 (d, J=5.3 Hz, 1H), 7.46 (dd, J=8.8, 1.5 Hz, 1H), 4.24 (d, J=4.4 Hz, 2H), 3.56 (t, J=6.7 Hz, 2H), 3.45 (m, 2H), 1.99 (m, 2H), 1.88 (m, 2H). MS (ESI⁺): m/z: 425.24 (M+H)⁺.

### Example 53. 4-((1H-Indazol-5-yl)ethynyl)-N-((2-methyltetrahydrofuran-2-yl)methyl)-[2,4′-bipyrimidin]-2′-amine (Ex. 53)

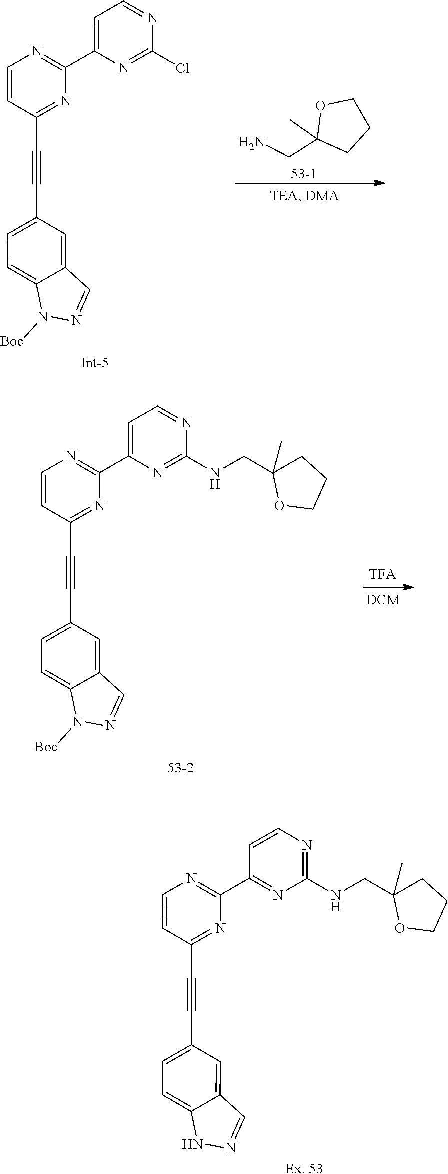

Ex. 53 was prepared from (2-methyltetrahydrofuran-2-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): δ (ppm): 8.87 (d, J=5.1 Hz, 1H), 8.47 (d, J=5.0 Hz, 1H), 8.08 (s, 1H), 8.03 (t, J=1.1 Hz, 1H), 7.62 (d, J=5.1 Hz, 1H), 7.50 (m, 2H), 7.48 (d, J=5.1 Hz, 1H), 3.85 (t, J=6.3 Hz, 2H), 3.65 (d, J=13.4 Hz, 2H), 3.49 (d, J=13.4 Hz, 1H), 1.96-1.84 (m, 3H), 1.70-1.60 (m, 1H), 1.25 (s, 3H). MS (ESI⁺): m/z: 412.26 (M+H)⁺.

### Example 54. N1-(4-((1H-Indazol-5-yl)ethynyl)-[2,4′-bipyrimidin]-2′-yl)-N2,N2-dimethylethane-1,2-diamine (Ex. 54)

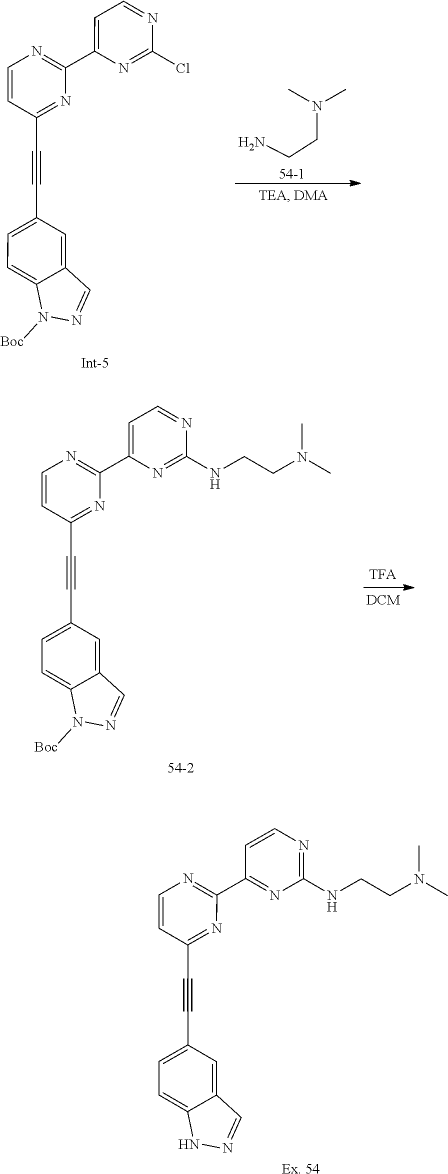

Ex. 54 was prepared from N1,N1-dimethylethane-1,2-diamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): δ (ppm): 11.48 (br, 1H), 8.90 (d, J=2.1 Hz, 1H), 8.53 (d, J=5.1 Hz, 1H), 8.14 (s, 1H), 8.01 (s, 1H), 7.66 (d, J=5.0 Hz, 1H), 7.53 (d, J=8.7 Hz, 1H), 7.49 (d, J=5.1 Hz, 1H), 7.43 (d, J=7.4 Hz, 1H), 6.44 (br, 1H), 3.58 (q, J=5.7 Hz, 2.0H), 2.58 (t, J=6.3 Hz), 2.28 (s, 6H). MS (ESI⁺): m/z: 385.23 (M+H)⁺.

### Example 55. 2-((4-((1H-Indazol-5-yl)ethynyl)-[2,4′-bipyrimidin]-2′-yl)amino)-N-methylacetamide (Ex. 55)

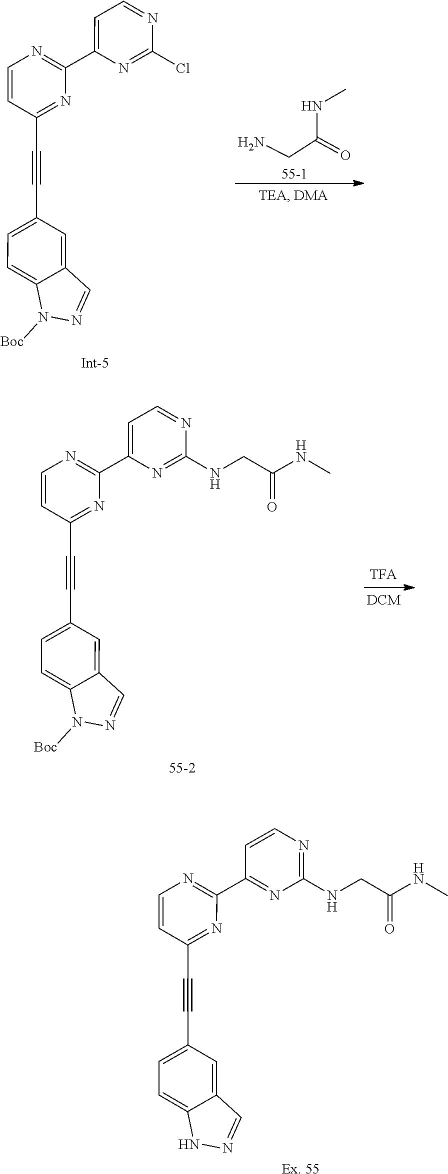

Ex. 55 was prepared from 2-amino-N-methylacetamide and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD): δ (ppm): 8.89 (d, J=5.1 Hz, 1H), 8.51 (d, J=5.1 Hz, 1H), 8.00 (d, J=10.8 Hz, 2H), 7.72 (d, J=4.9 Hz), 7.60 (m, 3H), 4.13 (s, 2H), 2.77 (s, 3H). MS (ESI⁺): m/z: 385.23 (M+H)⁺.

### Example 56. 4-((1H-Indazol-5-yl)ethynyl)-N-(2-(methylsulfonyl)ethyl)-[2,4′-bipyrimidin]-2′-amine (Ex. 56)

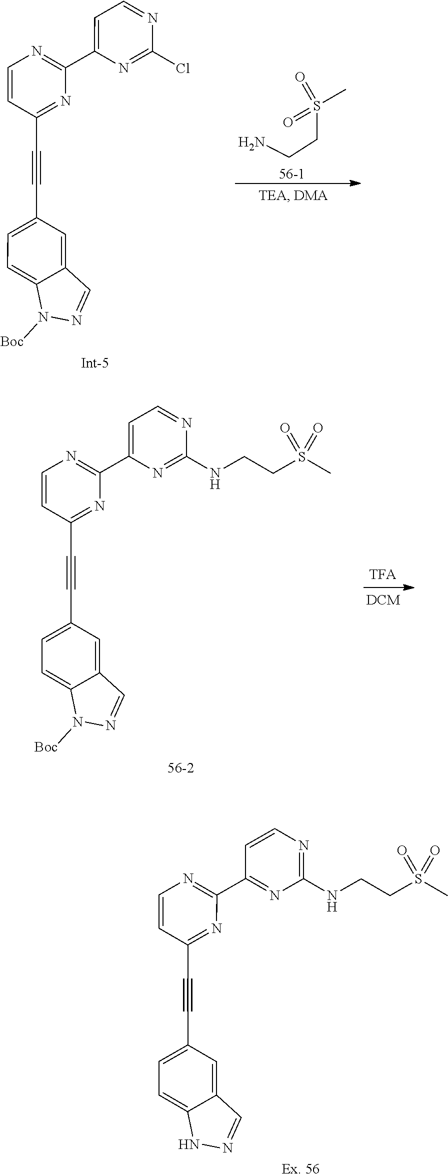

Ex. 56 was prepared from 2-(methylsulfonyl)ethan-1-amine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): δ (ppm): 8.93 (d, J=4.5 Hz, 1H), 8.61 (br, 1H), 8.17 (s, 1H), 8.10 (s, 1H), 7.84 (d, J=4.8 Hz, 1H), 7.54 (m, 3H), 4.10 (m, 2H), 3.48 (t, J=6.6 Hz, 2H), 3.06 (s, 3H). MS (ESI⁺): m/z: 420.20 (M+H)⁺.

### Example 57. N-((1H-Benzo[d]imidazol-2-yl)methyl)-4-((1H-indazol-5-yl)ethynyl)-[2,4′-bipyrimidin]-2′-amine (Ex. 57)

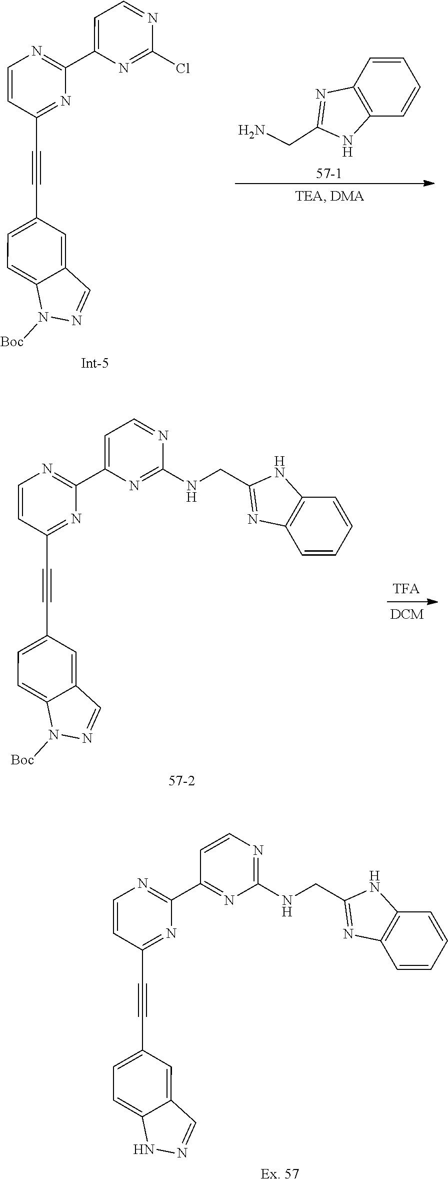

Ex. 57 was prepared from (1H-benzo[d]imidazol-2-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD): δ (ppm): 8.99 (d, J=5.1 Hz, 1H), 8.52 (d, J=5.1 Hz, 1H), 8.00 (s, 1H), 7.96 (s, 1H), 7.72 (m, 3H), 7.64 (m, 2H), 7.47 (s, 2H), 7.16 (q, J=3.0 Hz, 2H), 4.84 (s, 2H). MS (ESI⁺): m/z: 444.24 (M+H)⁺.

### Example 58. (S)-4-((1H-Indazol-5-yl)ethynyl)-N-((tetrahydrofuran-3-yl)methyl)-[2,4′-bipyrimidin]-2′-amine (Ex. 58)

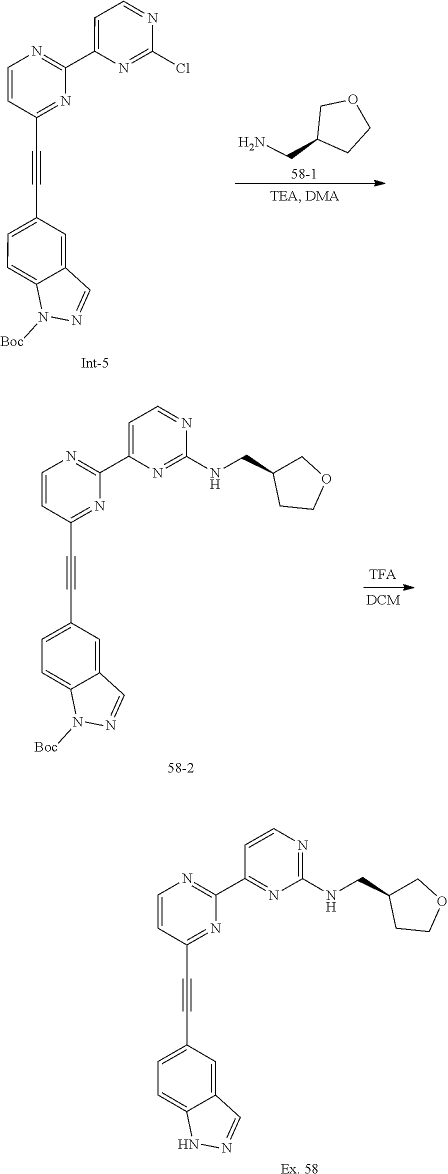

Ex. 58 was prepared from (S)-(tetrahydrofuran-3-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. MS (ESI⁺): m/z: 398.3 (M+H)⁺.

### Example 59. (R)-4-((1H-Indazol-5-yl)ethynyl)-N-(tetrahydrofuran-3-yl)-[2,4′-bipyrimidin]-2′-amine (Ex. 59)

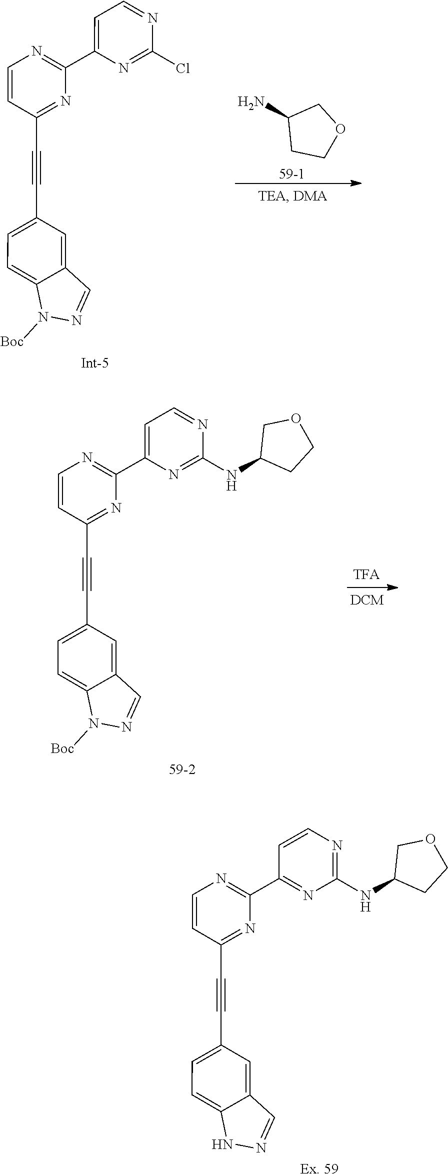

Ex. 59 was prepared from (R)-tetrahydrofuran-3-amine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD-CDCl₃): δ=8.89 (d, J=5.1 Hz, 1H), 8.47 (d, J=4.8 Hz, 1H), 8.11 (s, 1H), 8.08 (s, 1H), 7.65-7.58 (m, 4H), 3.92-3.85 (m, 1H), 3.80-3.72 (m, 1H), 3.65-3.60 (m, 1H), 3.50 (s, 1H), 2.69-2.60 (m, 1H), 2.15-2.09 (m, 1H), 1.79-1.70 (m, 1H). MS (ESI⁺): m/z: 384.23 (M+H)⁺.

### Example 60. 4-((3-Fluoro-1H-indazol-5-yl)ethynyl)-N-(2,3,5-trifluorobenzyl)-[2,4′-bipyrimidin]-2′-amine (Ex. 60)

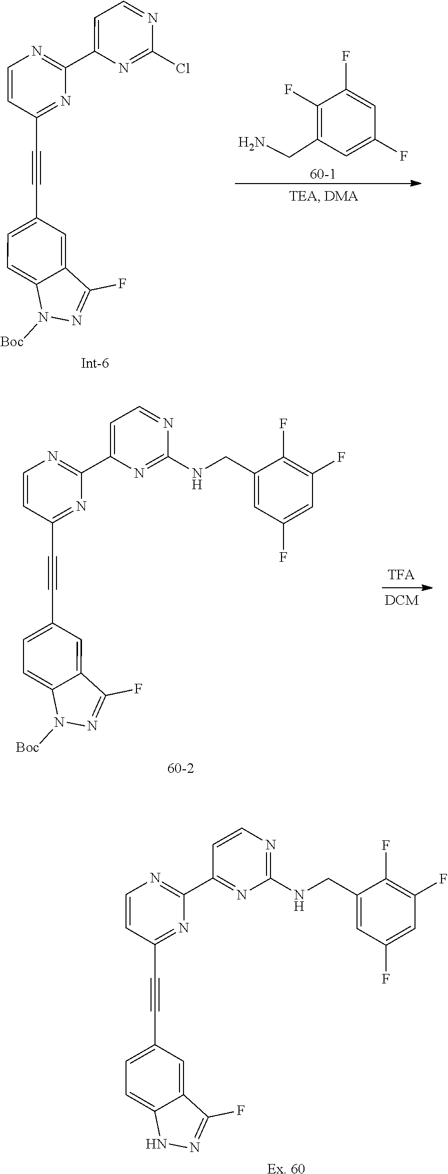

Ex. 60 was prepared in a manner analogous to the procedures described for Step 3 and Step 4 of Example 1 from (2,3,5-trifluorophenyl)methanamine and tert-butyl 5-((2′-chloro-[2,4′-bipyrimidin]-4-yl)ethynyl)-3-fluoro-1H-indazole-1-carboxylate (Int-6). ¹H-NMR (300 MHz, CD₃OD) δ 8.91 (d, J=6.8 Hz, 1H), 8.53 (d, J=6.8 Hz, 1H), 8.09 (s, 1H), 7.72 (d, J=6.4 Hz, 1H), 7.63-7.61 (m, 3H), 7.25 (m, 1H), 6.95 (m, 1H), 4.76 (s, 2H). MS (ESI⁺): m/z: 476.17 (M+H)⁺.

### Example 61. 4-((3-Fluoro-1H-indazol-5-yl)ethynyl)-N-((2-fluoropyridin-4-yl)methyl)-[2,4′-bipyrimidin]-2′-amine (Ex. 61)

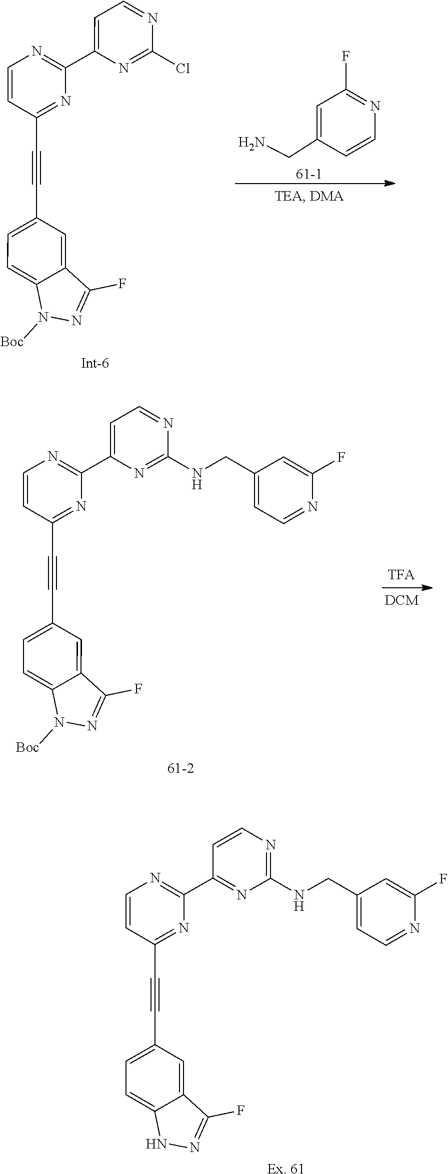

Ex. 61 was prepared in a manner analogous to the procedures described for Step 3 and Step 4 of Example 1 from (2-fluoropyridin-4-yl)methanamine and tert-butyl 5-((2′-chloro-[2,4′-bipyrimidin]-4-yl)ethynyl)-3-fluoro-1H-indazole-1-carboxylate (Int-6). ¹H-NMR (300 MHz, CD₃OD) δ 8.92 (d, J=6.8 Hz, 1H), 8.56 (d, J=6.8 Hz, 1H), 8.27 (s, 1H), 8.16 (s, 1H), 8.10 (m, 1H), 7.72-7.76 (m, 2H), 7.34 (m, 2H), 6.89 (m, 1H), 4.74 (s, 2H). MS (ESI⁺): m/z: 441.16 (M+H)⁺.

### Example 62. (S)-4-((1H-Indazol-5-yl)ethynyl)-N-(tetrahydrofuran-3-yl)-[2,4′-bipyrimidin]-2′-amine (Ex. 62)

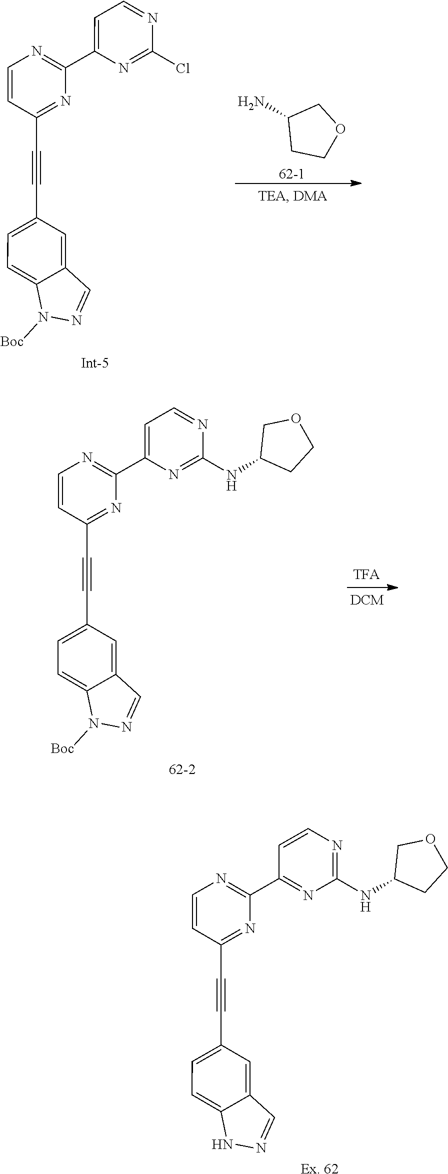

Ex. 62 was prepared in a manner analogous to the procedures described for Step 3 and Step 4 of Example 1 from (S)-tetrahydrofuran-3-amine and tert-butyl 5-((2′-chloro-[2,4′-bipyrimidin]-4-yl)ethynyl)-1H-indazole-1-carboxylate (Int-5). ¹H-NMR (300 MHz, CDCl₃): δ (ppm): 8.89 (d, J=5.1 Hz, 1H), 8.50 (d, J=5.0 Hz, 1H), 8.12 (t, J=1.0 Hz, 1H), 8.08 (d, J=0.7 Hz, 1H), 7.68 (d, J=5.1 Hz, 1H), 7.63-7.53 (m, 3H), 4.06-3.95 (m, 2H), 3.93-3.84 (m, 1H), 3.75 (dd, J=9.2, 3.4 Hz, 2H), 2.41-2.27 (m, 1H), 2.02-1.90 (m, 1H). MS (ESI⁺): m/z: 384.25 (M+H)⁺.

### Example 63. (1r,4r)-4-((4-((1H-Indazol-5-yl)ethynyl)-[2,4′-bipyrimidin]-2′-yl)amino)cyclohexanol (Ex. 63)

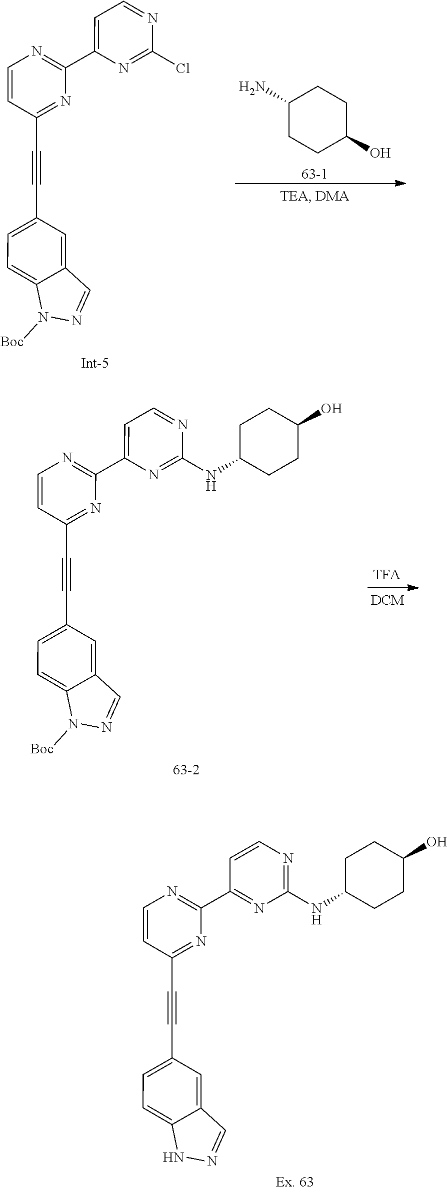

Ex. 63 was prepare in a manner analogous to the procedures described for Step 3 and Step 4 of Example 1 from (1r,4r)-4-aminocyclohexan-1-ol and tert-butyl 5-((2′-chloro-[2,4′-bipyrimidin]-4-yl)ethynyl)-1H-indazole-1-carboxylate (Int-5). ¹H-NMR (300 MHz, CDCl₃): δ (ppm): 8.84 (d, J=5.0 Hz, 1H), 8.45 (d, J=5.0 Hz, 1H), 8.07 (m, 1H), 8.05 (m, 1H), 7.59 (d, J=5.0 Hz, 1H), 7.56 (dd, J=8.8, 1.3 Hz, 1H), 7.50 (dt, J=8.8, 0.9 Hz, 1H), 7.48 (d, J=5.1 Hz, 1H), 3.82 (m, 1H), 3.60 (m, 1H), 2.11 (m, 2H), 1.97 (m, 2H), 1.50-1.36 (m, 2H), 1.35-1.18 (m, 2H). MS (ESI⁺): m/z: 412.26 (M+H)⁺.

### Example 64. 4-((1H-Indazol-5-yl)ethynyl)-N-((4-chloro-2-fluoropyridin-3-yl)methyl)-[2,4′-bipyrimidin]-2′-amine (Ex. 64)

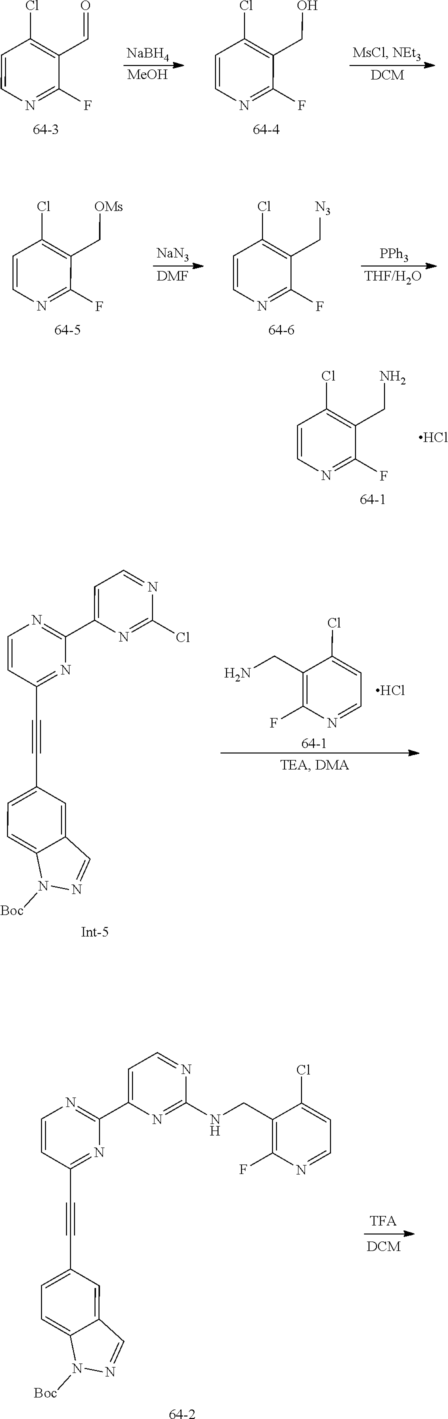

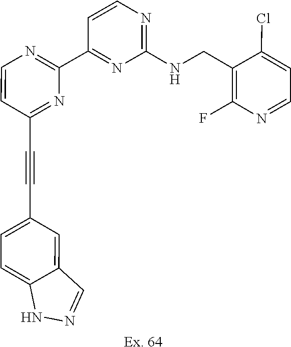 [SMILES: Fc1nccc(Cl)c1CNc1nccc(-c2nccc(C#Cc3ccc4[nH]ncc4c3)n2)n1 ; "Ex. 64"]

Step 1: (4-Chloro-2-fluoropyridin-3-yl)methanol (64-4): To a solution of 4-chloro-2-fluoronicotinaldehyde (64-3, 600 mg, 3.76 mmol) in 10 mL of MeOH cooled to 0° C., was added NaBH₄ (213 mg, 5.64 mmol). After an hour, TLC and LCMS indicated the reaction was complete. The reaction was quenched with acetone and evaporated to dryness. The residue was re-dissolved in ethyl acetate and water, and the organic layer was extracted with ethyl acetate twice. The combined organic phases were dried over Na₂SO₄, filtered, and the filtrate evaporated to dryness under reduced pressure to give the crude product 64-4, which was used directly without purification. MS (ESI⁺): m/z: 161.99 (M+H)⁺.

Step 2: (4-Chloro-2-fluoropyridin-3-yl)methyl methanesulfonate (64-5): A solution of (4-chloro-2-fluoropyridin-3-yl)methanol (64-4 from Step 1) and triethylamine (1.0 mL, 7.52 mmol) in 10 mL of DCM was cooled to −30° C., then methanesulfonyl chloride (0.45 mL, 5.64 mmol) was added. After stirring at rt for 1 h, TLC and LCMS indicated the reaction was complete. The reaction was quenched with water, and the organic layer was extracted with DCM twice. The organic layers were combined, dried over Na₂SO₄, filtered, and the filtrate evaporated to dryness under reduced pressure to afford the crude product 64-5, which was used directly in the next step without purification. MS (ESI⁺): m/z: 240.00 (M+H)⁺.

Step 3: 3-(Azidomethyl)-4-chloro-2-fluoropyridine (64-6): To a solution of (4-chloro-2-fluoropyridin-3-yl)methyl methanesulfonate (64-5 from Step 2) in 10 mL of DMF was added NaN₃ (366.6 mg, 5.64 mmol). The resulting mixture was stirred overnight at room temperature. TLC and LCMS indicated the reaction was complete. The reaction was quenched with water and extracted with DCM twice. The organic layers were combined, dried over Na₂SO₄, filtered, and the filtrate evaporated to dryness under reduced pressure to give the crude product 64-6, which was used directly in the next step without purification. MS (ESI⁺): m/z: 187.06 (M+H)⁺.

Step 4: (4-Chloro-2-fluoropyridin-3-yl)methanamine HCl salt (64-1): To a solution of 3-(azidomethyl)-4-chloro-2-fluoropyridine (64-6 from Step 3) in 6 mL of THE and 3 mL of water was added PPh₃ (1.48 g, 5.64 mmol). The resulting mixture was stirred at 45° C. overnight. TLC and LCMS indicated the reaction was complete. The solvent was removed under reduced pressure and the product was extracted twice into ether. After cooled to 0° C., HCl solution (4 M in dioxane) was added dropwise to convert the product into HCl salt to afford (4-chloro-2-fluoropyridin-3-yl)methanamine HCl salt (64-1, 160.7 mg, yield: 21.7% in 4 steps) as a white solid. MS (ESI⁺): m/z: 161.05 (M+H)⁺.

Step 5: tert-Butyl 5-((2′-(((4-chloro-2-fluoropyridin-3-yl)methyl)amino)-[2,4′-bipyrimidin]-4-yl)ethynyl)-1H-indazole-1-carboxylate (64-2): To a stirred mixture of tert-butyl 5-((2′-chloro-[2,4′-bipyrimidin]-4-yl)ethynyl)-1H-indazole-1-carboxylate (Int-5, 50 mg, 0.116 mmol) and (4-chloro-2-fluoropyridin-3-yl)methanamine (64-1, 45.7 mg, 0.232 mmol) in anhydrous dimethylacetamide (4 mL) was added triethylamine (0.1 mL, 0.717 mmol) dropwise at room temperature. The resulting mixture was stirred at 70° C. for 20 h. After cooling to rt, the reaction mixture was diluted with H₂O (5 mL) and with extracted with DCM (3×5 mL). The organic layers were combined, dried over Na₂SO₄, filtered, and the filtrate concentrated in vacuo. The crude product was purified by silica gel flash chromatography (ISCO) to afford the desired product tert-butyl 5-((2′-(((4-chloro-2-fluoropyridin-3-yl)methyl)amino)-[2,4′-bipyrimidin]-4-yl)ethynyl)-1H-indazole-1-carboxylate (64-2, 12 mg, yield: 18.6%) as a light yellow oil. MS (ESI⁺): m/z: 557.20 (M+H)⁺.

Step 6: 4-((1H-Indazol-5-yl)ethynyl)-N-((4-chloro-2-fluoropyridin-3-yl)methyl)-[2,4′-bipyrimidin]-2′-amine (Ex. 64): To a stirred solution of tert-butyl 5-((2′-((2-chloro-6-fluorobenzyl)amino)-[2,4′-bipyrimidin]-4-yl)ethynyl)-1H-indazole-1-carboxylate (64-2, 12 mg, 0.02 mmol) in DCM (1 mL) was added trifluoroacetic acid (0.5 mL) dropwise at room temperature. The resulting mixture was stirred at room temperature for 1 h. LC-MS showed the reaction was complete. The reaction mixture was concentrated in vacuo, neutralized with sat. NaHCO₃ solution, and extracted with ethyl acetate. The organic layers were combined, dried over sodium sulfate, filtered, and the filtrate concentrated to dryness. The crude product was purified by silica gel flash chromatography (ISCO) to afford 4-((1H-indazol-5-yl)ethynyl)-N-(2-chloro-6-fluorobenzyl)-[2,4′-bipyrimidin]-2′-amine (Ex. 64, 1.5 mg, yield: 15%) as a light yellow solid. ¹H-NMR (300 MHz, CDCl₃): δ (ppm): 8.84 (d, J=5.1 Hz, 1H), 8.52 (d, J=5.1 Hz, 1H), 8.07 (br s, 1H), 8.04 (d, J=0.6 Hz, 1H), 8.00 (d, J=5.4 Hz, 1H), 7.68 (d, J=5.1 Hz, 1H), 7.56 (dd, J=8.7, 1.2 Hz, 1H), 7.49 (d, J=8.7 Hz, 1H), 7.48 (d, J=5.1 Hz, 1H), 7.22 (d, J=5.4 Hz, 1H), 4.85 (s, 2H). MS (ESI⁺): m/z: 457.12 (M+H)⁺.

### Example 65. (1s,4s)-4-((4-((1H-Indazol-5-yl)ethynyl)-[2,4′-bipyrimidin]-2′-yl)amino)cyclohexanol (Ex. 65)

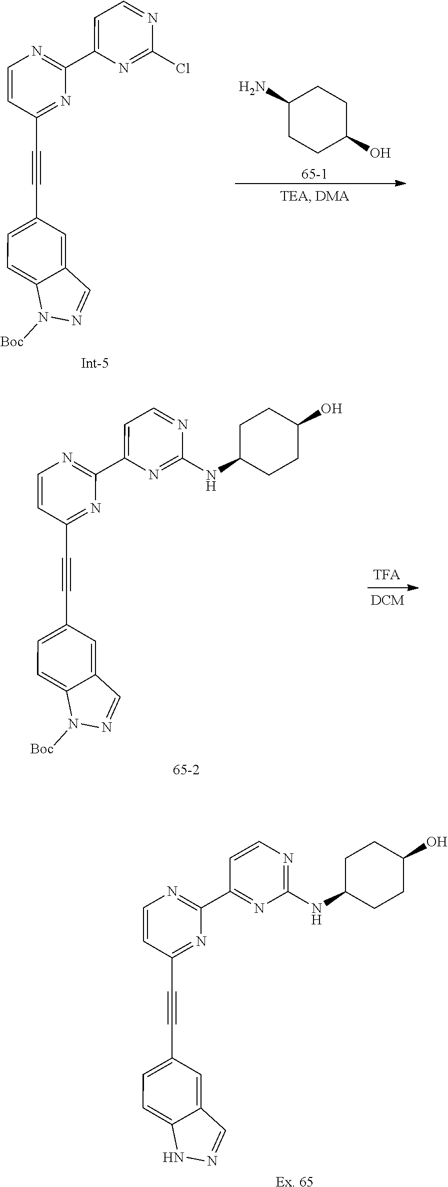

Ex. 65 was prepared from (1s,4s)-4-aminocyclohexan-1-ol and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD-CDCl₃): δ=8.88 (d, J=5.1 Hz, 1H), 8.45 (d, J=5.1 Hz, 1H), 8.11 (s, 1H), 8.08 (s, 1H), 7.61-7.57 (m, 4H), 3.97-3.94 (m, 1H), 3.84-3.80 (m, 1H), 1.82-1.71 (m, 8H). MS (ESI⁺): m/z: 412.26 (M+H)⁺.

### Example 66. 4-((1H-Indazol-5-yl)ethynyl)-N-(pyridin-2-ylmethyl-d₂)-[2,4′-bipyrimidin]-2′-amine (Ex. 66)

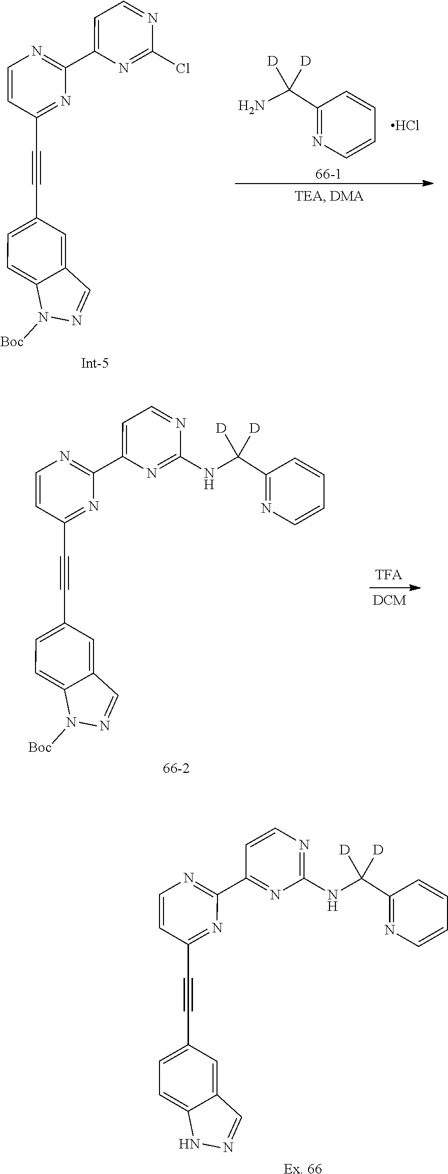

Ex. 66 was prepared from pyridin-2-ylmethan-d₂-amine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): δ (ppm): 8.91 (d, J=4.8 Hz, 1H), 8.55 (d, J=4.8 Hz, 2H), 8.11 (s, 1H), 8.01 (s, 1H), 7.72 (d, J=4.8 Hz, 1H), 7.63 (td, J₁=7.8 Hz, J₂=1.8 Hz, 1H), 7.51 (m, 2H), 7.38 (d, J=7.8 Hz, 2H), 7.18 (qd, J₁=5.1 Hz, J₂=1.2 Hz, 1H), 6.98 (br, 1H). MS (ESI⁺): m/z: 407.17 (M+H)⁺.

### Example 67. 4-((1H-Indazol-5-yl)ethynyl)-N-(1-(tetrahydrofuran-2-yl)ethyl)-[2,4′-bipyrimidin]-2′-amine (Ex. 67)

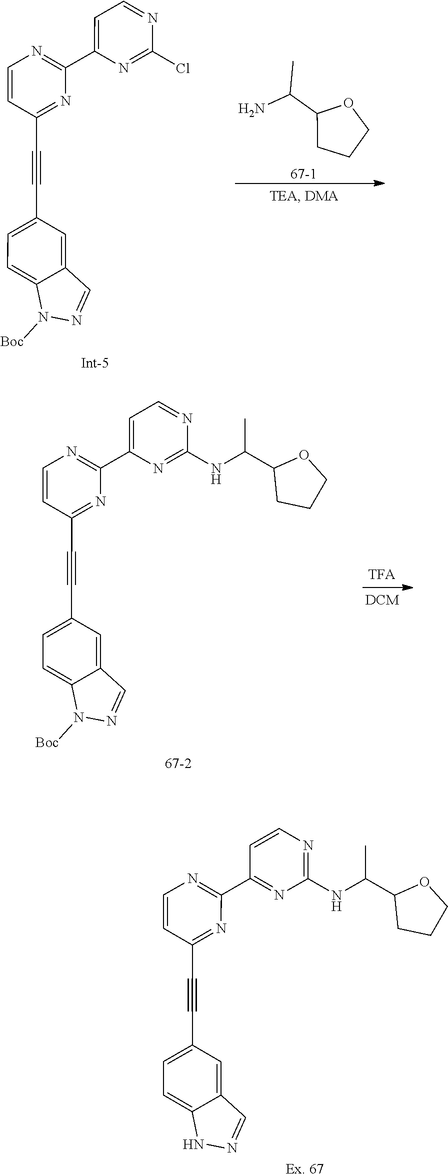

Ex. 67 was prepared from 1-(tetrahydrofuran-2-yl)ethan-1-amine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): δ (ppm): 8.92 (d, 5.1 Hz, 1H), 8.51 (s, 1H), 8.15 (s, 1H), 8.10 (s, 1H), 7.68 (m, 1H), 7.61 (d, J=8.7 Hz, 1H), 7.54 (s, 1H), 7.52 (m, 2H), 4.35 (br, 1H), 3.93 (m, 2H), 3.74 (m, 1H), 1.88 (m, 8H). MS (ESI⁺): m/z: 412.26 (M+H)⁺.

### Example 68. 4-((1H-Indazol-5-yl)ethynyl)-N-((5,5-dimethyltetrahydrofuran-2-yl)methyl)-[2,4′-bipyrimidin]-2′-amine (Ex. 68)

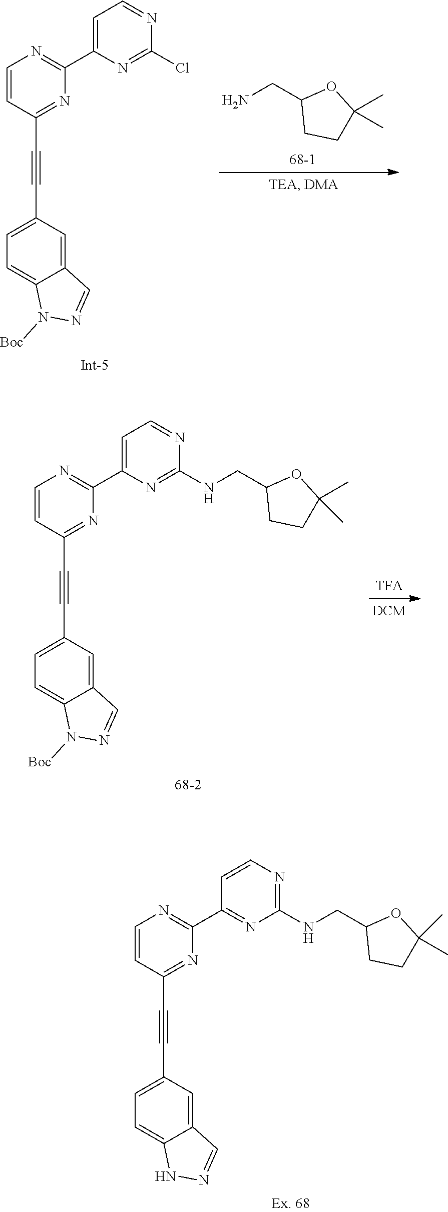

Ex. 68 was prepared from (5,5-dimethyltetrahydrofuran-2-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): δ (ppm): 10.90 (br, 1H), 8.90 (d, J=5.1 Hz), 8.52 (d, J=5.1 Hz, 1H), 8.14 (s, 1H), 8.05 (s, 1H), 7.66 (d, J=5.1 Hz, 1H), 7.50 (m, 3H), 6.16 (br, 1H), 4.20 (m, 1H), 3.68 (br, 1H), 3.58 (m, 1H), 3.00 (s, 1H), 2.93 (s, 1H), 2.07 (s, 1H), 2.03 (s, 1H), 1.76 (t, J=2.1 Hz, 2H), 1.26 (s, 3H), 1.22 (s, 3H). MS (ESI⁺): m/z: 426.26 (M+H)⁺.

### Example 69. (R)-4-((1H-Indazol-5-yl)ethynyl)-N-((tetrahydrofuran-3-yl)methyl)-[2,4′-bipyrimidin]-2′-amine (Ex. 69)

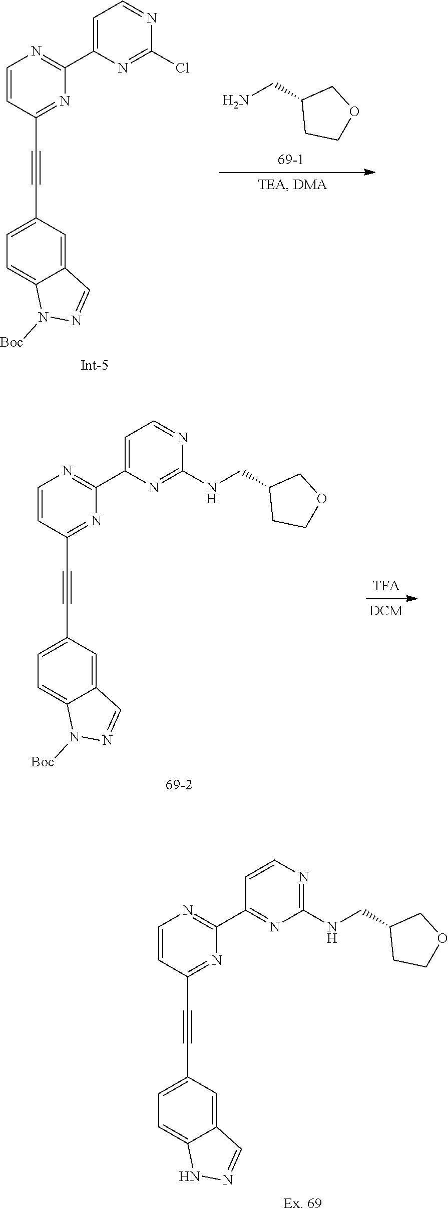 [SMILES: CC(C)(C)OC(=O)n1ncc2cc(C#Cc3ccnc(-c4ccnc(Cl)n4)n3)ccc21 ; NC[C@H]1CCOC1 ; CC(C)(C)OC(=O)n1ncc2cc(C#Cc3ccnc(-c4ccnc(NC[C@H]5CCOC5)n4)n3)ccc21 ; C(#Cc1ccnc(-c2ccnc(NC[C@H]3CCOC3)n2)n1)c1ccc2[nH]ncc2c1]

Ex. 69 was prepared from (R)-(tetrahydrofuran-2-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): 11.04 (br, 1H), 8.91 (d, J=5.1 Hz, 1H), 8.56 (d, J=5.4 Hz), 8.15 (s, 1H), 8.05 (s, 1H), 7.71 (d, J=5.1 Hz, 1H), 7.52 (m, 3H), 7.39 (d, J=9.0 Hz, 1H), 3.92 (m, 2H), 3.77 (q, J=7.2 Hz, 1H), 3.61 (dd, J₁=15.6 Hz, J₂=5.4 Hz, 1H), 3.55 (t, J=6.65 Hz, 2H), 2.63 (q, J=6.9 Hz, 1H), 2.09 (m, 1H), 1.70 (m, 1H). MS (ESI⁺): m/z: 398.21 (M+H)⁺.

### Example 70. 4-((1H-Indazol-5-yl)ethynyl)-N-((2-fluoro-4-methylpyridin-3-yl)methyl)-[2,4′-bipyrimidin]-2′-amine (Ex. 70)

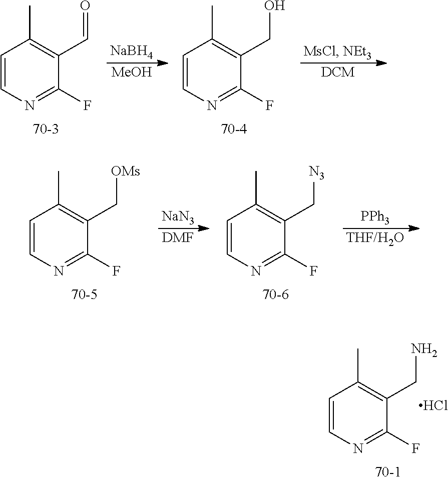

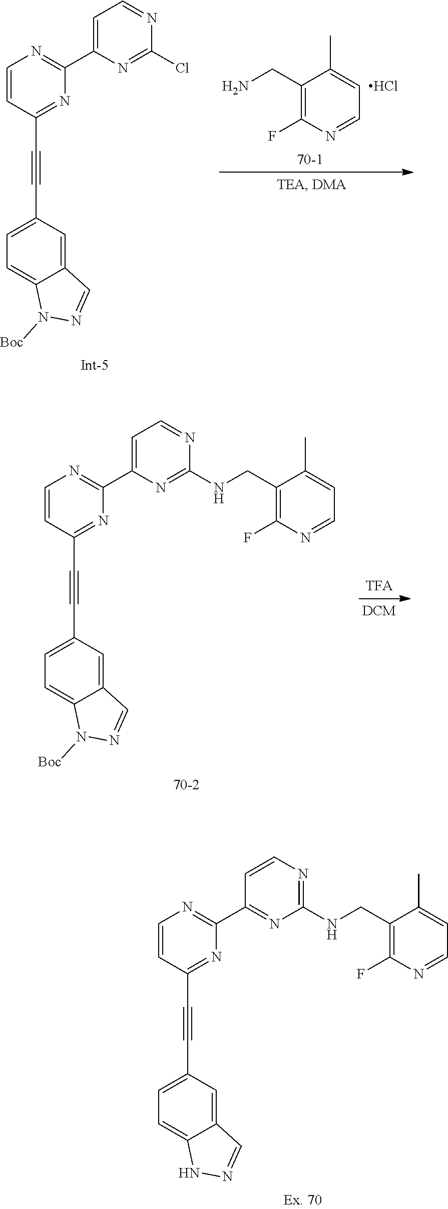

Ex. 70 was prepared from (2-fluoro-4-methylpyridin-3-yl)methanamine and tert-butyl 5-((2′-chloro-[2,4′-bipyrimidin]-4-yl)ethynyl)-1H-indazole-1-carboxylate (Int-5) in a manner analogous to the procedures described for Example 64. ¹H-NMR (300 MHz, CDCl₃): δ (ppm): 8.83 (d, J=5.0 Hz, 1H), 8.49 (d, J=5.0 Hz, 1H), 8.06 (t, J=1.1 Hz, 1H), 8.03 (d, J=0.7 Hz, 1H), 7.91 (d, J=5.1 Hz, 1H), 7.65 (d, J=5.0 Hz, 1H), 7.55 (dd, J=8.6, 1.5 Hz, 1H), 7.49 (dt, J=8.6, 0.9 Hz, 1H), 7.48 (d, J=5.1 Hz, 1H), 6.98 (d, J=5.1 Hz, 1H), 4.71 (s, 2H), 2.54 (s, 3H). MS (ESI⁺): m/z: 437.19 (M+H)⁺.

### Example 71. N-((5-Fluoropyridin-2-yl)methyl)-4-((3-methyl-1H-indazol-5-yl)ethynyl)-[2,4′-bipyrimidin]-2′-amine (Ex. 71)

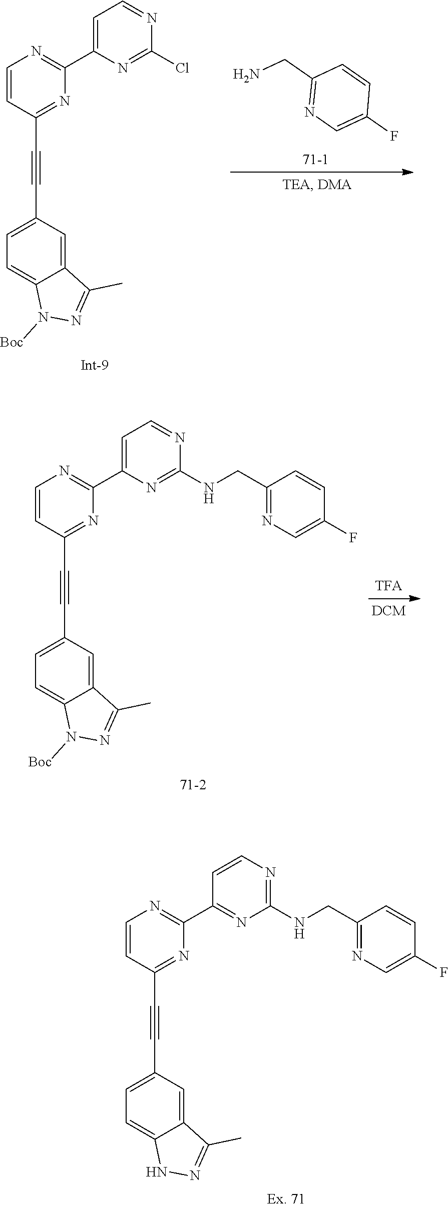 [SMILES: Cc1nn(C(=O)OC(C)(C)C)c2ccc(C#Cc3ccnc(-c4ccnc(Cl)n4)n3)cc12 ; NCc1ccc(F)cn1 ; Cc1nn(C(=O)OC(C)(C)C)c2ccc(C#Cc3ccnc(-c4ccnc(NCc5ccc(F)cn5)n4)n3)cc12 ; Cc1n[nH]c2ccc(C#Cc3ccnc(-c4ccnc(NCc5ccc(F)cn5)n4)n3)cc12]

Ex. 71 was prepared from (5-fluoropyridin-2-yl)methanamine and tert-butyl 5-((2′-chloro-[2,4′-bipyrimidin]-4-yl)ethynyl)-3-methyl-1H-indazole-1-carboxylate (Int-9) in a manner analogous to the procedures described for Step 3 and Step 4 of Example 1. ¹H-NMR (300 MHz, DMSO-d₆): δ (ppm): 9.02 (d, J=5.1 Hz, 1H), 8.51 (d, J=4.8 Hz, 2H), 8.19 (s, 1H), 7.79 (d, J=4.8 Hz, 1H), 7.70-7.63 (m, 2H), 7.58 (s, 2H), 7.50 (d, J=5.1 Hz, 1H), 4.68 (s, 2H), 2.53 (s, 3H). MS (ESI⁺): m/z: 437.19 (M+H)⁺.

### Example 72. 4-((1H-Indazol-5-yl)ethynyl)-N-((2-fluoro-5-methylpyridin-3-yl)methyl)-[2,4′-bipyrimidin]-2′-amine (Ex. 72)

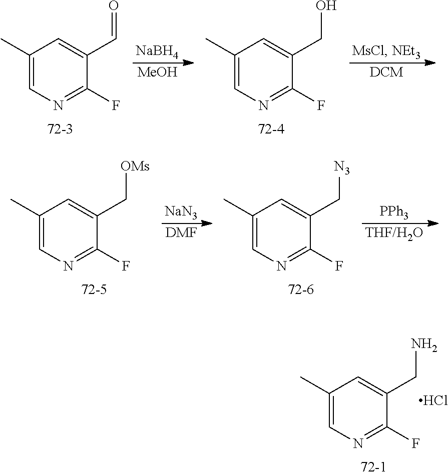

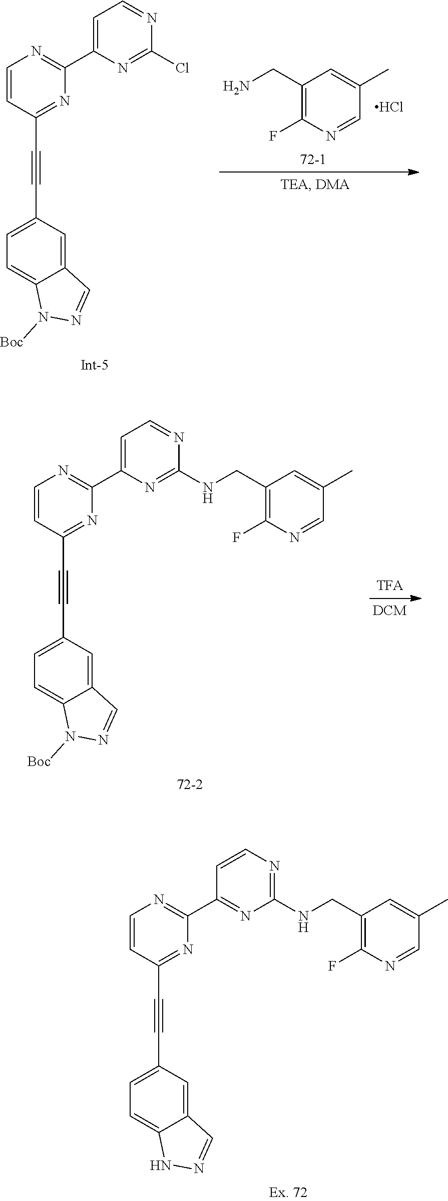

Ex. 72 was prepared from (2-fluoro-5-methylpyridin-3-yl)methanamine and tert-butyl 5-((2′-chloro-[2,4′-bipyrimidin]-4-yl)ethynyl)-1H-indazole-1-carboxylate (Int-5) in a manner analogous to the procedures described for Example 64. ¹H-NMR (300 MHz, CDCl₃): δ (ppm): 8.85 (d, J=5.1 Hz, 1H), 8.47 (d, J=5.1 Hz, 1H), 8.06 (t, J=1.1 Hz, 1H), 8.04 (br s, 1H), 7.79 (br s, 1H), 7.75-7.62 (m, 1H), 7.68 (d, J=5.0 Hz, 1H), 7.55 (dd, J=8.6, 1.3 Hz, 1H), 7.52-7.47 (m, 2H), 4.69 (s, 2H), 2.21 (s, 3H). MS (ESI⁺): m/z: 437.19 (M+H)⁺.

### Example 73. 2-((4-((1H-Indazol-5-yl)ethynyl)-[2,4′-bipyrimidin]-2′-yl)amino)-1-(4-methylpiperazin-1-yl)ethanone (Ex. 73)

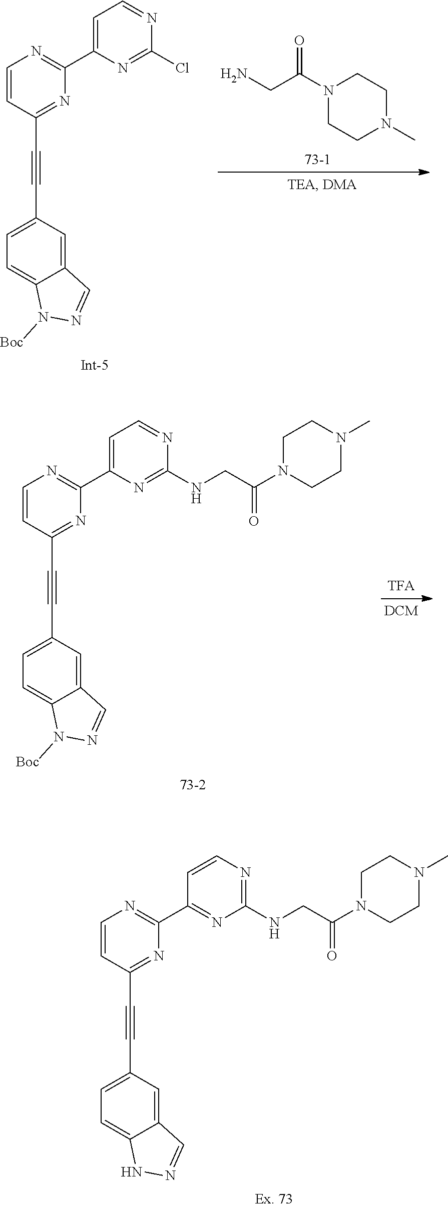 [SMILES: CC(C)(C)OC(=O)n1ncc2cc(C#Cc3ccnc(-c4ccnc(Cl)n4)n3)ccc21 ; CN1CCN(C(=O)CN)CC1 ; CN1CCN(C(=O)CNc2nccc(-c3nccc(C#Cc4ccc5c(cnn5C(=O)OC(C)(C)C)c4)n3)n2)CC1 ; CN1CCN(C(=O)CNc2nccc(-c3nccc(C#Cc4ccc5[nH]ncc5c4)n3)n2)CC1]

Ex. 73 was prepared from 2-amino-1-(4-methylpiperazin-1-yl)ethan-1-one and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, DMSO-d₆): δ (ppm): 13.44 (s, 1H), 9.03 (d, J=5.1 Hz, 1H), 8.53 (d, J=4.8 Hz, 1H), 8.23 (d, J=7.8 Hz, 2H), 7.82 (d, J=5.1 Hz, 1H), 7.66 (dd, J₁=8.7 Hz, J₂=16.2 Hz, 2H), 7.52 (d, J=5.1 Hz, 1H), 4.22 (d, J=4.8 Hz, 2H), 3.55-3.48 (m, 2H), 3.22-3.15 (m, 2H), 2.38-2.29 (m, 4H), 2.21 (s, 3H). MS (ESI⁺): m/z: 454.32 (M+H)⁺.

### Example 74. (1R,3S)-3-((4-((1H-indazol-5-yl)ethynyl)-[2,4′-bipyrimidin]-2′-yl)amino)cyclopentanol (Ex. 74)

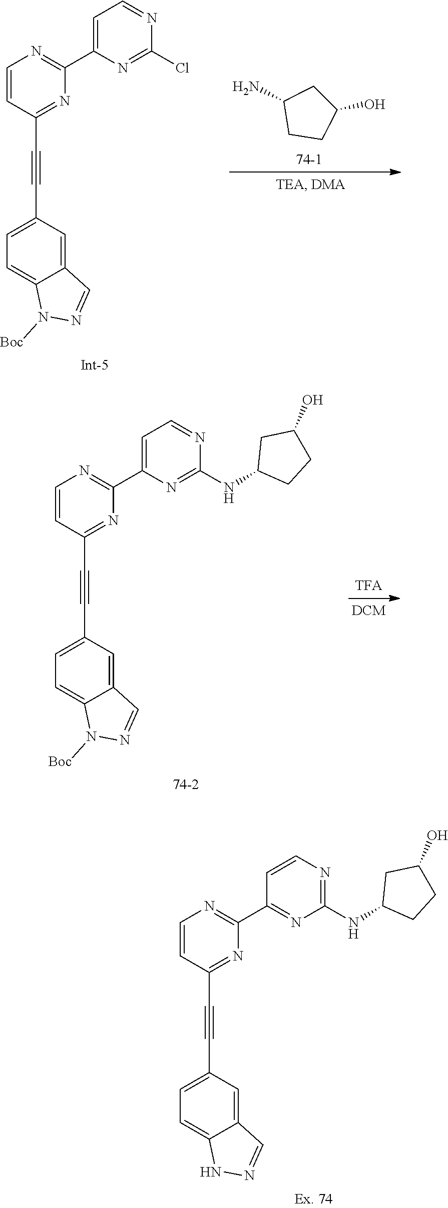 [SMILES: CC(C)(C)OC(=O)n1ncc2cc(C#Cc3ccnc(-c4ccnc(Cl)n4)n3)ccc21 ; N[C@H]1CC[C@@H](O)C1 ; CC(C)(C)OC(=O)n1ncc2cc(C#Cc3ccnc(-c4ccnc(N[C@H]5CC[C@@H](O)C5)n4)n3)ccc21 ; O[C@@H]1CC[C@H](Nc2nccc(-c3nccc(C#Cc4ccc5[nH]ncc5c4)n3)n2)C1]

Ex. 74 was prepared from (1R,3S)-3-aminocyclopentan-1-ol and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD-CDCl₃): δ=8.88 (d, J=5.1 Hz, 1H), 8.45 (d, J=5.1 Hz, 1H), 8.12 (s, 1H), 8.09 (s, 1H), 7.62-7.58 (m, 4H), 4.42-4.28 (m, 2H), 2.37-2.32 (m, 1H), 2.09-2.07 (m, 1H), 1.88-1.81 (m, 4H), 1.67-1.61 (m, 1H). MS (ESI⁺): m/z: 398.3 (M+H)⁺.

### Example 75. 4-((1H-Indazol-5-yl)ethynyl)-N-(2-methoxyethyl)-[2,4′-bipyrimidin]-2′-amine (Ex. 75)

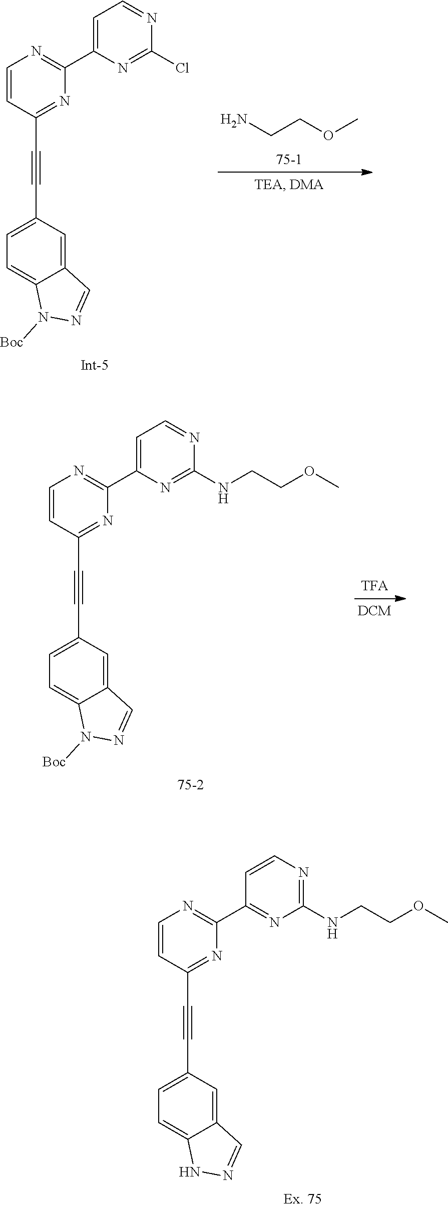

Ex. 75 was prepared from 2-methoxyethan-1-amine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): δ (ppm): 8.88 (d, J=5.1 Hz, 1H), 8.49 (d, J=5.1 Hz, 1H), 8.11 (s, 1H), 8.07 (t, J=1.1 Hz, 1H), 7.65 (d, J=5.1 Hz, 1H), 7.53 (s, 1H), 7.52 (s, 1H), 7.50 (d, J=5.0 Hz, 1H),), 3.48 (m, 2H), 3.62 (s, 3H), 3. 3.78 (m, 2H) MS (ESI⁺): m/z: 372.46 (M+H)⁺.

### Example 76. 4-((1H-Indazol-5-yl)ethynyl)-N-((tetrahydro-2H-pyran-2-yl)methyl)-[2,4′-bipyrimidin]-2′-amine (Ex. 76)

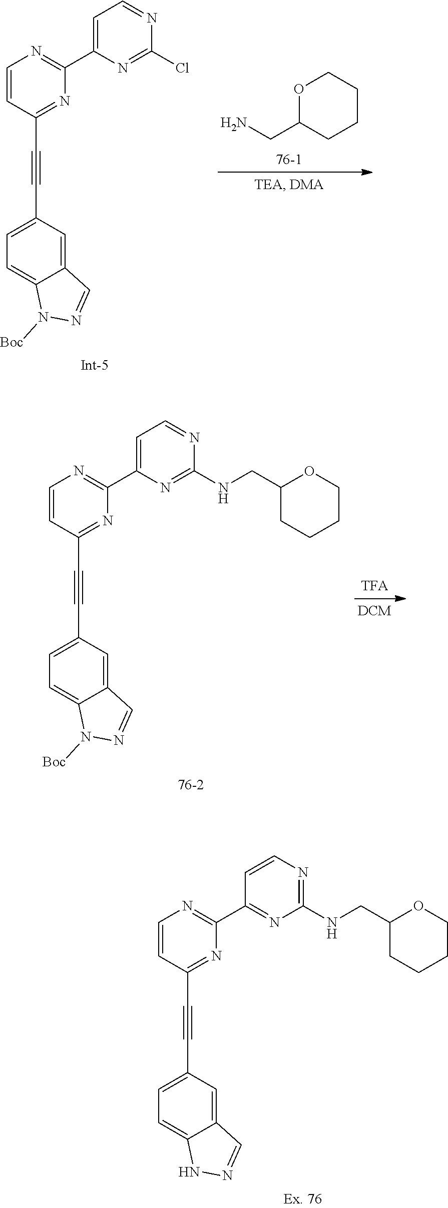 [SMILES: CC(C)(C)OC(=O)n1ncc2cc(C#Cc3ccnc(-c4ccnc(Cl)n4)n3)ccc21 ; NCC1CCCCO1 ; CC(C)(C)OC(=O)n1ncc2cc(C#Cc3ccnc(-c4ccnc(NCC5CCCCO5)n4)n3)ccc21 ; C(#Cc1ccnc(-c2ccnc(NCC3CCCCO3)n2)n1)c1ccc2[nH]ncc2c1]

Ex. 76 was prepared from (tetrahydro-2H-pyran-2-yl)methanamine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. MS (ESI⁺): m/z: 412.32 (M+H)⁺.

### Example 77. 2-((4-((3-Fluoro-1H-indazol-5-yl)ethynyl)-[2,4′-bipyrimidin]-2′-yl)amino)-1-morpholinoethanone (Ex. 77)

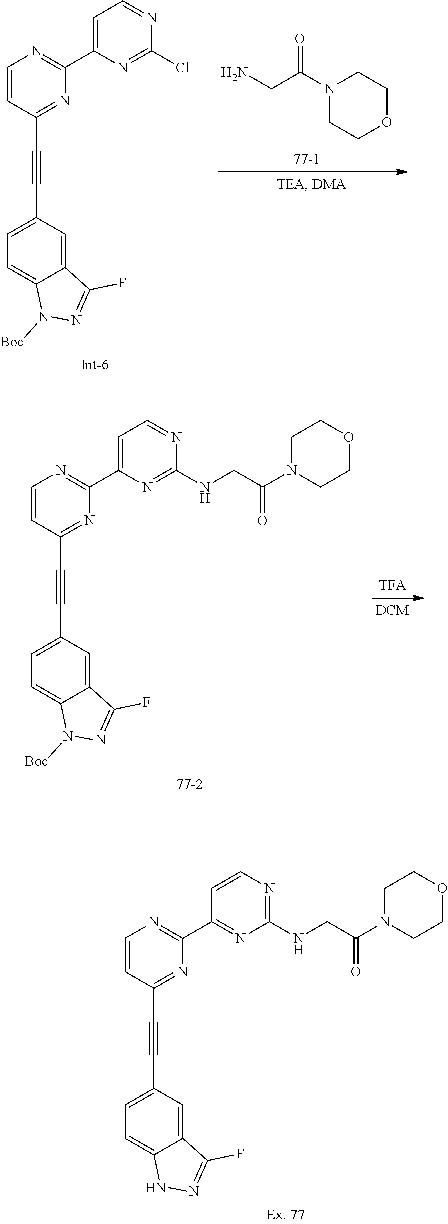 [SMILES: CC(C)(C)OC(=O)n1nc(F)c2cc(C#Cc3ccnc(-c4ccnc(Cl)n4)n3)ccc21 ; NCC(=O)N1CCOCC1 ; CC(C)(C)OC(=O)n1nc(F)c2cc(C#Cc3ccnc(-c4ccnc(NCC(=O)N5CCOCC5)n4)n3)ccc21 ; O=C(CNc1nccc(-c2nccc(C#Cc3ccc4[nH]nc(F)c4c3)n2)n1)N1CCOCC1]

Ex. 77 was prepared from 2-amino-1-morpholinoethan-1-one and tert-butyl 5-((2′-chloro-[2,4′-bipyrimidin]-4-yl)ethynyl)-3-fluoro-1H-indazole-1-carboxylate (Int-6) in a manner analogous to the procedures described for Step 3 and Step 4 of Example 1. ¹H-NMR (300 MHz, CDCl₃) δ 8.92 (d, J=6.8 Hz, 1H), 8.55 (d, J=6.8 Hz, 1H), 7.97 (s, 1H), 7.72 (d, J=6.8 Hz, 1H), 7.55-7.25 (m, 3H), 4.32 (d, J=4.5 Hz, 2H), 3.69 (m, 8H). MS (ESI⁺): m/z: 459.32 (M+H)⁺.

### Example 78. (1R,3R)-3-((4-((1H-Indazol-5-yl)ethynyl)-[2,4′-bipyrimidin]-2′-yl)amino)cyclopentanol (Ex. 78)

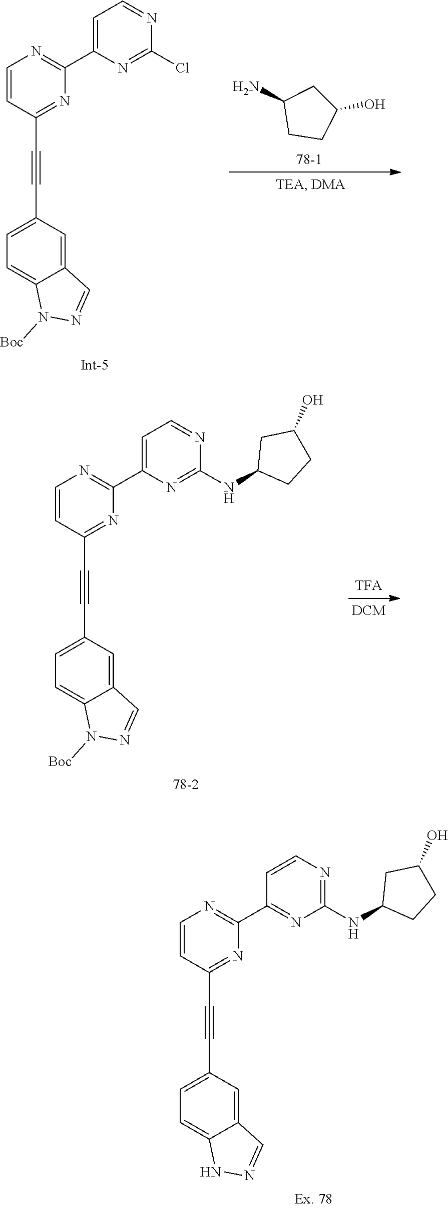 [SMILES: CC(C)(C)OC(=O)n1ncc2cc(C#Cc3ccnc(-c4ccnc(Cl)n4)n3)ccc21 ; N[C@@H]1CC[C@@H](O)C1 ; CC(C)(C)OC(=O)n1ncc2cc(C#Cc3ccnc(-c4ccnc(N[C@@H]5CC[C@@H](O)C5)n4)n3)ccc21 ; O[C@@H]1CC[C@@H](Nc2nccc(-c3nccc(C#Cc4ccc5[nH]ncc5c4)n3)n2)C1]

Ex. 78 was prepared from (1R,3R)-3-aminocyclopentan-1-ol and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): δ 8.77 (d, J=5.1 Hz, 1H), 8.37 (d, J=5.0 Hz, 1H), 8.01 (m, 1H), 7.98 (t, J=0.9 Hz, 1H), 7.54 (d, J=5.1 Hz, 1H), 7.49 (m, 1H), 7.46 (m, 1H), 7.44 (m, 1H), 4.45 (m, 1H), 4.29 (m, 1H), 2.27-2.13 (m, 1H), 2.10-1.94 (m, 2H), 1.70-1.51 (m, 2H), 1.49-1.35 (m, 1H). MS (ESI⁺): m/z: 398.22 (M+H)⁺.

### Example 79. (R)-1-((4-((1H-Indazol-5-yl)ethynyl)-[2,4′-bipyrimidin]-2′-yl)amino)propan-2-ol (Ex. 79)

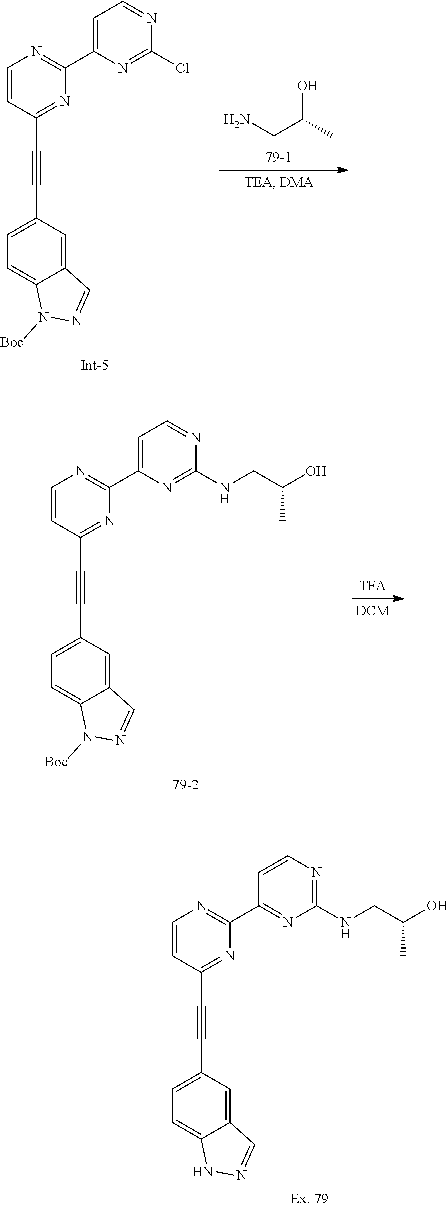

Ex. 79 was prepared from (R)-1-aminopropan-2-ol and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. MS (ESI⁺): m/z: 372.22 (M+H)⁺.

### Example 80. (S)-1-((4-((1H-Indazol-5-yl)ethynyl)-[2,4′-bipyrimidin]-2′-yl)amino)propan-2-ol (Ex. 80)

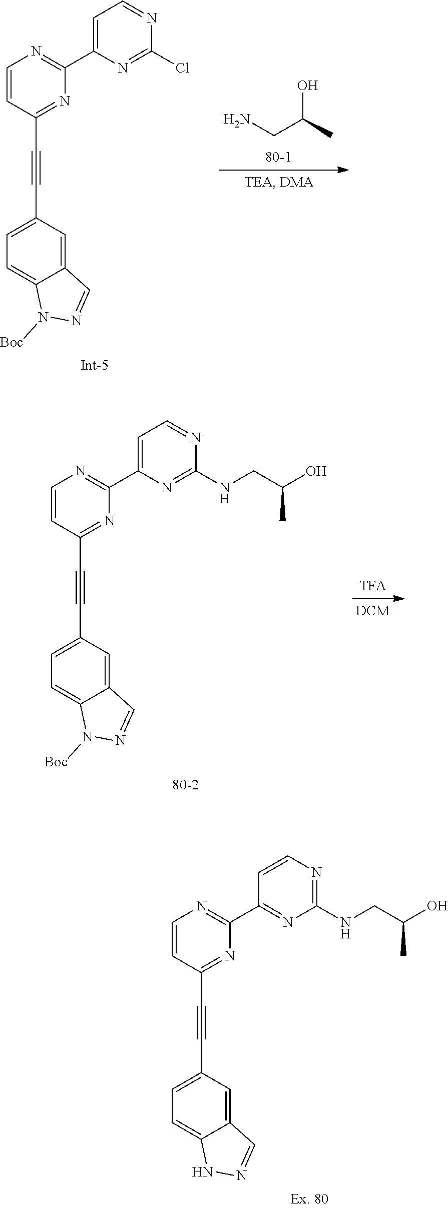

Ex. 80 was prepared from (S)-1-aminopropan-2-ol and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD): 8.94 (d, J=5.1 Hz, 1H), 8.51 (d, J=5.1 Hz, 1H), 8.17 (d, J=12.0 Hz, 2H), 7.68 (q, J=6.0 Hz, 2H), 7.64 (m, 2H), 3.99 (m, 1H), 3.56 (br, 1H), 3.37 (br, 1H), 1.23 (d, J=6.0 Hz, 2H). MS (ESI⁺): m/z: 372.25 (M+H)⁺.

### Example 81. 4-((1H-Indazol-5-yl)ethynyl)-N-(2-methoxy-2-methylpropyl)-[2,4′-bipyrimidin]-2′-amine (Ex. 81)

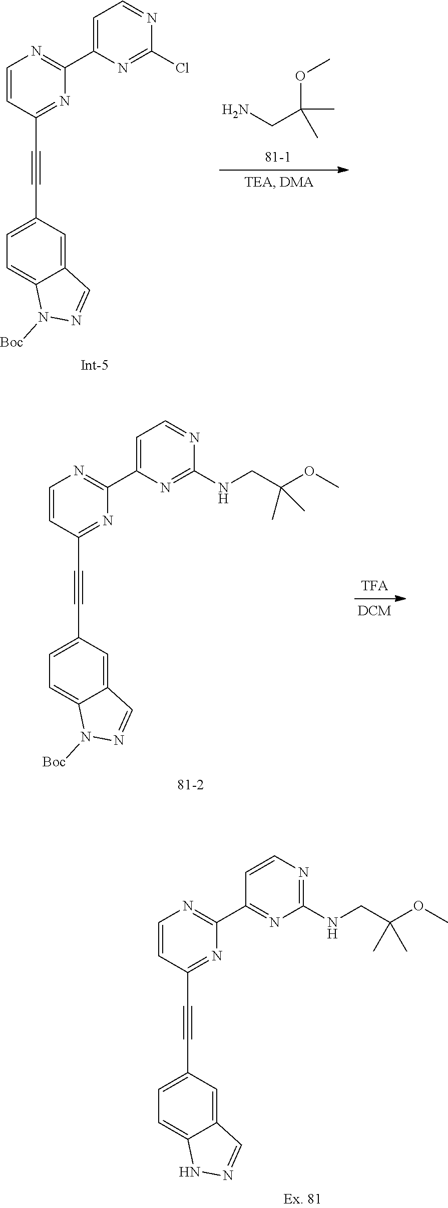

Ex. 81 was prepared from 2-methoxy-2-methylpropan-1-amine and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): 8.92 (d, J=5.1 Hz, 1H), 8.51 (br, 1H), 8.14 (s, 1H), 8.10 (s, 1H), 7.68 (d, J=5.1 Hz, 1H), 7.54 (m, 3H), 3.56 (br, 2H), 3.22 (s, 3H), 1.29 (s, 6H). MS (ESI⁺): m/z: 400.22 (M+H)⁺.

### Example 82. (1S,3R)-3-((4-((1H-Indazol-5-yl)ethynyl)-[2,4′-bipyrimidin]-2′-yl)amino)cyclopentanol (Ex. 82)

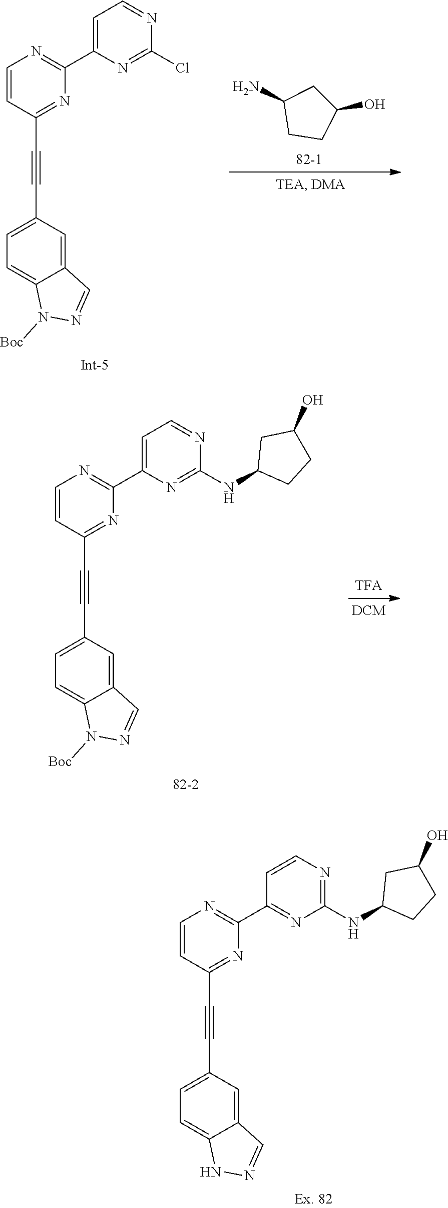 [SMILES: CC(C)(C)OC(=O)n1ncc2cc(C#Cc3ccnc(-c4ccnc(Cl)n4)n3)ccc21 ; N[C@@H]1CC[C@H](O)C1 ; CC(C)(C)OC(=O)n1ncc2cc(C#Cc3ccnc(-c4ccnc(N[C@@H]5CC[C@H](O)C5)n4)n3)ccc21 ; O[C@H]1CC[C@@H](Nc2nccc(-c3nccc(C#Cc4ccc5[nH]ncc5c4)n3)n2)C1]

Ex. 82 was prepared from (1S,3R)-3-aminocyclopentan-1-ol and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): δ (ppm): 8.88 (d, J=5.1 Hz, 1H), 8.49 (d, J=5.1 Hz, 1H), 8.11 (s, 1H), 8.07 (t, J=1.1 Hz, 1H), 7.65 (d, J=5.1 Hz, 1H), 7.53 (s, 1H), 7.52 (s, 1H), 7.50 (d, J=5.0 Hz, 1H), 4.40-4.32 (m, 2H), 2.26 (m, 1H), 2.01 (m, 2H), 1.86-1.75 (m, 3H). MS (ESI⁺): m/z: 398.26 (M+H)⁺.

### Example 83. (S)-3-((4-((1H-Indazol-5-yl)ethynyl)-[2,4′-bipyrimidin]-2′-yl)amino)tetrahydrothiophene 1,1-dioxide (Ex. 83)

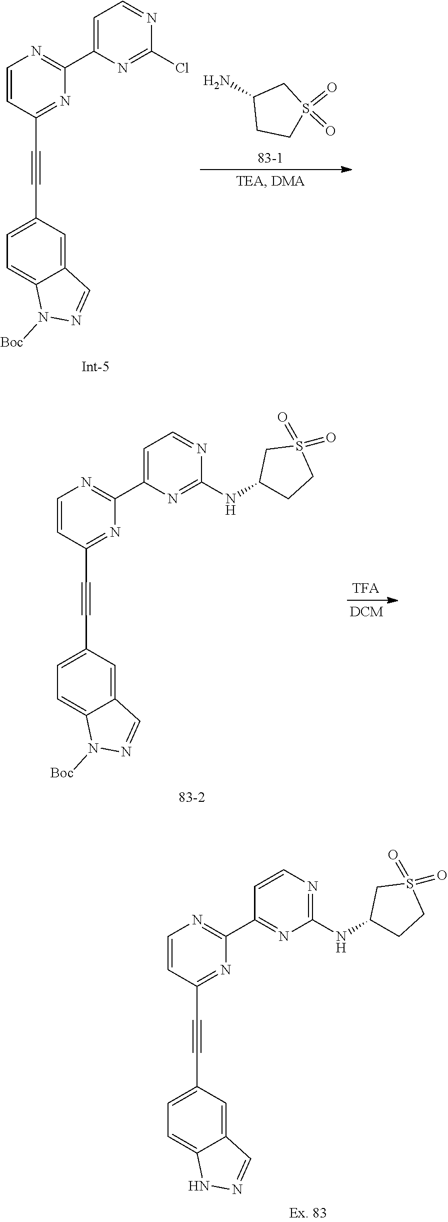

Ex. 83 was prepared from (S)-3-aminotetrahydrothiophene 1,1-dioxide and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): δ (ppm): 8.83 (d, J=5.0 Hz, 1H), 8.48 (d, J=5.1 Hz, 1H), 8.07-8.01 (m, 2H), 7.71 (d, J=5.0 Hz, 1H), 7.57-7.47 (m, 3H), 4.86 (s, 1H), 3.61 (dd, J=13.3, 7.3, Hz, 1H), 3.22-3.01 (m, 3H), 2.61 (m, 1H), 2.30 (dd, J=13.6, 8.2 Hz, 1H). MS (ESI⁺): m/z: 432.19 (M+H)⁺.

### Example 84. 2-((4-((1H-Indazol-5-yl)ethynyl)-[2,4′-bipyrimidin]-2′-yl)amino)-1-(3-hydroxy-3-methylazetidin-1-yl)ethanone (Ex. 84)

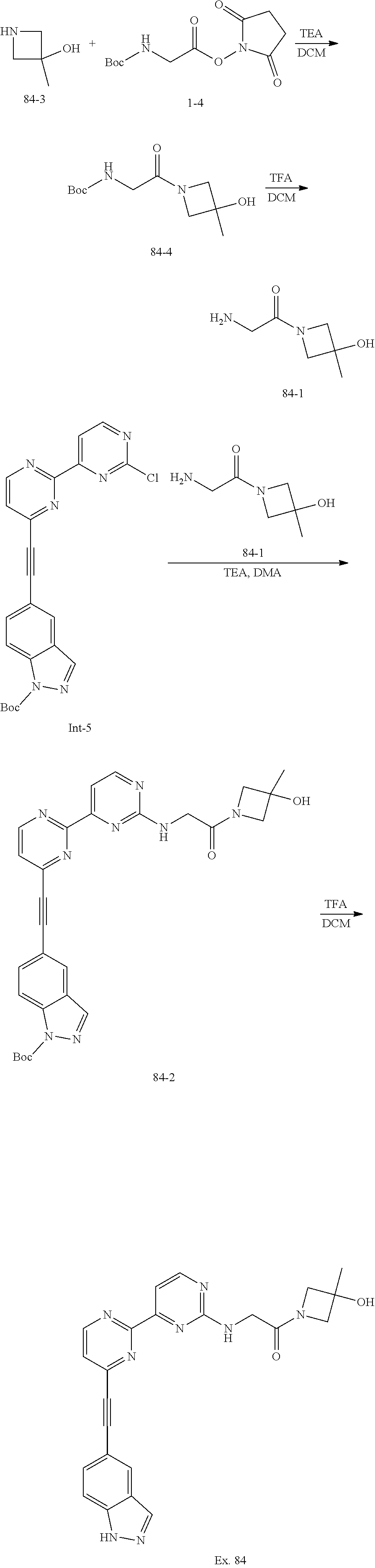

Ex. 84 was prepared from 2-amino-1-(3-hydroxy-3-methylazetidin-1-yl)ethan-1-one and tert-butyl 5-((2′-chloro-[2,4′-bipyrimidin]-4-yl)ethynyl)-1H-indazole-1-carboxylate (Int-5) in a manner analogous to the procedures described for Example 1. ¹H-NMR (300 MHz, CDCl₃): δ (ppm): 8.86 (d, J=5.0 Hz, 1H), 8.48 (d, J=5.0 Hz, 1H), 8.08 (t, J=1.1 Hz, 1H), 8.06 (s, 1H), 7.69 (d, J=5.1 Hz, 1H), 7.58 (dd, J=8.7, 1.3 Hz, 1H), 7.53-7.48 (m, 2H), 4.18-4.02 (m, 4H), 3.91 (s, 2H), 1.46 (s, 3H). MS (ESI⁺): m/z: 441.20 (M+H)⁺.

### Example 85. 4-(((4-((1H-Indazol-5-yl)ethynyl)-[2,4′-bipyrimidin]-2′-yl)amino)methyl)tetrahydro-2H-thiopyran 1,1-dioxide (Ex. 85)

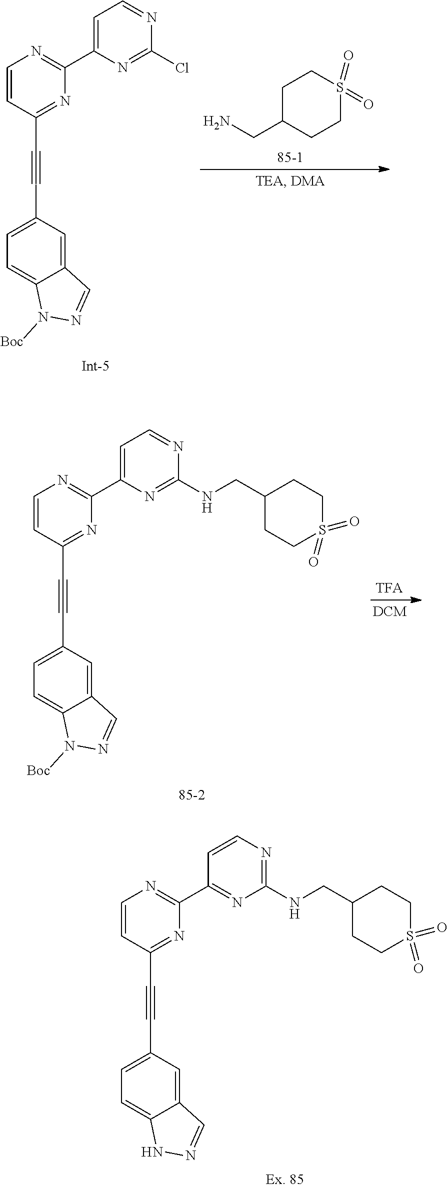 [SMILES: CC(C)(C)OC(=O)n1ncc2cc(C#Cc3ccnc(-c4ccnc(Cl)n4)n3)ccc21 ; NCC1CCS(=O)(=O)CC1 ; CC(C)(C)OC(=O)n1ncc2cc(C#Cc3ccnc(-c4ccnc(NCC5CCS(=O)(=O)CC5)n4)n3)ccc21 ; O=S1(=O)CCC(CNc2nccc(-c3nccc(C#Cc4ccc5[nH]ncc5c4)n3)n2)CC1]

Ex. 85 was prepared from 4-(aminomethyl)tetrahydro-2H-thiopyran 1,1-dioxide and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): δ (ppm): 8.92 (d, J=5.1 Hz, 1H), 8.56 (d, J=5.0 Hz, 1H), 8.17 (s, 1H), 7.97 (s, 1H), 7.75 (d, J=5.0 Hz, 1H), 7.56-7.52 (m, 2H), 7.12 (d, J=7.3 Hz, 1H), 3.50 (t, J=5.7 Hz, 2H), 3.16-2.88 (m, 4H), 2.30-2.18 (m, 2H), 2.04-1.88 (m, 3H). MS (ESI⁺): m/z: 460.37 (M+H)⁺.

### Example 86. (1r,4r)-Methyl 4-((4-((1H-indazol-5-yl)ethynyl)-[2,4′-bipyrimidin]-2′-yl)amino)cyclohexanecarboxylate (Ex. 86)

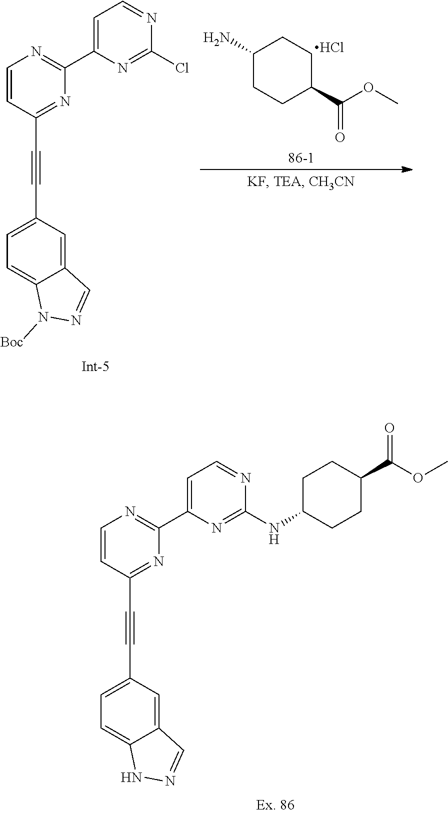 [SMILES: CC(C)(C)OC(=O)n1ncc2cc(C#Cc3ccnc(-c4ccnc(Cl)n4)n3)ccc21 ; COC(=O)[C@H]1CC[C@H](N)CC1 ; Cl ; COC(=O)[C@H]1CC[C@H](Nc2nccc(-c3nccc(C#Cc4ccc5[nH]ncc5c4)n3)n2)CC1]

To a stirred mixture of tert-butyl 5-((2′-chloro-[2,4′-bipyrimidin]-4-yl)ethynyl)-1H-indazole-1-carboxylate (Int-5, 50.0 mg, 0.1155 mmol) and methyl (1r,4r)-4-aminocyclohexane-1-carboxylate-hydrochloride (86-1, 26.8 mg, 0.1386 mmol) in acetonitrile (5 mL) was added Et₃N (40 μL, 0.276 mmol), potassium fluoride dihydrate (21.65 mg, 0.231 mmol) and 18-crown-6 (60.8 mg, 0.231 mmol) at room temperature. The reaction mixture was stirred under N₂ at 100° C. for 24 h. LC-MS showed the reaction was complete. After cooling to rt, the reaction mixture was concentrated under reduced pressure and the crude mixture was purified by column chromatography on silica gel by sequential elution with 20% EtOAc in hexanes, 50% EtOAc in hexanes, followed by 1% MeOH in EtOAc to afford (1r,4r)-methyl 4-((4-((1H-indazol-5-yl)ethynyl)-[2,4′-bipyrimidin]-2′-yl)amino)cyclohexanecarboxylate (Ex. 86, 29.0 mg, yield: 55%) as a pale brown solid. ¹H-NMR (300 MHz, DMSO-d₆: δ (ppm): 13.43 (s, 1H), 9.01 (d, J=5.1 Hz, 1H), 8.51 (d, J=4.5 Hz, 1H), 8.21 (s, 2H), 7.80 (d, J=5.1 Hz, 1H), 7.67 (d, J=8.4 Hz, 1H), 7.60 (d, J=8.1 Hz, 1H), 7.41 (d, J=4.8 Hz, 1H), 3.6 (s, 3H), 2.57-2.54 (m, 1H), 2.38-2.30 (m, 1H), 2.02-1.94 (m, 4H), 1.46-1.31 (m, 4H). MS (ESI⁺): m/z: 454.40 (M+H)⁺.

### Example 87. (1r,4r)-4-((4-((1H-Indazol-5-yl)ethynyl)-[2,4′-bipyrimidin]-2′-yl)amino)cyclohexanecarboxylic acid (Ex. 87)

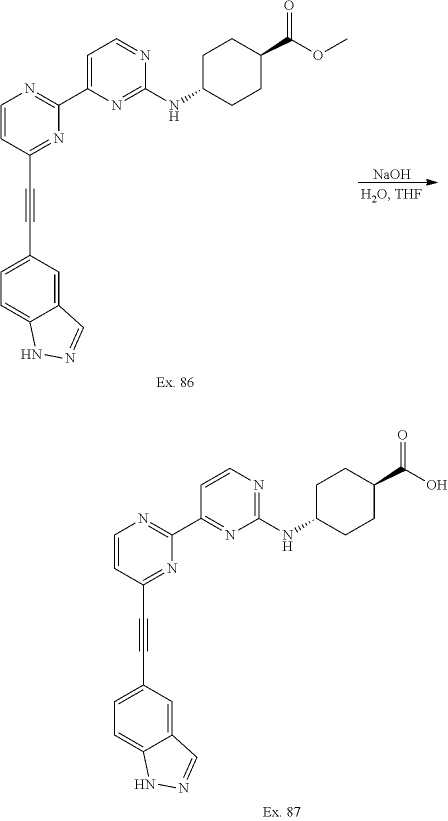 [SMILES: COC(=O)[C@H]1CC[C@H](Nc2nccc(-c3nccc(C#Cc4ccc5[nH]ncc5c4)n3)n2)CC1 ; "NaOH" ; O=C(O)[C@H]1CC[C@H](Nc2nccc(-c3nccc(C#Cc4ccc5[nH]ncc5c4)n3)n2)CC1]

To a solution of (1r,4r)-methyl 4-((4-((1H-indazol-5-yl)ethynyl)-[2,4′-bipyrimidin]-2′-yl)amino)cyclohexanecarboxylate (Ex. 86, 15.0 mg, 0.0331 mmol) in THE (2 mL) was added 4 N aq. NaOH (2 mL) and the mixture was stirred at rt for 4 h. LC-MS showed the reaction was complete. The reaction mixture was diluted with 10 mL of aq. NaOH and 10 mL of DCM. The layers were separated and the aqueous layer was acidified with citric acid. The aqueous layer was extracted with EtOAc (3×15 mL). The organic layers were combined, washed with brine (10 mL), and concentrated in vacuo to give (1r,4r)-4-((4-((1H-indazol-5-yl)ethynyl)-[2,4′-bipyrimidin]-2′-yl)amino)cyclohexanecarboxylic acid (Ex. 87, 10 mg, yield: 69%) as a yellow solid. ¹H-NMR (300 MHz, DMSO-d₆): δ (ppm): 13.2 (s, 1H), 11.8 (brs, 1H), 8.90 (d, J=4.5 Hz, 1H), 8.46 (d, J=4.4 Hz, 1H), 8.22 (s, 2H), 7.74 (d, J=5.1 Hz, 1H), 7.59 (d, J=8.4 Hz, 1H), 7.54 (d, J=8.4 Hz, 1H), 7.34 (d, J=4.2 Hz, 1H), 2.52-2.48 (m, 1H), 2.36-2.30 (m, 1H), 1.94-1.88 (m, 4H), 1.28-1.20 (m, 4H). MS (ESI⁺): m/z: 440.39 (M+H)⁺.

### Example 88. (R)-3-((4-((1H-Indazol-5-yl)ethynyl)-[2,4′-bipyrimidin]-2′-yl)amino)tetrahydrothiophene 1,1-dioxide (Ex. 88)

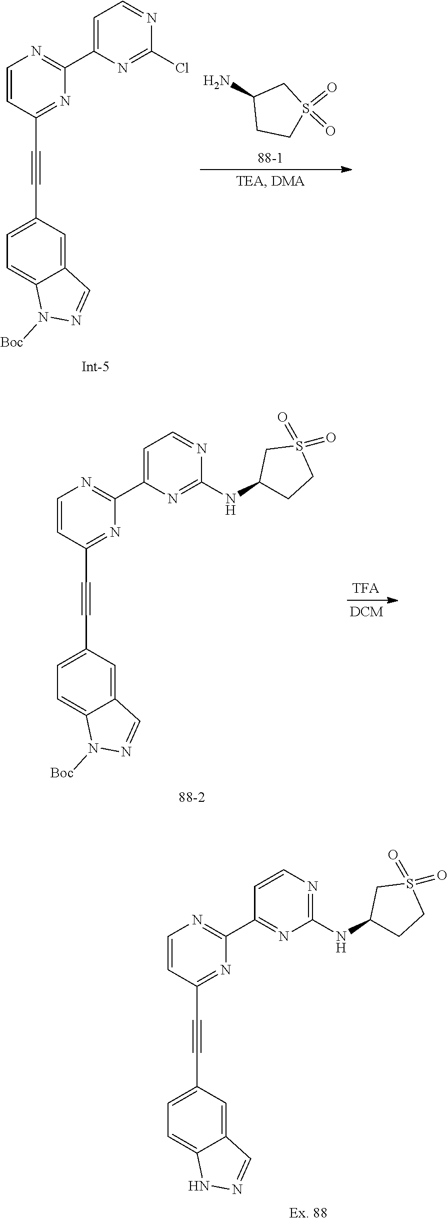

Ex. 88 was prepared from (R)-3-aminotetrahydrothiophene 1,1-dioxide and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): 8.93 (d, J=4.8 Hz, 1H), 8.62 (br, 1H), 8.16 (s, 1H), 8.11 (s, 1H), 7.86 (d, J=5.1 Hz), 7.55 (m, 3H), 4.92 (br, 1H), 3.67 (q, J=7.8 Hz, 1H), 3.40 (m, 1H), 3.17 (m, 3H), 2.67 (m, 1H), 2.39 (m, 1H). MS (ESI⁺): m/z: 432.40 (M+H)⁺.

### Example 89. N1-(4-((1H-Indazol-5-yl)ethynyl)-[2,4′-bipyrimidin]-2′-yl)-2-methylpropane-1,2-diamine (Ex. 89)

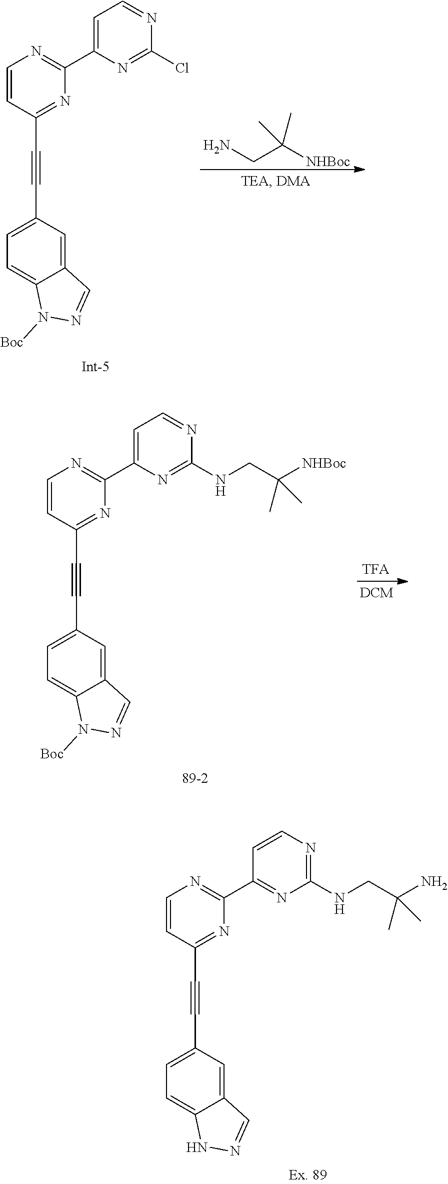 [SMILES: CC(C)(C)OC(=O)n1ncc2cc(C#Cc3ccnc(-c4ccnc(Cl)n4)n3)ccc21 ; CC(C)(CN)NC(=O)OC(C)(C)C ; CC(C)(CNc1nccc(-c2nccc(C#Cc3ccc4c(cnn4C(=O)OC(C)(C)C)c3)n2)n1)NC(=O)OC(C)(C)C ; CC(C)(N)CNc1nccc(-c2nccc(C#Cc3ccc4[nH]ncc4c3)n2)n1]

Ex. 89 was prepared from tert-butyl (1-amino-2-methylpropan-2-yl)carbamate and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CD₃OD): δ (ppm): 8.98 (d, J=5.1 Hz, 1H), 8.62 (d, J=5.0 Hz, 1H), 8.22 (t, J=1.0 Hz, 1H), 8.16 (s, 1H), 7.82 (d, J=5.0 Hz, 1H), 7.75 (d, J=5.3 Hz, 1H), 7.70-7.62 (m, 2H), 3.64 (s, 2H), 1.43 (s, 6H). MS (ESI⁺): m/z: 385.38 (M+H)⁺.

### Example 90. (1S,3S)-3-((4-((1H-Indazol-5-yl)ethynyl)-[2,4′-bipyrimidin]-2′-yl)amino)cyclopentanol (Ex. 90)

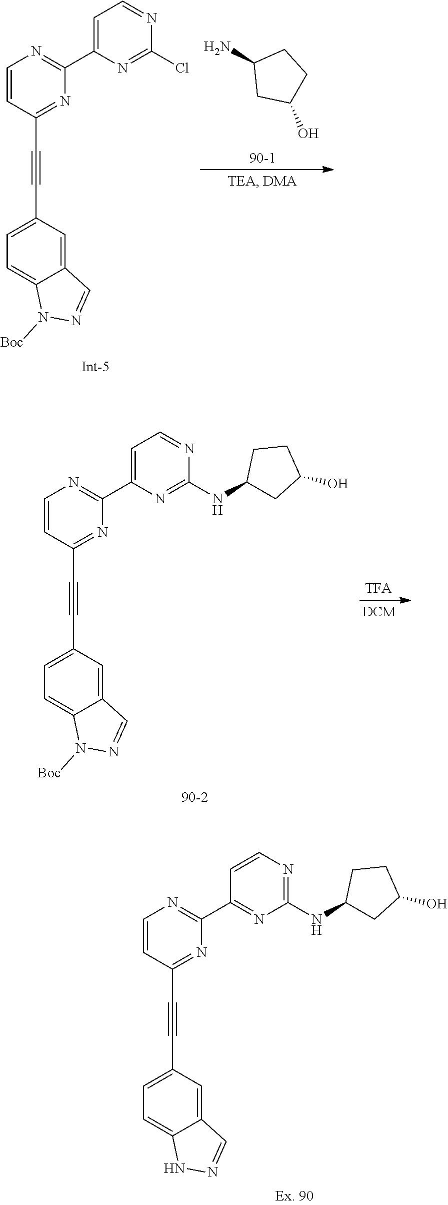 [SMILES: CC(C)(C)OC(=O)n1ncc2cc(C#Cc3ccnc(-c4ccnc(Cl)n4)n3)ccc21 ; N[C@H]1CC[C@H](O)C1 ; CC(C)(C)OC(=O)n1ncc2cc(C#Cc3ccnc(-c4ccnc(N[C@H]5CC[C@H](O)C5)n4)n3)ccc21 ; O[C@H]1CC[C@H](Nc2nccc(-c3nccc(C#Cc4ccc5[nH]ncc5c4)n3)n2)C1]

Ex. 90 was prepared from (1S,3S)-3-aminocyclopentan-1-ol and tert-butyl 5-((2′-chloro-[2,4′-bipyrimidin]-4-yl)ethynyl)-1H-indazole-1-carboxylate (Int-5) in a manner analogous to the procedures described for Step 3 and Step 4 of Example 1. ¹H-NMR (300 MHz, CDCl₃): δ (ppm): 8.77 (d, J=5.1 Hz, 1H), 8.37 (d, J=5.0 Hz, 1H), 8.01 (m, 1H), 7.98 (t, J=0.9 Hz, 1H), 7.54 (d, J=5.1 Hz, 1H), 7.49 (m, 1H), 7.46 (m, 1H), 7.44 (m, 1H), 4.45 (m, 1H), 4.29 (m, 1H), 2.27-2.13 (m, 1H), 2.10-1.94 (m, 2H), 1.70-1.51 (m, 2H), 1.49-1.35 (m, 1H). MS (ESI⁺): m/z: 398.41 (M+H)⁺.

### Example 91. (1s,4s)-Methyl 4-((4-((1H-indazol-5-yl)ethynyl)-[2,4′-bipyrimidin]-2′-yl)amino)cyclohexanecarboxylate (Ex. 91)

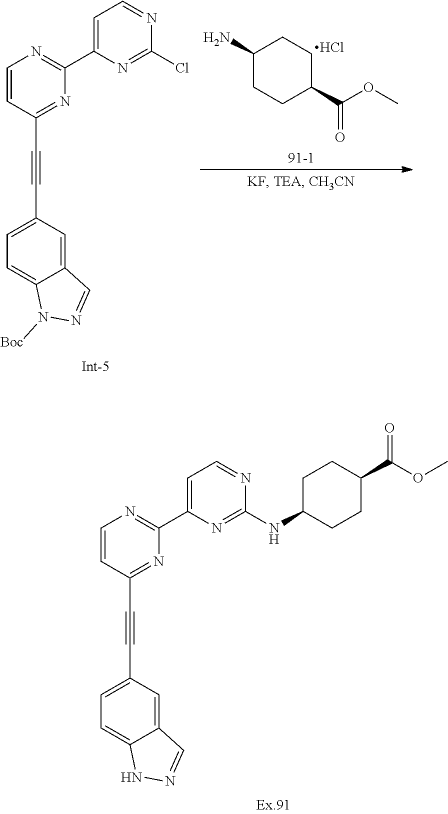 [SMILES: CC(C)(C)OC(=O)n1ncc2cc(C#Cc3ccnc(-c4ccnc(Cl)n4)n3)ccc21 ; COC(=O)[C@H]1CC[C@@H](N)CC1 ; Cl ; COC(=O)[C@H]1CC[C@@H](Nc2nccc(-c3nccc(C#Cc4ccc5[nH]ncc5c4)n3)n2)CC1]

Ex. 91 was prepared from methyl (1s,4s)-4-aminocyclohexane-1-carboxylate and tert-butyl 5-((2′-chloro-[2,4′-bipyrimidin]-4-yl)ethynyl)-1H-indazole-1-carboxylate (Int-5) in a manner analogous to the procedure described for Example 86 to provide the compound in 69% yield. ¹H-NMR (300 MHz, DMSO-d₆: δ (ppm): 13.39 (s, 1H), 9.04 (d, J=5.1 Hz, 1H), 8.50 (d, J=4.5 Hz, 1H), 8.16 (s, 2H), 7.79 (d, J=5.1 Hz, 1H), 7.60 (d, J=8.4 Hz, 1H), 7.54 (d, J=8.4 Hz, 1H), 7.35 (d, J=4.8 Hz, 1H), 3.60 (s, 3H), 2.50-2.48 (m, 1H), 2.32-2.28 (m, 1H), 1.98-1.92 (m, 4H), 1.44-1.31 (m, 4H). MS (ESI⁺): m/z: 454.40 (M+H)⁺.

### Example 92. (1s,4s)-4-((4-((1H-Indazol-5-yl)ethynyl)-[2,4′-bipyrimidin]-2′-yl)amino)cyclohexanecarboxylic acid (Ex. 92)

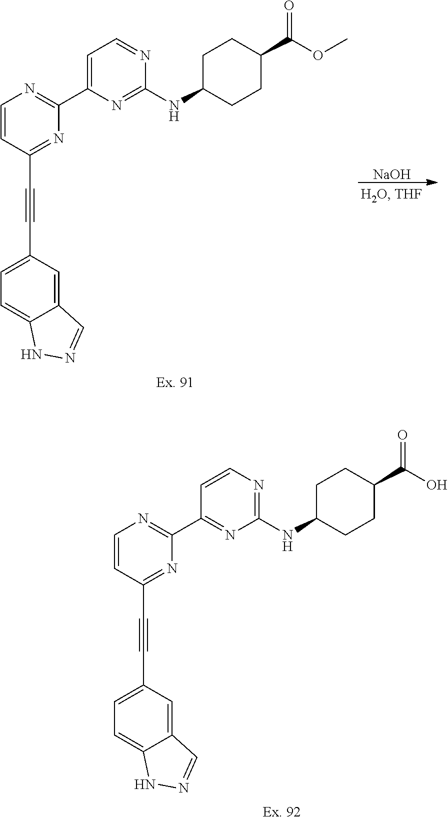 [SMILES: COC(=O)[C@H]1CC[C@@H](Nc2nccc(-c3nccc(C#Cc4ccc5[nH]ncc5c4)n3)n2)CC1 ; "NaOH" ; O=C(O)[C@H]1CC[C@@H](Nc2nccc(-c3nccc(C#Cc4ccc5[nH]ncc5c4)n3)n2)CC1]

Ex. 92 was prepared from (1s,4s)-methyl 4-((4-((1H-indazol-5-yl)ethynyl)-[2,4′-bipyrimidin]-2′-yl)amino)cyclohexanecarboxylate (Ex. 91) in a manner analogous to the procedure described for Example 87 to provide the compound in 55% yield. ¹H-NMR (300 MHz, DMSO-d₆): δ (ppm): 13.40 (s, 1H), 12.0 (brs, 1H), 8.98 (d, J=4.5 Hz, 1H), 8.47 (d, J=4.4 Hz, 1H), 8.18 (s, 2H), 7.76 (d, J=5.1 Hz, 1H), 7.63 (d, J=8.4 Hz, 1H), 7.56 (d, J=8.4 Hz, 1H), 7.38 (d, J=4.2 Hz, 1H), 2.50-2.44 (m, 1H), 2.32-2.28 (m, 1H), 1.96-1.80 (m, 4H), 1.38-1.20 (m, 4H). MS (ESI⁺): m/z: 440.34 (M+H)⁺.

### Example 93. (1s,4s)-4-((4-((3-Fluoro-1H-Indazol-5-yl)ethynyl)-[2,4′-bipyrimidin]-2′-yl)amino)cyclohexanol (Ex. 93)

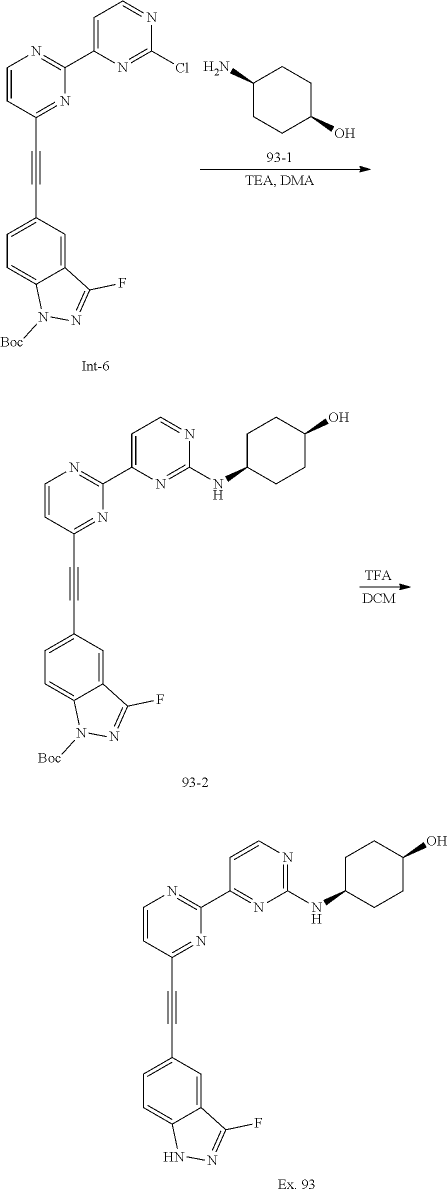

Ex. 93 was prepared from (1s,4s)-4-aminocyclohexan-1-ol and tert-butyl 5-((2′-chloro-[2,4′-bipyrimidin]-4-yl)ethynyl)-3-fluoro-1H-indazole-1-carboxylate (Int-6) in a manner analogous to the procedures described for Step 3 and Step 4 of Example 1. ¹H-NMR (300 MHz, DMSO-d₆) δ 8.93 (d, J=6.8 Hz, 1H), 8.52 (d, J=6.8 Hz, 1H), 7.87 (s, 1H), 7.69 (d, J=6.8 Hz, 1H), 7.55-7.25 (m, 3H), 5.29 (bs, 1H), 4.15-4.03 (m, 2H), 1.27-1.23 (m, 9H); MS (ESI⁺): m/z: 430.19 (M+H)⁺.

### Example 94. (1S,3R)-3-((4-((3-Fluoro-1H-Indazol-5-yl)ethynyl)-[2,4′-bipyrimidin]-2′-yl)amino)cyclopentanol (Ex. 94)

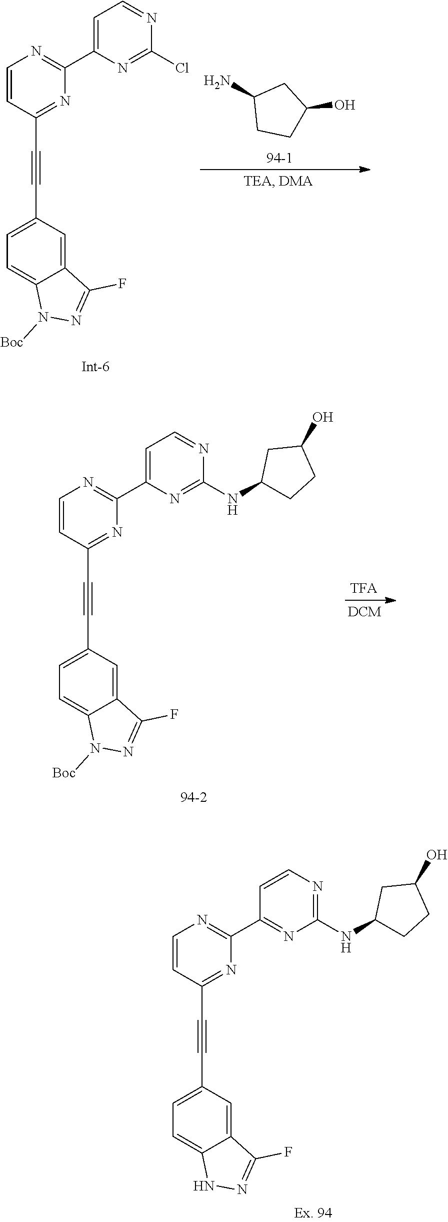

Ex. 94 was prepared from (1S,3R)-3-aminocyclopentan-1-ol and tert-butyl 5-((2′-chloro-[2,4′-bipyrimidin]-4-yl)ethynyl)-3-fluoro-1H-indazole-1-carboxylate (Int-6) in a manner analogous to the procedures described for Step 3 and Step 4 of Example 1. ¹H-NMR (300 MHz, CDCl₃): δ 8.77 (d, J=5.1 Hz, 1H), 8.36 (d, J=5.0 Hz, 1H), 7.98 (m, 1H), 7.54 (d, J=5.1 Hz, 1H), 7.49-7.43 (m, 3H), 4.29 (m, 1H), 2.27-2.13 (m, 1H), 2.10-1.94 (m, 2H), 1.70-1.51 (m, 2H), 1.49-1.35 (m, 1H). MS (ESI⁺): m/z: 416.24 (M+H)⁺.

### Example 95. (1s,4s)-4-((4-([1,2,4]Triazolo[4,3-a]pyridin-7-ylethynyl)-[2,4′-bipyrimidin]-2′-yl)amino)cyclohexan-1-ol (Ex. 95)

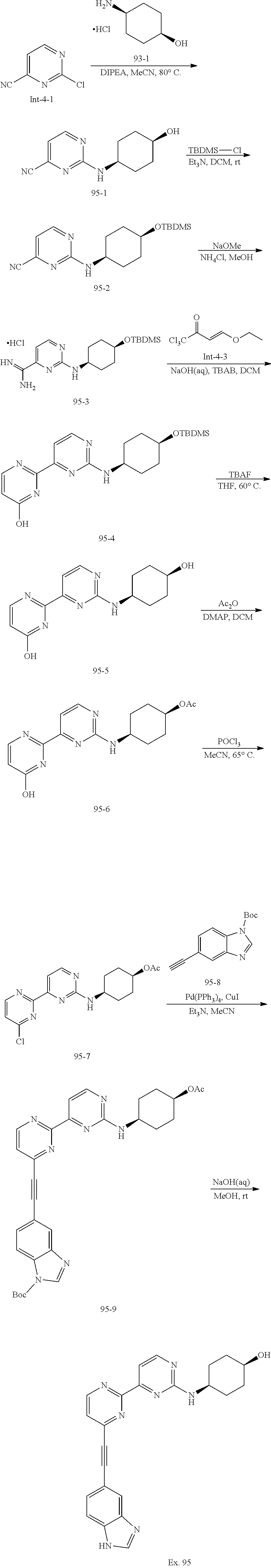

Step 1: 2-(((1s,4s)-4-Hydroxycyclohexyl)amino)pyrimidine-4-carbonitrile (95-1): To a solution of 2-chloropyrimidine-4-carbonitrile (Int-4-1, 8.88 g, 63.6 mmol) and (1s,4s)-4-aminocyclohexan-1-ol hydrochloride (93-1, 15.5 g, 63.6 mmol) in acetonitrile (400 mL) was added DIPEA (22.4 mL, 140 mmol). The reaction mixture was stirred at 80° C. for 4 h. The off-white solid thus formed was collected by filtration and the filtrate was evaporated to dryness to yield a brown solid. EtOAc (200 mL) was added to the solid and filtered to collect off-white solid. Filtrate was evaporated to dryness and suspended in acetone (200 mL). The solid was collected again by filtration. All three crops were combined to give 2-(((1s,4s)-4-hydroxycyclohexyl)amino)pyrimidine-4-carbonitrile (95-1, 13.2 g, yield: 95%). MS (ESI⁺): m/z: 219.6 (M+H)⁺.

Step 2: 2-(((1s,4s)-4-((tert-Butyldimethylsilyl)oxy)cyclohexyl)amino)pyrimidine-4-carbonitrile (95-2): 2-(((1s,4s)-4-Hydroxycyclohexyl)amino)pyrimidine-4-carbonitrile (95-1, 16.0 g, 73.3 mmol) and triethylamine (20.3 mL, 146.6 mmol) were dissolved in DCM (300 mL). tert-Butyldimethylsilyl chloride (TBDMSCl, 12.2 g, 80.6 mmol) was added slowely into the reaction mixture and the resulting mixture was stirred at room temperature for 12 h. The reaction mixture was washed with saturated sodium bicarbonate aqueous solution. The organic layers were collected, dried over sodium sulfate, filtered, and evaporated to dryness to give 2-(((1s,4s)-4-((tert-butyldimethylsilyl)oxy)cyclohexyl)amino)pyrimidine-4-carbonitrile (95-2, yield: quantitative). MS (ESI⁺): m/z: 333.6 (M+H)⁺.

Step 3: 2-(((1s,4s)-4-((tert-Butyldimethylsilyl)oxy)cyclohexyl)amino)pyrimidine-4-carboximidamide hydrochloride (95-3): To a solution of 2-(((1s,4s)-4-((tert-butyldimethylsilyl)oxy)cyclohexyl)amino)pyrimidine-4-carbonitrile (95-2, 24.2 g, 73.3 mmol) in MeOH (400 mL) was added NaOCH₃ (7.9 g, 146.6 mmol) at room temperature. The reaction mixture was stirred at room temperature for 4 h. Ammonium chloride (7.8 g, 146.6 mmol) was added to the reaction mixture and then the reaction was refluxed overnight. After cooled down to room temperature, the reaction mixture was concentrated in vacuo, the solid product was washed with methyl tert-butyl ether to give 2-(((1s,4s)-4-((tert-butyldimethylsilyl)oxy)cyclohexyl)amino)pyrimidine-4-carboximidamide hydrochloride as a white solid (95-3, 26.8 g, yield: 95%). MS (ESI⁺): m/z: 350.4 (M+H)⁺.

Step 4: 2′-(((1s,4s)-4-((tert-Butyldimethylsilyl)oxy)cyclohexyl)amino)-[2,4′-bipyrimidin]-4-ol (95-4): A solution of (E)-1,1,1-trichloro-4-ethoxybut-3-en-2-one (Int-4-3, 10.3 mL, 65 mmol) in DCM (500 mL) was added to a vigorously stirred mixture of 2-(((1s,4s)-4-((tert-butyldimethylsilyl)oxy)cyclohexyl)amino)pyrimidine-4-carboximidamide hydrochloride (95-3, 24.2 g, 63 mmol) in 2M solution of NaOH (aq, 200 mL) and tetrabutylammonium bromide (TBAB, cat. 0.27 g). The reaction mixture was stirred at room temperature overnight. The majority of solvents were removed to give a residue, which was collected by filtration after solidified. The solids were washed with water to afford 2′-(((1s,4s)-4-((tert-butyldimethylsilyl)oxy)cyclohexyl)amino)-[2,4′-bipyrimidin]-4-ol (95-4, 14 g, yield: 61%). MS (ESI⁺): m/z: 402.4 (M+H)⁺.

Step 5: 2′-(((1s,4s)-4-Hydroxycyclohexyl)amino)-[2,4′-bipyrimidin]-4-ol (95-5): 2′-(((1s,4s)-4-((tert-Butyldimethylsilyl)oxy)cyclohexyl)amino)-[2,4′-bipyrimidin]-4-ol (95-4, 14.0 g, 35 mmol) was dissolved in THE (200 mL). A 2.0-M solution of tetra-n-butylammonium fluoride (TBAF) in THE (26 mL, 52.5 mmol) was added slowly into the reaction mixture and the resulting mixture was stirred at 60° C. for 12 h. THE was evaporated and the crude mixture was treated with water and extracted with butanol. The organic layer was dried over sodium sulfate and evaporated to dryness to give 2′-(((1s,4s)-4-hydroxycyclohexyl)amino)-[2,4′-bipyrimidin]-4-ol (95-5, 8.5 g, yield: 85%). MS (ESI⁺): m/z: 288.4 (M+H)⁺.

Step 6: (1s,4s)-4-((4-Hydroxy-[2,4′-bipyrimidin]-2′-yl)amino)cyclohexyl acetate (95-6): 2′-(((1s,4s)-4-Hydroxycyclohexyl)amino)-[2,4′-bipyrimidin]-4-ol (95-5, 8.5 g, 29.6 mmol) and DMAP (0.4 g, 2.96 mmol) were dissolved in DCM and the solution was cooled to 0° C. Acetic anhydride (4.2 mL, 44.4 mmol) was added and the resulting mixture was stirred at room temperature for 5 h. The reaction mixture was washed with sodium bicarbonate solution (200 mL). The organic layer was separated, dried over sodium sulfate, filtered, and evaporated to dryness to give (1s,4s)-4-((4-hydroxy-[2,4′-bipyrimidin]-2′-yl)amino)cyclohexyl acetate (95-6, 8.3 g, yield: 85%). MS (ESI⁺): m/z: 330.2 (M+H)⁺.

Step 7: (1s,4s)-4-((4-Chloro-[2,4′-bipyrimidin]-2′-yl)amino)cyclohexyl acetate (95-7): (1s,4s)-4-((4-Hydroxy-[2,4′-bipyrimidin]-2′-yl)amino)cyclohexyl acetate (95-6, 8.3 g, 25.3 mmol) was dissolved in acetonitrile (150 mL). POCl₃ (7.0 mL, 75.7 mmol) was added dropwise in to the reaction. The resulting mixture was stirred at 65° C. for 1 h. Excess reagent and solvent were evaporated. The crude mixture was poured into ice and treated with sodium bicarbonate, and extracted with DCM (200 mL). The organic layer was dried over sodium sulfate and evaporated to dryness to give (1s,4s)-4-((4-chloro-[2,4′-bipyrimidin]-2′-yl)amino)cyclohexyl acetate (95-7, 6.7 g, yield: 76%). MS (ESI⁺): m/z: 348.6 (M+H)⁺.

Step 8: tert-Butyl 5-((2′-(((1s,4s)-4-acetoxycyclohexyl)amino)-[2,4′-bipyrimidin]-4-yl)ethynyl)-1H-benzo[d]imidazole-1-carboxylate (95-9): Compound 95-9 was prepared from 95-7 and 95-8 in a manner analogous to the procedures described for the synthesis of compound Int-5. Compound 95-9 was obtained as a brown solid (yield: 62%). MS (ESI⁺): m/z: 554.3 (M+H)⁺.

Step 9: (1s,4s)-4-((4-((1H-Benzo[d]imidazol-5-yl)ethynyl)-[2,4′-bipyrimidin]-2′-yl)amino)cyclohexanol (Ex. 95): tert-Butyl 5-((2′-(((1s,4s)-4-acetoxycyclohexyl)amino)-[2,4′-bipyrimidin]-4-yl)ethynyl)-1H-benzo[d]imidazole-1-carboxylate (95-9, 100 mg, 0.175 mmol) was dissolved in MeOH (5 mL). Aqueous NaOH (10%, 5 mL) was added and the reaction mixture was stirred at room temperature for 2 h. Solvent was evaporated and the crude mixture was extracted with DCM (20 mL). The organic layer was dried over sodium sulfate, concentrated in vacuo, and the crude material was purified using column chromatography over silica gel (3-5% MeOH in DCM) to give (1s,4s)-4-((4-((1H-benzo[d]imidazol-5-yl)ethynyl)-[2,4′-bipyrimidin]-2′-yl)amino)cyclohexan-1-ol as a light yellow solid (Ex. 95, 50 mg, yield: 70%). ¹H-NMR (300 MHz, CD₃OD-CDCl₃): δ=8.84 (d, J=5.4 Hz, 1H), 8.46 (d, J=5.4 Hz, 1H), 8.06 (s, 1H), 7.92 (s, 1H), 7.63-7.57 (m, 3H), 7.37 (d, J=8.4 Hz, 1H), 3.97-3.82 (m, 2H), 1.78-1.72 (m, 8H). MS (ESI⁺): m/z: 412.2 (M+H)⁺.

### Example 96. (1s,4s)-4-((4-(Imidazo[1,5-a]pyridin-6-ylethynyl)-[2,4′-bipyrimidin]-2′-yl)amino)cyclohexanol (Ex. 96)

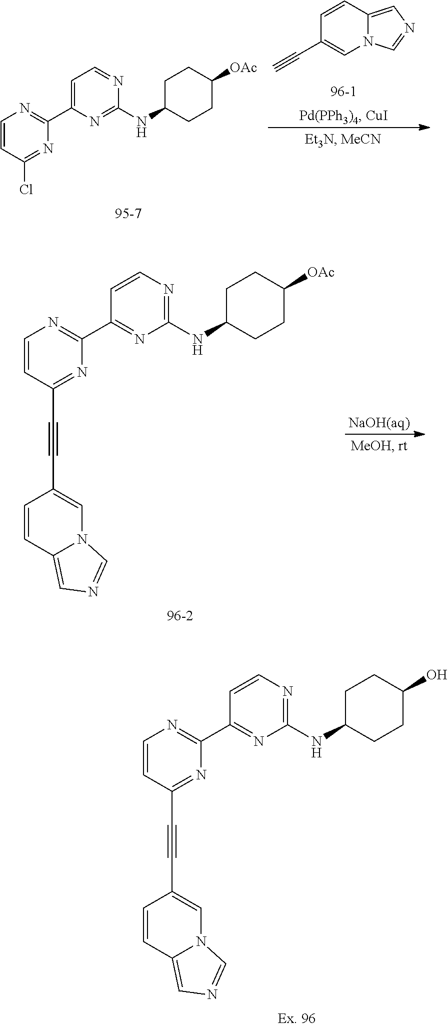

Ex. 96 was prepared from (1s,4s)-4-((4-chloro-[2,4′-bipyrimidin]-2′-yl)amino)cyclohexyl acetate (95-7) and compound 96-1 in a manner analogous to the procedures described for Step 8 and Step 9 of Example 95. Ex. 96 was obtained as a brown solid. ¹H NMR (300 MHz, CD₃OD) δ 8.96 (d, J=5.1 Hz, 1H), 8.71 (s, 1H), 8.49 (d, J=5.1 Hz, 1H), 8.42 (s, 1H), 7.68 (d, J=5.1 Hz, 1H), 7.61-7.58 (m, 2H), 7.44 (s, 1H), 6.93 (m, 1H), 3.99 (bs, 1H), 3.85 (m, 1H), 1.81-1.71 (m, 9H). MS (ESI⁺): m/z: 412.23 (M+H)⁺.

### Example 97. (1s,4s)-4-((4-([1,2,4]Triazolo[4,3-a]pyridin-7-ylethynyl)-[2,4′-bipyrimidin]-2′-yl)amino)cyclohexanol (Ex. 97)

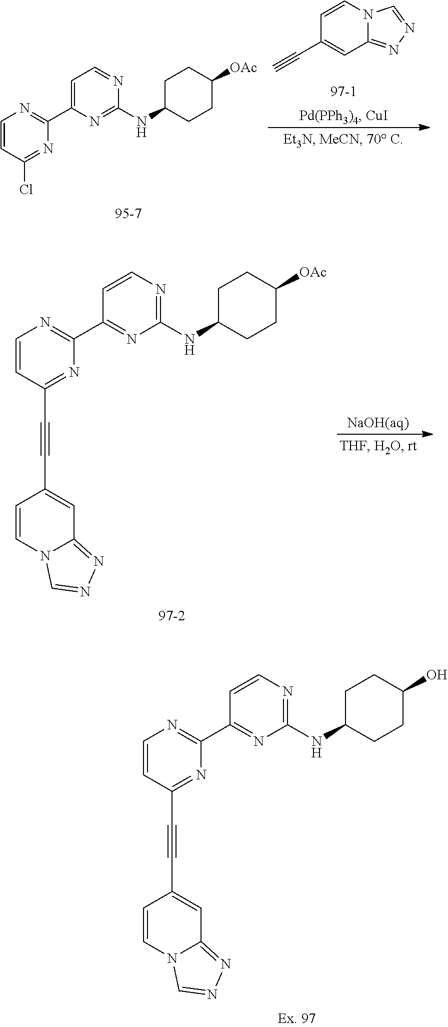

Step 1: (1s,4s)-4-((4-([1,2,4]Triazolo[4,3-a]pyridin-7-ylethynyl)-[2,4′-bipyrimidin]-2′-yl)amino)cyclohexyl acetate (97-2): A mixture of (1s,4s)-4-((4-chloro-[2,4′-bipyrimidin]-2′-yl)amino)cyclohexyl acetate (95-7, 255 mg, 0.733 mmol), 7-ethynyl-[1,2,4]triazolo[4,3-a]pyridine (97-1, 126 mg, 0.880 mmol, prepared in a manner analogous to the procedures described for the synthesis of Int-1), CuI (13.9 mg, 0.0733 mmol), and Pd(PPh₃)₄ (169 mg, 0.0147 mmol) in Et₃N (0.51 mL) and MeCN (8 mL) was purged with nitrogen gas at room temperature. The reaction mixture was stirred at 70° C. for 17 h. LC-MS indicated the reaction was complete. The reaction mixture was cooled to room temperature, concentrated in vacuo, and the crude product was purified by column chromatography over silica gel to afford (1s,4s)-4-((4-([1,2,4]triazolo[4,3-a]pyridin-7-ylethynyl)-[2,4′-bipyrimidin]-2′-yl)amino)cyclohexyl acetate as a yellow solid (97-2, yield: 54%). MS (ESI⁺): m/z: 455.4 (M+H)⁺.

Step 2: (1s,4s)-4-((4-([1,2,4]Triazolo[4,3-a]pyridin-7-ylethynyl)-[2,4′-bipyrimidin]-2′-yl)amino)cyclohexan-1-ol (Ex. 97): (1s,4s)-4-((4-([1,2,4]triazolo[4,3-a]pyridin-7-ylethynyl)-[2,4′-bipyrimidin]-2′-yl)amino)cyclohexyl acetate (97-2, 58.4 mg, 0.129 mmol) was suspended in THE (3.5 mL) and water (1.5 mL). NaOH (15.5 mg, 0.387 mmol) was added and the reaction mixture was stirred at room temperature for 5 h. The reaction mixture was concentrated under reduced pressure, the residue was purified by column chromatography over silica gel to afford (1s,4s)-4-((4-([1,2,4]triazolo[4,3-a]pyridin-7-ylethynyl)-[2,4′-bipyrimidin]-2′-yl)amino)cyclohexan-1-ol as a yellow solid (Ex. 97, 53.2 mg, yield: 60%). ¹H-NMR (300 MHz, CD₃OD-CDCl₃): δ=9.16 (d, J=0.8 Hz, 1H), 8.98 (d, J=5.1 Hz, 1H), 8.50-8.46 (m, 2H), 8.08 (d, J=1.0 Hz, 1H), 7.68 (d, J=5.0 Hz, 1H), 7.61 (d, J=5.0 Hz, 1H), 7.12 (dd, J=7.1 Hz, 1.4 Hz, 1H), 3.98 (m, 1H), 3.84 (m, 1H), 1.89-1.67 (m, 8H). MS (ESI⁺): m/z: 413.4 (M+H)⁺.

The foregoing are merely exemplary of synthetic routes to the compound of the disclosure. The foregoing compounds, compositions and methods of the disclosure are merely exemplary of aspects of the disclosure and are not limiting.

2) In Vitro Biological Activity:

1. ROCK1 and ROCK2 kinase assays: The ROCK1 and ROCK2 kinase binding affinities of compounds in this disclosure were determined by DiscoverX's KINOMEscan™ KdELECT technology (https://www.discoverx.com/kinomescan-elect-kinase-screening-and-profiling-services): Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce nonspecific binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 111× stocks in 100% DMSO. Kds were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 ml. The assay plates were incubated at room temperature with shaking for 1 hour and the affinity beads were washed with wash buffer (1×PBS, 0.05% Tween 20). The beads were then re-suspended in elution buffer (lx PBS, 0.05% Tween 20, 0.5 M non-biotinylated affinity ligand) and incubated at room temperature with shaking for 30 minutes. The kinase concentration in the eluates was measured by qPCR.

Results are presented in Table 4. Compounds having an activity designated as “A” provided a Kd of <0.01 μM; compounds having an activity designated as “B” provided a Kd of 0.01-0.099 μM; compounds having an activity designated as “C” provided a Kd of 0.1-0.99 μM; compounds having an activity designated as “D” provided a Kd of 1-9.9 μM; compounds having an activity designated as “E” provided a Kd of 10-100 μM. ROCK2 selectivity is indicated as follows: compounds which exhibited <50-fold selectivity for ROCK2 are designated *; compounds which exhibited 50 to 150-fold selectivity for ROCK2 are designated **; compounds which exhibited 151 to 500-fold selectivity for ROCK2 are designated ***; compounds which exhibited >500-fold selectivity for ROCK2 are designated ****.

TABLE 4
ROCK1 and ROCK2 Binding Affinities.

| | Kd (μM) | | ROCK2 |
|---|---|---|---|
| Compound | ROCK1 | ROCK2 | Selectivity |
| KD025 | E | C | ** |
| REDX10843 | D | B | ** |
| REDX10842 | D | A | *** |
| Compound A | E | C | *** |
| Ex. 1 | C | A | ** |
| Ex. 2 | C | A | *** |
| Ex. 3 | D | A | *** |
| Ex. 4 | D | C | * |
| Ex. 5 | C | B | * |
| Ex. 6 | B | A | * |
| Ex. 7 | C | B | * |
| Ex. 8 | D | B | *** |
| Ex. 9 | D | C | * |
| Ex. 10 | D | B | ** |
| Ex. 11 | C | B | ** |
| Ex. 12 | D | B | *** |
| Ex. 13 | E | C | ** |
| Ex. 14 | D | B | * |
| Ex. 15 | E | B | *** |
| Ex. 16 | C | A | * |
| Ex. 17 | C | A | *** |
| Ex. 18 | D | B | ** |
| Ex. 19 | C | B | ** |
| Ex. 20 | C | B | * |
| Ex. 21 | C | B | ** |
| Ex. 22 | D | B | ** |
| Ex. 23 | D | B | ** |
| Ex. 24 | C | A | ** |
| Ex. 25 | C | B | * |
| Ex. 26 | D | B | ** |
| Ex. 27 | E | C | ** |
| Ex. 28 | D | B | * |
| Ex. 29 | D | C | * |
| Ex. 30 | D | C | * |
| Ex. 31 | D | B | ** |
| Ex. 32 | D | B | ** |
| Ex. 33 | D | A | *** |
| Ex. 34 | C | A | *** |
| Ex. 35 | C | A | *** |
| Ex. 36 | D | B | ** |
| Ex. 37 | D | B | *** |
| Ex. 38 | C | A | ** |
| Ex. 39 | D | B | ** |
| Ex. 40 | C | B | * |
| Ex. 41 | D | A | *** |
| Ex. 42 | D | B | ** |
| Ex. 43 | E | B | *** |
| Ex. 44 | E | B | *** |
| Ex. 45 | E | B | **** |
| Ex. 46 | D | B | ** |
| Ex. 47 | D | B | ** |
| Ex. 48 | C | A | ** |
| Ex. 49 | D | A | *** |
| Ex. 50 | D | A | *** |
| Ex. 51 | C | A | *** |
| Ex. 52 | C | A | ** |
| Ex. 53 | D | B | ** |
| Ex. 54 | D | C | * |
| Ex. 55 | D | B | ** |
| Ex. 56 | D | A | **** |
| Ex. 57 | E | B | **** |
| Ex. 58 | D | A | **** |
| Ex. 59 | D | A | *** |
| Ex. 60 | E | D | * |
| Ex. 61 | E | B | *** |
| Ex. 62 | D | A | *** |
| Ex. 63 | D | A | *** |
| Ex. 64 | C | B | * |
| Ex. 65 | C | A | **** |
| Ex. 66 | C | A | ** |
| Ex. 67 | D | B | * |
| Ex. 68 | C | A | *** |
| Ex. 69 | D | A | *** |
| Ex. 70 | E | B | **** |
| Ex. 71 | C | C | * |
| Ex. 72 | E | E | * |
| Ex. 73 | C | B | * |
| Ex. 74 | C | A | **** |
| Ex. 75 | D | A | *** |
| Ex. 76 | D | A | *** |
| Ex. 77 | E | B | *** |
| Ex. 78 | D | A | *** |
| Ex. 79 | D | B | ** |
| Ex. 80 | D | C | ** |
| Ex. 81 | D | B | ** |
| Ex. 82 | C | A | **** |
| Ex. 83 | C | A | *** |
| Ex. 84 | D | B | * |
| Ex. 85 | D | A | *** |
| Ex. 86 | C | A | *** |
| Ex. 87 | D | C | * |
| Ex. 88 | C | B | * |
| Ex. 89 | D | C | * |
| Ex. 90 | D | A | *** |
| Ex. 91 | C | A | ** |
| Ex. 92 | C | B | * |
| Ex. 93 | D | A | **** |
| Ex. 94 | D | A | **** |
| Ex. 95 | E | B | *** |
| Ex. 96 | E | B | ** |
| Ex. 97 | E | E | * |

The data in Table 4 show compounds of this disclosure bind to both ROCK1 and ROCK2, especially the latter.

2. Gel Contraction Assay:

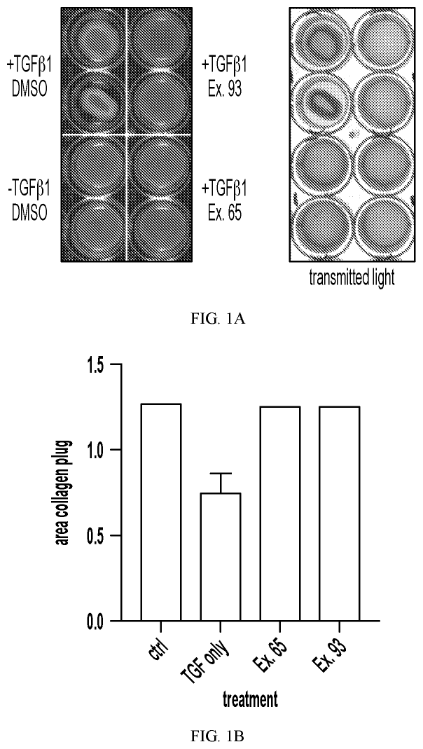

Hepatic stellate cells were plated onto collagen coated plates and pretreated with test compound (at 100 μM) for 1 hour. TGFβ1 is then added for 24 hours, and images are taken. Gel contraction images are shown in FIG. 1A) with quantification shown in FIG. 11B.

FIG. 1A and FIG. 1B demonstrate that TGFβ1-induced gel contraction in hepatic stellate cells was significantly attenuated in the presence of Ex. 65 or Ex. 93.

3. TGFβ1-Induced CTGF Production Assay:

Mouse embryonic fibroblasts (MEFs) were isolated, starved overnight, then pretreated with Ex. 65 or Ex. 93 for 1 hour prior to addition of TGFβ1. RNA was isolated from cells and real time PCR was performed for CTGF.

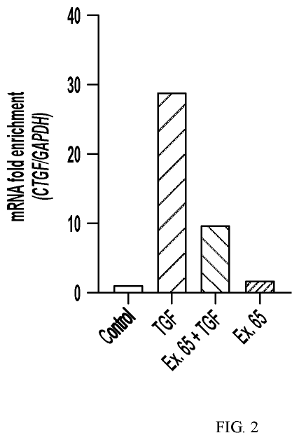

FIG. 2 demonstrates that CTGF production in embryonic fibroblasts was attenuated in the presence of Ex. 65.

3) In Vivo Biological Activity:

1. Pharmacokinetic Profile:

For assessing compound levels in the vena cava, 10-11 week old male C57BL/6 animals were dosed orally with 10 mg/kg of Ex. 65 (mesylate salt) in 0.5% carboxymethyl cellulose (CMC) in water. Serum samples were collected from the portal vein at 15, 30, 60 120, 240, 360 and 480 minutes after dosing. Samples were analyzed for compound concentration by LC-MS.

For assessing compound levels in the portal vein, 7-8 week old male C57BL/6 animals were dosed orally with 10 mg/kg compound of Ex. 65 (mesylate salt) in 0.5% carboxymethyl cellulose (CMC) in water. Serum samples were collected from the portal vein at 15, 30, 60 120 and 240 minutes after dosing. Samples were analyzed for compound by LC-MS.

TABLE 5
Pharmacokinetic Profile of Ex. 65.
C57BL/6 Mouse (po)
10 mg/kg in 0.5% CMC suspension

| In portal vein | | | In vena cava | | |
|---|---|---|---|---|---|
| | AUC | | | AUC | |
| Cₘₐₓ | (ng · hr/ | F | Cₘₐₓ | (ng · hr/ | F |
| (nM) | mL) | % | (nM) | mL) | % |
| 1543 | 697 | 22% | 258 | 145 | 4.6% |
| 1904*Kd | | | 318*Kd | | |

Table 5 demonstrates oral bioavailability of Ex. 65 with preferential liver entry in mice.

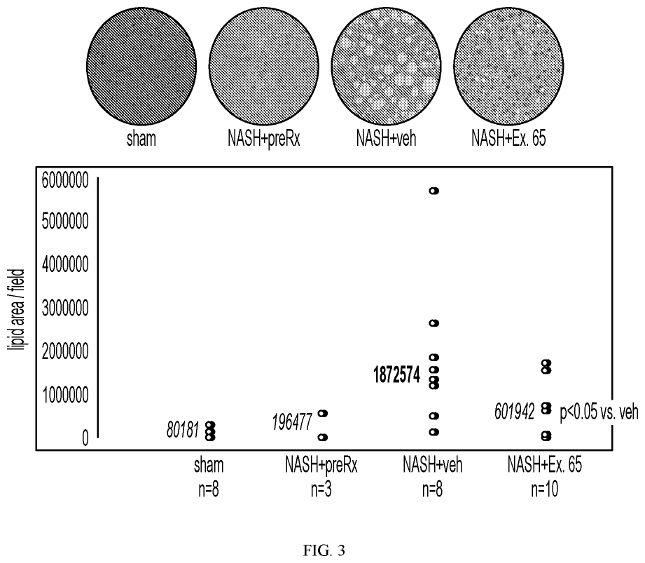

2. Pharmacodynamic and Efficacy Profile in Hepatic Diseases:
The activity of Ex. 65 was evaluated in a model of fast-food diet (FFD)+CCl4+sugar-induced NASH. Adult male mice were randomized to vehicle of 30 mg/kg test compound, IP, twice-daily for two weeks. Treatment with Ex. 65 reduced hepatic lipid accumulation (FIG. 3) and NAFLD activity score (NAS; FIG. 4).

MicroRNAs (miRNAs) from liver homogenates from the study described above were quantified. Compared to healthy livers, 65 differentially expressed miRNAs from NASH livers were identified, of which 22 were corrected by treatment with Ex. 65 (see Table 6). Table 6 also demonstrates that treatment with Ex. 65 reduced hepatic levels of oncomiR-425 and oncomiR-181a, which are involved in sorafenib resistance to HCC.

TABLE 6
Analysis of Differentially Expressed miRNAs in Hepatic Homogenates

| | | | | | Mean | SD |
|---|---|---|---|---|---|---|
| | Mean | SD | Mean | SD | (disease + | (disease + |
| miRNA | (control) | (control) | (disease) | (disease) | Ex. 65) | Ex. 65) |
| mmu-miR-99b-5p | 1421.33 | 293.76 | 1933.41 | 810.63 | 1099.61 | 323.63 |
| mmu-miR-125a-3p | 245.12 | 47.32 | 297.02 | 81.59 | 182.06 | 40.83 |
| mmu-miR-181a-5p | 3789.12 | 629.52 | 5124.26 | 1858.40 | 2941.29 | 1264.68 |
| mmu-miR-191-5p | 118228.91 | 22636.98 | 141957.68 | 19103.26 | 119365.71 | 24705.69 |
| mmu-miR-143-3p | 67983.70 | 14981.36 | 80209.00 | 19532.76 | 58204.03 | 29062.86 |
| mmu-miR-106a-5p | 115.29 | 25.85 | 149.51 | 39.18 | 114.11 | 13.91 |
| mmu-miR-27a-5p | 71.79 | 26.00 | 98.65 | 40.80 | 53.13 | 40.48 |
| mmu-miR-93-5p | 36188.01 | 3282.71 | 41626.66 | 7848.37 | 31184.61 | 3384.08 |
| mmu-miR-93-3p | 39.45 | 9.94 | 76.50 | 19.45 | 46.52 | 18.50 |
| mmu-miR-322-3p | 1115.59 | 239.04 | 1416.47 | 357.43 | 949.59 | 126.30 |
| mmu-miR-339-3p | 427.46 | 42.68 | 502.07 | 79.62 | 426.25 | 22.33 |
| mmu-miR-351-5p | 1248.83 | 104.60 | 1603.84 | 435.97 | 889.81 | 126.95 |
| mmu-miR-17-3p | 1200.81 | 202.76 | 1454.34 | 256.80 | 1161.55 | 46.23 |
| mmu-miR-320-3p | 4080.44 | 976.93 | 5204.90 | 1140.67 | 3896.99 | 417.76 |
| mmu-miR-425-5p | 1716.15 | 253.87 | 2037.69 | 385.43 | 1524.04 | 158.53 |
| mmu-miR-542-5p | 66.04 | 18.41 | 74.42 | 22.31 | 39.03 | 18.35 |
| mmu-miR-802-5p | 1094.11 | 499.92 | 614.54 | 255.31 | 899.47 | 375.73 |
| mmu-miR-501-3p | 364.92 | 68.22 | 489.94 | 153.77 | 296.78 | 88.11 |
| mmu-miR-871-3p | 4.08 | 2.31 | 1.53 | 1.31 | 7.98 | 6.91 |
| mmu-miR-5123 | 3.22 | 2.14 | 1.53 | 1.40 | 3.54 | 1.91 |
| mmu-let-7k | 9.04 | 5.16 | 13.85 | 4.32 | 7.72 | 3.36 |
| mmu-miR-7115-5p | 4.31 | 3.19 | 8.54 | 2.89 | 2.41 | 2.09 |

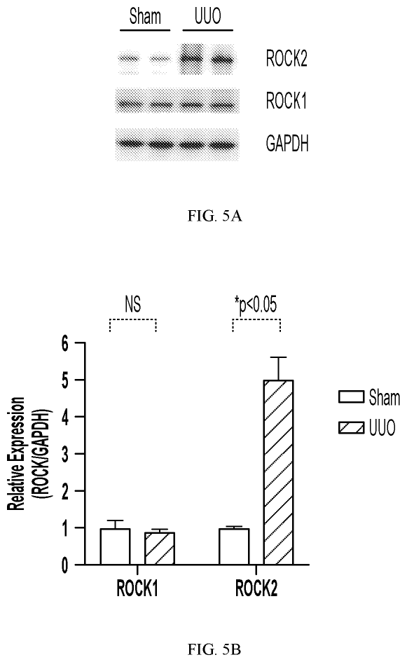

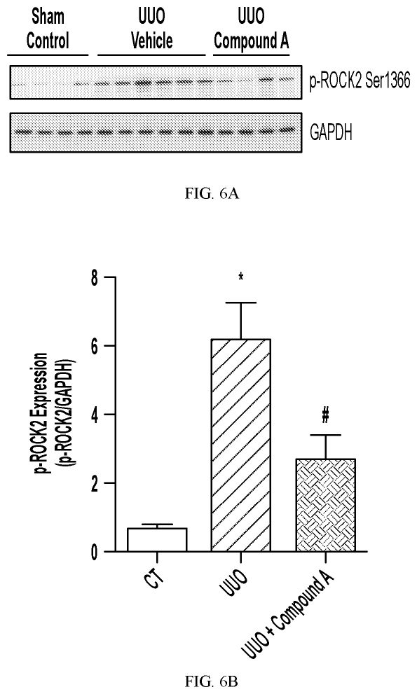

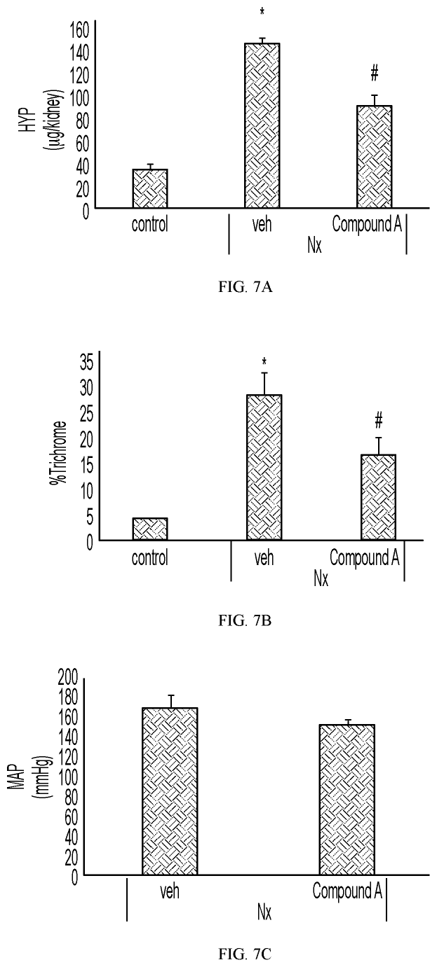

3. Pharmacodynamic and efficacy profile in renal diseases: The initial proof-of-concept studies were conducted with Compound A, a previous generation ROCK2 inhibitor disclosed in WO 2019/046795. Further studies will be conducted with compounds of the present disclosure. In adult male C57BL/6 mice submitted to unilateral ureteral obstruction (UUO), renal ROCK2 (but not ROCK1) expression (Western blot) was increased (7 days after UUO, diseased kidney) relative to the sham cohort (FIG. 5A and FIG. 5B). To determine the pharmacodynamic effects of Compound A, mice were dosed with Compound A (25 mg/kg, PO, BID) on day 7 after UUO. Administration of Compound A was associated with reduced phosphorylation of renal ROCK2 (diseased kidney, FIG. 6A and FIG. 6B). In SV129 mice subjected to subtotal nephrectomy, animals were randomized to vehicle or Compound A (25 mg/kg, PO, BID) from days 14 to 60 at which point mice were sacrificed. Treatment with Compound A was associated with a reduction in fibrosis (kidney hydroxyproline and Masson's trichrome, as shown in FIG. 7A and FIG. 7B, respectively) with no change in MAP, as shown in FIG. 7C.

1. A compound of Formula I: 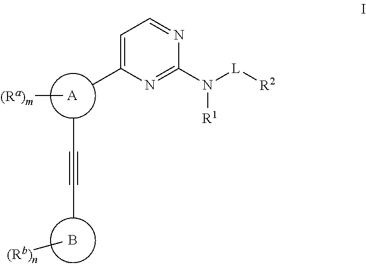
or a pharmaceutically acceptable salt thereof, wherein:
Ring A is selected from phenyl and a 6-membered heteroaryl ring comprising 1-3 nitrogen atoms;
Ring B is selected from phenyl, a 5- to 6-membered heteroaryl ring comprising 1-3 heteroatoms independently selected from nitrogen, oxygen, and sulfur, and a 9- to 10-membered heteroaryl ring comprising 1-4 heteroatoms independently selected from nitrogen, oxygen, and sulfur;
each R^{a} is independently selected from halogen, CN, CO₂R, C(O)NR₂, NR₂, OR, SR, and optionally substituted C₁₋₆ aliphatic;
each R^{b} is independently selected from halogen, CN, CO₂R, C(O)NR₂, NR₂, OR, SR, oxo and optionally substituted C₁₋₆ aliphatic;
R¹ is hydrogen or optionally substituted C₁₋₆ aliphatic;
L is a covalent bond or a bivalent C₁₋₆ straight or branched hydrocarbon chain;
R² is 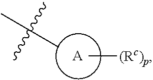
C(O)NR₂, NR₂, OR, or S(═O)ₓR;
Ring C is selected from a 3- to 7-membered cycloaliphatic ring, phenyl, a 3- to 7-membered heterocyclic ring comprising 1-3 heteroatoms independently selected from nitrogen, oxygen, and sulfur, a 5- to 6-membered heteroaryl ring comprising 1-4 heteroatoms independently selected from nitrogen, oxygen, and sulfur, and a 9- to 10-membered heteroaryl ring comprising 1-5 heteroatoms independently selected from nitrogen, oxygen, and sulfur
each R^{c} is independently selected from halogen, oxo, OR, CO₂R, C(O)N(R)₂, and optionally substituted C₁₋₆ aliphatic, or
two independent occurrences of R^{c}, taken together with their intervening atom(s), form an optionally substituted 5-to 8-membered heterocyclic ring comprising 1-4 heteroatoms independently selected from nitrogen, oxygen, and sulfur;
each R is independently selected from hydrogen and an optionally substituted group selected from C₁₋₆ aliphatic, phenyl, a 7- to 9-membered bridged bicyclic cycloaliphatic ring, and a 3- to 7-membered heterocyclic ring comprising 1-3 heteroatoms independently selected from nitrogen, oxygen, and sulfur; or:
two independent occurrences of R, taken together with the nitrogen atom to which they are attached, form an optionally substituted 3- to 7-membered heterocyclic ring comprising 0-3 additional heteroatoms independently selected from nitrogen, oxygen, and sulfur;
x is 0, 1, or 2; and
each of m, n, and p is independently 0-4.

2. The compound according to claim 1, wherein the compound is of formula I-a: 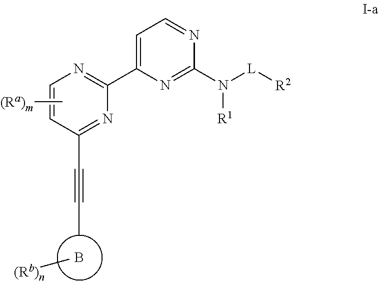
or a pharmaceutically acceptable salt thereof.

3. The compound according to claim 1 or 2, wherein the compound is of formula I-b: 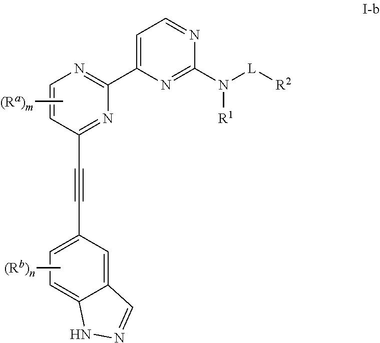
or a pharmaceutically acceptable salt thereof.

4. The compound according to any one of claims 1-3, wherein the compound is of formula I-c: 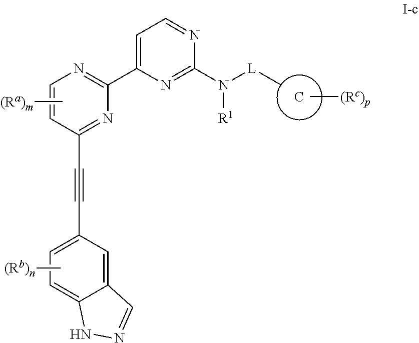
or a pharmaceutically acceptable salt thereof.

5. The compound according to any one of claims 1-3, wherein the compound is of formula I-d: 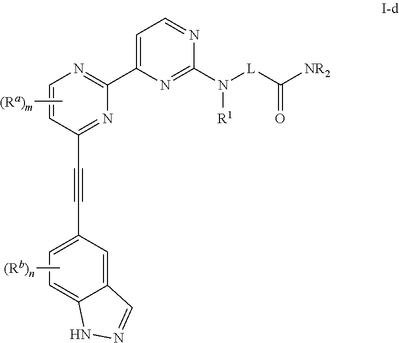
or a pharmaceutically acceptable salt thereof.

6. The compound according to claim 1, wherein Ring A is a 6-membered heteroaryl ring comprising 1-3 nitrogen atoms.

7. The compound according to claim 6, wherein Ring A is pyrimidinyl.

8. The compound according to claim 6 or 7, wherein Ring A is

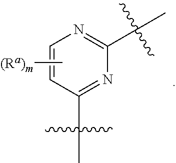

9. The compound according to claim 1 or 2, wherein Ring B is a 9- to 10-membered heteroaryl ring having 1-4 heteroatoms independently selected from nitrogen, oxygen, and sulfur.

10. The compound according to claim 9, wherein Ring B is indazolyl.

11. The compound according to claim 9 or 10, wherein Ring B is

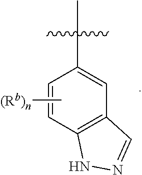

12. The compound according to any one of the preceding claims, each R^{b} is independently selected from halogen and optionally substituted C₁₋₆ aliphatic.

13. The compound according to claim 12, each R^{b} is halogen.

14. The compound according to claim 12 or 13, each R^{b} is fluoro.

15. The compound according to any one of the preceding claims, wherein R¹ is hydrogen.

16. The compound according to any one of claims 1-14, wherein R¹ is optionally substituted C₁₋₆ aliphatic.

17. The compound according to any one of the preceding claims, wherein L is a covalent bond.

18. The compound according to any one of claims 1-16, wherein L is a bivalent C₁₋₆ straight or branched hydrocarbon chain.

19. The compound according to claim 18, wherein L is —CH₂—.

20. The compound according to any one of claims 1-3 and 6-19, wherein R² is 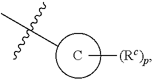
wherein Ring C is selected from a 3- to 7-membered cycloaliphatic ring, phenyl, a 3- to 7-membered heterocyclic ring comprising 1-3 heteroatoms independently selected from nitrogen, oxygen, and sulfur, a 5- to 6-membered heteroaryl ring comprising 1-4 heteroatoms independently selected from nitrogen, oxygen, and sulfur, and a 9- to 10-membered heteroaryl ring comprising 1-5 heteroatoms independently selected from nitrogen, oxygen, and sulfur.

21. The compound according to claim 20, wherein Ring C is a 3- to 7-membered cycloaliphatic ring.

22. The compound according to claim 21, wherein Ring C is cyclopentyl.

23. The compound according to claim 21 or 22, wherein Ring C is selected from

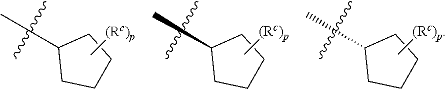

24. The compound according to claim 21, wherein Ring C is cyclohexyl.

25. The compound according to claim 21 or 24, wherein Ring C is selected from

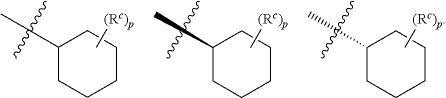

26. The compound according to claim 20, wherein Ring C is phenyl.

27. The compound according to claim 20, wherein Ring C is a 3- to 7-membered heterocyclic ring comprising 1-3 heteroatoms independently selected from nitrogen, oxygen, and sulfur.

28. The compound according to claim 27, wherein Ring C is a 5-membered heterocyclic ring comprising 1 heteroatom selected from nitrogen, oxygen, and sulfur.

29. The compound according to claim 27 or 28, wherein Ring C is tetrahydrofuranyl.

30. The compound according to any one of claims 27-29, wherein Ring C is selected from

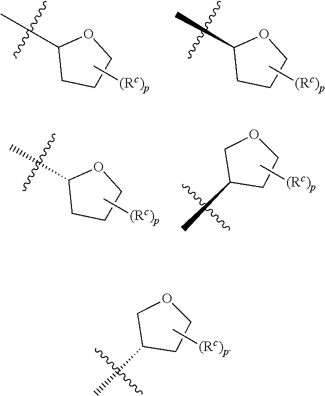

31. The compound according to claim 20, wherein Ring C is a 5- to 6-membered heteroaryl ring comprising 1-3 heteroatoms independently selected from nitrogen, oxygen, and sulfur.

32. The compound according to claim 20, wherein Ring C is a 9- to 10-membered heteroaryl ring comprising 1-5 heteroatoms independently selected from nitrogen, oxygen, and sulfur.

33. The compound according to any one of claims 1-3 and 6-19, wherein R² is C(O)NR₂,
wherein each R is independently selected from hydrogen or an optionally substituted group selected from C₁₋₆ aliphatic and a 7- to 9-membered bridged bicyclic cycloaliphatic ring, or two occurrences of R, taken together with the nitrogen atom to which they are attached, form an optionally substituted 3- to 7-membered heterocyclic ring comprising 0-3 additional heteroatoms independently selected from nitrogen, oxygen, and sulfur.

34. The compound according to any one of claims 1-3 and 6-19, wherein R² is selected from —OCH₃, —OH, —NH₂,

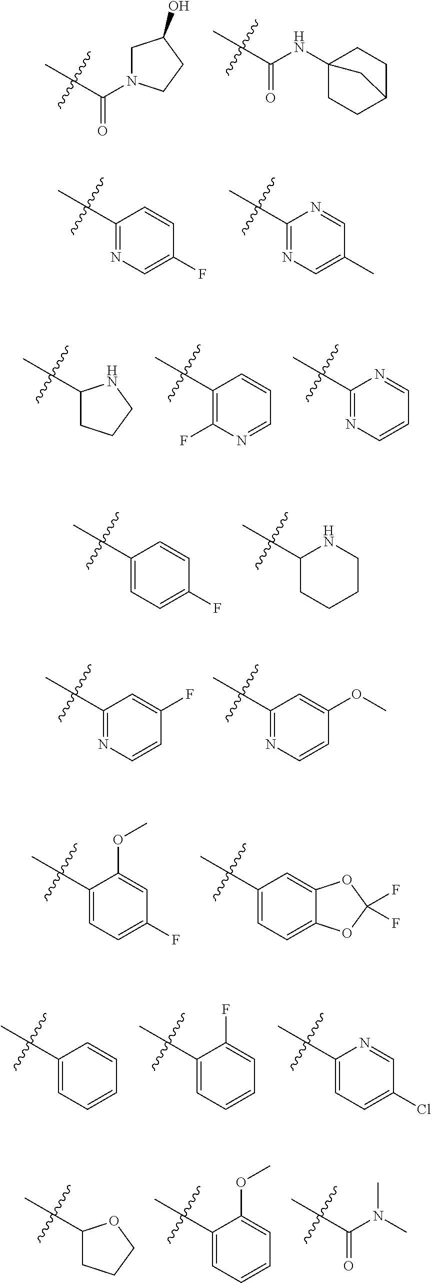

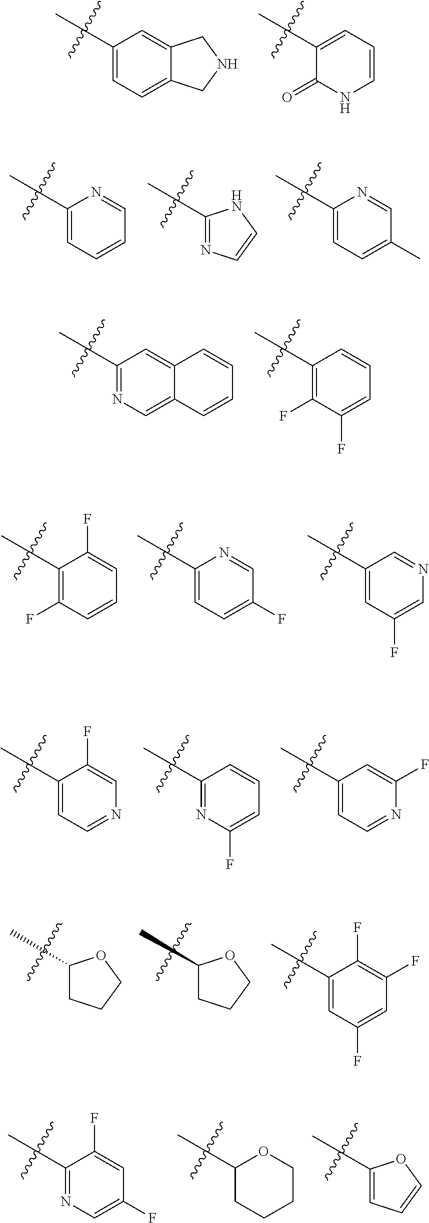

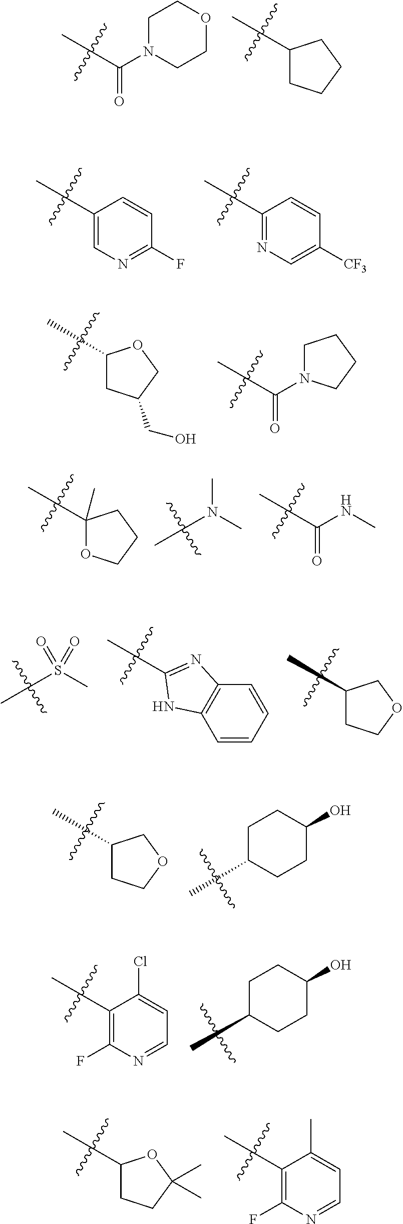

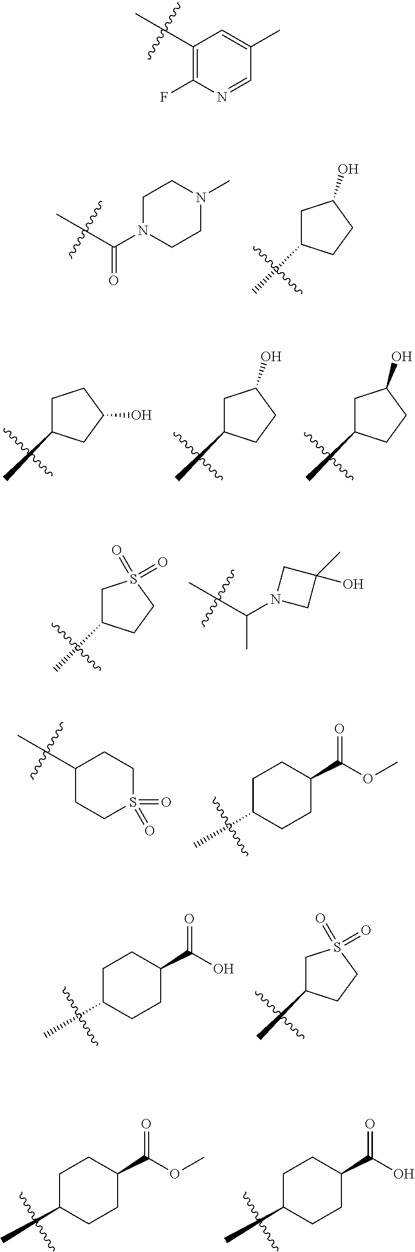

35. The compound according to any one of claims 1-3, wherein the compound is selected from the group consisting of 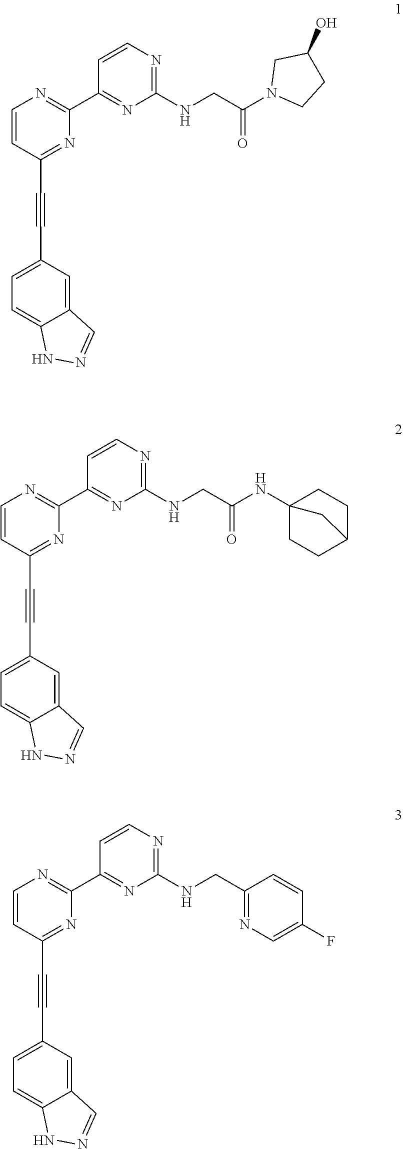 [SMILES: O=C(CNc1nccc(-c2nccc(C#Cc3ccc4[nH]ncc4c3)n2)n1)N1CC[C@H](O)C1 ; O=C(CNc1nccc(-c2nccc(C#Cc3ccc4[nH]ncc4c3)n2)n1)NC12CCC(CC1)C2 ; Fc1ccc(CNc2nccc(-c3nccc(C#Cc4ccc5[nH]ncc5c4)n3)n2)nc1] 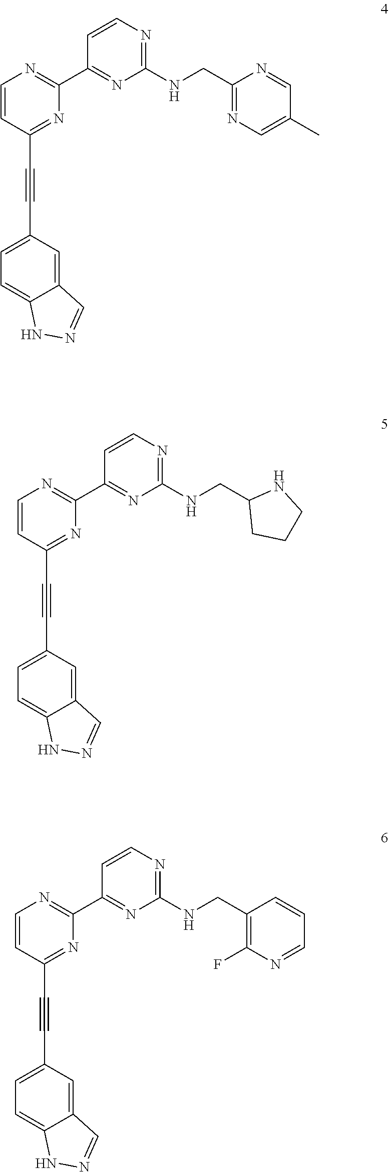 [SMILES: Cc1cnc(CNc2nccc(-c3nccc(C#Cc4ccc5[nH]ncc5c4)n3)n2)nc1 ; C(#Cc1ccnc(-c2ccnc(NCC3CCCN3)n2)n1)c1ccc2[nH]ncc2c1 ; Fc1ncccc1CNc1nccc(-c2nccc(C#Cc3ccc4[nH]ncc4c3)n2)n1] 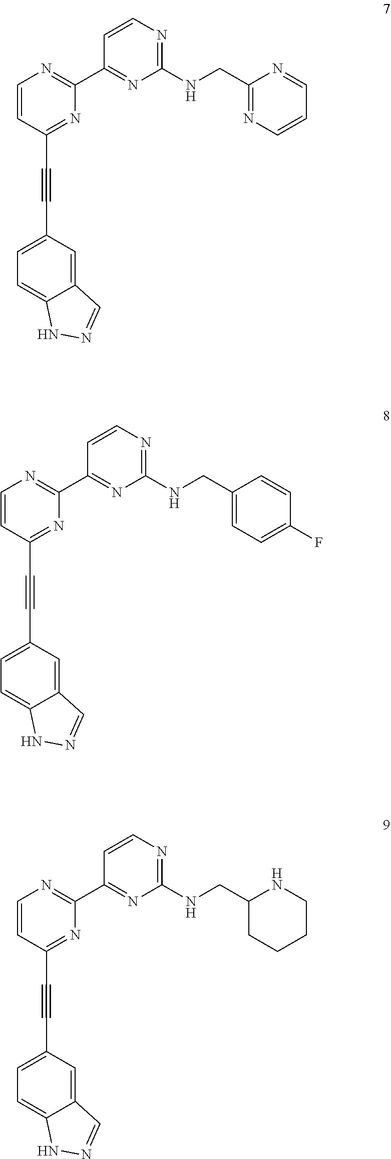 [SMILES: C(#Cc1ccnc(-c2ccnc(NCc3ncccn3)n2)n1)c1ccc2[nH]ncc2c1 ; Fc1ccc(CNc2nccc(-c3nccc(C#Cc4ccc5[nH]ncc5c4)n3)n2)cc1 ; C(#Cc1ccnc(-c2ccnc(NCC3CCCCN3)n2)n1)c1ccc2[nH]ncc2c1] 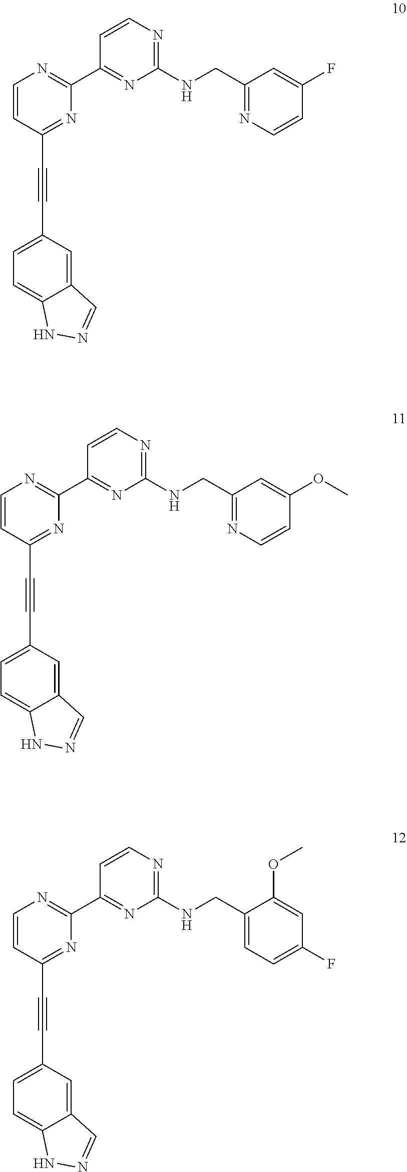 [SMILES: Fc1ccnc(CNc2nccc(-c3nccc(C#Cc4ccc5[nH]ncc5c4)n3)n2)c1 ; COc1ccnc(CNc2nccc(-c3nccc(C#Cc4ccc5[nH]ncc5c4)n3)n2)c1 ; COc1cc(F)ccc1CNc1nccc(-c2nccc(C#Cc3ccc4[nH]ncc4c3)n2)n1] 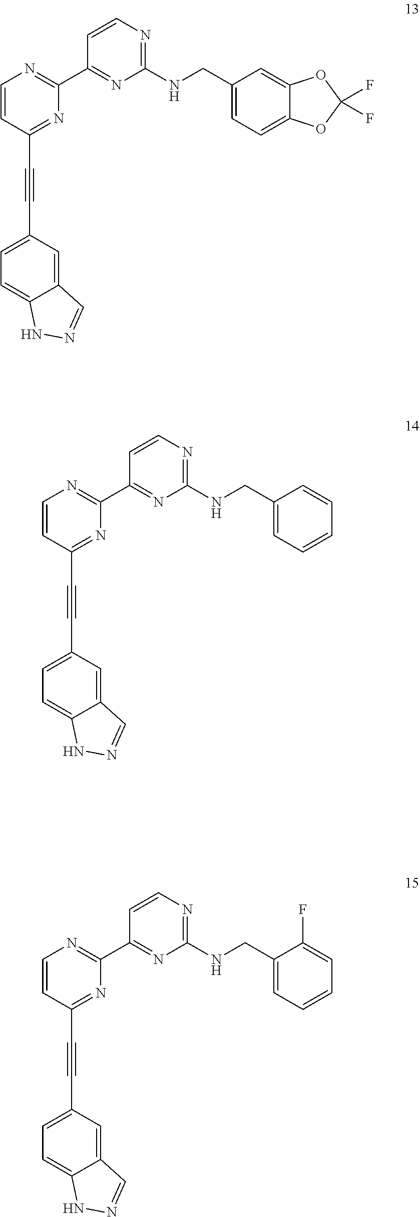 [SMILES: FC1(F)Oc2ccc(CNc3nccc(-c4nccc(C#Cc5ccc6[nH]ncc6c5)n4)n3)cc2O1 ; C(#Cc1ccnc(-c2ccnc(NCc3ccccc3)n2)n1)c1ccc2[nH]ncc2c1 ; Fc1ccccc1CNc1nccc(-c2nccc(C#Cc3ccc4[nH]ncc4c3)n2)n1] 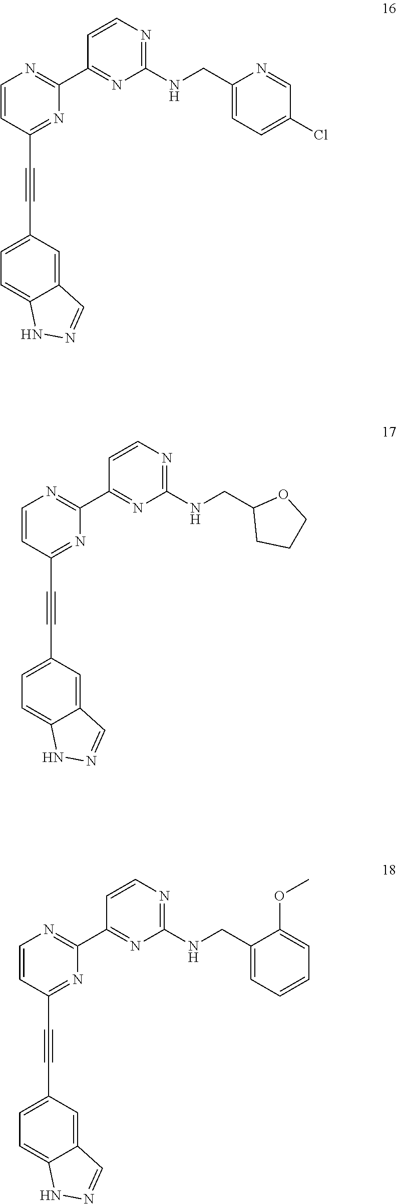 [SMILES: Clc1ccc(CNc2nccc(-c3nccc(C#Cc4ccc5[nH]ncc5c4)n3)n2)nc1 ; C(#Cc1ccnc(-c2ccnc(NCC3CCCO3)n2)n1)c1ccc2[nH]ncc2c1 ; COc1ccccc1CNc1nccc(-c2nccc(C#Cc3ccc4[nH]ncc4c3)n2)n1] 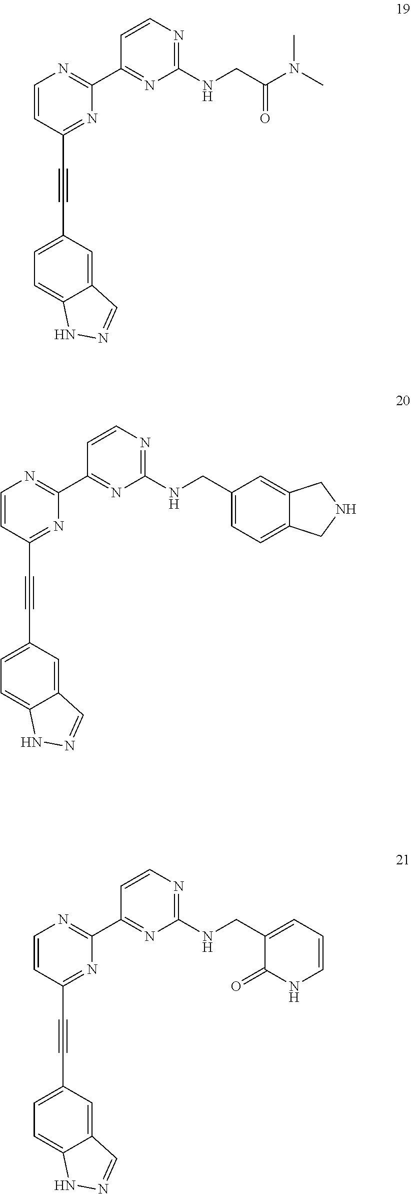 [SMILES: CN(C)C(=O)CNc1nccc(-c2nccc(C#Cc3ccc4[nH]ncc4c3)n2)n1 ; C(#Cc1ccnc(-c2ccnc(NCc3ccc4c(c3)CNC4)n2)n1)c1ccc2[nH]ncc2c1 ; O=c1[nH]cccc1CNc1nccc(-c2nccc(C#Cc3ccc4[nH]ncc4c3)n2)n1] 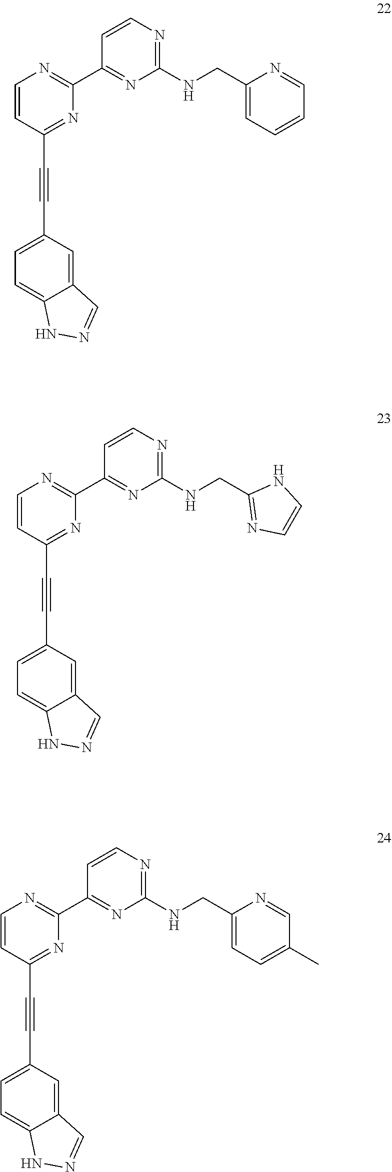 [SMILES: C(#Cc1ccnc(-c2ccnc(NCc3ccccn3)n2)n1)c1ccc2[nH]ncc2c1 ; C(#Cc1ccnc(-c2ccnc(NCc3ncc[nH]3)n2)n1)c1ccc2[nH]ncc2c1 ; Cc1ccc(CNc2nccc(-c3nccc(C#Cc4ccc5[nH]ncc5c4)n3)n2)nc1] 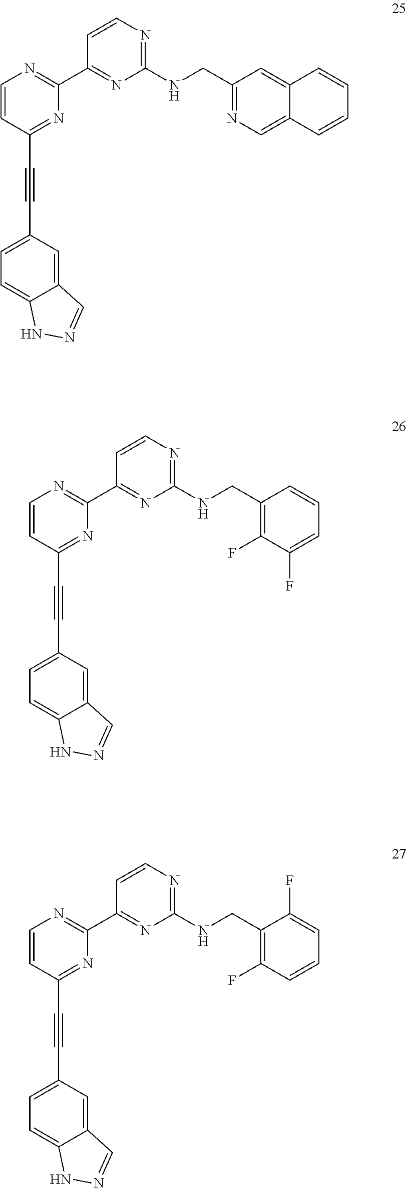 [SMILES: C(#Cc1ccnc(-c2ccnc(NCc3cc4ccccc4cn3)n2)n1)c1ccc2[nH]ncc2c1 ; Fc1cccc(CNc2nccc(-c3nccc(C#Cc4ccc5[nH]ncc5c4)n3)n2)c1F ; Fc1cccc(F)c1CNc1nccc(-c2nccc(C#Cc3ccc4[nH]ncc4c3)n2)n1] 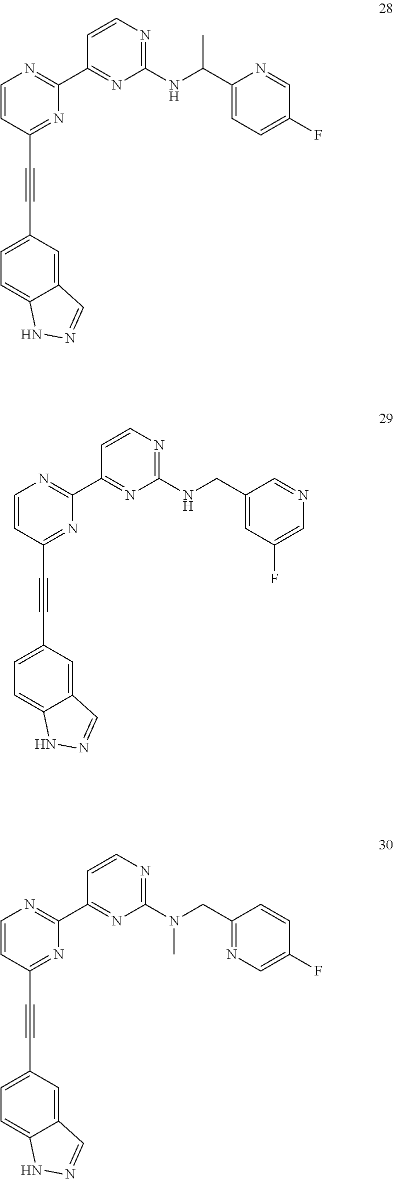 [SMILES: CC(Nc1nccc(-c2nccc(C#Cc3ccc4[nH]ncc4c3)n2)n1)c1ccc(F)cn1 ; Fc1cncc(CNc2nccc(-c3nccc(C#Cc4ccc5[nH]ncc5c4)n3)n2)c1 ; CN(Cc1ccc(F)cn1)c1nccc(-c2nccc(C#Cc3ccc4[nH]ncc4c3)n2)n1] 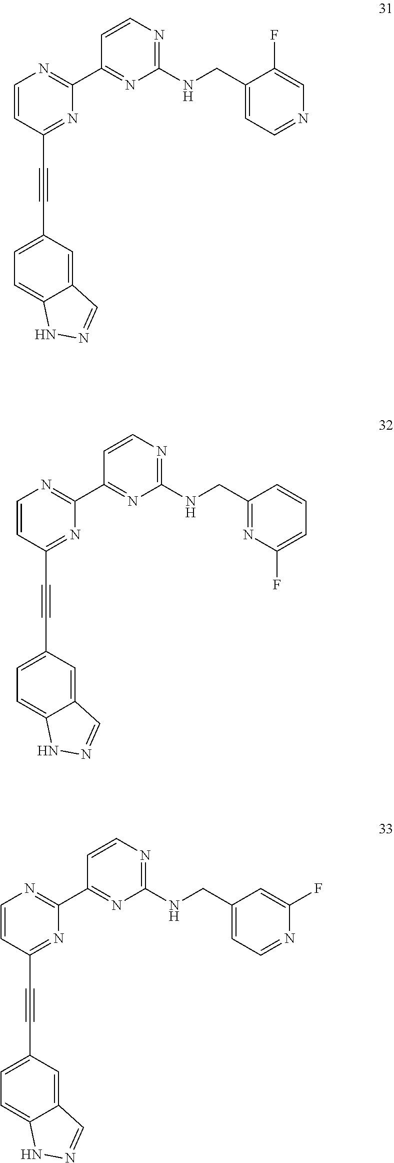 [SMILES: Fc1cnccc1CNc1nccc(-c2nccc(C#Cc3ccc4[nH]ncc4c3)n2)n1 ; Fc1cccc(CNc2nccc(-c3nccc(C#Cc4ccc5[nH]ncc5c4)n3)n2)n1 ; Fc1cc(CNc2nccc(-c3nccc(C#Cc4ccc5[nH]ncc5c4)n3)n2)ccn1] 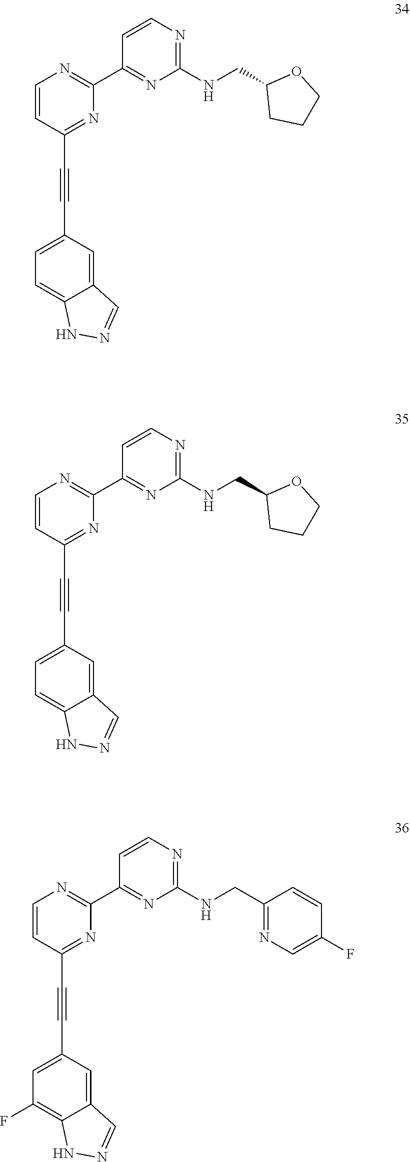 [SMILES: C(#Cc1ccnc(-c2ccnc(NC[C@H]3CCCO3)n2)n1)c1ccc2[nH]ncc2c1 ; C(#Cc1ccnc(-c2ccnc(NC[C@@H]3CCCO3)n2)n1)c1ccc2[nH]ncc2c1 ; Fc1ccc(CNc2nccc(-c3nccc(C#Cc4cc(F)c5[nH]ncc5c4)n3)n2)nc1] 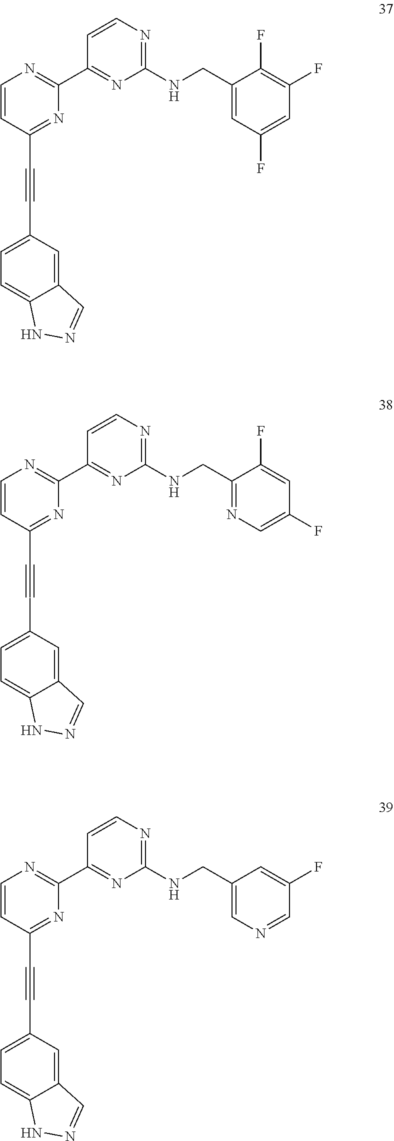 [SMILES: Fc1cc(F)c(F)c(CNc2nccc(-c3nccc(C#Cc4ccc5[nH]ncc5c4)n3)n2)c1 ; Fc1cnc(CNc2nccc(-c3nccc(C#Cc4ccc5[nH]ncc5c4)n3)n2)c(F)c1 ; Fc1cncc(CNc2nccc(-c3nccc(C#Cc4ccc5[nH]ncc5c4)n3)n2)c1] 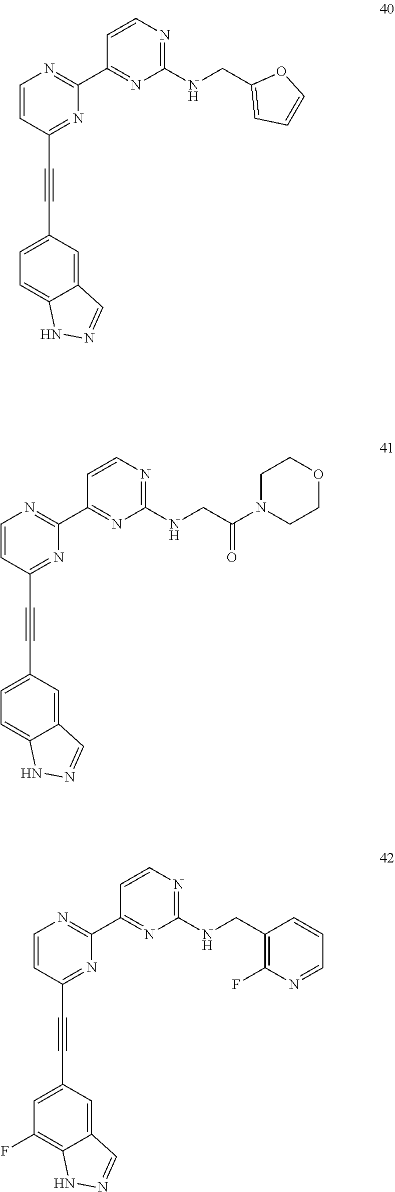 [SMILES: C(#Cc1ccnc(-c2ccnc(NCc3ccco3)n2)n1)c1ccc2[nH]ncc2c1 ; O=C(CNc1nccc(-c2nccc(C#Cc3ccc4[nH]ncc4c3)n2)n1)N1CCOCC1 ; Fc1ncccc1CNc1nccc(-c2nccc(C#Cc3cc(F)c4[nH]ncc4c3)n2)n1] 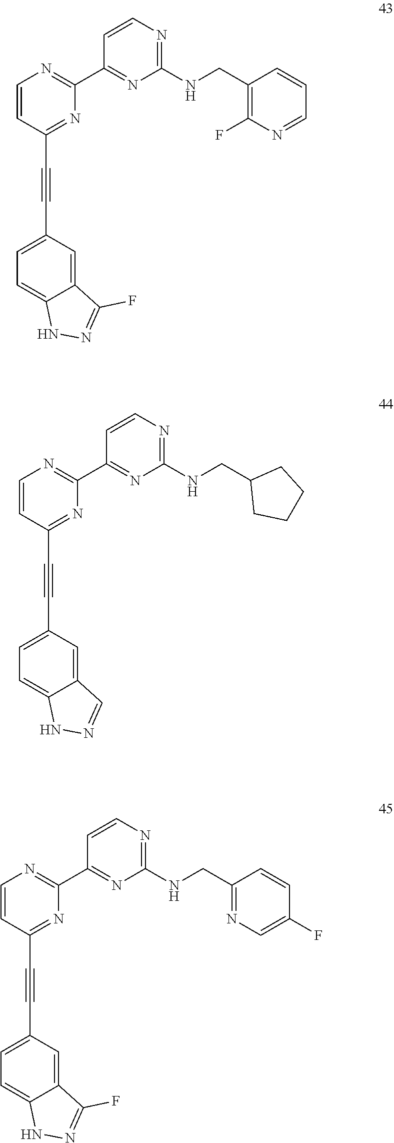 [SMILES: Fc1ncccc1CNc1nccc(-c2nccc(C#Cc3ccc4[nH]nc(F)c4c3)n2)n1 ; C(#Cc1ccnc(-c2ccnc(NCC3CCCC3)n2)n1)c1ccc2[nH]ncc2c1 ; Fc1ccc(CNc2nccc(-c3nccc(C#Cc4ccc5[nH]nc(F)c5c4)n3)n2)nc1] 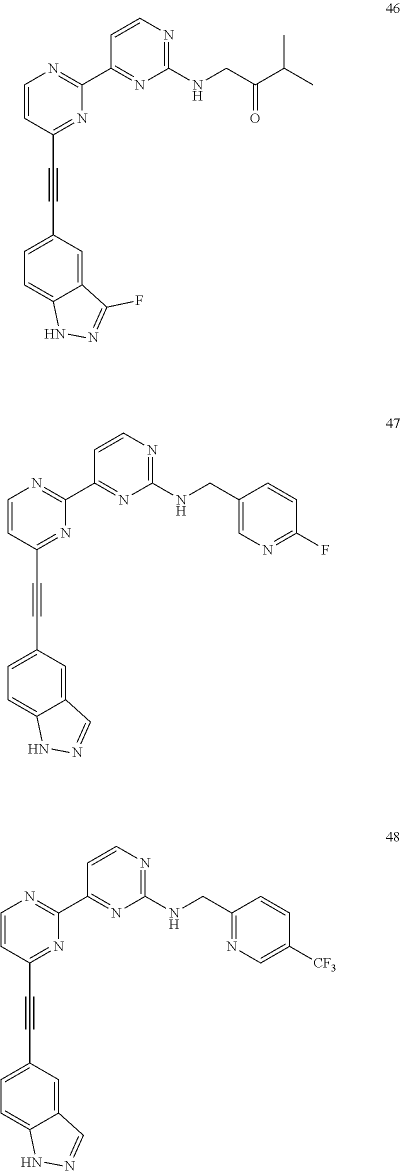 [SMILES: CC(C)C(=O)CNc1nccc(-c2nccc(C#Cc3ccc4[nH]nc(F)c4c3)n2)n1 ; Fc1ccc(CNc2nccc(-c3nccc(C#Cc4ccc5[nH]ncc5c4)n3)n2)cn1 ; FC(F)(F)c1ccc(CNc2nccc(-c3nccc(C#Cc4ccc5[nH]ncc5c4)n3)n2)nc1] 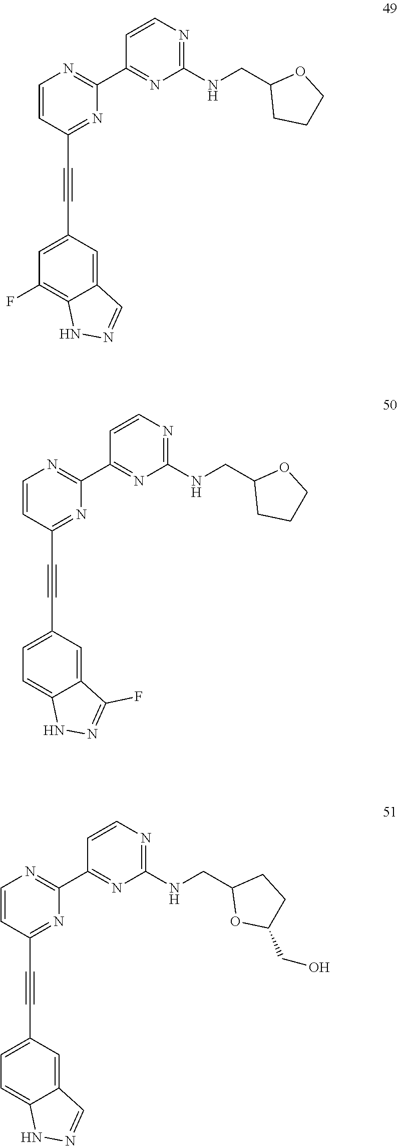 [SMILES: Fc1cc(C#Cc2ccnc(-c3ccnc(NCC4CCCO4)n3)n2)cc2cn[nH]c12 ; Fc1n[nH]c2ccc(C#Cc3ccnc(-c4ccnc(NCC5CCCO5)n4)n3)cc12 ; OC[C@H]1CCC(CNc2nccc(-c3nccc(C#Cc4ccc5[nH]ncc5c4)n3)n2)O1] 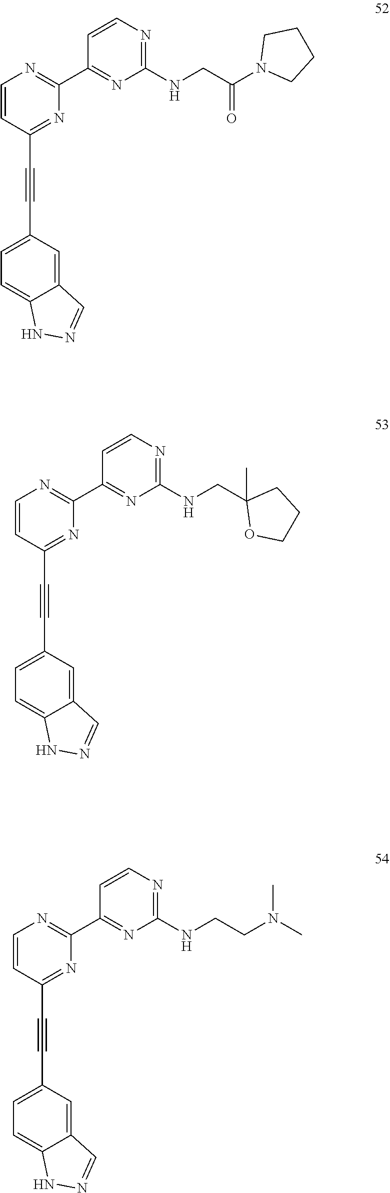 [SMILES: O=C(CNc1nccc(-c2nccc(C#Cc3ccc4[nH]ncc4c3)n2)n1)N1CCCC1 ; CC1(CNc2nccc(-c3nccc(C#Cc4ccc5[nH]ncc5c4)n3)n2)CCCO1 ; CN(C)CCNc1nccc(-c2nccc(C#Cc3ccc4[nH]ncc4c3)n2)n1] 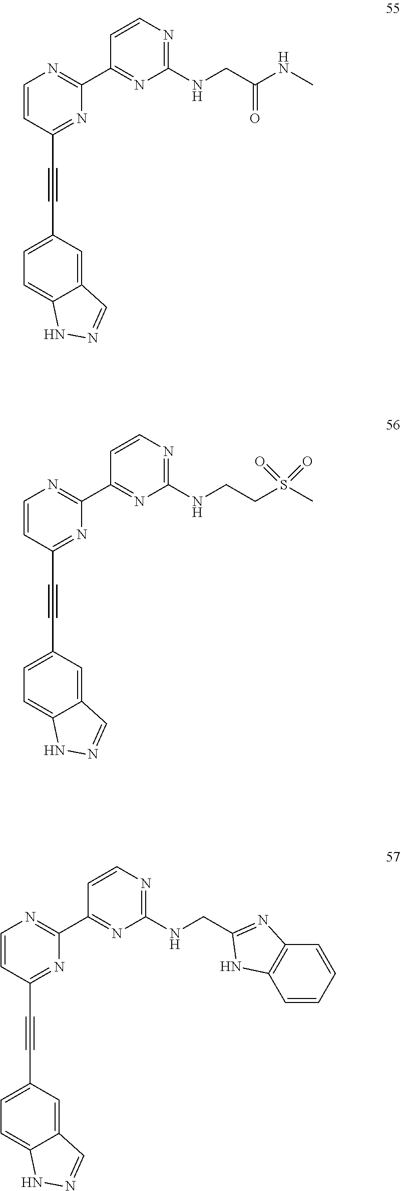 [SMILES: CNC(=O)CNc1nccc(-c2nccc(C#Cc3ccc4[nH]ncc4c3)n2)n1 ; CS(=O)(=O)CCNc1nccc(-c2nccc(C#Cc3ccc4[nH]ncc4c3)n2)n1 ; C(#Cc1ccnc(-c2ccnc(NCc3nc4ccccc4[nH]3)n2)n1)c1ccc2[nH]ncc2c1] 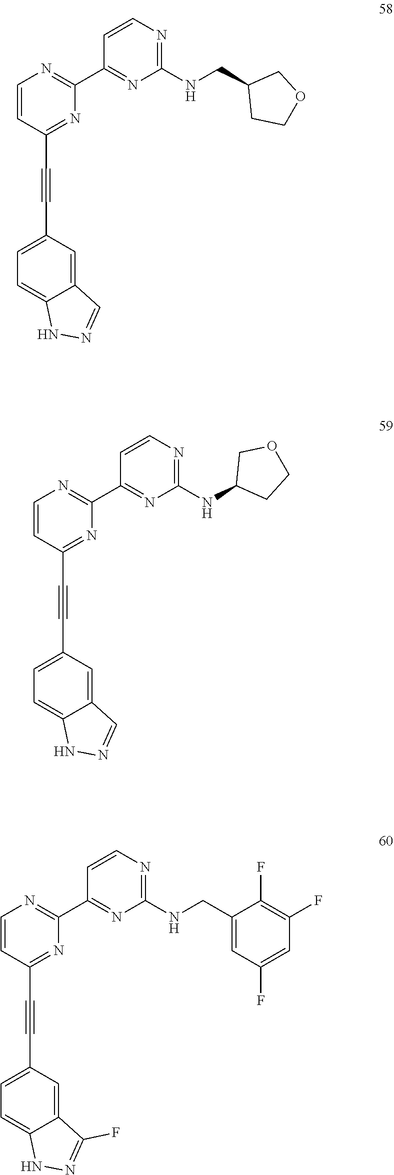 [SMILES: C(#Cc1ccnc(-c2ccnc(NC[C@@H]3CCOC3)n2)n1)c1ccc2[nH]ncc2c1 ; C(#Cc1ccnc(-c2ccnc(N[C@@H]3CCOC3)n2)n1)c1ccc2[nH]ncc2c1 ; Fc1cc(F)c(F)c(CNc2nccc(-c3nccc(C#Cc4ccc5[nH]nc(F)c5c4)n3)n2)c1] 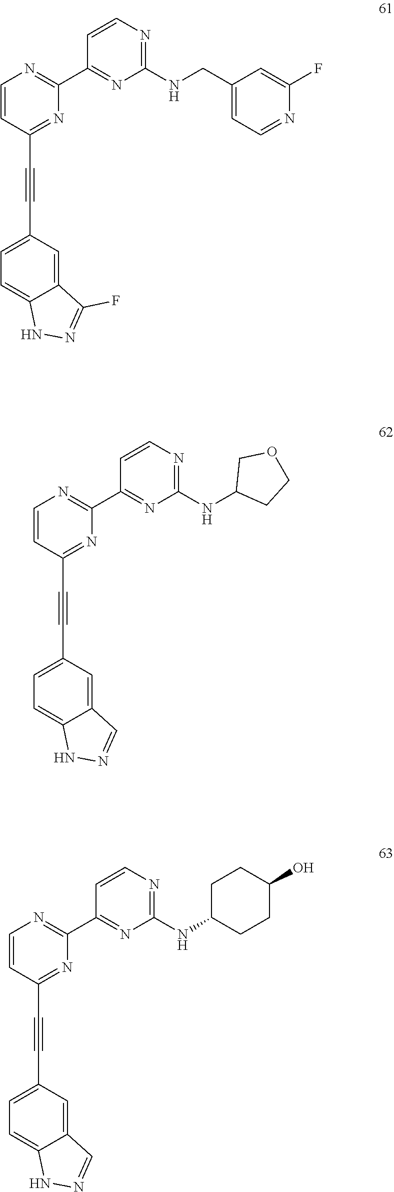 [SMILES: Fc1cc(CNc2nccc(-c3nccc(C#Cc4ccc5[nH]nc(F)c5c4)n3)n2)ccn1 ; C(#Cc1ccnc(-c2ccnc(NC3CCOC3)n2)n1)c1ccc2[nH]ncc2c1 ; O[C@H]1CC[C@H](Nc2nccc(-c3nccc(C#Cc4ccc5[nH]ncc5c4)n3)n2)CC1] 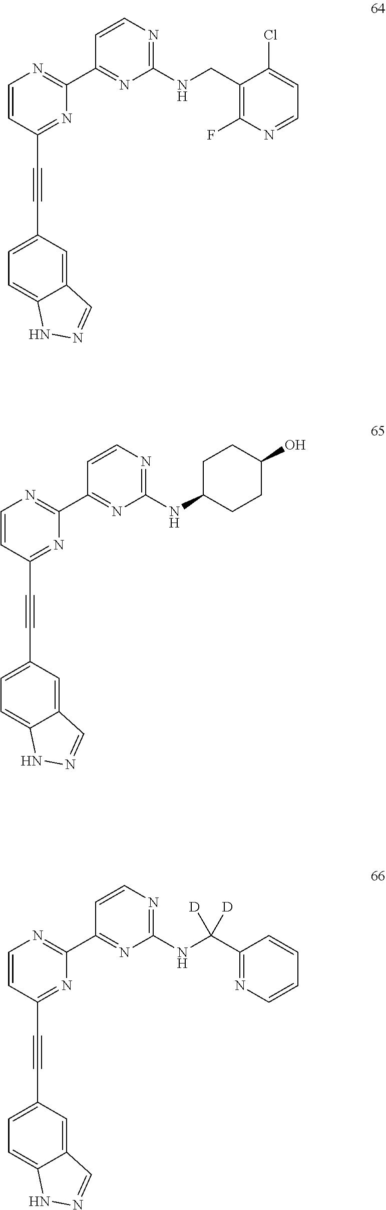 [SMILES: Fc1nccc(Cl)c1CNc1nccc(-c2nccc(C#Cc3ccc4[nH]ncc4c3)n2)n1 ; O[C@H]1CC[C@@H](Nc2nccc(-c3nccc(C#Cc4ccc5[nH]ncc5c4)n3)n2)CC1 ; [2H]C([2H])(Nc1nccc(-c2nccc(C#Cc3ccc4[nH]ncc4c3)n2)n1)c1ccccn1] 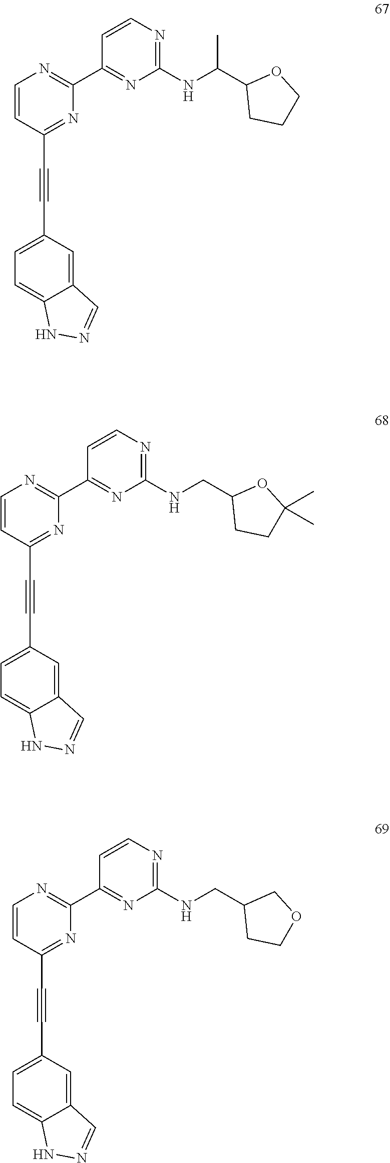 [SMILES: CC(Nc1nccc(-c2nccc(C#Cc3ccc4[nH]ncc4c3)n2)n1)C1CCCO1 ; CC1(C)CCC(CNc2nccc(-c3nccc(C#Cc4ccc5[nH]ncc5c4)n3)n2)O1 ; C(#Cc1ccnc(-c2ccnc(NCC3CCOC3)n2)n1)c1ccc2[nH]ncc2c1] 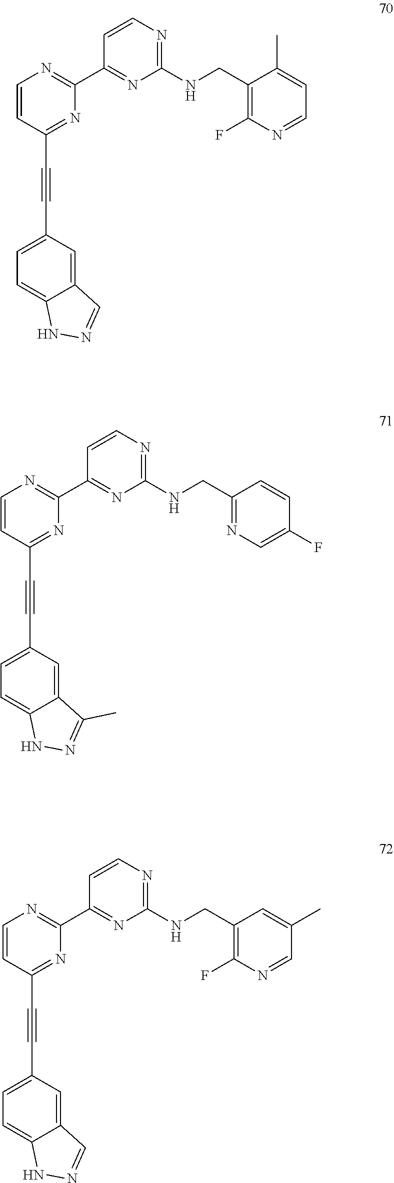 [SMILES: Cc1ccnc(F)c1CNc1nccc(-c2nccc(C#Cc3ccc4[nH]ncc4c3)n2)n1 ; Cc1n[nH]c2ccc(C#Cc3ccnc(-c4ccnc(NCc5ccc(F)cn5)n4)n3)cc12 ; Cc1cnc(F)c(CNc2nccc(-c3nccc(C#Cc4ccc5[nH]ncc5c4)n3)n2)c1] 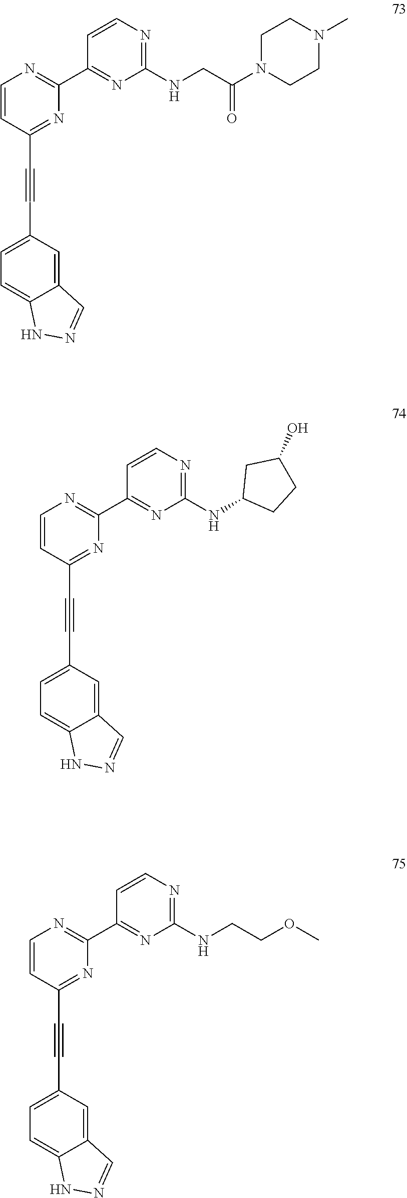 [SMILES: CN1CCN(C(=O)CNc2nccc(-c3nccc(C#Cc4ccc5[nH]ncc5c4)n3)n2)CC1 ; O[C@@H]1CC[C@H](Nc2nccc(-c3nccc(C#Cc4ccc5[nH]ncc5c4)n3)n2)C1 ; COCCNc1nccc(-c2nccc(C#Cc3ccc4[nH]ncc4c3)n2)n1] 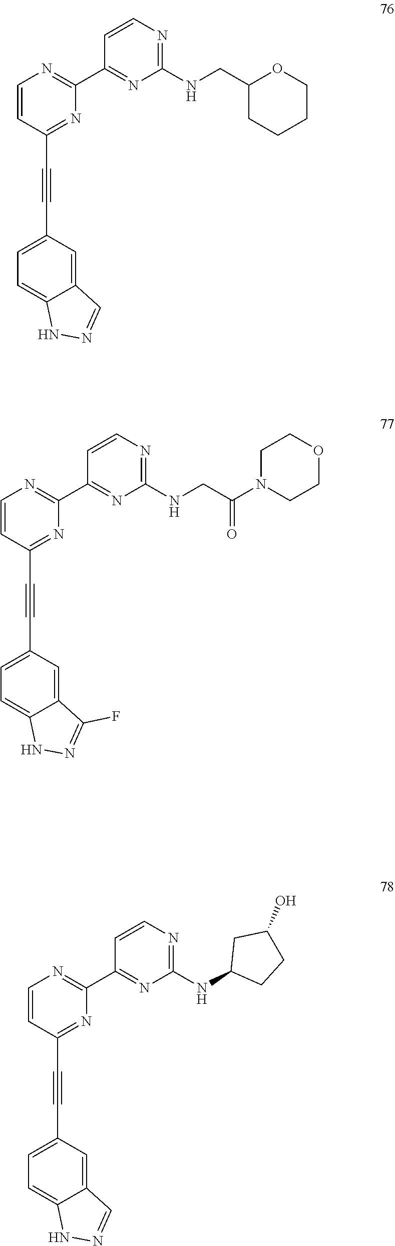 [SMILES: C(#Cc1ccnc(-c2ccnc(NCC3CCCCO3)n2)n1)c1ccc2[nH]ncc2c1 ; O=C(CNc1nccc(-c2nccc(C#Cc3ccc4[nH]nc(F)c4c3)n2)n1)N1CCOCC1 ; O[C@@H]1CC[C@@H](Nc2nccc(-c3nccc(C#Cc4ccc5[nH]ncc5c4)n3)n2)C1] 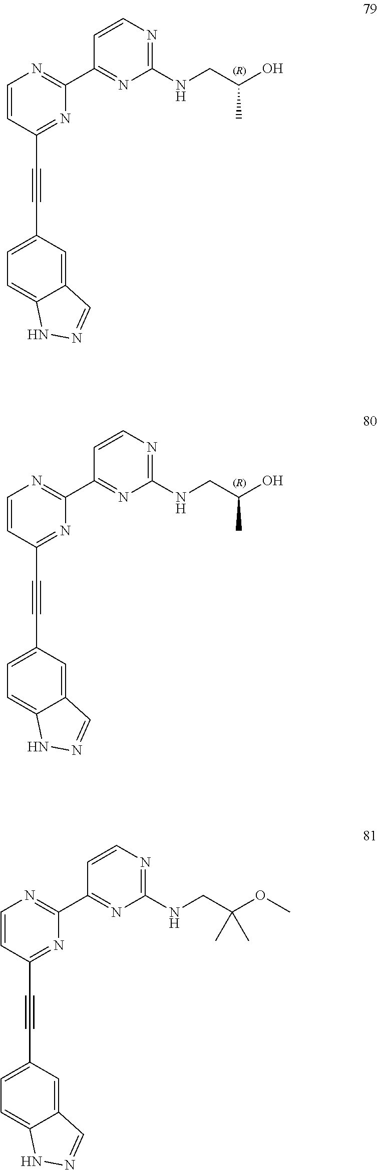 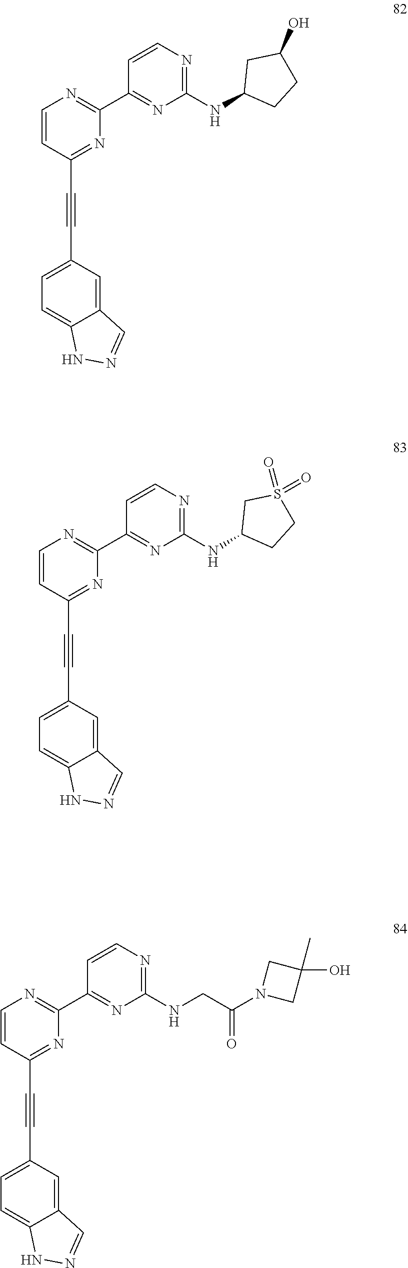 [SMILES: O[C@H]1CC[C@@H](Nc2nccc(-c3nccc(C#Cc4ccc5[nH]ncc5c4)n3)n2)C1 ; O=S1(=O)CC[C@H](Nc2nccc(-c3nccc(C#Cc4ccc5[nH]ncc5c4)n3)n2)C1 ; CC1(O)CN(C(=O)CNc2nccc(-c3nccc(C#Cc4ccc5[nH]ncc5c4)n3)n2)C1] 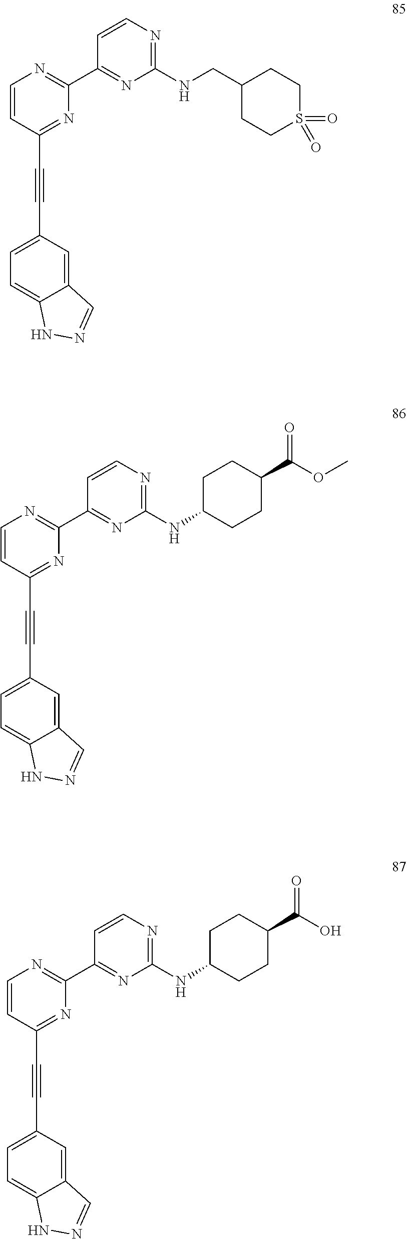 [SMILES: O=S1(=O)CCC(CNc2nccc(-c3nccc(C#Cc4ccc5[nH]ncc5c4)n3)n2)CC1 ; COC(=O)[C@H]1CC[C@H](Nc2nccc(-c3nccc(C#Cc4ccc5[nH]ncc5c4)n3)n2)CC1 ; O=C(O)[C@H]1CC[C@H](Nc2nccc(-c3nccc(C#Cc4ccc5[nH]ncc5c4)n3)n2)CC1] 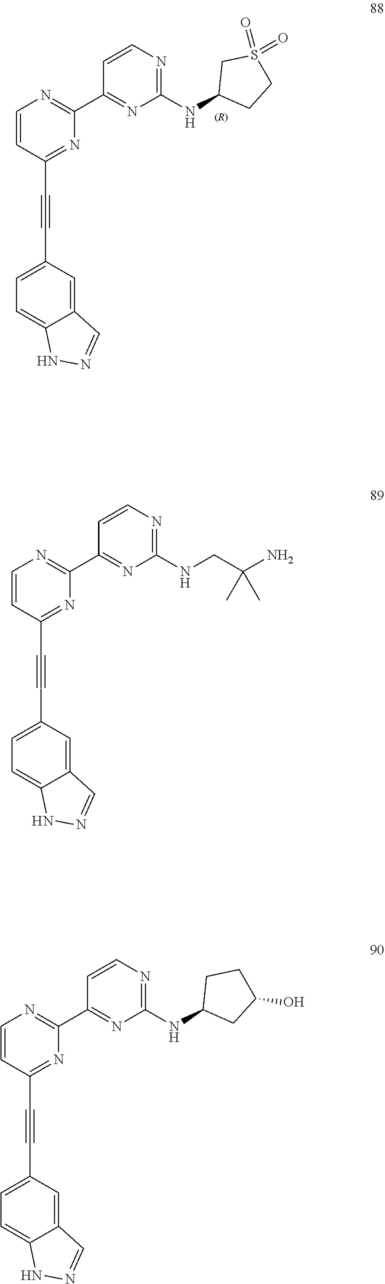 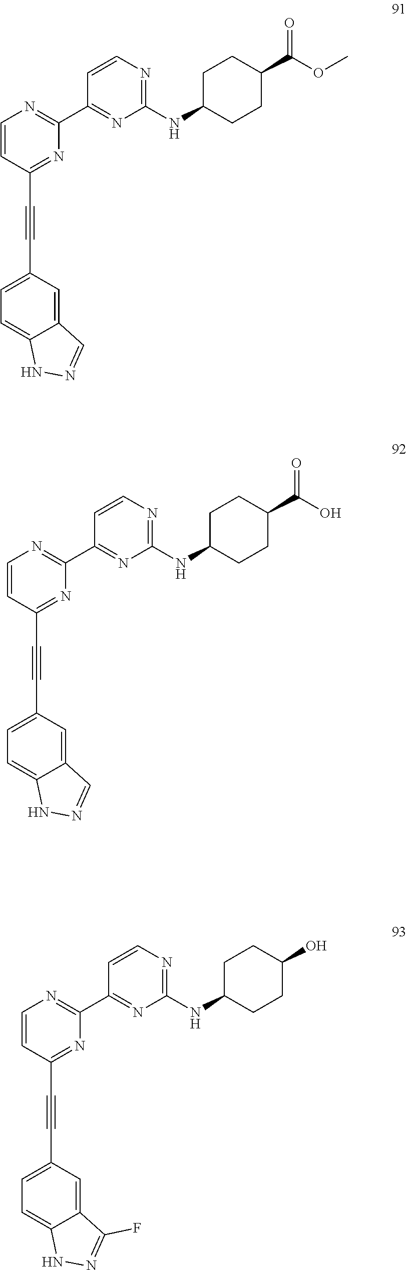 [SMILES: COC(=O)[C@H]1CC[C@@H](Nc2nccc(-c3nccc(C#Cc4ccc5[nH]ncc5c4)n3)n2)CC1 ; O=C(O)[C@H]1CC[C@@H](Nc2nccc(-c3nccc(C#Cc4ccc5[nH]ncc5c4)n3)n2)CC1 ; O[C@H]1CC[C@@H](Nc2nccc(-c3nccc(C#Cc4ccc5[nH]nc(F)c5c4)n3)n2)CC1] 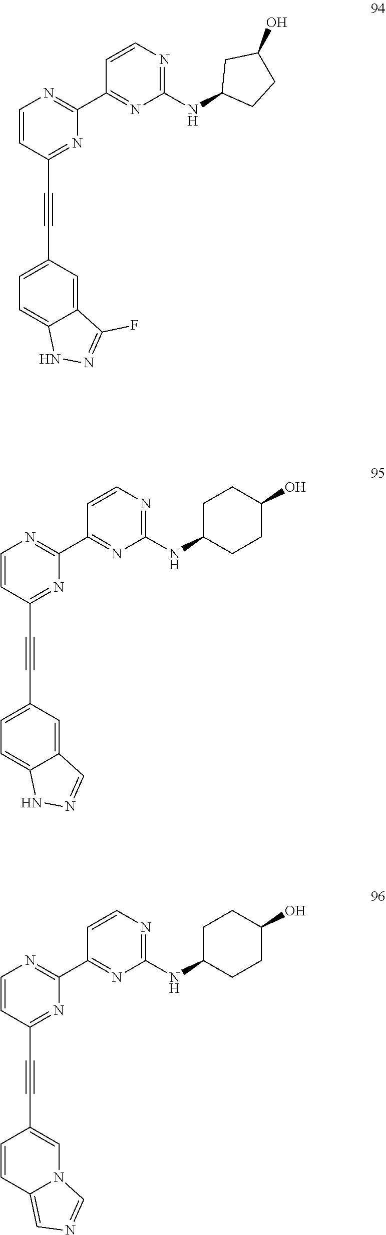 [SMILES: O[C@H]1CC[C@@H](Nc2nccc(-c3nccc(C#Cc4ccc5[nH]nc(F)c5c4)n3)n2)C1 ; O[C@H]1CC[C@@H](Nc2nccc(-c3nccc(C#Cc4ccc5[nH]ncc5c4)n3)n2)CC1 ; O[C@H]1CC[C@@H](Nc2nccc(-c3nccc(C#Cc4ccc5cncn5c4)n3)n2)CC1] 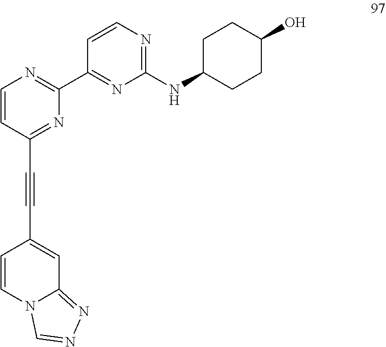 [SMILES: O[C@H]1CC[C@@H](Nc2nccc(-c3nccc(C#Cc4ccn5cnnc5c4)n3)n2)CC1]
or a pharmaceutically acceptable salt thereof.

36. A pharmaceutical composition comprising a compound according to any one of claims 1-35, or a pharmaceutically acceptable salt thereof, and a pharmaceutically acceptable carrier.

37. A method of inhibiting ROCK1 and/or ROCK2, the method comprising contacting a biological sample with a compound according to any one of claims 1-35, or a pharmaceutically acceptable salt thereof.

38. The method according to claim 37, wherein the compound is selective for ROCK2.

39. A method of treating or lessening the severity of a disease or disorder associated with or mediated by Rho-associated coiled-coil kinase (ROCK), the method comprising administering to a patient in need thereof a compound according to any one of claims 1-35, or a pharmaceutically acceptable salt thereof.

40. The method according to claim 39, wherein the compound is selective for ROCK2.

41. The method according to claim 39 or 40, wherein the disease or disorder is selected from a hepatic disease, a renal disease, a cerebral and/or cerebrovascular disease, a cardiac and/or cardiovascular disease, a pulmonary disease, a dermal disease, a gastrointestinal disease, an ischemic disease, and a fibrotic disease.
